(19) United States
(12) Patent Application Publication
(10) Pub. No.: US 2022/0048988 A1
(43) Pub. Date: Feb. 17, 2022

(54) CELLS EXPRESSING ANTIBODIES TARGETING HUMAN IMMUNODEFICIENCY VIRUS AND METHODS OF USING THE SAME

(71) Applicants: Children's National Medical Center, Washington, DC (US); The George Washington University, Washington, DC (US)

(72) Inventors: Catherine Mary Bollard, Bethesda, MD (US); Conrad Russell Y. Cruz, Bethesda, MD (US); Allison Powell, Arlington, VA (US); Brad Jones, Washington, DC (US); Douglas Nixon, Washingtona, DC (US)

(21) Appl. No.: 17/276,081

(22) PCT Filed: Sep. 13, 2019
(86) PCT No.: PCT/US2019/051187

§ 371 Date: Mar. 12, 2021

Related U.S. Application Data
(60) Provisional application No. 62/730,922, filed on Sep. 13, 2018.

(51) Int. Cl. C07K 16/28; C07K 16/10; C12N 15/86

(52) U.S. Cl. CPC C07K 16/283; C07K 16/1045; C12N 15/86; C12N 2740/13043; C07K 2317/76; C07K 2317/31; C07K 2317/622; C07K 2317/732; C07K 2317/21

(57) ABSTRACT

The present disclosure relates to genetically modified T-cells to secrete broadly neutralizing antibodies against HIV, and methods of preparing and uses thereof.

### BACKGROUND

Despite significant advances in the last few decades, HIV remains a problem around the world. There are an estimated 40 million people worldwide living with HIV. Although drugs targeting HIV viruses are in wide use and have shown effectiveness, toxicity and development of resistant strains have limited their usefulness. Current treatment for the disease—antiretroviral therapy, or ART—has dramatically increased overall survival rates of this population but it is not a cure and patients remain burdened by decreased quality of life and decreased life expectancy.

In the sera of human immunodeficiency virus type 1 (HIV-1) infected patients, antivirus antibodies can be detected over a certain period after infection without any clinical manifestations of the acquired immunodeficiency syndrome (AIDS). At this state of active immune response, high numbers of antigen-specific B-cells are expected in the circulation. These B-cells are used as fusion partners for the generation of human monoclonal anti-HIV antibodies. One major drawback to finding a vaccine composition suitable for more reliable prevention of human individuals from HIV-1 infection and/or for more successful therapeutic treatment of infected patients is the ability of the HIV-1 virus to escape antibody capture by genetic variation, which very often renders the remarkable efforts of the researchers almost useless. Such escape mutants may be characterized by a change of only one or several of the amino acids within one of the targeted antigenic determinants and may occur, for example, as a result of spontaneous or induced mutation. In addition to genetic variation, certain other properties of the HIV-1 envelope glycoprotein makes it difficult to elicit neutralizing antibodies making generation of undesirable non-neutralizing antibodies a major concern (see, Phogat S K and Wyatt R T, Curr Pharm Design 2007; 13(2):213-227).

HIV-1 is among the most genetically diverse viral pathogens. Of the three main branches of the HIV-1 phylogenetic tree, the M (main), N (new), and O (outlier) groups, group M viruses are the most widespread, accounting for over 99% of global infections. This group is presently divided into nine distinct genetic subtypes, or clades (A through K), based on full-length sequences. Env is the most variable HIV-1 gene, with up to 35% sequence diversity between clades, 20% sequence diversity within clades, and up to 10% sequence diversity in a single infected person (Shankarappa, R. et al. 1999. J. Virol. 73: 10489-10502). Clade B is dominant in Europe, the Americas, and Australia. Clade C is common in southern Africa, China, and India and presently infects more people worldwide than any other clade (McCutchan, F E. 2000. Understanding the genetic diversity of HIV-1. AIDS 14(Suppl. 3):531-544). Clades A and D are prominent in central and eastern Africa. [0153] Neutralizing antibodies (NAbs) against viral envelope proteins (Env) provide adaptive immune defense against human immunodeficiency virus type 1 (HIV-1) exposure by blocking the infection of susceptible cells (Kwong P D et al., 2002. Nature 420: 678-682). The efficacy of vaccines against several viruses has been attributed to their ability to elicit NAbs. However, despite enormous efforts, there has been limited progress toward an effective immunogen for HIV-1. (Burton, D. R. 2002. Nat. Rev. Immunol. 2:706-713).

HIV-1 has evolved with an extensive array of strategies to evade antibody-mediated neutralization. (Barouch, D. H. Nature 455, 613-619 (2008); Kwong, P. D. & Wilson, L A. Nat Immunol 10, 573-578 (2009); Karlsson Hedestam, G. B., et al. Nat Rev Microbiol 6, 143-155 (2008)). However, broadly neutralizing antibodies (bNAbs) develop over time in a proportion of HIV-1 infected individuals. (Leonidas Stamatatos, L. M., Dennis R Burton, and John Mascola. Nature Medicine (E-Pub: Jun. 14, 2009); PMID: 19525964.) A handful of broadly neutralizing monoclonal antibodies have been isolated from clade B infected donors. (Burton, D. R., et al. Science 266, 1024-1027 (1994); Trkola, A., et al. J Virol 69, 6609-6617 (1995); Stiegler, G., et al. AIDS Res Hum Retroviruses 17, 1757-1765 (2001)). These antibodies tend to display less breadth and potency against non-clade B viruses, and they recognize epitopes on the virus that have so far failed to elicit broadly neutralizing responses when incorporated into a diverse range of immunogens. (Phogat, S. & Wyatt, R. Curr Pharm Design 13, 213-227 (2007); Montero, M., van Houten, N. E., Wang, X. & Scott, J. K. Microbiol Mol Biol Rev 72, 54-84, table of contents (2008); Scanlan, C. N., Offer, J., Zitzmann, N. & Dwek, R. A. Nature 446, 1038-1045 (2007)). Despite the enormous diversity of the human immunodeficiency virus (HIV), all HIV viruses known to date interact with the same cellular receptors (CD4 and/or a co-receptor, CCR5 or CXCR4). Most neutralizing antibodies bind to functional regions involved in receptor interactions and cell membrane fusion. However, the vast majority of neutralizing antibodies isolated to date do not recognize more than one clade, therefore exhibiting limited protective efficacy in vitro or in vivo. (See Binley J M et al., 2004. J. Virol. 78(23): 13232-13252).

Several strategies have been tested to genetically modify lymphocytes for use in HIV therapy, however they all suffer from similar challenges including the heterogeneous and rapidly mutating nature of HIV leading to viral escape, poor persistence in vivo, the potential of acquiring resistance, and have, thus far, only shown transient efficacy in clinical trials. The role of T cells in mediating a cure has been previously shown in studies of elite controllers. These patients maintain undetectable levels of HIV, which has been associated with a significantly increased breadth of Gag-specific CD8+ T cell response, when compared to chronic progressors and individuals with ART suppressed HIV. In addition, contributions by the innate immune compartment, specifically natural killer cells, has been shown in the RV144 vaccine trails. Broadly neutralizing antibodies, derived from a subset of patients enrolled in the trial, were able to elicit antibody dependent cellular cytotoxicity against HIV infected targets. Caskey et al. (2017) have shown that broadly neutralizing antibodies are able to transiently decrease HIV RNA levels in a subset of the population leading us to believe that the combination of each of these anti-viral mechanisms is needed to have a lasting efficacy against HIV.

### SUMMARY OF THE DISCLOSURE

The disclosure relates to a composition comprising one or a plurality of T cells comprising a nucleic acid sequence encoding an antibody or antibody fragment, wherein the antibody or antibody fragment comprises one or a plurality of sequences VL and/or VH sequences and/or CDR amino acid sequences disclosed herein. The disclosure also relates to the method of treating HIV or preventing HIV infection by administering a therapeutically effective amount of a pharmaceutical composition comprising one or a plurality of T cells comprising a nucleic acid sequence encoding an antibody or antibody fragment, wherein the antibody or antibody fragment comprises one or a plurality of sequences VL and/or VH sequences and/or CDR amino acid sequences disclosed herein; or wherein the antibody is a Nab specific for HIV or certain strains thereof. In some embodiments, the nucleic acid seqeunce encoding an antibody or antibody fragment is part of a nucleic acid molecule comprising a regulatory seqeunce operably linked to the nucleic acid sequence encoding the antibody or antibody fragment. The disclosure also relates to the method of treating HIV or preventing HIV infection by administering a therapeutically effective amount of a pharmaceutical composition comprising media from a culture of one or a plurality of T cells comprising a nucleic acid sequence encoding an antibody or antibody fragment, wherein the antibody or antibody fragment comprises one or a plurality of sequences VL and/or VH sequences and/or CDR amino acid sequences disclosed herein; or wherein the antibody is a Nab specific for HIV or certain strains thereof.

In a first aspect, the disclosure provides an antibody, or an antigen-binding fragment thereof, comprising a) a first light chain comprising a first light chain variable region (VL) and a first heavy chain comprising a first heavy chain variable region (VH), wherein the first light chain and the first heavy chain are derived from a first antibody or an antigen-binding fragment thereof; and b) a second light chain comprising a second light chain variable region (VL) and a second heavy chain comprising a second heavy chain variable region (VH), wherein the second light chain and the second heavy chain are derived from a second antibody or an antigen-binding fragment thereof, wherein the first light chain binds epitopes of the envelope protein of human immunodeficiency virus-1 (HIV-1). In one embodiment, either the VH and/or the VL region at least partially binds to V3 glycan supersite of the HIV envelope protein. In another embodiment, the VH and the VL are positioned non-contiguously and connected by at least one hinge sequence. In another embodiment, the antibody, or antigen binding fragment, further comprises one or a plurality of amino acid sequences encoded by a nucleic acid sequence at least about 70% sequence identity to SEQ ID NO: 21 and/or SEQ ID NO: 22. In another embodiment, the antibody, or antigen binding fragment, further comprises at least one furin linker. In another embodiment, the antibody, or antigen binding fragment, further comprises a least one or more self-cleaving amino acid sequences chosen from: FMDV 2A (abbreviated herein as F2A), equine rhinitis A virus (ERAV) 2A (E2A), porcine teschovirus-1 2A (P2A) and Thoseaasigna virus 2A (T2A), or at least one internal ribosome entry sequence (IRES) separating construct domains. In another embodiment, internal ribosome entry sequences (IRES) take the place of self-cleaving amino acid sequences, allowing independent translation of the different fragments. In one embodiment, the VL comprises an amino acid sequence encoded by a nucleic acid having at least about 70% sequence identity to SEQ ID NO: 14. In another embodiment, the VH comprises an amino acid sequence encoded by a nucleic acid having at least about 70% sequence identity to SEQ ID NO: 16. In one embodiment, the antibody, or antigen binding fragment, further comprises at least one linker that is a single glycine (Gly) residue; a diglycine peptide (Gly-Gly); a tripeptide (Gly-Gly-Gly); a peptide with four glycine residues (Gly-Gly-Gly-Gly; SEQ ID NO: 37); a peptide with five glycine residues (Gly-Gly-Gly-Gly-Gly; SEQ ID NO: 38); a peptide with six glycine residues (Gly-Gly-Gly-Gly-Gly-Gly; SEQ ID NO: 39); a peptide with seven glycine residues (Gly-Gly-Gly-Gly-Gly-Gly-Gly; SEQ ID NO: 40); a peptide with eight glycine residues (Gly-Gly-Gly-Gly-Gly-Gly-Gly-Gly; SEQ ID NO: 41), the peptide Gly-Gly-Gly-Gly-Ser (SEQ ID NO: 42), the peptide Gly-Gly-Gly-Gly-Ser-Gly-Gly-Gly-Gly-Ser (SEQ ID NO: 43), the peptide Gly-Gly-Gly-Gly-Ser-Gly-Gly-Gly-Gly-Ser-Gly-Gly-Gly-Gly-Ser (SEQ ID NO: 44), a single Ser, a single Val, the dipeptide Arg-Thr, Gln-Pro, Ser-Ser, Thr-Lys, and Ser-Leu; Thr-Lys-Gly-Pro-Ser (SEQ ID NO: 45), Thr-Val-Ala-Ala-Pro (SEQ ID NO: 46), Gln-Pro-Lys-Ala-Ala (SEQ ID NO: 47), Gln-Arg-Ile-Glu-Gly (SEQ ID NO: 48), Ala-Ser-Thr-Lys-Gly-Pro-Ser (SEQ ID NO: 49), Arg-Thr-Val-Ala-Ala-Pro-Ser (SEQ ID NO: 50), Gly-Gln-Pro-Lys-Ala-Ala-Pro (SEQ ID NO: 51), and His-Ile-Asp-Ser-Pro-Asn-Lys (SEQ ID NO: 52). In one embodiment, the VL binds one of the following epitopes: the CD4-binding site, the V1/V2-glycan region, the V3-glycan region, the gp41 membrane proximal external region (MPER), or the gp120/gp41 interface of the envelope protein. In another embodiment, the VL comprises one or more of complementarity-determining regions (CDRs) that are at least about 70% identical to the amino acid sequences of SEQ ID NO: 25, SEQ ID NO: 26, a SEQ ID NO: 27, SEQ ID NO: 56, SEQ ID NO: 58, and SEQ ID NO: 60. In another embodiment, the VH comprises one of more of complementarity-determining regions (CDRs) that are at least about 70% identical to the amino acid sequences of SEQ ID NO: 28, SEQ ID NO: 29, SEQ ID NO: 30, SEQ ID NO: 67, SEQ ID NO:69, and SEQ ID NO: 71. In another embodiment, the antibody or antibody fragment is encoded by a nucleic acid sequence having at least about 70% sequence identity to SEQ ID NO: 11 and/or SEQ ID NO: 12. In one embodiment, the antigen binding fragment is a scFv of 10-1074. In one embodiment, the antibody or antigen binding fragment is free of a CD19 signal sequence.

In another aspect, the disclosure features a cell comprising a nucleic acid sequence encoding any of the one or plurality of antibodies or antigen binding fragments of any of the aspects and embodiments herein. In one embodiment, the cell is a T cell. In another embodiment, the cell further comprises a costimulatory molecule capable of binding an HIV antigen. In another embodiment, the cell is isolated form a subject diagnosed with or suspected of being infected with HIV.

In another aspect, the disclosure features a composition comprising an expressible nucleic acid sequence encoding an antibody or an antigen-binding fragment thereof, wherein the antibody or the antigen-binding fragment thereof comprises:
(i) a light chain comprising a first secretory signal followed by a light chain variable region (VL) of an anti-human immunodeficiency virus-1 (HIV-1) broadly neutralizing antibody;
(ii) a heavy chain comprising a second secretory signal followed by a heavy chain variable region (VH) of said anti-HIV-1 broadly neutralizing antibody, wherein the VL and the VH are positioned non-contiguously and connected by at least one self-cleaving amino acid sequence, and wherein the VL binds epitopes of the envelope protein of human immunodeficiency virus-1 (HIV-1).

In some embodiments, the light chain further comprises a light chain constant region of an immunoglobulin G (IgG). In some embodiments, the heavy chain further comprises a heavy chain constant region of an IgG. In other embodiments, the light chain constant region of an IgG comprises an amino acid sequence having at least about 70% sequence identity to the amino acid sequence of SEQ ID NO: 78. In some other embodiments, the the heavy chain constant region of an IgG comprises an amino acid sequence having at least about 70% sequence identity to the amino acid sequence of SEQ ID NO: 79.

In some embodiments, the VL comprises at least one complementarity-determining region (CDR) selected from the group consisting of SEQ ID NO: 25, SEQ ID NO: 26, SEQ ID NO: 27, SEQ ID NO: 56, SEQ ID NO: 58, and SEQ ID NO: 60. In some embodiments, the VL comprises:
a) a first CDR comprising the amino acid sequence of SEQ ID NO: 25 or 56, and at least a second CDR comprising the amino acid sequence of SEQ ID NO: 26, SEQ ID NO: 27, SEQ ID NO: 58, or SEQ ID NO: 60;
b) a first CDR comprising the amino acid sequence of SEQ ID NO: 26 or 58, and at least a second CDR comprising the amino acid sequence of SEQ ID NO: 25, SEQ ID NO: 27, SEQ ID NO: 56, or SEQ ID NO: 60;
c) a first CDR comprising the amino acid sequence of SEQ ID NO: 27 or 60, and at least a second CDR comprising the amino acid sequence of SEQ ID NO: 25, SEQ ID NO: 26, SEQ ID NO: 56, or SEQ ID NO: 58, or
d) a first CDR comprising the amino acid sequence of SEQ ID NO: 25 or 56, a second CDR comprising the amino acid sequence of SEQ ID NO: 26 or 58, and a third CDR comprising the amino acid sequence of SEQ ID NO: 27 or 60.

In some embodiments, the VH comprises at least one complementarity-determining region (CDR) selected from the group consisting of SEQ ID NO: 28, SEQ ID NO: 29, SEQ ID NO: 30, SEQ ID NO: 67, SEQ ID NO: 69, and SEQ ID NO: 71. In some embodiments, the VH comprises:
a) a first CDR comprising the amino acid sequence of SEQ ID NO: 28 or 67, and at least a second CDR comprising the amino acid sequence of SEQ ID NO: 29, SEQ ID NO: 30, SEQ ID NO: 69, or SEQ ID NO: 71;
b) a first CDR comprising the amino acid sequence of SEQ ID NO: 29 or 69, and at least a second CDR comprising the amino acid sequence of SEQ ID NO: 28, SEQ ID NO: 30, SEQ ID NO: 67, or SEQ ID NO: 71;
c) a first CDR comprising the amino acid sequence of SEQ ID NO: 30 or 71, and at least a second CDR comprising the amino acid sequence of SEQ ID NO: 28, SEQ ID NO: 29, SEQ ID NO: 67, or SEQ ID NO: 69; or
d) a first CDR comprising the amino acid sequence of SEQ ID NO: 28 or 67, a second CDR comprising the amino acid sequence of SEQ ID NO: 29 or 69, and a third CDR comprising the amino acid sequence of SEQ ID NO: 30 or 71.

In some embodiments, the VL further comprises at least one framework region (FR) selected from the group consisting of SEQ ID NO: 55, SEQ ID NO: 57, SEQ ID NO: 59, and SEQ ID NO: 61. In some embodiments, the heavy chain further comprises at least one FR selected from the group consisting of SEQ ID NO: 66, SEQ ID NO: 68, SEQ ID NO: 70, and SEQ ID NO: 72. In some embodiments, the VL comprises an amino acid sequence having at least about 70% sequence identity to the amin acid sequence of SEQ ID NO: 23 or 53. In some embodiments, the VH comprises an amino acid sequence having at least about 70% sequence identity to the amin acid sequence of SEQ ID NO: 24 or 64. In some embodiments, the light chain further comprises at least one amino acid sequence having at least about 70% sequence identity to SEQ ID NO: 78. In some embodiments, the heavy chain further comprises at least one amino acid sequence having at least about 70% sequence identity to SEQ ID NO: 79.

In some embodiments, the expressible nucleic acid sequence further comprises a nucleic acid sequence encoding a VL of CD16. In some embodiments, the expressible nucleic acid sequence further comprises a nucleic acid sequence encoding a VH of CD16.

In some embodiments, the antibody or the antigen-binding fragment thereof further comprises at least one furin linker. In some embodiments, the at least one self-cleaving amino acid sequence is selected from the group consisting of FMDV 2A (F2A), equine rhinitis A virus (ERAV) 2A (E2A), porcine teschovirus-1 2A (P2A), and Thoseaasigna virus 2A (T2A), or at least one internal ribosome entry sequence (IRES) separates construct domains. In another embodiment, internal ribosome entry sequences (IRES) take the place of self-cleaving amino acid sequences, allowing independent translation of the different fragments.

In some embodiments, the light chain comprises an amino acid sequence having at least about 70% sequence identity to the amino acid sequence encoded by the nucleic acid sequence of SEQ ID NO: 14. In some embodiments, the heavy chain comprises an amino acid sequence having at least about 70% sequence identity to the amino acid sequence encoded by the nucleic acid sequence of SEQ ID NO: 16.

In some embodiments, either the VL and/or VH at least partially binds to V3 glycan supersite of the HIV envelope protein. In some embodiments, wherein the VL binds one of the following epitopes: the CD4-binding site, the V1/V2-glycan region, the V3-glycan region, the gp41 membrane proximal external region (MPER), or the gp120/gp41 interface of the envelope protein.

In some embodiments, the expressible nucleic acid sequence further comprises at least one nucleic acid sequence encoding a linker selected from the group consisting of a single glycine (Gly) residue, a diglycine peptide (Gly-Gly), a tripeptide (Gly-Gly-Gly), a single Ser, a single Val, the dipeptide Arg-Thr, Gln-Pro, Ser-Ser, Thr-Lys, and Ser-Leu, and the amino acid sequences of SEQ ID NO: 37, 38, 39, 40, 41, 42, 43, 44, 45, 46, 47, 48, 49, 50, 51, and 52.

In some embodiments, the antigen binding fragment is a single-chain variable fragment (scFv) of antibody 10-1074 and comprises an amino acid sequence having at least about 70% sequence identity with the amino acid sequence of SEQ ID NO: 75. In some embodiments, the antibody or the antigen binding fragment is not the full length of antibody 10-1074 encoded by the nucleic acid sequence of SEQ ID NO: 12.

In another aspect, the disclosure features a cell comprising the composition of any of the aspects or embodiments herein. In some embodiments, the cell is isolated form a subject diagnosed with or suspected of being infected with HIV. In other embodiments, the cell further comprises a costimulatory molecule capable of binding an HIV antigen.

In another aspect, the disclosure features a pharmaceutical composition comprising (i) one or plurality of T cells of any of the aspects or embodiments herein; and (ii) a pharmaceutically acceptable carrier.

In another aspect, the disclosure features a method of treating and/or preventing an HIV infection, comprising administering to a subject in need thereof an effective amount of the cell of any of the aspects and embodiments herein or the pharmaceutical composition of any of the aspects or embodiments herein. In one embodiment, the method further comprises administering to the subject one or a plurality of latency reversing agent (LRA) molecules prior to, simultaneously with or after administering the cell or pharmaceutical composition. In another embodiment, the effective amount is sufficient to accomplish one or any combination of (i) neutralization of one or a plurality of retroviruses in the subject; (ii) induction of NK cell recruitment to a cell infected with HIV in the subject; and (iii) antigen-specific cytotoxicity of a cell infected with HIV in the subject.

In another aspect, the disclosure features a nucleic acid encoding the antibody or antigen binding fragment of any of the aspects or embodiments herein. In one embodiment, the disclosure features a vector comprising the nucleic acid of any of the aspects or embodiments herein.

In another aspect, the disclosure features a method for the preparation of a cell expressing the antigen or antigen-binding fragment, comprising the step of culturing the cell under conditions that allow transduction of the cell with the vector of any of the aspects or embodiments herein. In one embodiment, the method further comprises the step of isolating the cell by cell sorting.

In another aspect, the disclosure features an immunoconjugate comprising the antibody or antibody binding fragment of any of the aspects or embodiments herein, coupled to a cytotoxic agent.

In another aspect, the disclosure features a method of destroying a cell in a subject infected by latent HIV infection comprising exposing the pharmaceutical composition of any of the aspects or embodiments herein to the cell for a time period sufficient to cause cytotoxicity of the cell. In one embodiment, the cell is contemporaneously exposed to one or a plurality of LRAs.

In some embodiments, the cell of any of the aspects or embodiments herein is a T cell. In some embodiments, the cell of any of the aspects or embodiments herein is a T cell recognizing HIV antigens in the following combinations: (1) gag, (2) nef, (3) pol, (4) gag and nef, (5) gag and pol, (6) nef and pol, (7) gag, nef, and pol. In some embodiments, the T cell of any of the aspects or embodiments herein recognizes only a subset of antigens from HIV gag, nef, and pol. In some embodiments, the cell of any of the aspects or embodiments herein is a T cell recognizing EBV antigens in the following combinations: (1) BARF1, (2) BMLF1, (3) BMRF1, (4) BRLF1, (5) BZLF1, (6) EBNA-LP, (7) EBNA1, (8) EBNA2, (9) EBNA3a, (10) EBNA3b, (11) EBNA3c, (12) GP350, (13) GP340, (14) LMP1, (15) LMP2, (16) EBNA-LP, EBNA1, EBNA2, EBNA3a, EBNA3b, EBNA3c, (17) LMP1, LMP2, (18) BARF1, BMLF1, BMRF1, BRLF1, BZLF1, (19) EBNA-LP, (20) EBNA1, LMP2, and BZLF1, (21) EBNA1, EBNA2, BZLF1 LMP1, and LMP2, (22) EBNA-LP, EBNA1, EBNA2, EBNA3a, EBNA3b, EBNA3c, LMP1, LMP2, BARF1, BMLF1, BMRF1, BRLF1, BZLF1. In some embodiments, the T cell of any of the aspects or embodiments herein recognizes only a subset of antigens from EBV EBNA-LP, EBNA1, EBNA2, EBNA3a, EBNA3b, EBNA3c, LMP1, LMP2, BARF1, BMLF1, BMRF1, BRLF1, BZLF1. In some embodiments, the cell of any of the aspects or embodiments herein is a T cell recognizing HPV serotype 16, 18, or 31 antigens in the following combinations: (1) E6, (2) E7, (3) L1, (4) L2, (5) E1, (6) E4, (7) E5, (8) E6 and E6, (9) E1, E4, E5, E6, E7 L1, L2. In some embodiments, the T cell of any of the aspects or embodiments herein recognizes only a subset of antigens from HPV 16, 18, or 31 E1, E4, E5, E6, E7 L1, L2. In some embodiments, the cell of any of the aspects or embodiments herein is a T cell recognizing HHV8/KSHV antigens in the following combinations: (1) ORF8, (2) ORF11, (3) ORF25, (4) ORF33, (5) ORF37, (6) ORF41, (7) ORF46, (8) ORF47, (9) ORF57, (10) LANAI, (11) v-cyclin, (12) v-IL6, (13) v-GPCR, (14) v-FLIP, (15) v-IRF3, (16) ORF8, ORF11, ORF25, ORF33, ORF37, ORF41, ORF46, ORF47, ORF57, (17) ORF8, ORF11, ORF57, (18) ORF8 and ORF11, (19) LANAI, v-cyclin, v-IL6, v-GPCR, v-FLIP, v-IRF3, (20) VFLIP, VIRF3, V cyclin, VIL6, V GPCR, (21) ORF8, ORF11, ORF25, ORF33, ORF37, ORF41, ORF46, ORF47, ORF57, LANAI, v-cyclin, v-IL6, v-GPCR, v-FLIP, v-IRF3. In some embodiments, the T cell of any of the aspects or embodiments herein recognizes only a subset of antigens from HHV8/KSHV 16, 18, or 31 ORF8, ORF11, ORF25, ORF33, ORF37, ORF41, ORF46, ORF47, ORF57, LANAI, v-cyclin, v-IL6, v-GPCR, v-FLIP, v-IRF3. In some embodiments, the cell of any of the aspects or embodiments herein is a T cell recognizing endogenous retrovirus sequences from HERV-HF, HERV-H, HERV-F, HERV-RW, HERV-W, ERV9, HuERS-P, HuRRS-P, HERV-ER1, 4-1, 5-1, ERV3, RRHERV-I, HERV-T, S71, CRTK1, CRTK6, HERV-IP, RTVL-I, ERV-FTD, ERV-FRD, class II HERVs, HERV-K. In some embodiments, the T cell of any of the aspects or embodiments herein recognizes only a subset of antigens from HERV-HF, HERV-H, HERV-F, HERV-RW, HERV-W, ERV9, HuERS-P, HuRRS-P, HERV-ER1, 4-1, 5-1, ERV3, RRHERV-I, HERV-T, S71, CRTK1, CRTK6, HERV-IP, RTVL-I, ERV-FTD, ERV-FRD, class II HERVs, HERV-K.

### BRIEF DESCRIPTION OF THE DRAWINGS

FIG. 1A depicts the schematic of the 10-1074 Ab construct and FIG. 1B depicts the schematic of the 10-1074 BiKE construct. The plasmid map containing these constructs are shown in FIG. 1C (10-1074 Ab) and FIG. 1D (10-1074 BiKE). FIG. 1E depicts the antibody processed from these constructs.

FIG. 2A depicts the transduction efficiency of the 10-1074 Ab construct. FIG. 2B depicts that products in transduced and nontransduced cells contained mixed populations of CD4+ T cells and CD8+ T cells. FIG. 2C depicts that, for transduced cells, a median of 121.2 ng/mL of antibody in the supernatant collected after 24 hours from T cells plated at 1×10⁶/mL is detected (mean 147.2±80.1 ng/mL, range 66.7 to 341, n=12). Similar transduction efficiencies (FIG. 2D) as well as T cell phenotype (FIG. 2E) was observed with the 10-1074 BiKE construct.

FIG. 3 depicts that the T cell-secreted antibodies obtained from the supernatant of cells transduced by the 10-1074 Ab construct bind to envelope-expressing cells but not non-expressing cells.

FIG. 4A depicts the transduction efficiency of the 10-1074 Ab construct in cells Cells that were expanded to recognize the HIV antigens g=Gag, Pol, and Nef. FIG. 4B depicts that these cells were able to express 10-1074 antibodies. FIG. 4C depicts that genetic modification did not significantly alter the makeup of CD4⁺ vs CD8⁺ populations within the T cell populations. Similar results were observed with the 10-1074 BiKE construct (FIG. 4D).

FIG. 5A depicts that genetic modification of the HIV specific T cells with the 10-1074 Ab construct did not significantly affect their abilities to expansion in response to antigenic stimulation with gag, pol, and nef peptides. FIG. 5B depicts that these genetically modified T cell lines also retained specificity to HIV peptides Gag, Nef, and Pol, as measured by IFNγ ELISPOT. FIG. 5C depicts that no significant differences in the secretion of T cell cytokines including GM-CSF, TNFα, IL-17, and the monocyte chemoattractant protein 1 were observed between nontransduced and transduced T cells. Similar results were observed with the 10-1074 BiKE construct (FIG. 5D and FIG. 5E).

FIG. 6A depicts that, using Env-transduced and non-transduced HeLa cells as targets, the transduced cells bound antibody while the nontransduced cells did not. FIG. 6B depicts that a significant increase in NK cell killing is seen when the supernatants from nontransduced and transduced cells were used to target HIV-envelope expressing HeLa cells. FIG. 6C depicts that the increase in killing from ADCC was observed using supernatants from multiple transduced lines, comparable to the control, a purified 10-1074 antibody which had been produced from 1×10⁶ cells/mL. FIG. 6D depicts the specificity of this increase in cytotoxicity using control 10-1074 targeting non-Env expressing HeLa cells. FIG. 6E and FIG. 6F show similar results from T cells transduced with the 10-1074 BiKE construct.

FIG. 7 depicts that the 10-1074 antibody-secreting T cell lines contain between 1-10% of CD3-CD56+NK cells.

FIG. 8A, FIG. 8B, and FIG. 8C show significantly increased inhibition of viral replication by HIV-specific T cells over CD8+ nonspecific T cells in each donor.

FIG. 9A, FIG. 9B, and FIG. 9C show that the addition of autologous NK cells to the product did not seem to significantly alter viral inhibition in two of the three evaluable lines (although there is a trend towards decreased amounts of p24 in all three lines). Similar results were also observed from T cells transduced with the construct 10-1074 BiKE (FIG. 9D).

FIG. 10A, FIG. 10B, and FIG. 10C depict that addition of control 10-1074 antibody alone (in the absence of NK cells) did decrease viral inhibition (in two of three evaluable lines) above that observed with uninfected cells.

FIG. 11 depicts that antibody secreted by HIV specific T_{bnAb} cell lines specifically binds to HIV-infected cells. Primary CD4+ T-cells were infected with a high MOI of a patient reservoir virus isolate (top) or with a low MOI of the molecular clone HIV SF162 (bottom). Infected cells were co-cultured with supernatants from T_{bnAbs} and then stained with a fluorochrome conjugated anti-IgG secondary antibody. Shown are flow cytometry data (x-axis, antibody staining; y-axis Gag staining).

FIG. 12A depicts the schematic of the Genesis 605a construct and FIG. 12B depicts the schematic of the Genesis 605b construct.

FIG. 13 depicts the result of dHXTC transduction flow obtained from the 10-1074 BiKE construct.

### DETAILED DESCRIPTION

The present disclosure is based, at least in part, on the idea of engineering isolated HIV-specific T cells secreting broadly neutralizing antibodies to mediate a multifaceted immune response against HIV. The present disclosure provides, in part, that genetic modification of T cells to secrete broadly neutralizing antibodies against HIV will not only maintain their T cell effector functions through specific cytotoxicity against HIV infected target cells, but also engage the endogenous immune system through ADCC and directly neutralize cell-free virus. Thus, the present disclosure provides a treatment method that is able to elicit three anti-viral effector functions, each previously shown to have limited or transient efficacy against HIV individually, and that, in combination, will effectively inhibit HIV. According to certain embodiments, the engineered HIV-specific T cells are administered in combination with latency reversing agents (LRAs).

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to” or “including, without limitation.”

The term “or” is used herein to mean, and is used interchangeably with, the term “and/or,” unless context clearly indicates otherwise. For example, an amino acid sequence with a modified amino acid is understood to include the options of an amino acid with a modified sidechain, a an amino acid with a modified backbone, and an amino acid with a modified sidechain and a modified backbone.

The term “about” is used herein to mean within the typical ranges of tolerances in the art. For example, “about” can be understood as about 2 standard deviations from the mean. According to certain embodiments, about means ±10%, ±9%, ±8%, ±7%, ±6%, ±5%, ±4%, ±3%, ±2%, ±1%, ±0.9%, ±0.8%, ±0.7%, ±0.6%, ±0.5%, 0.4%, 0.3%, ±0.2%, +0.1% or +0.05%. According to certain embodiments, about means +5%. When “about” is present before a series of numbers or a range, it is understood that “about” can modify each of the numbers in the series or range.

The term “at least” prior to a number or series of numbers (e.g. “at least two”) is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “up to” as in “up to 10” is understood as up to and including 10, i.e., 0, 1, 2, 3, 4, 5, 6, 7, 8, 9, or 10.

Ranges provided herein are understood to include all individual integer values and all subranges within the ranges.

The term “broad neutralizing antibody” refers to an antibody which inhibits HIV-1 infection. In some embodiments, the antibody inhibits HIV-1 infection as defined by at least about 50% inhibition of infection in vitro, in more than about 50%, 60%, 70%, 80%, 90%, 95%, 99% or greater, of a large panel of (greater than 100) HIV-1 envelope pseudotyped viruses and/or viral isolates. In some embodiments, the broad neutralizing antibody is an antibody that inhibits HIV-1 infection as defined by at least about 70%, 80%, 90%, 91%, 92%, 93%, 94%, 95%, 96%, 97%, 98%, 99% or 100% inhibition of infection in vitro in more than about 50%, 60%, 70%, 80%, 90%, 95%, 99% or greater, of a large panel of (greater than 100) HIV-1 envelope pseudotyped viruses and/or viral isolates. In some embodiments, the disclosure relates to a composition or pharmaceutical composition comprising one ore a plurality of broad neutralizing antibodies. In one embodiment, the broadly neutralizing antibody is 10-1074.

As used herein, the term “in combination with,” is not intended to imply that the therapy or the therapeutic agents must be administered at the same time and/or formulated for delivery together, although these methods of delivery are within the scope described herein. The therapeutic agents can be administered concurrently with, prior to, or subsequent to, one or more other additional therapies or therapeutic agents.

The term “antibody”, as used herein, broadly refers to any immunoglobulin (Ig) molecule comprised of four polypeptide chains, two heavy (H) chains and two light (L) chains, or any functional fragment, mutant, variant, or derivative thereof, which retains the essential epitope binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, “conservative” amino acid substitutions may be defined as set out in Tables A, B, or C below. Antibodies, antibody-like molecules and derivative, mutants, variants and salts thereof include those amino acid sequence wherein conservative substitutions have been introduced by solid state chemistry and/or recombinant modification of nucleic acids that encode amino acid sequences disclosed herein. In some embodiments, the compositions and pharmaceutical compositions of the disclosure comprise, 1, 2, 3, 4, 5 or more conservative amino acid substitutions. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A.

TABLE A
Conservative Substitutions I

| Side Chain Characteristics | Amino Acid |
|---|---|
| Aliphatic | |
| Non-polar | G A P I L V F |
| Polar-uncharged | C S T M N Q |
| Polar-charged | D E K R |
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| Non-polar (hydrophobic) | |
| Aliphatic: | A L I V P. |
| Aromatic: | F W Y |
| Sulfur-containing: | M |
| Borderline: | G Y |
| Uncharged-polar | |
| Hydroxyl: | S T Y |
| Amides: | N Q |
| Sulfhydryl: | C |
| Borderline: | G Y |
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides comprising polypeptide sequences associated with the extracellular matrix described herein are intended to include polypeptides bearing one or more insertions, deletions, or substitutions, or any combination thereof, of amino acid residues as well as modifications other than insertions, deletions, or substitutions of amino acid residues.

As used herein, the term “CDR” refers to the complementarity determining region within antibody variable sequences. In some embodiments, there are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia et al., J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “fragment” is defined as a physically contiguous portion of the primary structure of a biomolecule. In the case of polypeptides, a fragment may be defined by a contiguous portion of the amino acid sequence of a protein and may be at least 3-5 amino acids, at least 6-10 amino acids, at least 11-15 amino acids, at least 16-24 amino acids, at least 25-30 amino acids, at least 30-45 amino acids and up to the full length of the protein minus a few amino acids. In the case of polynucleotides, a fragment is defined by a contiguous portion of the nucleic acid sequence of a polynucleotide and may be at least 9-15 nucleotides, at least 15-30 nucleotides, at least 31-45 nucleotides, at least 46-74 nucleotides, at least 75-90 nucleotides, and at least 90-130 nucleotides. In some embodiments, fragments of biomolecules are immunogenic fragments. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000 or more nucleotides or amino acids.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

The “variable domain” (variable domain of a light chain (VL), variable domain of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a beta-sheet conformation and the CDRs may form loops connecting the beta-sheet structure. The CDRs in each chain are held in their three-dimensional structure by the framework regions and form together with the CDRs from the other chain an antigen binding site. References to “VH” refer to the variable domain of an immunoglobulin heavy chain, including that of an antibody fragment, such as Fv, scFv, dsFv or Fab. References to “VL” refer to the variable domain of an immunoglobulin light chain, including that of an Fv, scFv, dsFv or Fab.

The term “antigen binding portion” or “antigen binding fragment” of an antibody (or simply “antibody portion” or “antibody fragment”), as used herein, refers to one or more fragments of an antibody that retain the ability to specifically bind to an antigen (e.g., hCD40). It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” or “antigen binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

Full length antibodies comprise immunoglobulin constant regions of one or more immunoglobulin classes. Immunoglobulin classes include IgG, IgM, IgA, IgD, and IgE isotypes and, in the case of IgG and IgA, their subtypes. In a preferred embodiment, an full length antibody of the disclosure has a constant domain structure of an IgG type antibody.

The terms “Kabat numbering”, “Kabat definitions and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen-binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

A “neutralizing antibody” may inhibit the entry of HIV-1 virus for example SF162 and/or JRCSF with a neutralization index >1.5 or >2.0. Broad and potent neutralizing antibodies may neutralize greater than about 50% of HIV-1 viruses (from diverse clades and different strains within a clade) in a neutralization assay. The inhibitory concentration of the monoclonal antibody may be less than about 25 mg/ml to neutralize about 50% of the input virus in the neutralization assay. In some embodiments, the disclosure relates to pharmaceutical compositions comprising T cells comprising a nucleic acid sequence that encodes a neutralizing antibody.

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The term “antigen” refers to a polypeptide that can stimulate the production of antibodies or a T cell response in an animal, including polypeptides that are injected or absorbed into an animal. An antigen reacts with the products of specific humoral or cellular immunity.

The term “antibody-Dependent Cell-mediated Cytotoxicity (ADCC)” as used herein refers to a mechanism by which antibody-coated target cells are killed by Fc Receptor expressing effector cells.

The term “HIV” is known to one skilled in the art to refer to Human Immunodeficiency Virus. There are two types of HIV: HIV-1 and HIV-2. There are many different strains of HIV-1. The strains of HIV-1 can be classified into three groups: the “major” group M, the “outlier” group 0 and the “new” group N. These three groups may represent three separate introductions of simian immunodeficiency virus into humans. Within the M-group there are at least ten subtypes or clades: e.g., clade A, B, C, D, E, F, G, H, I, J, and K. A “clade” is a group of organisms, such as a species, whose members share homologous features derived from a common ancestor. Any reference to HIV-1 in this application includes all of these strains.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, the term “expression” is meant to encompass production of an observable phenotype by a gene, usually by directing the synthesis of a protein. It includes the biosynthesis of mRNA, polypeptide biosynthesis, polypeptide activation, e.g., by post-translational modification, or an activation of expression by changing the subcellular location or by recruitment to chromatin.

As used herein, the term “flow cytometry” is meant to refer to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow analysis and differentiation of the cells is based on size, granularity, and whether a cell is carrying fluorescent molecules in the form of either antibodies or dyes. As the cell passes through the laser beam, light is scattered in all directions, and the light scattered in the forward direction at low angles (0.5-10°) from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007). Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

The term “host cell” as used herein is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. coli; mammalian cell lines CHO, HEK 293 and COS; the insect cell line Sf9; and the fungal cell Saccharomyces cerevisiae.

As used herein, the term “nucleic acid molecule” comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of antibodies or antibody fragments. In some embodiments, the disclosure relates to a pharmaceutical composition comprising a first, second, third or more nucleic acid molecule, each of which encoding one or a plurality of antibodies or antibody fragments and at least one of each plasmid comprising one or more of the nucleic acid sequences or amino acid sequences disclosed herein or those that comprise at least 70%, 80%, 90%, 95%, or 99% seqeunce homology to those the nucleic acid sequences or amino acid sequences disclosed herein.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

The term “T cell,” “T-cell,” “T lymphocyte” or “T-lymphocyte” is known to one skilled in the art to refer to the type of lymphocytes that are produced or processed by the thymus gland. T cells can be distinguished from other lymphocytes by the presence of a T-cell receptor on the cell surface.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

The term “inhibit” and its various grammatical forms is used to refer to a restraining, blocking, or limiting of the range or extent of a certain biological event or effect.

As used herein, the term “dose” is meant to refer to the quantity of a therapeutic substance prescribed to be taken at one time. The term “maximum tolerated dose” as used herein is meant to refer to the highest dose of a drug or treatment that does not cause unacceptable side effects.

The term “effective amount,” is used herein to include the amount of an agent (e.g. a cell comprising an antibody or antibody fragment of the disclosure) that, when administered to a patient for treating a subject infection, is sufficient to effect treatment of the disease (e.g., by diminishing, ameliorating or maintaining the existing disease or one or more symptoms of disease or its related comorbidities). The “effective amount” may vary depending on the agent, how it is administered, the disease and its severity and the history, age, weight, family history, genetic makeup, stage of pathological processes, the types of preceding or concomitant treatments, if any, and other individual characteristics of the patient to be treated. An effective amount includes an amount that results in a clinically relevant change or stabilization, as appropriate, of an indicator of a disease or condition. “Effective amount” refers to an amount of a compound, material, or composition, as described herein effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art. The effective amount of the composition may be dependent on any number of variables, including without limitation, the species, breed, size, height, weight, age, overall health of the subject, the type of formulation, the mode or manner or administration, the type and/or severity of the particular condition being treated, or the need to modulate the activity of the molecular pathway induced by association of the analog to its receptor. The appropriate effective amount can be routinely determined by those of skill in the art using routine optimization techniques and the skilled and informed judgment of the practitioner and other factors evident to those skilled in the art. An effective dose of the antibodies or mutants or variants described herein may provide partial or complete biological activity as compared to the biological activity induced by the wild-type or naturally occurring polypeptides upon which the antibodies or mutants or variants are derived. A therapeutically effective dose of the antibodies or mutants or variants described herein may provide a sustained biochemical or biological affect and/or an increased resistance to degradation when placed in solution as compared with the normal affect observed when the naturally occurring and fully processed translated protein is administered to the same subject. In certain embodiments, “therapeutically effective” means the amount of agent required to provide a meaningful patient benefit as understood by practitioners in the field of AIDS and HIV infection. In general, the goals of treatment are suppression of viral load, restoration and preservation of immunologic function, improved quality of life, and reduction of HIV-related morbidity and mortality.

An “immunoconjugate” is an antibody or multispecific antibody conjugated to one or more heterologous molecule(s), including but not limited to a cytotoxic agent.

The term “cytotoxicity” refers to the property of killing cells.

The term “cytotoxic agent” as used herein refers to a substance that inhibits or prevents a cellular function and/or causes cell death or destruction. Cytotoxic agents include, but are not limited to, radioactive isotopes; growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof.

The term “administer” as used herein means to give or to apply. The term “administering” as used herein includes in vivo administration.

The term “linker” refers to a chemical moiety that connects one peptide to another, e.g., one antibody to another. Linkers can also be used to attach antibodies to labels or solid substrates. A linker can include amino acids. Linkers can be straight or branched, saturated or unsaturated carbon chains. They can also include one or more heteroatoms within the chain. In some embodiments, there is at least one linker encoding a linker from about 3 to about 25 amino acids in length. In some embodiment, the linker sequence separate each antigen expression domain. In some embodiments, the nucleic acid sequence comprises 1, 2, 3, 4, 5, 6, 7, 8, 9, or 10 or more linkers. In some embodiments, the nucleic acid sequence comprises 1, 2, 3, 4, 5, 6, 7, 8, 9, or 10 or more linkers, at least one or more are comprise furin linkers. In some embodiments, the nucleic acid sequence comprises at least 2, 3, 4, 5, 6, 7, 8, 9, or 10 or more linker domains.

The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present disclosure can be administered by a variety of methods known in the art. As will be appreciated by the skilled artisan, the route and/or mode of administration will vary depending upon the desired results. To administer an antibody according to the disclosure by certain routes of administration, it may be necessary to coat the antibody with, or co-administer the antibody with, a material to prevent its inactivation. For example, the antibody may be administered to a subject in an appropriate carrier, for example, liposomes, or a diluent. Pharmaceutically acceptable diluents include saline and aqueous buffer solutions.

A “pharmaceutically acceptable carrier” refers to an ingredient in a pharmaceutical formulation, other than an active ingredient, which is nontoxic to a subject. Pharmaceutically acceptable carriers includes any and all solvents, dispersion media, coatings, antibacterial and antifungal agents, isotonic and absorption delaying agents, and the like that are physiologically compatible. In one preferred embodiment, the carrier is suitable for intravenous, intramuscular, subcutaneous, parenteral, spinal or epidermal administration (e.g. by injection or infusion).

The pharmaceutical compositions according to the disclosure may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The term “subject” is used throughout the specification to describe an animal to which one or more compositions comprising the antibody or antibodies disclosed herein. In some embodiment, the animal is a human. For diagnosis of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a human suspected of having or being identified as at risk to develop HIV infection. In some embodiments, the subject is suspected of having or has been diagnosed with HIV or HIV-1 infection or AIDS. In some embodiments, the subject may be a human suspected of having or being identified as at risk to develop AIDS or an AIDS-associated disorder. In some embodiments, the subject may be a mammal. In some embodiments, the subject may be a non-human animal. The term “mammal” encompasses both humans and non-humans and includes but is not limited to humans, non-human primates, canines, felines, murines, bovines, equines, and porcines. is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). In an embodiment, the subject is a human, such as a human being treated or assessed for an HIV infection; or a human having an HIV infection that would benefit from a multispecific antibody as described herein. In some embodiments, the subject is a subject in need thereof, meaning that the subject is need of the treatment being administered.

The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Salts include those formed from hydrochloric, hydrobromic, sulphuric, phosphoric, citric, tartaric, lactic, pyruvic, acetic, succinic, fumaric, maleic, methanesulphonic and benzenesulphonic acids. In some embodiments, salts of the compositions comprising either an antibody or antibody-like molecule may be formed by reacting the free base, or a salt, enantiomer or racemate thereof, with one or more equivalents of the appropriate acid. In some embodiments, pharmaceutical acceptable salts of the present disclosure refer to derivatives or amino acid sequences comprising at least one basic group or at least one basic radical. In some embodiments, pharmaceutical acceptable salts of the disclosed compositions comprise a free amino group, a free guanidino group, a pyrazinyl radical, or a pyridyl radical that forms acid addition salts. In some embodiments, the pharmaceutical acceptable salts of the present disclosure refer to modified amino acids that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present disclosure may be found in their anhydrous form or as in hydrated crystalline form (i.e., complexed or crystallized with one or more molecules of water). In some embodiments, the compositions or pharmaceutical compositions comprise crystalline forms or lyophilized forms of the antibodies, antibody-like molecules or salts thereof. In some embodiments, the disclosure relates to pharmaceutical compostions comprising an antibody or antigen binding fragment or their respective salts thereof.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

The term “potency” as used herein refers to the neutralization capacity, i.e. the IC₅₀ or IC₈₀ of the antibody, or fragment thereof.

Humanization and primatization refer to in cases where the tri-specific fusion antibody or the three antibodies forming the tri-specific fusion antibody are non-human antibodies, the antibody can be “humanized” to reduce immunogenicity to a human recipient. Methods for humanizing non-human antibodies have been described in the art. See, e.g., Jones et al., Nature 321:522-525 (1986); Riechmann et al, Nature 332:323-327 (1988); Verhoeyen et al., Science 239: 1534-1536 (1988), and U.S. Pat. No. 4,816,567. Generally, residues from the variable domain of a non-human antibody are “imported” into a human immunoglobulin molecule, resulting in antibodies in which some hypervariable region residues and possibly some FR residues of a human antibody are substituted by residues from analogous sites of non-human antibodies. It is important to humanize a non-human antibody while retaining high affinity for the antigen. To this end, three dimensional immunoglobulin models are commonly available and suitable for use in analyzing proposed humanized sequences in comparison to the parental non-human antibodies. Such analysis permits identification of residues likely involved in recognition and binding of the antigen, and therefore rational design of humanized sequences that retain the specificity and affinity for the antigen.

By “affinity maturation” is meant when one or more hypervariable region residues of an antibody can be substituted to select for variants that have improved biological properties relative to the parent antibody by employing, e.g., affinity maturation using phage or yeast display. For example, the Fab region of an anti-HIV antibody can be mutated at several sites selected based on available structural information to generate all possible amino substitutions at each site. The antibody variants thus generated are displayed in a monovalent fashion from phage particles or on the surface of yeast cells. The displayed variants are then screened for their biological activity (e.g. binding affinity).

The term “IC₅₀” as used herein refers to the concentration of an inhibitor, such as a multispecific antibody, where the response or biological activity is reduced by half.

The term “IC₈₀” as used herein refers to the concentration of an inhibitor (e.g. a multispecific antibody) where the response or biological activity is reduced by eighty percent.

The term “latency reversing agent” as used herein includes, but is not limited to Protein Kinase C (PKC) agonists, bromo and external (BET) bromodomain inhibitors, histone deacetylase (HDAC) inhibitors, acetaldehyde dehydrogenase inhibitors, activators of nuclear factor kappa-light chain-enhancer of activated B cells (NF-κB) and the AKT pathway. In certain embodiments, the PKC agonist is biyostatin-1, prostratin, ingenol-3-angelate, ingenol mimic, or DAG mimic. In certain embodiments, the acetaldehyde dehydrogenase inhibitor, activator of F-κB is disulfiram. In certain embodiments, the HDAC inhibitor is selected from the group consisting of vorinostat, panobinostat, and romidepsin. In other embodiments, the HDAC inhibitor is selected from 4-phenylbutyrohydroxamic acid, Acetyldinaline, APHA, Apicidin, AR-42, Belinostat, CUDC-101, CUDC-907, Dacinostat, Depudecin, Droxinostat, Entinostat, Givinostat, HC-Toxin, ITF-2357, JNJ-26481585, KD 5170, LAQ-824, LMK 235, M344, MC1568, MGCD-0103, Mocetinostat, NCH 51, Niltubacin, NSC3852, Oxamflatin, Panobinostat, PCI-24781, PCI-34051, Pracinostat, Pyroxamide, Resminostat, RG2833, RGFP966, Rocilinostat, Romidepsin, SBHA, Scriptaid, Suberohydroxamic acid, Tacedinaline, TC-H 106, TCS HDAC6 20b, Tacedinaline, TMP269, Trichostatin A, Tubacin, Tubastatin A, Valproic acid, or Vorinostat. In certain embodiments, the bromodomain inhibitor is JQ1. In other embodiments, the BET inhibitor is selected from CPI 203, 1-BET151, 1-BET762, JQ1, MS417, MS436, OTX-015, PFi-1, or RVX-208. In certain embodiments, the latency reversing drug combinations comprise acetaldehyde dehydrogenase inhibitor, activator of NF-κB and the AKT pathway with HDAC inhibitors.

Anti-HIV Broadly Neutralizing Antibodies (bNAbs)

In some embodiments, the present disclosure involves anti-HIV-1 broadly neutralizing antibodies (or “bNAbs”). In some embodiments, a broadly neutralizing antibody is defined as a bNAb that neutralizes HIV-1 species belonging to two or more different clades. In some embodiments the different clades are selected from the group consisting of clades A, B, C, D, E, AE, AG, G or F. In some embodiments the HIV-1 strains from two or more clades comprise virus from non-B clades. In some embodiments, bNAbs target conserved sites of vulnerability on the HIV-1 envelope (env). In some embodiments, the bNAb any anti-HIV-1 antibody that is sufficient to neutralize or bind up to or at least about 50%, 60%, 70%, 80%, 85%, 86%, 87%, 88%, 89%, 90%, 91%, 92%, 93%, 94%, 95%, 96%, 97%, 98%, 99% or up to 100% of viral isolates in culture or in a subject.

In some embodiments, the bNAb is selected based on its neutralization activity. In one embodiments, the bNAb is selected based on its ability to bind HIV-1 infected cells (predictive of ADCC).

Various bNAbs are known in the art and can be used according to this disclosure. In some embodiments, the present disclosure comprises a composition or cell comprising bispecific, trispecific or tetraspecific anti-HIV bNAbs. Examples include but are not limited to those described in U.S. Pat. No. 8,673,307, WO2014063059, WO2012158948, WO2015/117008, and PCT/US2015/41272, including antibodies 3BNC117, 3BNC60, 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al, Nature, 2012. 492(7427): p. 118-22, Horwitz et al, Proc Natl Acad Sci USA, 2013. 110(41): p. 16538-43, Scheid, et al. 2011. Science, 333: 1633-1637, Scheid, et al. 2009. Nature, 458:636-640, Eroshkin et al, Nucleic Acids Res. 2014 January; 42133-9, Mascola et al. Immunol Rev. 2013 July; 254(1):225-44.

Certain bNAbs target conserved sites of vulnerability on the HIV-1 envelope (ENV) such as the CD4 binding site (CD4bs). The b12 monoclonal antibody was for many years considered the prototype and optimal CD4bs bNAb, although it was only able to neutralize about 40% of HIV-1 strains. In 2010, a new group of CD4bs antibodies named VRC01, VRC02, and VRC03 was disclosed. Of these, VRC01 was the most potent and broad. In a large neutralization panel (190 viruses), VRC01 neutralized 91% of viruses with an IC₅₀ less than 50 μg/ml and 72% of viruses with an IC50 less than 1 μg/ml (Wu et al., Science, 329(5993):856-861, 2010). Structural analyses have explained VRC01's high potency and breadth: VRC01 partially mimics the CD4 interaction with gp120. Specifically, the majority of the gp120 area targeted by VRC01 is the highly conserved site of initial CD4 attachment in the outer domain of gp120, which allows VRC01 to bypass conformational and glycan masking that impaired previously identified CD4bs bNAbs. Both the heavy and light chain of VRC01 contribute to the binding of gp120, with the CDRH2 providing the primary interaction, and CDRL1, CDRL3, CDRH1, and CDRH3 providing additional contact points. It has been shown that passive transfer of VRC01 protects against intrarectal or intravaginal simian-HIV (SHIV) challenge in non-human primates.

VRC01 is a monoclonal antibody that specifically binds to gp120 and is neutralizes a broad range of HIV viruses. The amino acid sequences of the variable heavy (VH) chain and variable light (VL) chain of VRC01 have been described in Wu et al., Science, 329(5993):856-861, 2010, and PCT publication WO2012/154312, incorporated by reference herein in its entirety. VRC01-like antibodies are described, for example in US20170267748, incorporated by reference herein in its entirety. Generally, these antibodies bind to the CD4 binding surface of gp120 in substantially the same orientation as VRC01, and are broadly neutralizing VRC01-like antibodies, with several of the important contacts between CD4 and gp120 mimicked by the VRC01-like antibodies. Several VRC01-like antibodies are available, including VRC01-like antibodies, heavy chains and light chains disclosed in PCT International Application No. PCT/US2010/050295, filed Sep. 24, 2010, which is incorporated by reference herein and Wu et al., “Rational design of envelope identifies broadly neutralizing human monoclonal antibodies to HIV-1,” Science, 329(5993):856-861, 2010, which is incorporated by reference herein. These include heavy and light chains of the VRC01, VRC02, VRC03, VRC06, VRC07, 3BNC117, IOMA and N6. The amino acid sequences of the heavy and light variable regions of VRC03 have been described in Wu et al., (Science. 2010 Aug. 13; 329(5993):856-61; PMID 20616233). The amino acid sequences of the heavy and light variable regions of VRC06 have been described in Li et al., (J Virol. 2012 October; 86(20):11231-41; PMID 22875963). The amino acid sequences of the heavy and light variable regions of VRC07 have been described in Rudicell et al., (J Virol. 2014 November; 88(21):12669-82; PMID 25142607). The amino acid sequences of the heavy and light variable regions of 3BNC117 have been described in Scheid et al., (Science. 2011 Sep. 16; 333(6049):1633-7; PMID 21764753). The amino acid sequences of the heavy and light variable regions of IOMA have been described in Gristick et al., (Nat Struct Mol Biol. 2016 October; 23(10):906-915; PMID 27617431). The amino acid sequences of the heavy and light variable regions of N6 have been described in Huang et al., (Immunity. 2016 Nov. 15; 45(5):1108-1121; PMID 27851912).

PGT121, PGT122, PGT123, PGT127, PGT128, PGT135, 10-1074 and BG18 are a family of neutralizing monoclonal antibodies that specifically bind to the V1/V2 and V3 regions of HIV-1 Env and can inhibit HIV-1 infection of target cells. PGT121, PGT122, and PGT123 mAbs and methods of producing them are described in, for example, Walker et al., Nature, 477:466-470, 2011, and Int. Pub. No. WO 2012/030904, each of which is incorporated by reference herein. PGT127 and PGT128 are described in, for example Pejchal et al. (Science, 2011 Nov. 25, 334 (6059): 1097-103). PGT135 is described, for example, in Kong et al. (Nature Structural and Molecular Biology, 2013 July, 20:796-803). The amino acid sequences of the heavy and light variable regions of 10-1074 have been described in Mouquet et al. ((2012) Proc. Natl. Acad. Sci. USA 109: E3268-E3277). The amino acid sequences of the heavy and light variable regions of BG18 have been described in Freund et al. ((2012) Sci Transl Med. 2017 Jan. 18; 9(373); PMID 28100831). The amino acid sequences of the heavy and light variable regions of PGT135 have been described in Kong et al. (Nat Struct Mol Biol. 2013 July; 20(7):796-80; PMID 23708606). The amino acid sequences of the heavy and light variable regions of PGT122 have been described in Julien et al. (PLoS Pathog. 2013; 9(5):e1003342; PMID 23658524). The amino acid sequences of the heavy and light variable regions of PGT128 have been described in Lee et al. (Structure. 2015 Oct. 6; 23(10):1943-51; PMID 26388028).

35022, N123-VRC34.01, 3BC315, and PGT151 are broadly neutralizing monoclonal antibodies that specifically bind to the gp120/gp41 interface of HIV-1 Env in its prefusion mature (cleaved) conformation, and which can inhibit HIV-1 infection of target cells. PGT151 antibody and methods of producing this antibody are described in, for example, Blattner et al., Immunity, 40, 669-680, 2014, and Falkowska et al., Immunity, 40, 657-668, 2014, each of which is incorporated by reference herein in its entirety). The amino acid sequences of the heavy and light variable regions of the PGT151 mAb are known and have been deposited in GenBank as Nos. KJ700282.1 (PGT151 VH) and KJ700290.1 (PGT151 VL), each of which is incorporated by reference herein in its entirety). The amino acid sequences of the heavy and light variable regions of N123-VRC34.01 have been described in Kong et al., (Science 352 (6287), 828-833 (2016)). The amino acid sequences of the heavy and light variable regions of 3BC315 have been described in Lee et al. (Nat Commun. 2015 Sep. 25; 6:8167; PMID 26404402). The amino acid sequences of the heavy and light variable regions of PGT151 have been described in Blattner et al. (Immunity. 2014 May 15; 40(5):669-80; PMID 24768348).

10E8, 10E8v4, 10E8v4 S100cF, Dh511.2 k3, Z13, 4E10, and 2F5 are broadly neutralizing monoclonal antibody that primarily targets a HIV Env membrane proximal external region (MPER) helix spanning residues 671-683. The amino acid sequences of the heavy and light variable regions of 10E8v4 have been described in Kwon et al. (J Virol. 2016 Jun. 10; 90(13):5899-914; PMID PMC4907239). The amino acid sequences of the heavy and light variable regions of 10E8v4 S100cF have been described in PCT/US2016/060390 and WO2017079479. The amino acid sequences of the heavy and light variable regions of DH511.2 k3 have been described in Williams et al. (Sci Immunol. 2017 Jan. 27; 2(7); PMID 28783671). The amino acid sequences of the heavy and light variable regions of 4E10 have been described in Rujas et al. (J Virol. 2015 December; 89(23):11975-89; PMID 26378169). The amino acid sequences of the heavy and light variable regions of 2F5 have been described in Julien et al. J Mol Biol. 2008 Dec. 12; 384(2):377-92; PMID 18824005).

PGT141, PGT142, PGT143, and PGT145 are a family of broadly neutralizing monoclonal antibodies that specifically bind to the V1/V2 domain of the HIV-1 Env ectodomain trimer in its prefusion mature closed conformation, and which can inhibit HIV-1 infection of target cells. PGT141, PGT142, PGT143, and PGT145 mAbs and methods of producing them are described in, for example, Walker et al., Nature, 477:466-470, 2011, and Int. Pub. No. WO2012/030904, each of which is incorporated by reference herein). The amino acid sequences of the heavy and light variable regions of the PGT141, PGT142, PGT143, PGT144, and PGT145 mAbs are known and have been deposited in GenBank as Nos. JN201906.1 (PGT141 VH), JN201923.1 (PGT141 VL), JN201907.1 (PGT142 VH), JN201924.1 (PGT142 VL), JN201908.1 (PGT143 VH), JN201925.1 (PGT143 VL), JN201909.1 (PGT144 VH), JN201926.1 (PGT144 VL), JN201910.1 (PGT145 VH), and JN201927.1 (PGT145 VL), each of which is incorporated by reference herein in its entirety).

The HIV-1 neutralizing single domain antibody JM4 (Matz J, Kessler P, Bouchet J, Combes O, Ramos O H, Barin F, Baty D, Martin L, Benichou S, Chames P. Straightforward selection of broadly neutralizing single-domain antibodies targeting the conserved CD4 and coreceptor binding sites of HIV-1 gp120. J Virol. 2013 January; 87(2):1137-49. doi: 10.1128/JVI.00461-12. Epub 2012 Nov. 14) is another neutralizing antibody that can be used in the present disclosure.

In some embodiments, the bNAb is selected from 10-1074, VRC01, VRC07, 3BNC117, N6, PCT121, 2G12, GDM1400, CAP256, PG16, 10E8, 2F5, 4E10, PG9, JM4, and VRC01.

In one embodiment, the bNAb is PG9 or a salt thereof. In another embodiment, the bNAb is JM4 or a salt thereof.

In one embodiment, the bNAb is 10-1074 or a salt thereof (Mouquet, et al., Proc. Natl. Acad. Sci. U.S.A, 109(47):E3268-E3277, 20 Nov. 2012, incorporated by reference in its entirety herein). Monoclonal antibody 10-1074 targets the V3 glycan supersite on the HIV-1 envelope (Env) protein. It is among the most potent anti-HIV-1 neutralizing antibodies isolated to date. Table 1, below, sets forth nucleic acid sequences of 10-1074 and PG9.

TABLE 1

| SEQ | | |
|---|---|---|
| ID NO | Description | Sequence |
| 1 | PG9 Full | CGTTACATAACTTACGGTAAATGGCCCGCCTGGCTGACCGCCCAACGACCCCC |
| | length | GCCCATTGACGTCAATAATGACGTATGTTCCCATAGTAACGCCAATAGGGACT |
| | Antibody- | TTCCATTGACGTCAATGGGTGGAGTATTTACGGTAAACTGCCCACTTGGCAGT |
| | nucleic acid | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACCATGGGATGGTCATGTATCA |
| | | TCCTTTTTCTAGTAGCAACTGCAACCGGTGTACATTCACAGTCTGCCCTGACT |
| | | CAGCCTGCCTCCGTGTCTGGGTCTCCTGGACAGTCGATCACCATCTCCTGCAA |
| | | TGGAACCAGCAATGATGTTGGTGGCTATGAATCTGTCTCCTGGTACCAACAAC |
| | | ATCCCGGCAAAGCCCCCAAAGTCGTGATTTATGATGTCAGTAAACGGCCCTCA |
| | | GGGGTTTCTAATCGCTTCTCTGGCTCCAAGTCCGGCAACACGGCCTCCCTGAC |
| | | CATCTCTGGGCTCCAGGCTGAGGACGAGGGTGACTATTACTGCAAGTCTCTGA |
| | | CAAGCACGAGACGTCGGGTTTTCGGCACTGGGACCAAGCTGACCGTTCTAACC |
| | | GTGGCGGCGCCGAGCGTGTTTATTTTTCCGCCGAGCGATGAACAGCTGAAAAG |
| | | CGGCACCGCGAGCGTGGTGTGCCTGCTGAACAACTTTTATCCGCGCGAAGCGA |
| | | AAGTGCAGTGGAAAGTGGATAACGCGCTGCAGAGCGGCAACAGCCAGGAAAGC |
| | | GTGACCGAACAGGATAGCAAAGATAGCACCTATAGCCTGAGCAGCACCCTGAC |
| | | CCTGAGCAAAGCGGATTATGAAAAACATAAAGTGTATGCGTGCGAAGTGACCC |
| | | ATCAGGGCCTGAGCAGCCCGGTGACCAAAAGCTTTAACCGCGGCGAATGCCGC |
| | | AAACGCCGCGGCAGCGGCGCGACCAACTTTAGCCTGCTGAAACAGGCGGGCGA |
| | | TGTGGAAGAAAACCCGGGCCCGATGGGATGGTCATGTATCATCCTTTTTCTAG |
| | | TAGCAACTGCAACCGGTGTACATTCACAGCGATTAGTGGAGTCTGGGGGAGGC |
| | | GTGGTCCAGCCTGGGTCGTCCCTGAGACTCTCCTGTGCAGCGTCCGGATTCGA |
| | | CTTCAGTAGACAAGGCATGCACTGGGTCCGCCAGGCTCCAGGCCAGGGGCTGG |
| | | AGTGGGTGGCATTTATTAAATATGATGGAAGTGAGAAATATCATGCTGACTCC |
| | | GTATGGGGCCGACTCAGCATCTCCAGAGACAATTCCAAGGATACGCTTTATCT |
| | | CCAAATGAATAGCCTGAGAGTCGAGGACACGGCTACATATTTTTGTGTGAGAG |
| | | AGGCTGGTGGGCCCGACTACCGTAATGGGTACAACTATTACGATTTCTATGAT |
| | | GGTTATTATAACTACCACTATATGGACGTCTGGGGCAAAGGGACCACGGTCAC |
| | | CGTCTCGAGCGCGAGCACCAAAGGCCCGAGCGTGTTTCCGCTGGCGCCGTGCA |
| | | GCCGCAGCACCAGCGGCGGCACCGCGGCGCTGGGCTGCCTGGTGAAAGATTAT |
| | | TTTCCGGAACCGGTGACCGTGAGCTGGAACAGCGGCGCGCTGACCAGCGGCGT |
| | | GCATACCTTTCCGGCGGTGCTGCAGAGCAGCGGCCTGTATAGCCTGAGCAGCG |
| | | TGGTGACCGTGCCGAGCAGCAGCCTGGGCACCCAGACCTATACCTGCAACGTG |
| | | AACCATAAACCGAGCAACACCAAAGTGGATAAACGCGTGGAACTGAAAACCCC |
| | | GCTGGGCGATACCACCCATACCTGCCCGCGCTGCCCGGAACCGAAAAGCTGCG |
| | | ATACCCCGCCGCCGTGCCCGCGCTGCCCGGAACCGAAAAGCTGCGATACCCCG |
| | | CCGCCGTGCCCGCGCTGCCCGGAACCGAAAAGCTGCGATACCCCGCCGCCGTG |
| | | CCCGCGCTGCCCGGCGCCGGAACTGCTGGGCGGCCCGAGCGTGTTTCTGTTTC |
| | | CGCCGAAACCGAAAGATACCCTGATGATTAGCCGCACCCCGGAAGTGACCTGC |
| | | GTGGTGGTGGATGTGAGCCATGAAGATCCGGAAGTGCAGTTTAAATGGTATGT |
| | | GGATGGCGTGGAAGTGCATAACGCGAAAACCAAACCGCGCGAAGAACAGTATA |
| | | ACAGCACCTTTCGCGTGGTGAGCGTGCTGACCGTGCTGCATCAGGATTGGCTG |
| | | AACGGCAAAGAATATAAATGCAAAGTGAGCAACAAAGCGCTGCCGGCGCCGAT |
| | | TGAAAAAACCATTAGCAAAACCAAAGGCCAGCCGCGCGAACCGCAGGTGTATA |
| | | CCCTGCCGCCGAGCCGCGAAGAAATGACCAAAAACCAGGTGAGCCTGACCTGC |
| | | CTGGTGAAAGGCTTTTATCCGAGCGATATTGCGGTGGAATGGGAAAGCAGCGG |
| | | CCAGCCGGAAAACAACTATAACACCACCCCGCCGATGCTGGATAGCGATGGCA |
| | | GCTTTTTTCTGTATAGCAAACTGACCGTGGATAAAAGCCGCTGGCAGCAGGGC |
| | | AACATTTTTAGCTGCAGCGTGATGCATGAAGCGCTGCATAACCGCTTTACCCA |
| | | GAAAAGCCTGAGCCTGAGCCCGGGCAAACGCAAACGCCGCGGCAGCGGCGCGA |
| | | CCAACTTTAGCCTGCTGAAACAGGCGGGCGATGTGGAAGAAAACCCGGGCCCG |
| | | ATGCCACCTCCTCGCCTCCTCTTCTTCCTCCTCTTCCTCACCCCCATGGAAGT |
| | | CAGGCCCGAGGAACCTCTAGTGGTGAAGGTGGAAGAGGGAGATAACGCTGTGC |
| | | TGCAGTGCCTCAAGGGGACCTCAGATGGCCCCACTCAGCAGCTGACCTGGTCT |
| | | CGGGAGTCCCCGCTTAAACCCTTCTTAAAACTCAGCCTGGGGCTGCCAGGCCT |
| | | GGGAATCCACATGAGGCCCCTGGCCATCTGGCTTTTCATCTTCAACGTCTCTC |
| | | AACAGATGGGGGGCTTCTACCTGTGCCAGCCGGGGCCCCCCTCTGAGAAGGCC |
| | | TGGCAGCCTGGCTGGACAGTCAATGTGGAGGGCAGCGGGGAGCTGTTCCGGTG |
| | | GAATGTTTCGGACCTAGGTGGCCTGGGCTGTGGCCTGAAGAACAGGTCCTCAG |
| | | AGGGCCCCAGCTCCCCTTCCGGGAAGCTCATGAGCCCCAAGCTGTATGTGTGG |
| | | GCCAAAGACCGCCCTGAGATCTGGGAGGGAGAGCCTCCGTGTCTCCCACCGAG |
| | | GGACAGCCTGAACCAGAGCCTCAGCCAGGACCTCACCATGGCCCCTGGCTCCA |
| | | CACTCTGGCTGTCCTGTGGGGTACCCCCTGACTCTGTGTCCAGGGGCCCCCTC |
| | | TCCTGGACCCATGTGCACCCCAAGGGGCCTAAGTCATTGCTGAGCCTAGAGCT |
| | | GAAGGACGATCGCCCGGCCAGAGATATGTGGGTAATGGAGACGGGTCTGTTGT |
| | | TGCCCCGGGCCACAGCTCAAGACGCTGGAAAGTATTATTGTCACCGTGGCAAC |
| | | CTGACCATGTCATTCCACCTGGAGATCACTGCTCGGCCAGTACTATGGCACTG |
| | | GCTGCTGAGGACTGGTGGCTGGAAGGTCTCAGCTGTGACTTTGGCTTATCTGA |
| | | TCTTCTGCCTGTGTTCCCTTGTGGGCATTCTTCATCTTCAAAGAGCCCTGGTC |
| | | CTGAGGAGGAAAAGAAAGCGAATGACTGACCCCACCAGGAGATTC |
| 2 | PG9 Full | ATGGGATGGTCATGTATCATCCTTTTTCTAGTAGCAACTGCAACCGGTGTACA |
| | length | TTCACAGTCTGCCCTGACTCAGCCTGCCTCCGTGTCTGGGTCTCCTGGACAGT |
| | Antibody- | CGATCACCATCTCCTGCAATGGAACCAGCAATGATGTTGGTGGCTATGAATCT |
| | nucleic acid | GTCTCCTGGTACCAACAACATCCCGGCAAAGCCCCCAAAGTCGTGATTTATGA |
| | encoded | TGTCAGTAAACGGCCCTCAGGGGTTTCTAATCGCTTCTCTGGCTCCAAGTCCG |
| | region | GCAACACGGCCTCCCTGACCATCTCTGGGCTCCAGGCTGAGGACGAGGGTGAC |
| | | TATTACTGCAAGTCTCTGACAAGCACGAGACGTCGGGTTTTCGGCACTGGGAC |
| | | CAAGCTGACCGTTCTAACCGTGGCGGCGCCGAGCGTGTTTATTTTTCCGCCGA |
| | | GCGATGAACAGCTGAAAAGCGGCACCGCGAGCGTGGTGTGCCTGCTGAACAAC |
| | | TTTTATCCGCGCGAAGCGAAAGTGCAGTGGAAAGTGGATAACGCGCTGCAGAG |
| | | CGGCAACAGCCAGGAAAGCGTGACCGAACAGGATAGCAAAGATAGCACCTATA |
| | | GCCTGAGCAGCACCCTGACCCTGAGCAAAGCGGATTATGAAAAACATAAAGTG |
| | | TATGCGTGCGAAGTGACCCATCAGGGCCTGAGCAGCCCGGTGACCAAAAGCTT |
| | | TAACCGCGGCGAATGCCGCAAACGCCGCGGCAGCGGCGCGACCAACTTTAGCC |
| | | TGCTGAAACAGGCGGGCGATGTGGAAGAAAACCCGGGCCCGATGGGATGGTCA |
| | | TGTATCATCCTTTTTCTAGTAGCAACTGCAACCGGTGTACATTCACAGCGATT |
| | | AGTGGAGTCTGGGGGAGGCGTGGTCCAGCCTGGGTCGTCCCTGAGACTCTCCT |
| | | GTGCAGCGTCCGGATTCGACTTCAGTAGACAAGGCATGCACTGGGTCCGCCAG |
| | | GCTCCAGGCCAGGGGCTGGAGTGGGTGGCATTTATTAAATATGATGGAAGTGA |
| | | GAAATATCATGCTGACTCCGTATGGGGCCGACTCAGCATCTCCAGAGACAATT |
| | | CCAAGGATACGCTTTATCTCCAAATGAATAGCCTGAGAGTCGAGGACACGGCT |
| | | ACATATTTTTGTGTGAGAGAGGCTGGTGGGCCCGACTACCGTAATGGGTACAA |
| | | CTATTACGATTTCTATGATGGTTATTATAACTACCACTATATGGACGTCTGGG |
| | | GCAAAGGGACCACGGTCACCGTCTCGAGCGCGAGCACCAAAGGCCCGAGCGTG |
| | | TTTCCGCTGGCGCCGTGCAGCCGCAGCACCAGCGGCGGCACCGCGGCGCTGGG |
| | | CTGCCTGGTGAAAGATTATTTTCCGGAACCGGTGACCGTGAGCTGGAACAGCG |
| | | GCGCGCTGACCAGCGGCGTGCATACCTTTCCGGCGGTGCTGCAGAGCAGCGGC |
| | | CTGTATAGCCTGAGCAGCGTGGTGACCGTGCCGAGCAGCAGCCTGGGCACCCA |
| | | GACCTATACCTGCAACGTGAACCATAAACCGAGCAACACCAAAGTGGATAAAC |
| | | GCGTGGAACTGAAAACCCCGCTGGGCGATACCACCCATACCTGCCCGCGCTGC |
| | | CCGGAACCGAAAAGCTGCGATACCCCGCCGCCGTGCCCGCGCTGCCCGGAACC |
| | | GAAAAGCTGCGATACCCCGCCGCCGTGCCCGCGCTGCCCGGAACCGAAAAGCT |
| | | GCGATACCCCGCCGCCGTGCCCGCGCTGCCCGGCGCCGGAACTGCTGGGCGGC |
| | | CCGAGCGTGTTTCTGTTTCCGCCGAAACCGAAAGATACCCTGATGATTAGCCG |
| | | CACCCCGGAAGTGACCTGCGTGGTGGTGGATGTGAGCCATGAAGATCCGGAAG |
| | | TGCAGTTTAAATGGTATGTGGATGGCGTGGAAGTGCATAACGCGAAAACCAAA |
| | | CCGCGCGAAGAACAGTATAACAGCACCTTTCGCGTGGTGAGCGTGCTGACCGT |
| | | GCTGCATCAGGATTGGCTGAACGGCAAAGAATATAAATGCAAAGTGAGCAACA |
| | | AAGCGCTGCCGGCGCCGATTGAAAAAACCATTAGCAAAACCAAAGGCCAGCCG |
| | | CGCGAACCGCAGGTGTATACCCTGCCGCCGAGCCGCGAAGAAATGACCAAAAA |
| | | CCAGGTGAGCCTGACCTGCCTGGTGAAAGGCTTTTATCCGAGCGATATTGCGG |
| | | TGGAATGGGAAAGCAGCGGCCAGCCGGAAAACAACTATAACACCACCCCGCCG |
| | | ATGCTGGATAGCGATGGCAGCTTTTTTCTGTATAGCAAACTGACCGTGGATAA |
| | | AAGCCGCTGGCAGCAGGGCAACATTTTTAGCTGCAGCGTGATGCATGAAGCGC |
| | | TGCATAACCGCTTTACCCAGAAAAGCCTGAGCCTGAGCCCGGGCAAACGCAAA |
| | | CGCCGCGGCAGCGGCGCGACCAACTTTAGCCTGCTGAAACAGGCGGGCGATGT |
| | | GGAAGAAAACCCGGGCCCGATGCCACCTCCTCGCCTCCTCTTCTTCCTCCTCT |
| | | TCCTCACCCCCATGGAAGTCAGGCCCGAGGAACCTCTAGTGGTGAAGGTGGAA |
| | | GAGGGAGATAACGCTGTGCTGCAGTGCCTCAAGGGGACCTCAGATGGCCCCAC |
| | | TCAGCAGCTGACCTGGTCTCGGGAGTCCCCGCTTAAACCCTTCTTAAAACTCA |
| | | GCCTGGGGCTGCCAGGCCTGGGAATCCACATGAGGCCCCTGGCCATCTGGCTT |
| | | TTCATCTTCAACGTCTCTCAACAGATGGGGGGCTTCTACCTGTGCCAGCCGGG |
| | | GCCCCCCTCTGAGAAGGCCTGGCAGCCTGGCTGGACAGTCAATGTGGAGGGCA |
| | | GCGGGGAGCTGTTCCGGTGGAATGTTTCGGACCTAGGTGGCCTGGGCTGTGGC |
| | | CTGAAGAACAGGTCCTCAGAGGGCCCCAGCTCCCCTTCCGGGAAGCTCATGAG |
| | | CCCCAAGCTGTATGTGTGGGCCAAAGACCGCCCTGAGATCTGGGAGGGAGAGC |
| | | CTCCGTGTCTCCCACCGAGGGACAGCCTGAACCAGAGCCTCAGCCAGGACCTC |
| | | ACCATGGCCCCTGGCTCCACACTCTGGCTGTCCTGTGGGGTACCCCCTGACTC |
| | | TGTGTCCAGGGGCCCCCTCTCCTGGACCCATGTGCACCCCAAGGGGCCTAAGT |
| | | CATTGCTGAGCCTAGAGCTGAAGGACGATCGCCCGGCCAGAGATATGTGGGTA |
| | | ATGGAGACGGGTCTGTTGTTGCCCCGGGCCACAGCTCAAGACGCTGGAAAGTA |
| | | TTATTGTCACCGTGGCAACCTGACCATGTCATTCCACCTGGAGATCACTGCTC |
| | | GGCCAGTACTATGGCACTGGCTGCTGAGGACTGGTGGCTGGAAGGTCTCAGCT |
| | | GTGACTTTGGCTTATCTGATCTTCTGCCTGTGTTCCCTTGTGGGCATTCTTCA |
| | | TCTTCAAAGAGCCCTGGTCCTGAGGAGGAAAAGAAAGCGAATGACTGACCCCA |
| | | CCAGGAGATTC |
| 3 | PG9 light | CGTTACATAACTTACGGTAAATGGCCCGCCTGGCTGACCGCCCAACGACCCCC |
| | chain | GCCCATTGACGTCAATAATGACGTATGTTCCCATAGTAACGCCAATAGGGACT |
| | nucleic acid | TTCCATTGACGTCAATGGGTGGAGTATTTACGGTAAACTGCCCACTTGGCAGT |
| | sequence | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACCATGGGATGGTCATGTATCA |
| | | TCCTTTTTCTAGTAGCAACTGCAACCGGTGTACATTCACAGTCTGCCCTGACT |
| | | CAGCCTGCCTCCGTGTCTGGGTCTCCTGGACAGTCGATCACCATCTCCTGCAA |
| | | TGGAACCAGCAATGATGTTGGTGGCTATGAATCTGTCTCCTGGTACCAACAAC |
| | | ATCCCGGCAAAGCCCCCAAAGTCGTGATTTATGATGTCAGTAAACGGCCCTCA |
| | | GGGGTTTCTAATCGCTTCTCTGGCTCCAAGTCCGGCAACACGGCCTCCCTGAC |
| | | CATCTCTGGGCTCCAGGCTGAGGACGAGGGTGACTATTACTGCAAGTCTCTGA |
| | | CAAGCACGAGACGTCGGGTTTTCGGCACTGGGACCAAGCTGACCGTTCTAACC |
| | | GTGGCGGCGCCGAGCGTGTTTATTTTTCCGCCGAGCGATGAACAGCTGAAAAG |
| | | CGGCACCGCGAGCGTGGTGTGCCTGCTGAACAACTTTTATCCGCGCGAAGCGA |
| | | AAGTGCAGTGGAAAGTGGATAACGCGCTGCAGAGCGGCAACAGCCAGGAAAGC |
| | | GTGACCGAACAGGATAGCAAAGATAGCACCTATAGCCTGAGCAGCACCCTGAC |
| | | CCTGAGCAAAGCGGATTATGAAAAACATAAAGTGTATGCGTGCGAAGTGACCC |
| | | ATCAGGGCCTGAGCAGCCCGGTGACCAAAAGCTTTAACCGCGGCGAATGCCGC |
| | | AAACGCCGCGGCAGCGGCGCGACCAACTTTAGCCTGCTGAAACAGGCGGGCGA |
| | | TGTGGAAGAAAACCCGGGCCCGATGCCACCTCCTCGCCTCCTCTTCTTCCTCC |
| | | TCTTCCTCACCCCCATGGAAGTCAGGCCCGAGGAACCTCTAGTGGTGAAGGTG |
| | | GAAGAGGGAGATAACGCTGTGCTGCAGTGCCTCAAGGGGACCTCAGATGGCCC |
| | | CACTCAGCAGCTGACCTGGTCTCGGGAGTCCCCGCTTAAACCCTTCTTAAAAC |
| | | TCAGCCTGGGGCTGCCAGGCCTGGGAATCCACATGAGGCCCCTGGCCATCTGG |
| | | CTTTTCATCTTCAACGTCTCTCAACAGATGGGGGGCTTCTACCTGTGCCAGCC |
| | | GGGGCCCCCCTCTGAGAAGGCCTGGCAGCCTGGCTGGACAGTCAATGTGGAGG |
| | | GCAGCGGGGAGCTGTTCCGGTGGAATGTTTCGGACCTAGGTGGCCTGGGCTGT |
| | | GGCCTGAAGAACAGGTCCTCAGAGGGCCCCAGCTCCCCTTCCGGGAAGCTCAT |
| | | GAGCCCCAAGCTGTATGTGTGGGCCAAAGACCGCCCTGAGATCTGGGAGGGAG |
| | | AGCCTCCGTGTCTCCCACCGAGGGACAGCCTGAACCAGAGCCTCAGCCAGGAC |
| | | CTCACCATGGCCCCTGGCTCCACACTCTGGCTGTCCTGTGGGGTACCCCCTGA |
| | | CTCTGTGTCCAGGGGCCCCCTCTCCTGGACCCATGTGCACCCCAAGGGGCCTA |
| | | AGTCATTGCTGAGCCTAGAGCTGAAGGACGATCGCCCGGCCAGAGATATGTGG |
| | | GTAATGGAGACGGGTCTGTTGTTGCCCCGGGCCACAGCTCAAGACGCTGGAAA |
| | | GTATTATTGTCACCGTGGCAACCTGACCATGTCATTCCACCTGGAGATCACTG |
| | | CTCGGCCAGTACTATGGCACTGGCTGCTGAGGACTGGTGGCTGGAAGGTCTCA |
| | | GCTGTGACTTTGGCTTATCTGATCTTCTGCCTGTGTTCCCTTGTGGGCATTCT |
| | | TCATCTTCAAAGAGCCCTGGTCCTGAGGAGGAAAAGAAAGCGAATGACTGACC |
| | | CCACCAGGAGATTC |
| 4 | PG9 light | ATGGGATGGTCATGTATCATCCTTTTTCTAGTAGCAACTGCAACCGGTGTACA |
| | chain | TTCACAGTCTGCCCTGACTCAGCCTGCCTCCGTGTCTGGGTCTCCTGGACAGT |
| | nucleic acid | CGATCACCATCTCCTGCAATGGAACCAGCAATGATGTTGGTGGCTATGAATCT |
| | sequence- | GTCTCCTGGTACCAACAACATCCCGGCAAAGCCCCCAAAGTCGTGATTTATGA |
| | encoded | TGTCAGTAAACGGCCCTCAGGGGTTTCTAATCGCTTCTCTGGCTCCAAGTCCG |
| | region | GCAACACGGCCTCCCTGACCATCTCTGGGCTCCAGGCTGAGGACGAGGGTGAC |
| | | TATTACTGCAAGTCTCTGACAAGCACGAGACGTCGGGTTTTCGGCACTGGGAC |
| | | CAAGCTGACCGTTCTAACCGTGGCGGCGCCGAGCGTGTTTATTTTTCCGCCGA |
| | | GCGATGAACAGCTGAAAAGCGGCACCGCGAGCGTGGTGTGCCTGCTGAACAAC |
| | | TTTTATCCGCGCGAAGCGAAAGTGCAGTGGAAAGTGGATAACGCGCTGCAGAG |
| | | CGGCAACAGCCAGGAAAGCGTGACCGAACAGGATAGCAAAGATAGCACCTATA |
| | | GCCTGAGCAGCACCCTGACCCTGAGCAAAGCGGATTATGAAAAACATAAAGTG |
| | | TATGCGTGCGAAGTGACCCATCAGGGCCTGAGCAGCCCGGTGACCAAAAGCTT |
| | | TAACCGCGGCGAATGCCGCAAACGCCGCGGCAGCGGCGCGACCAACTTTAGCC |
| | | TGCTGAAACAGGCGGGCGATGTGGAAGAAAACCCGGGCCCGATGCCACCTCCT |
| | | CGCCTCCTCTTCTTCCTCCTCTTCCTCACCCCCATGGAAGTCAGGCCCGAGGA |
| | | ACCTCTAGTGGTGAAGGTGGAAGAGGGAGATAACGCTGTGCTGCAGTGCCTCA |
| | | AGGGGACCTCAGATGGCCCCACTCAGCAGCTGACCTGGTCTCGGGAGTCCCCG |
| | | CTTAAACCCTTCTTAAAACTCAGCCTGGGGCTGCCAGGCCTGGGAATCCACAT |
| | | GAGGCCCCTGGCCATCTGGCTTTTCATCTTCAACGTCTCTCAACAGATGGGGG |
| | | GCTTCTACCTGTGCCAGCCGGGGCCCCCCTCTGAGAAGGCCTGGCAGCCTGGC |
| | | TGGACAGTCAATGTGGAGGGCAGCGGGGAGCTGTTCCGGTGGAATGTTTCGGA |
| | | CCTAGGTGGCCTGGGCTGTGGCCTGAAGAACAGGTCCTCAGAGGGCCCCAGCT |
| | | CCCCTTCCGGGAAGCTCATGAGCCCCAAGCTGTATGTGTGGGCCAAAGACCGC |
| | | CCTGAGATCTGGGAGGGAGAGCCTCCGTGTCTCCCACCGAGGGACAGCCTGAA |
| | | CCAGAGCCTCAGCCAGGACCTCACCATGGCCCCTGGCTCCACACTCTGGCTGT |
| | | CCTGTGGGGTACCCCCTGACTCTGTGTCCAGGGGCCCCCTCTCCTGGACCCAT |
| | | GTGCACCCCAAGGGGCCTAAGTCATTGCTGAGCCTAGAGCTGAAGGACGATCG |
| | | CCCGGCCAGAGATATGTGGGTAATGGAGACGGGTCTGTTGTTGCCCCGGGCCA |
| | | CAGCTCAAGACGCTGGAAAGTATTATTGTCACCGTGGCAACCTGACCATGTCA |
| | | TTCCACCTGGAGATCACTGCTCGGCCAGTACTATGGCACTGGCTGCTGAGGAC |
| | | TGGTGGCTGGAAGGTCTCAGCTGTGACTTTGGCTTATCTGATCTTCTGCCTGT |
| | | GTTCCCTTGTGGGCATTCTTCATCTTCAAAGAGCCCTGGTCCTGAGGAGGAAA |
| | | AGAAAGCGAATGACTGACCCCACCAGGAGATTC |
| 5 | PG9 heavy | CGTTACATAACTTACGGTAAATGGCCCGCCTGGCTGACCGCCCAACGACCCCC |
| | chain | GCCCATTGACGTCAATAATGACGTATGTTCCCATAGTAACGCCAATAGGGACT |
| | nucleic acid | TTCCATTGACGTCAATGGGTGGAGTATTTACGGTAAACTGCCCACTTGGCAGT |
| | sequence | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACCATGGGATGGTCATGTATCA |
| | | TCCTTTTTCTAGTAGCAACTGCAACCGGTGTACATTCACAGCGATTAGTGGAG |
| | | TCTGGGGGAGGCGTGGTCCAGCCTGGGTCGTCCCTGAGACTCTCCTGTGCAGC |
| | | GTCCGGATTCGACTTCAGTAGACAAGGCATGCACTGGGTCCGCCAGGCTCCAG |
| | | GCCAGGGGCTGGAGTGGGTGGCATTTATTAAATATGATGGAAGTGAGAAATAT |
| | | CATGCTGACTCCGTATGGGGCCGACTCAGCATCTCCAGAGACAATTCCAAGGA |
| | | TACGCTTTATCTCCAAATGAATAGCCTGAGAGTCGAGGACACGGCTACATATT |
| | | TTTGTGTGAGAGAGGCTGGTGGGCCCGACTACCGTAATGGGTACAACTATTAC |
| | | GATTTCTATGATGGTTATTATAACTACCACTATATGGACGTCTGGGGCAAAGG |
| | | GACCACGGTCACCGTCTCGAGCGCGAGCACCAAAGGCCCGAGCGTGTTTCCGC |
| | | TGGCGCCGTGCAGCCGCAGCACCAGCGGCGGCACCGCGGCGCTGGGCTGCCTG |
| | | GTGAAAGATTATTTTCCGGAACCGGTGACCGTGAGCTGGAACAGCGGCGCGCT |
| | | GACCAGCGGCGTGCATACCTTTCCGGCGGTGCTGCAGAGCAGCGGCCTGTATA |
| | | GCCTGAGCAGCGTGGTGACCGTGCCGAGCAGCAGCCTGGGCACCCAGACCTAT |
| | | ACCTGCAACGTGAACCATAAACCGAGCAACACCAAAGTGGATAAACGCGTGGA |
| | | ACTGAAAACCCCGCTGGGCGATACCACCCATACCTGCCCGCGCTGCCCGGAAC |
| | | CGAAAAGCTGCGATACCCCGCCGCCGTGCCCGCGCTGCCCGGAACCGAAAAGC |
| | | TGCGATACCCCGCCGCCGTGCCCGCGCTGCCCGGAACCGAAAAGCTGCGATAC |
| | | CCCGCCGCCGTGCCCGCGCTGCCCGGCGCCGGAACTGCTGGGCGGCCCGAGCG |
| | | TGTTTCTGTTTCCGCCGAAACCGAAAGATACCCTGATGATTAGCCGCACCCCG |
| | | GAAGTGACCTGCGTGGTGGTGGATGTGAGCCATGAAGATCCGGAAGTGCAGTT |
| | | TAAATGGTATGTGGATGGCGTGGAAGTGCATAACGCGAAAACCAAACCGCGCG |
| | | AAGAACAGTATAACAGCACCTTTCGCGTGGTGAGCGTGCTGACCGTGCTGCAT |
| | | CAGGATTGGCTGAACGGCAAAGAATATAAATGCAAAGTGAGCAACAAAGCGCT |
| | | GCCGGCGCCGATTGAAAAAACCATTAGCAAAACCAAAGGCCAGCCGCGCGAAC |
| | | CGCAGGTGTATACCCTGCCGCCGAGCCGCGAAGAAATGACCAAAAACCAGGTG |
| | | AGCCTGACCTGCCTGGTGAAAGGCTTTTATCCGAGCGATATTGCGGTGGAATG |
| | | GGAAAGCAGCGGCCAGCCGGAAAACAACTATAACACCACCCCGCCGATGCTGG |
| | | ATAGCGATGGCAGCTTTTTTCTGTATAGCAAACTGACCGTGGATAAAAGCCGC |
| | | TGGCAGCAGGGCAACATTTTTAGCTGCAGCGTGATGCATGAAGCGCTGCATAA |
| | | CCGCTTTACCCAGAAAAGCCTGAGCCTGAGCCCGGGCAAACGCAAACGCCGCG |
| | | GCAGCGGCGCGACCAACTTTAGCCTGCTGAAACAGGCGGGCGATGTGGAAGAA |
| | | AACCCGGGCCCGATGCCACCTCCTCGCCTCCTCTTCTTCCTCCTCTTCCTCAC |
| | | CCCCATGGAAGTCAGGCCCGAGGAACCTCTAGTGGTGAAGGTGGAAGAGGGAG |
| | | ATAACGCTGTGCTGCAGTGCCTCAAGGGGACCTCAGATGGCCCCACTCAGCAG |
| | | CTGACCTGGTCTCGGGAGTCCCCGCTTAAACCCTTCTTAAAACTCAGCCTGGG |
| | | GCTGCCAGGCCTGGGAATCCACATGAGGCCCCTGGCCATCTGGCTTTTCATCT |
| | | TCAACGTCTCTCAACAGATGGGGGGCTTCTACCTGTGCCAGCCGGGGCCCCCC |
| | | TCTGAGAAGGCCTGGCAGCCTGGCTGGACAGTCAATGTGGAGGGCAGCGGGGA |
| | | GCTGTTCCGGTGGAATGTTTCGGACCTAGGTGGCCTGGGCTGTGGCCTGAAGA |
| | | ACAGGTCCTCAGAGGGCCCCAGCTCCCCTTCCGGGAAGCTCATGAGCCCCAAG |
| | | CTGTATGTGTGGGCCAAAGACCGCCCTGAGATCTGGGAGGGAGAGCCTCCGTG |
| | | TCTCCCACCGAGGGACAGCCTGAACCAGAGCCTCAGCCAGGACCTCACCATGG |
| | | CCCCTGGCTCCACACTCTGGCTGTCCTGTGGGGTACCCCCTGACTCTGTGTCC |
| | | AGGGGCCCCCTCTCCTGGACCCATGTGCACCCCAAGGGGCCTAAGTCATTGCT |
| | | GAGCCTAGAGCTGAAGGACGATCGCCCGGCCAGAGATATGTGGGTAATGGAGA |
| | | CGGGTCTGTTGTTGCCCCGGGCCACAGCTCAAGACGCTGGAAAGTATTATTGT |
| | | CACCGTGGCAACCTGACCATGTCATTCCACCTGGAGATCACTGCTCGGCCAGT |
| | | ACTATGGCACTGGCTGCTGAGGACTGGTGGCTGGAAGGTCTCAGCTGTGACTT |
| | | TGGCTTATCTGATCTTCTGCCTGTGTTCCCTTGTGGGCATTCTTCATCTTCAA |
| | | AGAGCCCTGGTCCTGAGGAGGAAAAGAAAGCGAATGACTGACCCCACCAGGAG |
| | | ATTC |
| 6 | PG9 heavy | ATGGGATGGTCATGTATCATCCTTTTTCTAGTAGCAACTGCAACCGGTGTACA |
| | chain | TTCACAGCGATTAGTGGAGTCTGGGGGAGGCGTGGTCCAGCCTGGGTCGTCCC |
| | nucleic acid | TGAGACTCTCCTGTGCAGCGTCCGGATTCGACTTCAGTAGACAAGGCATGCAC |
| | sequence- | TGGGTCCGCCAGGCTCCAGGCCAGGGGCTGGAGTGGGTGGCATTTATTAAATA |
| | encoded | TGATGGAAGTGAGAAATATCATGCTGACTCCGTATGGGGCCGACTCAGCATCT |
| | region | CCAGAGACAATTCCAAGGATACGCTTTATCTCCAAATGAATAGCCTGAGAGTC |
| | | GAGGACACGGCTACATATTTTTGTGTGAGAGAGGCTGGTGGGCCCGACTACCG |
| | | TAATGGGTACAACTATTACGATTTCTATGATGGTTATTATAACTACCACTATA |
| | | TGGACGTCTGGGGCAAAGGGACCACGGTCACCGTCTCGAGCGCGAGCACCAAA |
| | | GGCCCGAGCGTGTTTCCGCTGGCGCCGTGCAGCCGCAGCACCAGCGGCGGCAC |
| | | CGCGGCGCTGGGCTGCCTGGTGAAAGATTATTTTCCGGAACCGGTGACCGTGA |
| | | GCTGGAACAGCGGCGCGCTGACCAGCGGCGTGCATACCTTTCCGGCGGTGCTG |
| | | CAGAGCAGCGGCCTGTATAGCCTGAGCAGCGTGGTGACCGTGCCGAGCAGCAG |
| | | CCTGGGCACCCAGACCTATACCTGCAACGTGAACCATAAACCGAGCAACACCA |
| | | AAGTGGATAAACGCGTGGAACTGAAAACCCCGCTGGGCGATACCACCCATACC |
| | | TGCCCGCGCTGCCCGGAACCGAAAAGCTGCGATACCCCGCCGCCGTGCCCGCG |
| | | CTGCCCGGAACCGAAAAGCTGCGATACCCCGCCGCCGTGCCCGCGCTGCCCGG |
| | | AACCGAAAAGCTGCGATACCCCGCCGCCGTGCCCGCGCTGCCCGGCGCCGGAA |
| | | CTGCTGGGCGGCCCGAGCGTGTTTCTGTTTCCGCCGAAACCGAAAGATACCCT |
| | | GATGATTAGCCGCACCCCGGAAGTGACCTGCGTGGTGGTGGATGTGAGCCATG |
| | | AAGATCCGGAAGTGCAGTTTAAATGGTATGTGGATGGCGTGGAAGTGCATAAC |
| | | GCGAAAACCAAACCGCGCGAAGAACAGTATAACAGCACCTTTCGCGTGGTGAG |
| | | CGTGCTGACCGTGCTGCATCAGGATTGGCTGAACGGCAAAGAATATAAATGCA |
| | | AAGTGAGCAACAAAGCGCTGCCGGCGCCGATTGAAAAAACCATTAGCAAAACC |
| | | AAAGGCCAGCCGCGCGAACCGCAGGTGTATACCCTGCCGCCGAGCCGCGAAGA |
| | | AATGACCAAAAACCAGGTGAGCCTGACCTGCCTGGTGAAAGGCTTTTATCCGA |
| | | GCGATATTGCGGTGGAATGGGAAAGCAGCGGCCAGCCGGAAAACAACTATAAC |
| | | ACCACCCCGCCGATGCTGGATAGCGATGGCAGCTTTTTTCTGTATAGCAAACT |
| | | GACCGTGGATAAAAGCCGCTGGCAGCAGGGCAACATTTTTAGCTGCAGCGTGA |
| | | TGCATGAAGCGCTGCATAACCGCTTTACCCAGAAAAGCCTGAGCCTGAGCCCG |
| | | GGCAAACGCAAACGCCGCGGCAGCGGCGCGACCAACTTTAGCCTGCTGAAACA |
| | | GGCGGGCGATGTGGAAGAAAACCCGGGCCCGATGCCACCTCCTCGCCTCCTCT |
| | | TCTTCCTCCTCTTCCTCACCCCCATGGAAGTCAGGCCCGAGGAACCTCTAGTG |
| | | GTGAAGGTGGAAGAGGGAGATAACGCTGTGCTGCAGTGCCTCAAGGGGACCTC |
| | | AGATGGCCCCACTCAGCAGCTGACCTGGTCTCGGGAGTCCCCGCTTAAACCCT |
| | | TCTTAAAACTCAGCCTGGGGCTGCCAGGCCTGGGAATCCACATGAGGCCCCTG |
| | | GCCATCTGGCTTTTCATCTTCAACGTCTCTCAACAGATGGGGGGCTTCTACCT |
| | | GTGCCAGCCGGGGCCCCCCTCTGAGAAGGCCTGGCAGCCTGGCTGGACAGTCA |
| | | ATGTGGAGGGCAGCGGGGAGCTGTTCCGGTGGAATGTTTCGGACCTAGGTGGC |
| | | CTGGGCTGTGGCCTGAAGAACAGGTCCTCAGAGGGCCCCAGCTCCCCTTCCGG |
| | | GAAGCTCATGAGCCCCAAGCTGTATGTGTGGGCCAAAGACCGCCCTGAGATCT |
| | | GGGAGGGAGAGCCTCCGTGTCTCCCACCGAGGGACAGCCTGAACCAGAGCCTC |
| | | AGCCAGGACCTCACCATGGCCCCTGGCTCCACACTCTGGCTGTCCTGTGGGGT |
| | | ACCCCCTGACTCTGTGTCCAGGGGCCCCCTCTCCTGGACCCATGTGCACCCCA |
| | | AGGGGCCTAAGTCATTGCTGAGCCTAGAGCTGAAGGACGATCGCCCGGCCAGA |
| | | GATATGTGGGTAATGGAGACGGGTCTGTTGTTGCCCCGGGCCACAGCTCAAGA |
| | | CGCTGGAAAGTATTATTGTCACCGTGGCAACCTGACCATGTCATTCCACCTGG |
| | | AGATCACTGCTCGGCCAGTACTATGGCACTGGCTGCTGAGGACTGGTGGCTGG |
| | | AAGGTCTCAGCTGTGACTTTGGCTTATCTGATCTTCTGCCTGTGTTCCCTTGT |
| | | GGGCATTCTTCATCTTCAAAGAGCCCTGGTCCTGAGGAGGAAAAGAAAGCGAA |
| | | TGACTGACCCCACCAGGAGATTC |
| 7 | Homo sapiens | CAGTCTGCCCTGACTCAGCCTGCCTCCGTGTCTGGGTCTCCTGGACAGTCGAT |
| | isolate PG9 | CACCATCTCCTGCAATGGAACCAGCAATGATGTTGGTGGCTATGAATCTGTCT |
| | anti-HIV | CCTGGTACCAACAACATCCCGGCAAAGCCCCCAAAGTCGTGATTTATGATGTC |
| | immuno- | AGTAAACGGCCCTCAGGGGTTTCTAATCGCTTCTCTGGCTCCAAGTCCGGCAA |
| | globulin | CACGGCCTCCCTGACCATCTCTGGGCTCCAGGCTGAGGACGAGGGTGACTATT |
| | light chain | ACTGCAAGTCTCTGACAAGCACGAGACGTCGGGTTTTCGGCACTGGGACCAAG |
| | variable | CTGACCGTTCTA |
| | region mRNA | |
| 8 | Homo sapiens | CAGCGATTAGTGGAGTCTGGGGGAGGCGTGGTCCAGCCTGGGTCGTCCCTGAG |
| | isolate PG9 | ACTCTCCTGTGCAGCGTCCGGATTCGACTTCAGTAGACAAGGCATGCACTGGG |
| | anti-HIV | TCCGCCAGGCTCCAGGCCAGGGGCTGGAGTGGGTGGCATTTATTAAATATGAT |
| | immuno- | GGAAGTGAGAAATATCATGCTGACTCCGTATGGGGCCGACTCAGCATCTCCAG |
| | globulin | AGACAATTCCAAGGATACGCTTTATCTCCAAATGAATAGCCTGAGAGTCGAGG |
| | heavy chain | ACACGGCTACATATTTTTGTGTGAGAGAGGCTGGTGGGCCCGACTACCGTAAT |
| | variable | GGGTACAACTATTACGATTTCTATGATGGTTATTATAACTACCACTATATGGA |
| | region mRNA | CGTCTGGGGCAAAGGGACCACGGTCACCGTCTCGAGC |
| 9 | PG9 scFv | CGTTACATAACTTACGGTAAATGGCCCGCCTGGCTGACCGCCCAACGACCCCC |
| | nucleic acid | GCCCATTGACGTCAATAATGACGTATGTTCCCATAGTAACGCCAATAGGGACT |
| | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACCATGGATTGGATTTGGCGCA |
| | | TTCTGTTTCTGGTGGGCGCGGCGACCGGCGCGCATAGCGAAGTGCAGCTGGTG |
| | | GAAAGCGGCGGCGGCGTGGTGCGCCCGGGCGGCAGCCTGCGCCTGAGCTGCGC |
| | | GGCGAGCGGCTTTACCTTTGATGATTATGGCATGAGCTGGGTGCGCCAGGCGC |
| | | CGGGCAAAGGCCTGGAATGGGTGAGCGGCATTAACTGGAACGGCGGCAGCACC |
| | | GGCTATGCGGATAGCGTGAAAGGCCGCTTTACCATTAGCCGCGATAACGCGAA |
| | | AAACAGCCTGTATCTGCAGATGAACAGCCTGCGCGCGGAAGATACCGCGGTGT |
| | | ATTATTGCGCGCGCGGCCGCAGCCTGCTGTTTGATTATTGGGGCCAGGGCACC |
| | | CTGGTGACCGTGAGCCGCGGCGGCGGCGGCAGCGGCGGCGGCGGCAGCGGCGG |
| | | CGGCGGCAGCGGCGGCGGCGGCAGCAGCAGCGAACTGACCCAGGATCCGGCGG |
| | | TGAGCGTGGCGCTGGGCCAGACCGTGCGCATTACCTGCCAGGGCGATAGCCTG |
| | | CGCAGCTATTATGCGAGCTGGTATCAGCAGAAACCGGGCCAGGCGCCGGTGCT |
| | | GGTGATTTATGGCAAAAACAACCGCCCGAGCGGCATTCCGGATCGCTTTAGCG |
| | | GCAGCAGCAGCGGCAACACCGCGAGCCTGACCATTACCGGCGCGCAGGCGGAA |
| | | GATGAAGCGGATTATTATTGCAACAGCCGCGATAGCAGCGGCAACCATGTGGT |
| | | GTTTGGCGGCGGCACCAAACTGACCGTGGGCAGCGGCGGCGGCGGCAGCCAGC |
| | | GATTAGTGGAGTCTGGGGGAGGCGTGGTCCAGCCTGGGTCGTCCCTGAGACTC |
| | | TCCTGTGCAGCGTCCGGATTCGACTTCAGTAGACAAGGCATGCACTGGGTCCG |
| | | CCAGGCTCCAGGCCAGGGGCTGGAGTGGGTGGCATTTATTAAATATGATGGAA |
| | | GTGAGAAATATCATGCTGACTCCGTATGGGGCCGACTCAGCATCTCCAGAGAC |
| | | AATTCCAAGGATACGCTTTATCTCCAAATGAATAGCCTGAGAGTCGAGGACAC |
| | | GGCTACATATTTTTGTGTGAGAGAGGCTGGTGGGCCCGACTACCGTAATGGGT |
| | | ACAACTATTACGATTTCTATGATGGTTATTATAACTACCACTATATGGACGTC |
| | | TGGGGCAAAGGGACCACGGTCACCGTCTCGAGCGGCGGCGGCGGCAGCGGCGG |
| | | CGGCGGCAGCGGCGGCGGCGGCAGCGGCGGCGGCGGCAGCCAGTCTGCCCTGA |
| | | CTCAGCCTGCCTCCGTGTCTGGGTCTCCTGGACAGTCGATCACCATCTCCTGC |
| | | AATGGAACCAGCAATGATGTTGGTGGCTATGAATCTGTCTCCTGGTACCAACA |
| | | ACATCCCGGCAAAGCCCCCAAAGTCGTGATTTATGATGTCAGTAAACGGCCCT |
| | | CAGGGGTTTCTAATCGCTTCTCTGGCTCCAAGTCCGGCAACACGGCCTCCCTG |
| | | ACCATCTCTGGGCTCCAGGCTGAGGACGAGGGTGACTATTACTGCAAGTCTCT |
| | | GACAAGCACGAGACGTCGGGTTTTCGGCACTGGGACCAAGCTGACCGTTCTAC |
| | | GCAAACGCCGCGGCAGCGGCGCGACCAACTTTAGCCTGCTGAAACAGGCGGGC |
| | | GATGTGGAAGAAAACCCGGGCCCGATGCCACCTCCTCGCCTCCTCTTCTTCCT |
| | | CCTCTTCCTCACCCCCATGGAAGTCAGGCCCGAGGAACCTCTAGTGGTGAAGG |
| | | TGGAAGAGGGAGATAACGCTGTGCTGCAGTGCCTCAAGGGGACCTCAGATGGC |
| | | CCCACTCAGCAGCTGACCTGGTCTCGGGAGTCCCCGCTTAAACCCTTCTTAAA |
| | | ACTCAGCCTGGGGCTGCCAGGCCTGGGAATCCACATGAGGCCCCTGGCCATCT |
| | | GGCTTTTCATCTTCAACGTCTCTCAACAGATGGGGGGCTTCTACCTGTGCCAG |
| | | CCGGGGCCCCCCTCTGAGAAGGCCTGGCAGCCTGGCTGGACAGTCAATGTGGA |
| | | GGGCAGCGGGGAGCTGTTCCGGTGGAATGTTTCGGACCTAGGTGGCCTGGGCT |
| | | GTGGCCTGAAGAACAGGTCCTCAGAGGGCCCCAGCTCCCCTTCCGGGAAGCTC |
| | | ATGAGCCCCAAGCTGTATGTGTGGGCCAAAGACCGCCCTGAGATCTGGGAGGG |
| | | AGAGCCTCCGTGTCTCCCACCGAGGGACAGCCTGAACCAGAGCCTCAGCCAGG |
| | | ACCTCACCATGGCCCCTGGCTCCACACTCTGGCTGTCCTGTGGGGTACCCCCT |
| | | GACTCTGTGTCCAGGGGCCCCCTCTCCTGGACCCATGTGCACCCCAAGGGGCC |
| | | TAAGTCATTGCTGAGCCTAGAGCTGAAGGACGATCGCCCGGCCAGAGATATGT |
| | | GGGTAATGGAGACGGGTCTGTTGTTGCCCCGGGCCACAGCTCAAGACGCTGGA |
| | | AAGTATTATTGTCACCGTGGCAACCTGACCATGTCATTCCACCTGGAGATCAC |
| | | TGCTCGGCCAGTACTATGGCACTGGCTGCTGAGGACTGGTGGCTGGAAGGTCT |
| | | CAGCTGTGACTTTGGCTTATCTGATCTTCTGCCTGTGTTCCCTTGTGGGCATT |
| | | CTTCATCTTCAAAGAGCCCTGGTCCTGAGGAGGAAAAGAAAGCGAATGACTGA |
| | | CCCCACCAGGAGATTC |
| 10 | PG9 scFv- | ATGGATTGGATTTGGCGCATTCTGTTTCTGGTGGGCGCGGCGACCGGCGCGCA |
| | nucleic | TAGCGAAGTGCAGCTGGTGGAAAGCGGCGGCGGCGTGGTGCGCCCGGGCGGCA |
| | acid- | GCCTGCGCCTGAGCTGCGCGGCGAGCGGCTTTACCTTTGATGATTATGGCATG |
| | encoded | AGCTGGGTGCGCCAGGCGCCGGGCAAAGGCCTGGAATGGGTGAGCGGCATTAA |
| | region | CTGGAACGGCGGCAGCACCGGCTATGCGGATAGCGTGAAAGGCCGCTTTACCA |
| | | TTAGCCGCGATAACGCGAAAAACAGCCTGTATCTGCAGATGAACAGCCTGCGC |
| | | GCGGAAGATACCGCGGTGTATTATTGCGCGCGCGGCCGCAGCCTGCTGTTTGA |
| | | TTATTGGGGCCAGGGCACCCTGGTGACCGTGAGCCGCGGCGGCGGCGGCAGCG |
| | | GCGGCGGCGGCAGCGGCGGCGGCGGCAGCGGCGGCGGCGGCAGCAGCAGCGAA |
| | | CTGACCCAGGATCCGGCGGTGAGCGTGGCGCTGGGCCAGACCGTGCGCATTAC |
| | | CTGCCAGGGCGATAGCCTGCGCAGCTATTATGCGAGCTGGTATCAGCAGAAAC |
| | | CGGGCCAGGCGCCGGTGCTGGTGATTTATGGCAAAAACAACCGCCCGAGCGGC |
| | | ATTCCGGATCGCTTTAGCGGCAGCAGCAGCGGCAACACCGCGAGCCTGACCAT |
| | | TACCGGCGCGCAGGCGGAAGATGAAGCGGATTATTATTGCAACAGCCGCGATA |
| | | GCAGCGGCAACCATGTGGTGTTTGGCGGCGGCACCAAACTGACCGTGGGCAGC |
| | | GGCGGCGGCGGCAGCCAGCGATTAGTGGAGTCTGGGGGAGGCGTGGTCCAGCC |
| | | TGGGTCGTCCCTGAGACTCTCCTGTGCAGCGTCCGGATTCGACTTCAGTAGAC |
| | | AAGGCATGCACTGGGTCCGCCAGGCTCCAGGCCAGGGGCTGGAGTGGGTGGCA |
| | | TTTATTAAATATGATGGAAGTGAGAAATATCATGCTGACTCCGTATGGGGCCG |
| | | ACTCAGCATCTCCAGAGACAATTCCAAGGATACGCTTTATCTCCAAATGAATA |
| | | GCCTGAGAGTCGAGGACACGGCTACATATTTTTGTGTGAGAGAGGCTGGTGGG |
| | | CCCGACTACCGTAATGGGTACAACTATTACGATTTCTATGATGGTTATTATAA |
| | | CTACCACTATATGGACGTCTGGGGCAAAGGGACCACGGTCACCGTCTCGAGCG |
| | | GCGGCGGCGGCAGCGGCGGCGGCGGCAGCGGCGGCGGCGGCAGCGGCGGCGGC |
| | | GGCAGCCAGTCTGCCCTGACTCAGCCTGCCTCCGTGTCTGGGTCTCCTGGACA |
| | | GTCGATCACCATCTCCTGCAATGGAACCAGCAATGATGTTGGTGGCTATGAAT |
| | | CTGTCTCCTGGTACCAACAACATCCCGGCAAAGCCCCCAAAGTCGTGATTTAT |
| | | GATGTCAGTAAACGGCCCTCAGGGGTTTCTAATCGCTTCTCTGGCTCCAAGTC |
| | | CGGCAACACGGCCTCCCTGACCATCTCTGGGCTCCAGGCTGAGGACGAGGGTG |
| | | ACTATTACTGCAAGTCTCTGACAAGCACGAGACGTCGGGTTTTCGGCACTGGG |
| | | ACCAAGCTGACCGTTCTACGCAAACGCCGCGGCAGCGGCGCGACCAACTTTAG |
| | | CCTGCTGAAACAGGCGGGCGATGTGGAAGAAAACCCGGGCCCGATGCCACCTC |
| | | CTCGCCTCCTCTTCTTCCTCCTCTTCCTCACCCCCATGGAAGTCAGGCCCGAG |
| | | GAACCTCTAGTGGTGAAGGTGGAAGAGGGAGATAACGCTGTGCTGCAGTGCCT |
| | | CAAGGGGACCTCAGATGGCCCCACTCAGCAGCTGACCTGGTCTCGGGAGTCCC |
| | | CGCTTAAACCCTTCTTAAAACTCAGCCTGGGGCTGCCAGGCCTGGGAATCCAC |
| | | ATGAGGCCCCTGGCCATCTGGCTTTTCATCTTCAACGTCTCTCAACAGATGGG |
| | | GGGCTTCTACCTGTGCCAGCCGGGGCCCCCCTCTGAGAAGGCCTGGCAGCCTG |
| | | GCTGGACAGTCAATGTGGAGGGCAGCGGGGAGCTGTTCCGGTGGAATGTTTCG |
| | | GACCTAGGTGGCCTGGGCTGTGGCCTGAAGAACAGGTCCTCAGAGGGCCCCAG |
| | | CTCCCCTTCCGGGAAGCTCATGAGCCCCAAGCTGTATGTGTGGGCCAAAGACC |
| | | GCCCTGAGATCTGGGAGGGAGAGCCTCCGTGTCTCCCACCGAGGGACAGCCTG |
| | | AACCAGAGCCTCAGCCAGGACCTCACCATGGCCCCTGGCTCCACACTCTGGCT |
| | | GTCCTGTGGGGTACCCCCTGACTCTGTGTCCAGGGGCCCCCTCTCCTGGACCC |
| | | ATGTGCACCCCAAGGGGCCTAAGTCATTGCTGAGCCTAGAGCTGAAGGACGAT |
| | | CGCCCGGCCAGAGATATGTGGGTAATGGAGACGGGTCTGTTGTTGCCCCGGGC |
| | | CACAGCTCAAGACGCTGGAAAGTATTATTGTCACCGTGGCAACCTGACCATGT |
| | | CATTCCACCTGGAGATCACTGCTCGGCCAGTACTATGGCACTGGCTGCTGAGG |
| | | ACTGGTGGCTGGAAGGTCTCAGCTGTGACTTTGGCTTATCTGATCTTCTGCCT |
| | | GTGTTCCCTTGTGGGCATTCTTCATCTTCAAAGAGCCCTGGTCCTGAGGAGGA |
| | | AAAGAAAGCGAATGACTGACCCCACCAGGAGATTC |
| 11 | 10-1074 full | CGTTACATAACTTACGGTAAATGGCCCGCCTGGCTGACCGCCCAACGACCCCC |
| | length | GCCCATTGACGTCAATAATGACGTATGTTCCCATAGTAACGCCAATAGGGACT |
| | nucleic acid | TTCCATTGACGTCAATGGGTGGAGTATTTACGGTAAACTGCCCACTTGGCAGT |
| | sequence | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACCATGGGATGGTCATGTATCA |
| | | TCCTTTTTCTAGTAGCAACTGCAACCGGTGTACATTCATCCTATGTCAGGCCA |
| | | CTGTCCGTCGCACTGGGGGAGACCGCAAGAATTAGCTGTGGGAGGCAGGCACT |
| | | GGGGAGCAGGGCTGTCCAGTGGTACCAGCACCGACCAGGACAGGCACCAATCC |
| | | TGCTGATCTACAACAATCAGGACCGGCCTTCAGGCATCCCCGAGAGATTCAGC |
| | | GGAACACCCGATATTAACTTTGGCACTAGAGCTACCCTGACAATCAGCGGAGT |
| | | GGAGGCAGGCGACGAAGCCGATTACTATTGCCATATGTGGGACTCCAGGTCTG |
| | | GGTTCAGTTGGTCATTTGGCGGAGCAACTCGACTGACCGTGCTGACCGTGGCG |
| | | GCGCCGAGCGTGTTTATTTTTCCGCCGAGCGATGAACAGCTGAAAAGCGGCAC |
| | | CGCGAGCGTGGTGTGCCTGCTGAACAACTTTTATCCGCGCGAAGCGAAAGTGC |
| | | AGTGGAAAGTGGATAACGCGCTGCAGAGCGGCAACAGCCAGGAAAGCGTGACC |
| | | GAACAGGATAGCAAAGATAGCACCTATAGCCTGAGCAGCACCCTGACCCTGAG |
| | | CAAAGCGGATTATGAAAAACATAAAGTGTATGCGTGCGAAGTGACCCATCAGG |
| | | GCCTGAGCAGCCCGGTGACCAAAAGCTTTAACCGCGGCGAATGCCGCAAACGC |
| | | CGCGGCAGCGGCGCGACCAACTTTAGCCTGCTGAAACAGGCGGGCGATGTGGA |
| | | AGAAAACCCGGGCCCGATGGGATGGTCATGTATCATCCTTTTTCTAGTAGCAA |
| | | CTGCAACCGGTGTACATTCACAGGTGCAGCTGCAGGAATCTGGGCCTGGACTG |
| | | GTCAAACCCTCCGAGACTCTGAGCGTCACTTGTTCTGTGAGCGGCGACTCTAT |
| | | GAACAATTACTATTGGACATGGATCCGACAGAGCCCAGGCAAGGGGCTGGAGT |
| | | GGATCGGCTACATTTCTGACAGAGAAAGTGCTACTTATAACCCTAGCCTGAAT |
| | | TCCAGGGTGGTCATTTCACGCGACACCAGCAAGAACCAGCTGTCCCTGAAACT |
| | | GAATTCTGTGACCCCCGCAGATACAGCCGTCTACTATTGCGCCACCGCTCGGA |
| | | GAGGACAGCGGATCTACGGCGTGGTCAGCTTCGGGGAGTTCTTTTACTACTAC |
| | | TCAATGGATGTCTGGGGGAAGGGGACTACAGTGACCGTCTCAAGCGCCTCGAC |
| | | CAAGGCGAGCACCAAAGGCCCGAGCGTGTTTCCGCTGGCGCCGTGCAGCCGCA |
| | | GCACCAGCGGCGGCACCGCGGCGCTGGGCTGCCTGGTGAAAGATTATTTTCCG |
| | | GAACCGGTGACCGTGAGCTGGAACAGCGGCGCGCTGACCAGCGGCGTGCATAC |
| | | CTTTCCGGCGGTGCTGCAGAGCAGCGGCCTGTATAGCCTGAGCAGCGTGGTGA |
| | | CCGTGCCGAGCAGCAGCCTGGGCACCCAGACCTATACCTGCAACGTGAACCAT |
| | | AAACCGAGCAACACCAAAGTGGATAAACGCGTGGAACTGAAAACCCCGCTGGG |
| | | CGATACCACCCATACCTGCCCGCGCTGCCCGGAACCGAAAAGCTGCGATACCC |
| | | CGCCGCCGTGCCCGCGCTGCCCGGAACCGAAAAGCTGCGATACCCCGCCGCCG |
| | | TGCCCGCGCTGCCCGGAACCGAAAAGCTGCGATACCCCGCCGCCGTGCCCGCG |
| | | CTGCCCGGCGCCGGAACTGCTGGGCGGCCCGAGCGTGTTTCTGTTTCCGCCGA |
| | | AACCGAAAGATACCCTGATGATTAGCCGCACCCCGGAAGTGACCTGCGTGGTG |
| | | GTGGATGTGAGCCATGAAGATCCGGAAGTGCAGTTTAAATGGTATGTGGATGG |
| | | CGTGGAAGTGCATAACGCGAAAACCAAACCGCGCGAAGAACAGTATAACAGCA |
| | | CCTTTCGCGTGGTGAGCGTGCTGACCGTGCTGCATCAGGATTGGCTGAACGGC |
| | | AAAGAATATAAATGCAAAGTGAGCAACAAAGCGCTGCCGGCGCCGATTGAAAA |
| | | AACCATTAGCAAAACCAAAGGCCAGCCGCGCGAACCGCAGGTGTATACCCTGC |
| | | CGCCGAGCCGCGAAGAAATGACCAAAAACCAGGTGAGCCTGACCTGCCTGGTG |
| | | AAAGGCTTTTATCCGAGCGATATTGCGGTGGAATGGGAAAGCAGCGGCCAGCC |
| | | GGAAAACAACTATAACACCACCCCGCCGATGCTGGATAGCGATGGCAGCTTTT |
| | | TTCTGTATAGCAAACTGACCGTGGATAAAAGCCGCTGGCAGCAGGGCAACATT |
| | | TTTAGCTGCAGCGTGATGCATGAAGCGCTGCATAACCGCTTTACCCAGAAAAG |
| | | CCTGAGCCTGAGCCCGGGCAAACGCAAACGCCGCGGCAGCGGCGCGACCAACT |
| | | TTAGCCTGCTGAAACAGGCGGGCGATGTGGAAGAAAACCCGGGCCCGATGCCA |
| | | CCTCCTCGCCTCCTCTTCTTCCTCCTCTTCCTCACCCCCATGGAAGTCAGGCC |
| | | CGAGGAACCTCTAGTGGTGAAGGTGGAAGAGGGAGATAACGCTGTGCTGCAGT |
| | | GCCTCAAGGGGACCTCAGATGGCCCCACTCAGCAGCTGACCTGGTCTCGGGAG |
| | | TCCCCGCTTAAACCCTTCTTAAAACTCAGCCTGGGGCTGCCAGGCCTGGGAAT |
| | | CCACATGAGGCCCCTGGCCATCTGGCTTTTCATCTTCAACGTCTCTCAACAGA |
| | | TGGGGGGCTTCTACCTGTGCCAGCCGGGGCCCCCCTCTGAGAAGGCCTGGCAG |
| | | CCTGGCTGGACAGTCAATGTGGAGGGCAGCGGGGAGCTGTTCCGGTGGAATGT |
| | | TTCGGACCTAGGTGGCCTGGGCTGTGGCCTGAAGAACAGGTCCTCAGAGGGCC |
| | | CCAGCTCCCCTTCCGGGAAGCTCATGAGCCCCAAGCTGTATGTGTGGGCCAAA |
| | | GACCGCCCTGAGATCTGGGAGGGAGAGCCTCCGTGTCTCCCACCGAGGGACAG |
| | | CCTGAACCAGAGCCTCAGCCAGGACCTCACCATGGCCCCTGGCTCCACACTCT |
| | | GGCTGTCCTGTGGGGTACCCCCTGACTCTGTGTCCAGGGGCCCCCTCTCCTGG |
| | | ACCCATGTGCACCCCAAGGGGCCTAAGTCATTGCTGAGCCTAGAGCTGAAGGA |
| | | CGATCGCCCGGCCAGAGATATGTGGGTAATGGAGACGGGTCTGTTGTTGCCCC |
| | | GGGCCACAGCTCAAGACGCTGGAAAGTATTATTGTCACCGTGGCAACCTGACC |
| | | ATGTCATTCCACCTGGAGATCACTGCTCGGCCAGTACTATGGCACTGGCTGCT |
| | | GAGGACTGGTGGCTGGAAGGTCTCAGCTGTGACTTTGGCTTATCTGATCTTCT |
| | | GCCTGTGTTCCCTTGTGGGCATTCTTCATCTTCAAAGAGCCCTGGTCCTGAGG |
| | | AGGAAAAGAAAGCGAATGACTGACCCCACCAGGAGATTC |
| 12 | 10-1074 full | ATGGGATGGTCATGTATCATCCTTTTTCTAGTAGCAACTGCAACCGGTGTACA |
| | length | TTCATCCTATGTCAGGCCACTGTCCGTCGCACTGGGGGAGACCGCAAGAATTA |
| | nucleic acid | GCTGTGGGAGGCAGGCACTGGGGAGCAGGGCTGTCCAGTGGTACCAGCACCGA |
| | sequence- | CCAGGACAGGCACCAATCCTGCTGATCTACAACAATCAGGACCGGCCTTCAGG |
| | encoded | CATCCCCGAGAGATTCAGCGGAACACCCGATATTAACTTTGGCACTAGAGCTA |
| | region | CCCTGACAATCAGCGGAGTGGAGGCAGGCGACGAAGCCGATTACTATTGCCAT |
| | | ATGTGGGACTCCAGGTCTGGGTTCAGTTGGTCATTTGGCGGAGCAACTCGACT |
| | | GACCGTGCTGACCGTGGCGGCGCCGAGCGTGTTTATTTTTCCGCCGAGCGATG |
| | | AACAGCTGAAAAGCGGCACCGCGAGCGTGGTGTGCCTGCTGAACAACTTTTAT |
| | | CCGCGCGAAGCGAAAGTGCAGTGGAAAGTGGATAACGCGCTGCAGAGCGGCAA |
| | | CAGCCAGGAAAGCGTGACCGAACAGGATAGCAAAGATAGCACCTATAGCCTGA |
| | | GCAGCACCCTGACCCTGAGCAAAGCGGATTATGAAAAACATAAAGTGTATGCG |
| | | TGCGAAGTGACCCATCAGGGCCTGAGCAGCCCGGTGACCAAAAGCTTTAACCG |
| | | CGGCGAATGCCGCAAACGCCGCGGCAGCGGCGCGACCAACTTTAGCCTGCTGA |
| | | AACAGGCGGGCGATGTGGAAGAAAACCCGGGCCCGATGGGATGGTCATGTATC |
| | | ATCCTTTTTCTAGTAGCAACTGCAACCGGTGTACATTCACAGGTGCAGCTGCA |
| | | GGAATCTGGGCCTGGACTGGTCAAACCCTCCGAGACTCTGAGCGTCACTTGTT |
| | | CTGTGAGCGGCGACTCTATGAACAATTACTATTGGACATGGATCCGACAGAGC |
| | | CCAGGCAAGGGGCTGGAGTGGATCGGCTACATTTCTGACAGAGAAAGTGCTAC |
| | | TTATAACCCTAGCCTGAATTCCAGGGTGGTCATTTCACGCGACACCAGCAAGA |
| | | ACCAGCTGTCCCTGAAACTGAATTCTGTGACCCCCGCAGATACAGCCGTCTAC |
| | | TATTGCGCCACCGCTCGGAGAGGACAGCGGATCTACGGCGTGGTCAGCTTCGG |
| | | GGAGTTCTTTTACTACTACTCAATGGATGTCTGGGGGAAGGGGACTACAGTGA |
| | | CCGTCTCAAGCGCCTCGACCAAGGCGAGCACCAAAGGCCCGAGCGTGTTTCCG |
| | | CTGGCGCCGTGCAGCCGCAGCACCAGCGGCGGCACCGCGGCGCTGGGCTGCCT |
| | | GGTGAAAGATTATTTTCCGGAACCGGTGACCGTGAGCTGGAACAGCGGCGCGC |
| | | TGACCAGCGGCGTGCATACCTTTCCGGCGGTGCTGCAGAGCAGCGGCCTGTAT |
| | | AGCCTGAGCAGCGTGGTGACCGTGCCGAGCAGCAGCCTGGGCACCCAGACCTA |
| | | TACCTGCAACGTGAACCATAAACCGAGCAACACCAAAGTGGATAAACGCGTGG |
| | | AACTGAAAACCCCGCTGGGCGATACCACCCATACCTGCCCGCGCTGCCCGGAA |
| | | CCGAAAAGCTGCGATACCCCGCCGCCGTGCCCGCGCTGCCCGGAACCGAAAAG |
| | | CTGCGATACCCCGCCGCCGTGCCCGCGCTGCCCGGAACCGAAAAGCTGCGATA |
| | | CCCCGCCGCCGTGCCCGCGCTGCCCGGCGCCGGAACTGCTGGGCGGCCCGAGC |
| | | GTGTTTCTGTTTCCGCCGAAACCGAAAGATACCCTGATGATTAGCCGCACCCC |
| | | GGAAGTGACCTGCGTGGTGGTGGATGTGAGCCATGAAGATCCGGAAGTGCAGT |
| | | TTAAATGGTATGTGGATGGCGTGGAAGTGCATAACGCGAAAACCAAACCGCGC |
| | | GAAGAACAGTATAACAGCACCTTTCGCGTGGTGAGCGTGCTGACCGTGCTGCA |
| | | TCAGGATTGGCTGAACGGCAAAGAATATAAATGCAAAGTGAGCAACAAAGCGC |
| | | TGCCGGCGCCGATTGAAAAAACCATTAGCAAAACCAAAGGCCAGCCGCGCGAA |
| | | CCGCAGGTGTATACCCTGCCGCCGAGCCGCGAAGAAATGACCAAAAACCAGGT |
| | | GAGCCTGACCTGCCTGGTGAAAGGCTTTTATCCGAGCGATATTGCGGTGGAAT |
| | | GGGAAAGCAGCGGCCAGCCGGAAAACAACTATAACACCACCCCGCCGATGCTG |
| | | GATAGCGATGGCAGCTTTTTTCTGTATAGCAAACTGACCGTGGATAAAAGCCG |
| | | CTGGCAGCAGGGCAACATTTTTAGCTGCAGCGTGATGCATGAAGCGCTGCATA |
| | | ACCGCTTTACCCAGAAAAGCCTGAGCCTGAGCCCGGGCAAACGCAAACGCCGC |
| | | GGCAGCGGCGCGACCAACTTTAGCCTGCTGAAACAGGCGGGCGATGTGGAAGA |
| | | AAACCCGGGCCCGATGCCACCTCCTCGCCTCCTCTTCTTCCTCCTCTTCCTCA |
| | | CCCCCATGGAAGTCAGGCCCGAGGAACCTCTAGTGGTGAAGGTGGAAGAGGGA |
| | | GATAACGCTGTGCTGCAGTGCCTCAAGGGGACCTCAGATGGCCCCACTCAGCA |
| | | GCTGACCTGGTCTCGGGAGTCCCCGCTTAAACCCTTCTTAAAACTCAGCCTGG |
| | | GGCTGCCAGGCCTGGGAATCCACATGAGGCCCCTGGCCATCTGGCTTTTCATC |
| | | TTCAACGTCTCTCAACAGATGGGGGGCTTCTACCTGTGCCAGCCGGGGCCCCC |
| | | CTCTGAGAAGGCCTGGCAGCCTGGCTGGACAGTCAATGTGGAGGGCAGCGGGG |
| | | AGCTGTTCCGGTGGAATGTTTCGGACCTAGGTGGCCTGGGCTGTGGCCTGAAG |
| | | AACAGGTCCTCAGAGGGCCCCAGCTCCCCTTCCGGGAAGCTCATGAGCCCCAA |
| | | GCTGTATGTGTGGGCCAAAGACCGCCCTGAGATCTGGGAGGGAGAGCCTCCGT |
| | | GTCTCCCACCGAGGGACAGCCTGAACCAGAGCCTCAGCCAGGACCTCACCATG |
| | | GCCCCTGGCTCCACACTCTGGCTGTCCTGTGGGGTACCCCCTGACTCTGTGTC |
| | | CAGGGGCCCCCTCTCCTGGACCCATGTGCACCCCAAGGGGCCTAAGTCATTGC |
| | | TGAGCCTAGAGCTGAAGGACGATCGCCCGGCCAGAGATATGTGGGTAATGGAG |
| | | ACGGGTCTGTTGTTGCCCCGGGCCACAGCTCAAGACGCTGGAAAGTATTATTG |
| | | TCACCGTGGCAACCTGACCATGTCATTCCACCTGGAGATCACTGCTCGGCCAG |
| | | TACTATGGCACTGGCTGCTGAGGACTGGTGGCTGGAAGGTCTCAGCTGTGACT |
| | | TTGGCTTATCTGATCTTCTGCCTGTGTTCCCTTGTGGGCATTCTTCATCTTCA |
| | | AAGAGCCCTGGTCCTGAGGAGGAAAAGAAAGCGAATGACTGACCCCACCAGGA |
| | | GATTC |
| 13 | 10-1074 | CGTTACATAACTTACGGTAAATGGCCCGCCTGGCTGACCGCCCAACGACCCCC |
| | light chain | GCCCATTGACGTCAATAATGACGTATGTTCCCATAGTAACGCCAATAGGGACT |
| | nucleic acid | TTCCATTGACGTCAATGGGTGGAGTATTTACGGTAAACTGCCCACTTGGCAGT |
| | sequence | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACCATGGGATGGTCATGTATCA |
| | | TCCTTTTTCTAGTAGCAACTGCAACCGGTGTACATTCATCCTATGTCAGGCCA |
| | | CTGTCCGTCGCACTGGGGGAGACCGCAAGAATTAGCTGTGGGAGGCAGGCACT |
| | | GGGGAGCAGGGCTGTCCAGTGGTACCAGCACCGACCAGGACAGGCACCAATCC |
| | | TGCTGATCTACAACAATCAGGACCGGCCTTCAGGCATCCCCGAGAGATTCAGC |
| | | GGAACACCCGATATTAACTTTGGCACTAGAGCTACCCTGACAATCAGCGGAGT |
| | | GGAGGCAGGCGACGAAGCCGATTACTATTGCCATATGTGGGACTCCAGGTCTG |
| | | GGTTCAGTTGGTCATTTGGCGGAGCAACTCGACTGACCGTGCTGACCGTGGCG |
| | | GCGCCGAGCGTGTTTATTTTTCCGCCGAGCGATGAACAGCTGAAAAGCGGCAC |
| | | CGCGAGCGTGGTGTGCCTGCTGAACAACTTTTATCCGCGCGAAGCGAAAGTGC |
| | | AGTGGAAAGTGGATAACGCGCTGCAGAGCGGCAACAGCCAGGAAAGCGTGACC |
| | | GAACAGGATAGCAAAGATAGCACCTATAGCCTGAGCAGCACCCTGACCCTGAG |
| | | CAAAGCGGATTATGAAAAACATAAAGTGTATGCGTGCGAAGTGACCCATCAGG |
| | | GCCTGAGCAGCCCGGTGACCAAAAGCTTTAACCGCGGCGAATGCCGCAAACGC |
| | | CGCGGCAGCGGCGCGACCAACTTTAGCCTGCTGAAACAGGCGGGCGATGTGGA |
| | | AGAAAACCCGGGCCCGATGCCACCTCCTCGCCTCCTCTTCTTCCTCCTCTTCC |
| | | TCACCCCCATGGAAGTCAGGCCCGAGGAACCTCTAGTGGTGAAGGTGGAAGAG |
| | | GGAGATAACGCTGTGCTGCAGTGCCTCAAGGGGACCTCAGATGGCCCCACTCA |
| | | GCAGCTGACCTGGTCTCGGGAGTCCCCGCTTAAACCCTTCTTAAAACTCAGCC |
| | | TGGGGCTGCCAGGCCTGGGAATCCACATGAGGCCCCTGGCCATCTGGCTTTTC |
| | | ATCTTCAACGTCTCTCAACAGATGGGGGGCTTCTACCTGTGCCAGCCGGGGCC |
| | | CCCCTCTGAGAAGGCCTGGCAGCCTGGCTGGACAGTCAATGTGGAGGGCAGCG |
| | | GGGAGCTGTTCCGGTGGAATGTTTCGGACCTAGGTGGCCTGGGCTGTGGCCTG |
| | | AAGAACAGGTCCTCAGAGGGCCCCAGCTCCCCTTCCGGGAAGCTCATGAGCCC |
| | | CAAGCTGTATGTGTGGGCCAAAGACCGCCCTGAGATCTGGGAGGGAGAGCCTC |
| | | CGTGTCTCCCACCGAGGGACAGCCTGAACCAGAGCCTCAGCCAGGACCTCACC |
| | | ATGGCCCCTGGCTCCACACTCTGGCTGTCCTGTGGGGTACCCCCTGACTCTGT |
| | | GTCCAGGGGCCCCCTCTCCTGGACCCATGTGCACCCCAAGGGGCCTAAGTCAT |
| | | TGCTGAGCCTAGAGCTGAAGGACGATCGCCCGGCCAGAGATATGTGGGTAATG |
| | | GAGACGGGTCTGTTGTTGCCCCGGGCCACAGCTCAAGACGCTGGAAAGTATTA |
| | | TTGTCACCGTGGCAACCTGACCATGTCATTCCACCTGGAGATCACTGCTCGGC |
| | | CAGTACTATGGCACTGGCTGCTGAGGACTGGTGGCTGGAAGGTCTCAGCTGTG |
| | | ACTTTGGCTTATCTGATCTTCTGCCTGTGTTCCCTTGTGGGCATTCTTCATCT |
| | | TCAAAGAGCCCTGGTCCTGAGGAGGAAAAGAAAGCGAATGACTGACCCCACCA |
| | | GGAGATTC |
| 14 | 10-1074 | ATGGGATGGTCATGTATCATCCTTTTTCTAGTAGCAACTGCAACCGGTGTACA |
| | light chain | TTCATCCTATGTCAGGCCACTGTCCGTCGCACTGGGGGAGACCGCAAGAATTA |
| | nucleic acid | GCTGTGGGAGGCAGGCACTGGGGAGCAGGGCTGTCCAGTGGTACCAGCACCGA |
| | sequence- | CCAGGACAGGCACCAATCCTGCTGATCTACAACAATCAGGACCGGCCTTCAGG |
| | encoded | CATCCCCGAGAGATTCAGCGGAACACCCGATATTAACTTTGGCACTAGAGCTA |
| | region | CCCTGACAATCAGCGGAGTGGAGGCAGGCGACGAAGCCGATTACTATTGCCAT |
| | | ATGTGGGACTCCAGGTCTGGGTTCAGTTGGTCATTTGGCGGAGCAACTCGACT |
| | | GACCGTGCTGACCGTGGCGGCGCCGAGCGTGTTTATTTTTCCGCCGAGCGATG |
| | | AACAGCTGAAAAGCGGCACCGCGAGCGTGGTGTGCCTGCTGAACAACTTTTAT |
| | | CCGCGCGAAGCGAAAGTGCAGTGGAAAGTGGATAACGCGCTGCAGAGCGGCAA |
| | | CAGCCAGGAAAGCGTGACCGAACAGGATAGCAAAGATAGCACCTATAGCCTGA |
| | | GCAGCACCCTGACCCTGAGCAAAGCGGATTATGAAAAACATAAAGTGTATGCG |
| | | TGCGAAGTGACCCATCAGGGCCTGAGCAGCCCGGTGACCAAAAGCTTTAACCG |
| | | CGGCGAATGCCGCAAACGCCGCGGCAGCGGCGCGACCAACTTTAGCCTGCTGA |
| | | AACAGGCGGGCGATGTGGAAGAAAACCCGGGCCCGATGCCACCTCCTCGCCTC |
| | | CTCTTCTTCCTCCTCTTCCTCACCCCCATGGAAGTCAGGCCCGAGGAACCTCT |
| | | AGTGGTGAAGGTGGAAGAGGGAGATAACGCTGTGCTGCAGTGCCTCAAGGGGA |
| | | CCTCAGATGGCCCCACTCAGCAGCTGACCTGGTCTCGGGAGTCCCCGCTTAAA |
| | | CCCTTCTTAAAACTCAGCCTGGGGCTGCCAGGCCTGGGAATCCACATGAGGCC |
| | | CCTGGCCATCTGGCTTTTCATCTTCAACGTCTCTCAACAGATGGGGGGCTTCT |
| | | ACCTGTGCCAGCCGGGGCCCCCCTCTGAGAAGGCCTGGCAGCCTGGCTGGACA |
| | | GTCAATGTGGAGGGCAGCGGGGAGCTGTTCCGGTGGAATGTTTCGGACCTAGG |
| | | TGGCCTGGGCTGTGGCCTGAAGAACAGGTCCTCAGAGGGCCCCAGCTCCCCTT |
| | | CCGGGAAGCTCATGAGCCCCAAGCTGTATGTGTGGGCCAAAGACCGCCCTGAG |
| | | ATCTGGGAGGGAGAGCCTCCGTGTCTCCCACCGAGGGACAGCCTGAACCAGAG |
| | | CCTCAGCCAGGACCTCACCATGGCCCCTGGCTCCACACTCTGGCTGTCCTGTG |
| | | GGGTACCCCCTGACTCTGTGTCCAGGGGCCCCCTCTCCTGGACCCATGTGCAC |
| | | CCCAAGGGGCCTAAGTCATTGCTGAGCCTAGAGCTGAAGGACGATCGCCCGGC |
| | | CAGAGATATGTGGGTAATGGAGACGGGTCTGTTGTTGCCCCGGGCCACAGCTC |
| | | AAGACGCTGGAAAGTATTATTGTCACCGTGGCAACCTGACCATGTCATTCCAC |
| | | CTGGAGATCACTGCTCGGCCAGTACTATGGCACTGGCTGCTGAGGACTGGTGG |
| | | CTGGAAGGTCTCAGCTGTGACTTTGGCTTATCTGATCTTCTGCCTGTGTTCCC |
| | | TTGTGGGCATTCTTCATCTTCAAAGAGCCCTGGTCCTGAGGAGGAAAAGAAAG |
| | | CGAATGACTGACCCCACCAGGAGATTC |
| 15 | 10-1074 | CGTTACATAACTTACGGTAAATGGCCCGCCTGGCTGACCGCCCAACGACCCCC |
| | heavy chain | GCCCATTGACGTCAATAATGACGTATGTTCCCATAGTAACGCCAATAGGGACT |
| | nucleic acid | TTCCATTGACGTCAATGGGTGGAGTATTTACGGTAAACTGCCCACTTGGCAGT |
| | sequence | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACCATGGGATGGTCATGTATCA |
| | | TCCTTTTTCTAGTAGCAACTGCAACCGGTGTACATTCACAGGTGCAGCTGCAG |
| | | GAATCTGGGCCTGGACTGGTCAAACCCTCCGAGACTCTGAGCGTCACTTGTTC |
| | | TGTGAGCGGCGACTCTATGAACAATTACTATTGGACATGGATCCGACAGAGCC |
| | | CAGGCAAGGGGCTGGAGTGGATCGGCTACATTTCTGACAGAGAAAGTGCTACT |
| | | TATAACCCTAGCCTGAATTCCAGGGTGGTCATTTCACGCGACACCAGCAAGAA |
| | | CCAGCTGTCCCTGAAACTGAATTCTGTGACCCCCGCAGATACAGCCGTCTACT |
| | | ATTGCGCCACCGCTCGGAGAGGACAGCGGATCTACGGCGTGGTCAGCTTCGGG |
| | | GAGTTCTTTTACTACTACTCAATGGATGTCTGGGGGAAGGGGACTACAGTGAC |
| | | CGTCTCAAGCGCCTCGACCAAGGCGAGCACCAAAGGCCCGAGCGTGTTTCCGC |
| | | TGGCGCCGTGCAGCCGCAGCACCAGCGGCGGCACCGCGGCGCTGGGCTGCCTG |
| | | GTGAAAGATTATTTTCCGGAACCGGTGACCGTGAGCTGGAACAGCGGCGCGCT |
| | | GACCAGCGGCGTGCATACCTTTCCGGCGGTGCTGCAGAGCAGCGGCCTGTATA |
| | | GCCTGAGCAGCGTGGTGACCGTGCCGAGCAGCAGCCTGGGCACCCAGACCTAT |
| | | ACCTGCAACGTGAACCATAAACCGAGCAACACCAAAGTGGATAAACGCGTGGA |
| | | ACTGAAAACCCCGCTGGGCGATACCACCCATACCTGCCCGCGCTGCCCGGAAC |
| | | CGAAAAGCTGCGATACCCCGCCGCCGTGCCCGCGCTGCCCGGAACCGAAAAGC |
| | | TGCGATACCCCGCCGCCGTGCCCGCGCTGCCCGGAACCGAAAAGCTGCGATAC |
| | | CCCGCCGCCGTGCCCGCGCTGCCCGGCGCCGGAACTGCTGGGCGGCCCGAGCG |
| | | TGTTTCTGTTTCCGCCGAAACCGAAAGATACCCTGATGATTAGCCGCACCCCG |
| | | GAAGTGACCTGCGTGGTGGTGGATGTGAGCCATGAAGATCCGGAAGTGCAGTT |
| | | TAAATGGTATGTGGATGGCGTGGAAGTGCATAACGCGAAAACCAAACCGCGCG |
| | | AAGAACAGTATAACAGCACCTTTCGCGTGGTGAGCGTGCTGACCGTGCTGCAT |
| | | CAGGATTGGCTGAACGGCAAAGAATATAAATGCAAAGTGAGCAACAAAGCGCT |
| | | GCCGGCGCCGATTGAAAAAACCATTAGCAAAACCAAAGGCCAGCCGCGCGAAC |
| | | CGCAGGTGTATACCCTGCCGCCGAGCCGCGAAGAAATGACCAAAAACCAGGTG |
| | | AGCCTGACCTGCCTGGTGAAAGGCTTTTATCCGAGCGATATTGCGGTGGAATG |
| | | GGAAAGCAGCGGCCAGCCGGAAAACAACTATAACACCACCCCGCCGATGCTGG |
| | | ATAGCGATGGCAGCTTTTTTCTGTATAGCAAACTGACCGTGGATAAAAGCCGC |
| | | TGGCAGCAGGGCAACATTTTTAGCTGCAGCGTGATGCATGAAGCGCTGCATAA |
| | | CCGCTTTACCCAGAAAAGCCTGAGCCTGAGCCCGGGCAAACGCAAACGCCGCG |
| | | GCAGCGGCGCGACCAACTTTAGCCTGCTGAAACAGGCGGGCGATGTGGAAGAA |
| | | AACCCGGGCCCGATGCCACCTCCTCGCCTCCTCTTCTTCCTCCTCTTCCTCAC |
| | | CCCCATGGAAGTCAGGCCCGAGGAACCTCTAGTGGTGAAGGTGGAAGAGGGAG |
| | | ATAACGCTGTGCTGCAGTGCCTCAAGGGGACCTCAGATGGCCCCACTCAGCAG |
| | | CTGACCTGGTCTCGGGAGTCCCCGCTTAAACCCTTCTTAAAACTCAGCCTGGG |
| | | GCTGCCAGGCCTGGGAATCCACATGAGGCCCCTGGCCATCTGGCTTTTCATCT |
| | | TCAACGTCTCTCAACAGATGGGGGGCTTCTACCTGTGCCAGCCGGGGCCCCCC |
| | | TCTGAGAAGGCCTGGCAGCCTGGCTGGACAGTCAATGTGGAGGGCAGCGGGGA |
| | | GCTGTTCCGGTGGAATGTTTCGGACCTAGGTGGCCTGGGCTGTGGCCTGAAGA |
| | | ACAGGTCCTCAGAGGGCCCCAGCTCCCCTTCCGGGAAGCTCATGAGCCCCAAG |
| | | CTGTATGTGTGGGCCAAAGACCGCCCTGAGATCTGGGAGGGAGAGCCTCCGTG |
| | | TCTCCCACCGAGGGACAGCCTGAACCAGAGCCTCAGCCAGGACCTCACCATGG |
| | | CCCCTGGCTCCACACTCTGGCTGTCCTGTGGGGTACCCCCTGACTCTGTGTCC |
| | | AGGGGCCCCCTCTCCTGGACCCATGTGCACCCCAAGGGGCCTAAGTCATTGCT |
| | | GAGCCTAGAGCTGAAGGACGATCGCCCGGCCAGAGATATGTGGGTAATGGAGA |
| | | CGGGTCTGTTGTTGCCCCGGGCCACAGCTCAAGACGCTGGAAAGTATTATTGT |
| | | CACCGTGGCAACCTGACCATGTCATTCCACCTGGAGATCACTGCTCGGCCAGT |
| | | ACTATGGCACTGGCTGCTGAGGACTGGTGGCTGGAAGGTCTCAGCTGTGACTT |
| | | TGGCTTATCTGATCTTCTGCCTGTGTTCCCTTGTGGGCATTCTTCATCTTCAA |
| | | AGAGCCCTGGTCCTGAGGAGGAAAAGAAAGCGAATGACTGACCCCACCAGGAG |
| | | ATTC |
| 16 | 10-1074 | ATGGGATGGTCATGTATCATCCTTTTTCTAGTAGCAACTGCAACCGGTGTACA |
| | heavy chain | TTCACAGGTGCAGCTGCAGGAATCTGGGCCTGGACTGGTCAAACCCTCCGAGA |
| | nucleic acid | CTCTGAGCGTCACTTGTTCTGTGAGCGGCGACTCTATGAACAATTACTATTGG |
| | sequence- | ACATGGATCCGACAGAGCCCAGGCAAGGGGCTGGAGTGGATCGGCTACATTTC |
| | encoded | TGACAGAGAAAGTGCTACTTATAACCCTAGCCTGAATTCCAGGGTGGTCATTT |
| | region | CACGCGACACCAGCAAGAACCAGCTGTCCCTGAAACTGAATTCTGTGACCCCC |
| | | GCAGATACAGCCGTCTACTATTGCGCCACCGCTCGGAGAGGACAGCGGATCTA |
| | | CGGCGTGGTCAGCTTCGGGGAGTTCTTTTACTACTACTCAATGGATGTCTGGG |
| | | GGAAGGGGACTACAGTGACCGTCTCAAGCGCCTCGACCAAGGCGAGCACCAAA |
| | | GGCCCGAGCGTGTTTCCGCTGGCGCCGTGCAGCCGCAGCACCAGCGGCGGCAC |
| | | CGCGGCGCTGGGCTGCCTGGTGAAAGATTATTTTCCGGAACCGGTGACCGTGA |
| | | GCTGGAACAGCGGCGCGCTGACCAGCGGCGTGCATACCTTTCCGGCGGTGCTG |
| | | CAGAGCAGCGGCCTGTATAGCCTGAGCAGCGTGGTGACCGTGCCGAGCAGCAG |
| | | CCTGGGCACCCAGACCTATACCTGCAACGTGAACCATAAACCGAGCAACACCA |
| | | AAGTGGATAAACGCGTGGAACTGAAAACCCCGCTGGGCGATACCACCCATACC |
| | | TGCCCGCGCTGCCCGGAACCGAAAAGCTGCGATACCCCGCCGCCGTGCCCGCG |
| | | CTGCCCGGAACCGAAAAGCTGCGATACCCCGCCGCCGTGCCCGCGCTGCCCGG |
| | | AACCGAAAAGCTGCGATACCCCGCCGCCGTGCCCGCGCTGCCCGGCGCCGGAA |
| | | CTGCTGGGCGGCCCGAGCGTGTTTCTGTTTCCGCCGAAACCGAAAGATACCCT |
| | | GATGATTAGCCGCACCCCGGAAGTGACCTGCGTGGTGGTGGATGTGAGCCATG |
| | | AAGATCCGGAAGTGCAGTTTAAATGGTATGTGGATGGCGTGGAAGTGCATAAC |
| | | GCGAAAACCAAACCGCGCGAAGAACAGTATAACAGCACCTTTCGCGTGGTGAG |
| | | CGTGCTGACCGTGCTGCATCAGGATTGGCTGAACGGCAAAGAATATAAATGCA |
| | | AAGTGAGCAACAAAGCGCTGCCGGCGCCGATTGAAAAAACCATTAGCAAAACC |
| | | AAAGGCCAGCCGCGCGAACCGCAGGTGTATACCCTGCCGCCGAGCCGCGAAGA |
| | | AATGACCAAAAACCAGGTGAGCCTGACCTGCCTGGTGAAAGGCTTTTATCCGA |
| | | GCGATATTGCGGTGGAATGGGAAAGCAGCGGCCAGCCGGAAAACAACTATAAC |
| | | ACCACCCCGCCGATGCTGGATAGCGATGGCAGCTTTTTTCTGTATAGCAAACT |
| | | GACCGTGGATAAAAGCCGCTGGCAGCAGGGCAACATTTTTAGCTGCAGCGTGA |
| | | TGCATGAAGCGCTGCATAACCGCTTTACCCAGAAAAGCCTGAGCCTGAGCCCG |
| | | GGCAAACGCAAACGCCGCGGCAGCGGCGCGACCAACTTTAGCCTGCTGAAACA |
| | | GGCGGGCGATGTGGAAGAAAACCCGGGCCCGATGCCACCTCCTCGCCTCCTCT |
| | | TCTTCCTCCTCTTCCTCACCCCCATGGAAGTCAGGCCCGAGGAACCTCTAGTG |
| | | GTGAAGGTGGAAGAGGGAGATAACGCTGTGCTGCAGTGCCTCAAGGGGACCTC |
| | | AGATGGCCCCACTCAGCAGCTGACCTGGTCTCGGGAGTCCCCGCTTAAACCCT |
| | | TCTTAAAACTCAGCCTGGGGCTGCCAGGCCTGGGAATCCACATGAGGCCCCTG |
| | | GCCATCTGGCTTTTCATCTTCAACGTCTCTCAACAGATGGGGGGCTTCTACCT |
| | | GTGCCAGCCGGGGCCCCCCTCTGAGAAGGCCTGGCAGCCTGGCTGGACAGTCA |
| | | ATGTGGAGGGCAGCGGGGAGCTGTTCCGGTGGAATGTTTCGGACCTAGGTGGC |
| | | CTGGGCTGTGGCCTGAAGAACAGGTCCTCAGAGGGCCCCAGCTCCCCTTCCGG |
| | | GAAGCTCATGAGCCCCAAGCTGTATGTGTGGGCCAAAGACCGCCCTGAGATCT |
| | | GGGAGGGAGAGCCTCCGTGTCTCCCACCGAGGGACAGCCTGAACCAGAGCCTC |
| | | AGCCAGGACCTCACCATGGCCCCTGGCTCCACACTCTGGCTGTCCTGTGGGGT |
| | | ACCCCCTGACTCTGTGTCCAGGGGCCCCCTCTCCTGGACCCATGTGCACCCCA |
| | | AGGGGCCTAAGTCATTGCTGAGCCTAGAGCTGAAGGACGATCGCCCGGCCAGA |
| | | GATATGTGGGTAATGGAGACGGGTCTGTTGTTGCCCCGGGCCACAGCTCAAGA |
| | | CGCTGGAAAGTATTATTGTCACCGTGGCAACCTGACCATGTCATTCCACCTGG |
| | | AGATCACTGCTCGGCCAGTACTATGGCACTGGCTGCTGAGGACTGGTGGCTGG |
| | | AAGGTCTCAGCTGTGACTTTGGCTTATCTGATCTTCTGCCTGTGTTCCCTTGT |
| | | GGGCATTCTTCATCTTCAAAGAGCCCTGGTCCTGAGGAGGAAAAGAAAGCGAA |
| | | TGACTGACCCCACCAGGAGATTC |
| 17 | 10-1074- | TCCTATGTCAGGCCACTGTCCGTCGCACTGGGGGAGACCGCAAGAATTAGCTG |
| | LC_1012F- | TGGGAGGCAGGCACTGGGGAGCAGGGCTGTCCAGTGGTACCAGCACCGACCAG |
| | light chain | GACAGGCACCAATCCTGCTGATCTACAACAATCAGGACCGGCCTTCAGGCATC |
| | variable | CCCGAGAGATTCAGCGGAACACCCGATATTAACTTTGGCACTAGAGCTACCCT |
| | region | GACAATCAGCGGAGTGGAGGCAGGCGACGAAGCCGATTACTATTGCCATATGT |
| | | GGGACTCCAGGTCTGGGTTCAGTTGGTCATTTGGCGGAGCAACTCGACTGACC |
| | | GTGCTG |
| 18 | 10-1074- | CAGGTGCAGCTGCAGGAATCTGGGCCTGGACTGGTCAAACCCTCCGAGACTCT |
| | LC_1012F- | GAGCGTCACTTGTTCTGTGAGCGGCGACTCTATGAACAATTACTATTGGACAT |
| | heavy chain | GGATCCGACAGAGCCCAGGCAAGGGGCTGGAGTGGATCGGCTACATTTCTGAC |
| | variable | AGAGAAAGTGCTACTTATAACCCTAGCCTGAATTCCAGGGTGGTCATTTCACG |
| | region | CGACACCAGCAAGAACCAGCTGTCCCTGAAACTGAATTCTGTGACCCCCGCAG |
| | | ATACAGCCGTCTACTATTGCGCCACCGCTCGGAGAGGACAGCGGATCTACGGC |
| | | GTGGTCAGCTTCGGGGAGTTCTTTTACTACTACTCAATGGATGTCTGGGGGAA |
| | | GGGGACTACAGTGACCGTCTCAAGCGCCTCGACCAAG |
| 19 | 10-1074 scFv | CGTTACATAACTTACGGTAAATGGCCCGCCTGGCTGACCGCCCAACGACCCCC |
| | nucleic acid | GCCCATTGACGTCAATAATGACGTATGTTCCCATAGTAACGCCAATAGGGACT |
| | sequence | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACCATGGATTGGATTTGGCGCA |
| | | TTCTGTTTCTGGTGGGCGCGGCGACCGGCGCGCATAGCGAAGTGCAGCTGGTG |
| | | GAAAGCGGCGGCGGCGTGGTGCGCCCGGGCGGCAGCCTGCGCCTGAGCTGCGC |
| | | GGCGAGCGGCTTTACCTTTGATGATTATGGCATGAGCTGGGTGCGCCAGGCGC |
| | | CGGGCAAAGGCCTGGAATGGGTGAGCGGCATTAACTGGAACGGCGGCAGCACC |
| | | GGCTATGCGGATAGCGTGAAAGGCCGCTTTACCATTAGCCGCGATAACGCGAA |
| | | AAACAGCCTGTATCTGCAGATGAACAGCCTGCGCGCGGAAGATACCGCGGTGT |
| | | ATTATTGCGCGCGCGGCCGCAGCCTGCTGTTTGATTATTGGGGCCAGGGCACC |
| | | CTGGTGACCGTGAGCCGCGGCGGCGGCGGCAGCGGCGGCGGCGGCAGCGGCGG |
| | | CGGCGGCAGCGGCGGCGGCGGCAGCAGCAGCGAACTGACCCAGGATCCGGCGG |
| | | TGAGCGTGGCGCTGGGCCAGACCGTGCGCATTACCTGCCAGGGCGATAGCCTG |
| | | CGCAGCTATTATGCGAGCTGGTATCAGCAGAAACCGGGCCAGGCGCCGGTGCT |
| | | GGTGATTTATGGCAAAAACAACCGCCCGAGCGGCATTCCGGATCGCTTTAGCG |
| | | GCAGCAGCAGCGGCAACACCGCGAGCCTGACCATTACCGGCGCGCAGGCGGAA |
| | | GATGAAGCGGATTATTATTGCAACAGCCGCGATAGCAGCGGCAACCATGTGGT |
| | | GTTTGGCGGCGGCACCAAACTGACCGTGGGCAGCGGCGGCGGCGGCAGCCAGG |
| | | TGCAGCTGCAGGAATCTGGGCCTGGACTGGTCAAACCCTCCGAGACTCTGAGC |
| | | GTCACTTGTTCTGTGAGCGGCGACTCTATGAACAATTACTATTGGACATGGAT |
| | | CCGACAGAGCCCAGGCAAGGGGCTGGAGTGGATCGGCTACATTTCTGACAGAG |
| | | AAAGTGCTACTTATAACCCTAGCCTGAATTCCAGGGTGGTCATTTCACGCGAC |
| | | ACCAGCAAGAACCAGCTGTCCCTGAAACTGAATTCTGTGACCCCCGCAGATAC |
| | | AGCCGTCTACTATTGCGCCACCGCTCGGAGAGGACAGCGGATCTACGGCGTGG |
| | | TCAGCTTCGGGGAGTTCTTTTACTACTACTCAATGGATGTCTGGGGGAAGGGG |
| | | ACTACAGTGACCGTCTCAAGCGCCTCGACCAAGGGCGGCGGCGGCAGCGGCGG |
| | | CGGCGGCAGCGGCGGCGGCGGCAGCGGCGGCGGCGGCAGCTCCTATGTCAGGC |
| | | CACTGTCCGTCGCACTGGGGGAGACCGCAAGAATTAGCTGTGGGAGGCAGGCA |
| | | CTGGGGAGCAGGGCTGTCCAGTGGTACCAGCACCGACCAGGACAGGCACCAAT |
| | | CCTGCTGATCTACAACAATCAGGACCGGCCTTCAGGCATCCCCGAGAGATTCA |
| | | GCGGAACACCCGATATTAACTTTGGCACTAGAGCTACCCTGACAATCAGCGGA |
| | | GTGGAGGCAGGCGACGAAGCCGATTACTATTGCCATATGTGGGACTCCAGGTC |
| | | TGGGTTCAGTTGGTCATTTGGCGGAGCAACTCGACTGACCGTGCTGCGCAAAC |
| | | GCCGCGGCAGCGGCGCGACCAACTTTAGCCTGCTGAAACAGGCGGGCGATGTG |
| | | GAAGAAAACCCGGGCCCGATGCCACCTCCTCGCCTCCTCTTCTTCCTCCTCTT |
| | | CCTCACCCCCATGGAAGTCAGGCCCGAGGAACCTCTAGTGGTGAAGGTGGAAG |
| | | AGGGAGATAACGCTGTGCTGCAGTGCCTCAAGGGGACCTCAGATGGCCCCACT |
| | | CAGCAGCTGACCTGGTCTCGGGAGTCCCCGCTTAAACCCTTCTTAAAACTCAG |
| | | CCTGGGGCTGCCAGGCCTGGGAATCCACATGAGGCCCCTGGCCATCTGGCTTT |
| | | TCATCTTCAACGTCTCTCAACAGATGGGGGGCTTCTACCTGTGCCAGCCGGGG |
| | | CCCCCCTCTGAGAAGGCCTGGCAGCCTGGCTGGACAGTCAATGTGGAGGGCAG |
| | | CGGGGAGCTGTTCCGGTGGAATGTTTCGGACCTAGGTGGCCTGGGCTGTGGCC |
| | | TGAAGAACAGGTCCTCAGAGGGCCCCAGCTCCCCTTCCGGGAAGCTCATGAGC |
| | | CCCAAGCTGTATGTGTGGGCCAAAGACCGCCCTGAGATCTGGGAGGGAGAGCC |
| | | TCCGTGTCTCCCACCGAGGGACAGCCTGAACCAGAGCCTCAGCCAGGACCTCA |
| | | CCATGGCCCCTGGCTCCACACTCTGGCTGTCCTGTGGGGTACCCCCTGACTCT |
| | | GTGTCCAGGGGCCCCCTCTCCTGGACCCATGTGCACCCCAAGGGGCCTAAGTC |
| | | ATTGCTGAGCCTAGAGCTGAAGGACGATCGCCCGGCCAGAGATATGTGGGTAA |
| | | TGGAGACGGGTCTGTTGTTGCCCCGGGCCACAGCTCAAGACGCTGGAAAGTAT |
| | | TATTGTCACCGTGGCAACCTGACCATGTCATTCCACCTGGAGATCACTGCTCG |
| | | GCCAGTACTATGGCACTGGCTGCTGAGGACTGGTGGCTGGAAGGTCTCAGCTG |
| | | TGACTTTGGCTTATCTGATCTTCTGCCTGTGTTCCCTTGTGGGCATTCTTCAT |
| | | CTTCAAAGAGCCCTGGTCCTGAGGAGGAAAAGAAAGCGAATGACTGACCCCAC |
| | | CAGGAGATTC |
| 20 | 10-1074 scFv- | ATGGATTGGATTTGGCGCATTCTGTTTCTGGTGGGCGCGGCGACCGGCGCGCA |
| | nucleic acid | TAGCGAAGTGCAGCTGGTGGAAAGCGGCGGCGGCGTGGTGCGCCCGGGCGGCA |
| | sequence- | GCCTGCGCCTGAGCTGCGCGGCGAGCGGCTTTACCTTTGATGATTATGGCATG |
| | encoded | AGCTGGGTGCGCCAGGCGCCGGGCAAAGGCCTGGAATGGGTGAGCGGCATTAA |
| | region | CTGGAACGGCGGCAGCACCGGCTATGCGGATAGCGTGAAAGGCCGCTTTACCA |
| | | TTAGCCGCGATAACGCGAAAAACAGCCTGTATCTGCAGATGAACAGCCTGCGC |
| | | GCGGAAGATACCGCGGTGTATTATTGCGCGCGCGGCCGCAGCCTGCTGTTTGA |
| | | TTATTGGGGCCAGGGCACCCTGGTGACCGTGAGCCGCGGCGGCGGCGGCAGCG |
| | | GCGGCGGCGGCAGCGGCGGCGGCGGCAGCGGCGGCGGCGGCAGCAGCAGCGAA |
| | | CTGACCCAGGATCCGGCGGTGAGCGTGGCGCTGGGCCAGACCGTGCGCATTAC |
| | | CTGCCAGGGCGATAGCCTGCGCAGCTATTATGCGAGCTGGTATCAGCAGAAAC |
| | | CGGGCCAGGCGCCGGTGCTGGTGATTTATGGCAAAAACAACCGCCCGAGCGGC |
| | | ATTCCGGATCGCTTTAGCGGCAGCAGCAGCGGCAACACCGCGAGCCTGACCAT |
| | | TACCGGCGCGCAGGCGGAAGATGAAGCGGATTATTATTGCAACAGCCGCGATA |
| | | GCAGCGGCAACCATGTGGTGTTTGGCGGCGGCACCAAACTGACCGTGGGCAGC |
| | | GGCGGCGGCGGCAGCCAGGTGCAGCTGCAGGAATCTGGGCCTGGACTGGTCAA |
| | | ACCCTCCGAGACTCTGAGCGTCACTTGTTCTGTGAGCGGCGACTCTATGAACA |
| | | ATTACTATTGGACATGGATCCGACAGAGCCCAGGCAAGGGGCTGGAGTGGATC |
| | | GGCTACATTTCTGACAGAGAAAGTGCTACTTATAACCCTAGCCTGAATTCCAG |
| | | GGTGGTCATTTCACGCGACACCAGCAAGAACCAGCTGTCCCTGAAACTGAATT |
| | | CTGTGACCCCCGCAGATACAGCCGTCTACTATTGCGCCACCGCTCGGAGAGGA |
| | | CAGCGGATCTACGGCGTGGTCAGCTTCGGGGAGTTCTTTTACTACTACTCAAT |
| | | GGATGTCTGGGGGAAGGGGACTACAGTGACCGTCTCAAGCGCCTCGACCAAGG |
| | | GCGGCGGCGGCAGCGGCGGCGGCGGCAGCGGCGGCGGCGGCAGCGGCGGCGGC |
| | | GGCAGCTCCTATGTCAGGCCACTGTCCGTCGCACTGGGGGAGACCGCAAGAAT |
| | | TAGCTGTGGGAGGCAGGCACTGGGGAGCAGGGCTGTCCAGTGGTACCAGCACC |
| | | GACCAGGACAGGCACCAATCCTGCTGATCTACAACAATCAGGACCGGCCTTCA |
| | | GGCATCCCCGAGAGATTCAGCGGAACACCCGATATTAACTTTGGCACTAGAGC |
| | | TACCCTGACAATCAGCGGAGTGGAGGCAGGCGACGAAGCCGATTACTATTGCC |
| | | ATATGTGGGACTCCAGGTCTGGGTTCAGTTGGTCATTTGGCGGAGCAACTCGA |
| | | CTGACCGTGCTGCGCAAACGCCGCGGCAGCGGCGCGACCAACTTTAGCCTGCT |
| | | GAAACAGGCGGGCGATGTGGAAGAAAACCCGGGCCCGATGCCACCTCCTCGCC |
| | | TCCTCTTCTTCCTCCTCTTCCTCACCCCCATGGAAGTCAGGCCCGAGGAACCT |
| | | CTAGTGGTGAAGGTGGAAGAGGGAGATAACGCTGTGCTGCAGTGCCTCAAGGG |
| | | GACCTCAGATGGCCCCACTCAGCAGCTGACCTGGTCTCGGGAGTCCCCGCTTA |
| | | AACCCTTCTTAAAACTCAGCCTGGGGCTGCCAGGCCTGGGAATCCACATGAGG |
| | | CCCCTGGCCATCTGGCTTTTCATCTTCAACGTCTCTCAACAGATGGGGGGCTT |
| | | CTACCTGTGCCAGCCGGGGCCCCCCTCTGAGAAGGCCTGGCAGCCTGGCTGGA |
| | | CAGTCAATGTGGAGGGCAGCGGGGAGCTGTTCCGGTGGAATGTTTCGGACCTA |
| | | GGTGGCCTGGGCTGTGGCCTGAAGAACAGGTCCTCAGAGGGCCCCAGCTCCCC |
| | | TTCCGGGAAGCTCATGAGCCCCAAGCTGTATGTGTGGGCCAAAGACCGCCCTG |
| | | AGATCTGGGAGGGAGAGCCTCCGTGTCTCCCACCGAGGGACAGCCTGAACCAG |
| | | AGCCTCAGCCAGGACCTCACCATGGCCCCTGGCTCCACACTCTGGCTGTCCTG |
| | | TGGGGTACCCCCTGACTCTGTGTCCAGGGGCCCCCTCTCCTGGACCCATGTGC |
| | | ACCCCAAGGGGCCTAAGTCATTGCTGAGCCTAGAGCTGAAGGACGATCGCCCG |
| | | GCCAGAGATATGTGGGTAATGGAGACGGGTCTGTTGTTGCCCCGGGCCACAGC |
| | | TCAAGACGCTGGAAAGTATTATTGTCACCGTGGCAACCTGACCATGTCATTCC |
| | | ACCTGGAGATCACTGCTCGGCCAGTACTATGGCACTGGCTGCTGAGGACTGGT |
| | | GGCTGGAAGGTCTCAGCTGTGACTTTGGCTTATCTGATCTTCTGCCTGTGTTC |
| | | CCTTGTGGGCATTCTTCATCTTCAAAGAGCCCTGGTCCTGAGGAGGAAAAGAA |
| | | AGCGAATGACTGACCCCACCAGGAGATTC |

The amino acid sequences of the heavy and light variable regions of 10-1074 have been described in Mouquet et al. ((2012) Proc. Natl. Acad. Sci. USA 109: E3268-E3277).

In some embodiments, if a composition comprises two or more bNAbs, the mixture may be heterogeneous, meaning that there are two different species of bNAbs in the same composition. In some embodiments, if a composition comprises two or more bNAbs, the mixture may be homogeneous, meaning that there is a single species of bNAb in the same composition. All embodiments of combinations of bNAbs are contemplated by this disclosure. In some embodiments, the pharmaceutical compositions disclosed herein comprises a combination of two, three, four, five, or six or more different species of antibodies disclosed herein.

In one embodiment, the bNAb has an IC₅₀ less than 0.1 μg/ml. In one embodiment, the bNAb has an IC₅₀ less than 0.09 μg/ml. In one embodiment, the bNAb has an IC₅₀ less than 0.08 μg/ml. In one embodiment, the bNAb has an IC₅₀ less than 0.07 μg/ml. In one embodiment, the bNAb has an IC₅₀ less than 0.06 μg/ml. In one embodiment, the bNAb has an IC₅₀ less than 0.05 μg/ml. In one embodiment, the bNAb has an IC₅₀ less than 0.04 μg/ml. In one embodiment, the bNAb has an IC₅₀ less than 0.03 μg/ml. In one embodiment, the bNAb has an IC₅₀ less than 0.02 μg/ml. In one embodiment, the bNAb has an IC₅₀ less than 0.01 μg/ml. In one embodiment, the bNAb has an IC₅₀ between 0.01 and 0.1 μg/ml. In one embodiment, the bNAb has an IC₅₀ from about 0.01 and to about 0.3 μg/ml.

In one embodiment, the bNAb has an IC₈₀ less than about 0.3 μg/ml. In one embodiment, the bNAb has an IC₈₀ less than 0.2 μg/ml. In one embodiment, the bNAb has an IC₈₀ less than 0.1 μg/ml. In one embodiment, the bNAb has an IC₈₀ from about 0.1 to about 0.3 μg/ml.

In one embodiment, the bNAb has an IC₅₀ between 1 and 250 nM. In one embodiment, the bNAb has an IC₅₀ between 1 and 200 nM. In one embodiment, the bNAb has an IC₅₀ from about 1 to about 150 nM. In one embodiment, the bNAb has an IC₅₀ from about 1 to about 100 nM. In one embodiment, the bNAb has an IC₅₀ from about 1 to about 50 nM. In one embodiment, the bNAb has an IC₅₀ from about 1 about 25 nM. In one embodiment, the bNAb has an IC₅₀ from about 1 to about 10 nM. In one embodiment, the bNAb has an IC₅₀ between 1 and 5 nM. In one embodiment, the bNAb has an IC₅₀ less than 1 nM. In one embodiment, the bNAb has an IC₅₀ from about 10 to about 250 nM. In one embodiment, the bNAb has an IC₅₀ between 25 and 250 nM. In one embodiment, the bNAb has an IC₅₀ between 50 and 250 nM. In one embodiment, the bNAb has an IC₅₀ between 100 and 250 nM. In one embodiment, the bNAb has an IC₅₀ between 150 and 250 nM. In one embodiment, the bNAb has an IC₅₀ between 200 and 250 nM. In one embodiment, the bNAb has an IC₅₀ from about 10 to about 200 nM. In one embodiment, the bNAb has an IC₅₀ between 50 and 200 nM. In one embodiment, the bNAb has an IC₅₀ between 100 and 200 nM. In one embodiment, the bNAb has an IC₅₀ between 5 and 10 nM. In one embodiment, the bNAb has an IC₅₀ less than about 250 nM. In one embodiment, the bNAb has an IC₅₀ less than about 200 nM. In one embodiment, the bNAb has an IC₅₀ less than about 150 nM. In one embodiment, the bNAb has an IC₅₀ less than about 100 nM. In one embodiment, the bNAb has an IC₅₀ less than about 50 nM. In one embodiment, the bNAb has an IC₅₀ less than about 25 nM. In one embodiment, the bNAb has an IC₅₀ less than 10 nM. In one embodiment, the bNAb has an IC₅₀ less than 5 nM.

In one embodiment, the bNAb has an IC₈₀ between 1 and 250 nM. In one embodiment, the bNAb has an IC₈₀ between 1 and 200 nM. In one embodiment, the bNAb has an IC₈₀ between 1 and 150 nM. In one embodiment, the bNAb has an IC₈₀ between 1 and 100 nM. In one embodiment, the bNAb has an IC₈₀ between 1 and 50 nM. In one embodiment, the bNAb has an IC₈₀ between 1 and 25 nM. In one embodiment, the bNAb has an IC₈₀ between 1 and 10 nM. In one embodiment, the bNAb has an IC₈₀ less than 1 nM.

In one embodiment, the bNAb comprises the nucleic acid sequence of SEQ ID NO: 1. In some embodiments, the bNAb comprises a nucleic acid sequence that is at least about 88%, 89%, 90%, 91%, 92%, 93%, 94%, 95%, 96%, 97%, 98%, 99% identical to SEQ ID NO: 1. In one embodiment, the bNAb comprises the nucleic acid sequence of SEQ ID NO: 2. In some embodiments, the bNAb comprises a nucleic acid sequence that is at least about 88%, 89%, 90%, 91%, 92%, 93%, 94%, 95%, 96%, 97%, 98%, 99% identical to SEQ ID NO: 2. In one embodiment, the bNAb comprises the nucleic acid sequence of SEQ ID NO: 11. In some embodiments, the bNAb comprises a nucleic acid sequence that is at least about 88%, 89%, 90%, 91%, 92%, 93%, 94%, 95%, 96%, 97%, 98%, 99% identical to SEQ ID NO: 11. In one embodiment, the bNAb comprises the nucleic acid sequence of SEQ ID NO: 12. In some embodiments, the bNAb comprises a nucleic acid sequence that is at least about 88%, 89%, 90%, 91%, 92%, 93%, 94%, 95%, 96%, 97%, 98%, 99% identical to SEQ ID NO: 12.

In some embodiments, the antibody comprises a light chain nucleic acid sequence that is at least about 88%, 89%, 90%, 91%, 92%, 93%, 94%, 95%, 96%, 97%, 98%, 99% identical to SEQ ID NO: 3. In some embodiments, the antibody comprises a light chain nucleic acid sequence that is at least about 88%, 89%, 90%, 91%, 92%, 93%, 94%, 95%, 96%, 97%, 98%, 99% identical to SEQ ID NO: 4. In some embodiments, the antibody comprises a light chain variable region nucleic acid sequence that is at least about 88%, 89%, 90%, 91%, 92%, 93%, 94%, 95%, 96%, 97%, 98%, 99% identical to SEQ ID NO: 7. In some embodiments, the antibody comprises a heavy chain nucleic acid sequence that is at least about 88%, 89%, 90%, 91%, 92%, 93%, 94%, 95%, 96%, 97%, 98%, 99% identical to SEQ ID NO: 5. In some embodiments, the antibody comprises a heavy chain nucleic acid sequence that is at least about 88%, 89%, 90%, 91%, 92%, 93%, 94%, 95%, 96%, 97%, 98%, 99% identical to SEQ ID NO: 6. In some embodiments, the antibody comprises a heavy chain variable region nucleic acid sequence that is at least about 88%, 89%, 90%, 91%, 92%, 93%, 94%, 95%, 96%, 97%, 98%, 99% identical to SEQ ID NO: 8.

In some embodiments, the antibody comprises a light chain nucleic acid sequence that is at least about 88%, 89%, 90%, 91%, 92%, 93%, 94%, 95%, 96%, 97%, 98%, 99% identical to SEQ ID NO: 13. In some embodiments, the antibody comprises a light chain nucleic acid sequence that is at least about 88%, 89%, 90%, 91%, 92%, 93%, 94%, 95%, 96%, 97%, 98%, 99% identical to SEQ ID NO: 14. In some embodiments, the antibody comprises a light chain variable region nucleic acid sequence that is at least about 88%, 89%, 90%, 91%, 92%, 93%, 94%, 95%, 96%, 97%, 98%, 99% identical to SEQ ID NO: 17. In some embodiments, the antibody comprises a heavy chain nucleic acid sequence that is at least about 88%, 89%, 90%, 91%, 92%, 93%, 94%, 95%, 96%, 97%, 98%, 99% identical to SEQ ID NO: 15. In some embodiments, the antibody comprises a heavy chain nucleic acid sequence that is at least about 88%, 89%, 90%, 91%, 92%, 93%, 94%, 95%, 96%, 97%, 98%, 99% identical to SEQ ID NO: 16. In some embodiments, the antibody comprises a heavy chain variable region nucleic acid sequence that is at least about 88%, 89%, 90%, 91%, 92%, 93%, 94%, 95%, 96%, 97%, 98%, 99% identical to SEQ ID NO: 18.

In some embodiments, the antibody comprises nucleic acid sequences that are at least about 88%, 89%, 90%, 91%, 92%, 93%, 94%, 95%, 96%, 97%, 98%, 99% identical to 1, 2, and/or 3 CDR sequences from the variable light and/or heavy chain of PG9.

In some embodiments, the antibody comprises nucleic acid sequences that are at least about 88%, 89%, 90%, 91%, 92%, 93%, 94%, 95%, 96%, 97%, 98%, 99% identical to 1, 2, and/or 3 CDR sequences from the variable light and/or heavy chain of 10-1074.

In one embodiment, the disclosure provides an antibody, or an antigen-binding fragment thereof, (or a cell comprising the same) that comprises a heavy chain variable region and a light chain variable region as shown in Table 2.

TABLE 2

| SEQ ID | | |
|---|---|---|
| NO | Description | Sequence |
| 23 | 10-1074 | SYVRPLSVALGETARISCGRQALGSRAVQWYQHRPGQAPILLIYNNQDRPS |
| | variable | GIPERFSGTPDINFGTRATLTISGVEAGDEADYYCHMWDSRSGFSWSFGGA |
| | light (VL) | TRLTVL |
| | chain amino | |
| | acid | |
| | sequence | |
| 53 | 10-1074 | SYVRPLSVALGETARISCGRQALGSRAVQWYQHRPGQAPILLIYNNQDRPS |
| | variable | GIPERFSGTPDINFGTRATLTISGVEAGDEADYY |
| | light (VL) | |
| | chain amino | |
| | acid | |
| | sequence- | |
| | short | |
| | version | |
| 54 | Nucleic acid | tcctacgtgcggccactgtccgtggccctgggagagaccgcaaggatctcc |
| | encoding | tgcggcagacaggccctgggatctagggccgtgcagtggtatcagcacagg |
| | short | ccaggacaggcaccaatcctgctgatctacaacaatcaggaccggccttct |
| | version of | ggcatcccagagagattcagcggcacccccgatatcaactttggcacaaga |
| | 10-1074 | gccaccctgacaatcagcggagtggaggcaggcgacgaggcagattactat |
| | variable | tgtcacatgtgggacagcaggtccggcttctcttggagctttggcggagca |
| | light (VL) | acaaggctgaccgtgctg |
| | chain | |
| 55 | VL-FR1 | SYVRPLSVALGETARISCGRQ |
| 25 | VL-CDR1.1 | GRQALGSRAVQ |
| 56 | VL-CDR1.2 | ALGSRA |
| 57 | VL-FR2 | VQWYQHRPGQAPILLIY |
| 26 | VL-CDR2.1 | NNQDRPS |
| 58 | VL-CDR2.2 | NNQ |
| 59 | VL-FR3 | DRPSGIPERFSGTPDINFGTRATLTISGVEAGDEAD |
| 27 | VL-CDR3.1 | HMWDSRSGFSWS |
| 60 | VL-CDR3.2 | YYCHMWDSRSGFSWS |
| 61 | VL-FR4 | FGGATRLTVL |
| 62 | Nucleic acid | Gtggcagcaccatccgtgttcatotttccoccttctgatgagcagctgaag |
| | encoding 10- | tccggcaccgcctctgtggtgtgcctgctgaacaatttctatcctagggag |
| | 1074 | gccaaggtgcagtggaaggtggacaacgccctgcagagcggcaattcccag |
| | constant | gagtctgtgaccgagcaggacagcaaggattccacatactctctgtctagc |
| | light (CL) | accctgacactgagcaaggccgattatgagaagcacaaggtgtacgcctgt |
| | chain | gaggtgacccaccagggcctgtcctctcctgtgacaaagtccttcaacagg |
| | | ggagagtgc |
| 63 | 10-1074 | VAAPSVFIFPPSDEQLKSGTASVVCLLNNFYPREAKVQWKVDNALQSGNSQ |
| | constant | ESVTEQDSKDSTYSLSSTLTLSKADYEKHKVYACEVTHQGLSSPVTKSFNR |
| | light (CL) | GEC |
| | chain amino | |
| | acid | |
| | sequence | |
| 24 | 10-1074 | QVQLQESGPGLVKPSETLSVTCSVSGDSMNNYYWTWIRQSPGKGLEWIGYI |
| | variable | SDRESATYNPSLNSRVVISRDTSKNQLSLKLNSVTPADTAVYYCATARRGQ |
| | heavy (VH) | RIYGVVSFGEFFYYYSMDVWGKGTTVTVSS |
| | chain amino | |
| | acid | |
| | sequence | |
| 64 | 10-1074 | QVQLQESGPGLVKPSETLSVTCSVSGDSMNNYYWTWIRQSPGKGLEWIGYI |
| | variable | SDRESATYNPSLNSRVVISRDTSKNQLSLKLNSVTPADTAVYYCATARRGQ |
| | heavy (VH) | RIYGVVSFGEFFYYYSMDVWGKGTTVTVSSASTK |
| | chain amino | |
| | acid | |
| | sequence- | |
| | long version | |
| 65 | Nucleic acid | caggtgcagctgcaggagtccggaccaggactggtgaagcctagcgagacc |
| | encoding | ctgtccgtgacatgctccgtgtctggcgatagcatgaacaattactattgg |
| | long version | acctggatcaggcagtcccctggcaagggactggagtggatcggctatatc |
| | of 10-1074 | tctgacagagagagcgccacctacaacccaagcctgaatagccgggtggtc |
| | variable | atctcccgcgatacatctaagaaccagctgtctctgaagctgaatagcgtg |
| | heavy (VH) | acccccgccgacacagccgtgtactattgcgcaacagcaaggaggggacag |
| | chain | aggatctatggcgtggtgagcttcggcgagttcttttactattactccatg |
| | | gacgtgtggggcaagggcaccacagtgaccgtgagctccgccagcaccaag |
| 66 | VH-FR1 | QVQLQESGPGLVKPSETLSVTCSVS |
| 28 | VH-CDR1.1 | NYYWT |
| 67 | VH-CDR1.2 | GDSMNNYY |
| 68 | VH-FR2 | WTWIRQSPGKGLEWIGY |
| 29 | VH-CDR2.1 | YISDRESATYNPSLNS |
| 69 | VH-CDR2.2 | ISDRESA |
| 70 | VH-FR3 | TYNPSLNSRVVISRDTSKNQLSLKLNSVTPADTAVYYC |
| 30 | VH-CDR3.1 | ARRGQRIYGVVSFGEFFYYYSMDV |
| 71 | VH-CDR3.2 | ATARRGQRIYGVVSFGEFFYYYSMDV |
| 72 | VH-FR4 | WGKGTTVTVSS |
| 73 | Nucleic acid | gcctccacaaagggccctagcgtgtttccactggcaccatgcagccgctcc |
| | encoding 10- | acctctggaggcacagccgccctgggctgtctggtgaaggactacttcccc |
| | 1074 | gagcctgtgaccgtgtcttggaacagcggcgccctgaccagcggagtgcac |
| | constant | acatttccagccgtgctgcagtctagcggcctgtattccctgtcctctgtg |
| | heavy (CH) | gtgacagtgcccagctcctctctgggcacccagacatacacctgtaacgtg |
| | chain | aatcacaagcctagcaataccaaggtggacaagagggtggagctgaagacc |
| | | cctctgggcgataccacacacacatgcccacggtgtccagagcccaagtct |
| | | tgcgacaccccacccccttgccccagatgtcctgagccaaagagctgtgat |
| | | acaccacccccttgccctaggtgtcccgagcctaagtcctgcgacacccca |
| | | ccaccttgcccaaggtgtccagcaccagagctgctgggaggaccatccgtg |
| | | ttcctgtttccacccaagcctaaggatacactgatgatctctcgcacccca |
| | | gaggtgacatgcgtggtggtggacgtgagccacgaggatcccgaggtgcag |
| | | ttcaagtggtacgtggacggcgtggaggtgcacaacgccaagaccaagccc |
| | | cgggaggagcagtacaattccacctttagagtggtgtctgtgctgacagtg |
| | | ctgcaccaggattggctgaacggcaaggagtacaagtgtaaggtgtccaat |
| | | aaggccctgcctgccccaatcgagaagaccatctctaagacaaagggccag |
| | | cctcgggagccacaggtgtataccctgcctccatccagagaggagatgacc |
| | | aagaaccaggtgtctctgacatgcctggtgaagggcttctaccccagcgat |
| | | atcgcagtggagtgggagagctccggacagcctgagaacaattataatacc |
| | | acaccccctatgctggactccgatggctctttctttctgtactctaagctg |
| | | accgtggacaagagccggtggcagcagggcaacatcttcagctgttccgtg |
| | | atgcacgaggccctgcacaatcggtttacacagaagtctctgagcctgtcc |
| | | cccggcaag |
| 74 | 10-1074 | ASTKGPSVFPLAPCSRSTSGGTAALGCLVKDYFPEPVTVSWNSGALTSGVH |
| | constant | TFPAVLQSSGLYSLSSVVTVPSSSLGTQTYTCNVNHKPSNTKVDKRVELKT |
| | heavy (HL) | PLGDTTHTCPRCPEPKSCDTPPPCPRCPEPKSCDTPPPCPRCPEPKSCDTP |
| | chain amino | PPCPRCPAPELLGGPSVFLFPPKPKDTLMISRTPEVTCVVVDVSHEDPEVQ |
| | acid | FKWYVDGVEVHNAKTKPREEQYNSTFRVVSVLTVLHQDWLNGKEYKCKVSN |
| | sequence | KALPAPIEKTISKTKGQPREPQVYTLPPSREEMTKNQVSLTCLVKGFYPSD |
| | | IAVEWESSGQPENNYNTTPPMLDSDGSFFLYSKLTVDKSRWQQGNIFSCSV |
| | | MHEALHNRFTQKSLSLSPGK |
| 75 | Variant BiKE | QVQLQESGPGLVKPSETLSVTCSVSGDSMNNYYWTWIRQSPGKGLEWIGYI |
| | amino acid | SDRESATYNPSLNSRVVISRDTSKNQLSLKLNSVTPADTAVYYCATARRGQ |
| | sequence | RIYGVVSFGEFFYYYSMDVWGKGTTVTVSSASTKGGGGSGGGGSGGGGSGG |
| | (containing | GGSSYVRPLSVALGETARISCGRQALGSRAVQWYQHRPGQAPILLIYNNQD |
| | 10-1074 | RPSGIPERFSGT |
| | scFv) | PDINFGTRATLTISGVEAGDEADYY |

In one embodiment, the disclosure provides an antibody, or an antigen-binding fragment thereof, that comprises a heavy chain having a variable domain comprising an amino acid sequence as set forth in SEQ ID NO: 23 or 53. In one embodiment, the disclosure provides an antibody, or an antigen-binding fragment thereof, that comprises a light chain having a variable domain comprising an amino acid sequence as set forth in SEQ ID NO: 24 or 64.

In one embodiment, the present disclosure provides an antibody or antigen-binding fragment that has a heavy chain variable domain sequence which is at least about 70% identical, at least about 75% identical, at least about 80% identical, at least about 85% identical, at least about 90% identical, at least about 95% identical, at least about 96% identical, at least about 97% identical, at least about 98% identical, or at least about 99% identical, or identical, to SEQ ID NO: 23 or 53, and has a light chain variable domain sequence that is at least about 70% identical, at least about 75% identical, at least about 80% identical, at least about 85% identical, at least about 90% identical at least about 95% identical, at least about 96% identical, at least about 97% identical, at least about 98% identical, or at least about 99% identical, or identical to SEQ ID NO: 24 or 64.

Complementarity determining regions (CDRs) are known as hypervariable regions both in the light chain and the heavy chain variable domains of an antibody. The more highly conserved portions of variable domains are called the framework (FR). Complementarity determining regions (CDRs) and framework regions (FR) of a given antibody may be identified using systems known in the art, such as those described by Kabat et al. supra; Lefranc et al., supra and/or Honegger and Pluckthun, supra. For example, the numbering system described in Kabat et al. (1991, NIH Publication 91-3242, National Technical Information Service, Springfield, Va.) is well known to those in the art. Kabat et al. defined a numbering system for variable domain sequences that is applicable to any antibody. One of ordinary skill in the art can unambiguously assign this system of “Kabat numbering” to any variable domain amino acid sequence, without reliance on any experimental data beyond the sequence itself.

In certain embodiments, the present disclosure provides an antibody comprising the CDRs of the heavy and light chain variable domains described in Table 2. For example, the disclosure provides an antibody, or antigen-binding fragment thereof, comprising a heavy chain variable region having the CDRs described in an amino acid sequence as set forth in SEQ ID NO: 24 or 64. In one embodiment, the disclosure provides an antibody, or antigen-binding fragment thereof, comprising a light chain variable region having the CDRs described in an amino acid sequence as set forth in SEQ ID NO: 23 or 53.

In one embodiment, the present disclosure features an antibody, or an antigen-binding fragment thereof, comprising a heavy chain variable domain comprising a heavy chain CDR set (CDR1, CDR2, and CDR3) selected from the group consisting of SEQ ID NO: 28, SEQ ID NO: 29, SEQ ID NO: 30, SEQ ID NO: 67, SEQ ID NO: 69, and SEQ ID NO: 71, and a light chain variable domain comprising a light chain CDR set (CDR1, CDR2, and CDR3) selected from the group consisting of SEQ ID NO: 25, SEQ ID NO: 26, SEQ ID NO: 27, SEQ ID NO: 56, SEQ ID NO: 58, and SEQ ID NO: 60.

In some embodiments, the present disclosure features an antibody, or an antigen-binding fragment thereof, comprising a heavy chain variable domain comprising:
a) a first CDR comprising the amino acid sequence of SEQ ID NO: 28 or 67, and at least a second CDR comprising the amino acid sequence of SEQ ID NO: 29, SEQ ID NO: 30, SEQ ID NO: 69, or SEQ ID NO: 71;
b) a first CDR comprising the amino acid sequence of SEQ ID NO: 29 or 69, and at least a second CDR comprising the amino acid sequence of SEQ ID NO: 28, SEQ ID NO: 30, SEQ ID NO: 67, or SEQ ID NO: 71;
c) a first CDR comprising the amino acid sequence of SEQ ID NO: 30 or 71, and at least a second CDR comprising the amino acid sequence of SEQ ID NO: 28, SEQ ID NO: 29, SEQ ID NO: 67, or SEQ ID NO: 69; or
d) a first CDR comprising the amino acid sequence of SEQ ID NO: 28 or 67, a second CDR comprising the amino acid sequence of SEQ ID NO: 29 or 69, and a third CDR comprising the amino acid sequence of SEQ ID NO: 30 or 71.

In some embodiments, the present disclosure features an antibody, or an antigen-binding fragment thereof, comprising a light chain variable domain comprising:
a) a first CDR comprising the amino acid sequence of SEQ ID NO: 25 or 56, and at least a second CDR comprising the amino acid sequence of SEQ ID NO: 26, SEQ ID NO: 27, SEQ ID NO: 58, or SEQ ID NO: 60;
b) a first CDR comprising the amino acid sequence of SEQ ID NO: 26 or 58, and at least a second CDR comprising the amino acid sequence of SEQ ID NO: 25, SEQ ID NO: 27, SEQ ID NO: 56, or SEQ ID NO: 60;
c) a first CDR comprising the amino acid sequence of SEQ ID NO: 27 or 60, and at least a second CDR comprising the amino acid sequence of SEQ ID NO: 25, SEQ ID NO: 26, SEQ ID NO: 56, or SEQ ID NO: 58, or
d) a first CDR comprising the amino acid sequence of SEQ ID NO: 25 or 56, a second CDR comprising the amino acid sequence of SEQ ID NO: 26 or 58, and a third CDR comprising the amino acid sequence of SEQ ID NO: 27 or 60.

In one embodiment, the antibody of the disclosure comprises a heavy chain CDR set/light chain CDR set selected from the group consisting of the heavy chain variable domain CDR set of SEQ ID NO: 28, SEQ ID NO: 29 and SEQ ID NO: 30/the light chain variable domain CDR set of SEQ ID NO: 25, SEQ ID NO: 26 and SEQ ID NO: 27. In one embodiment, the antibody of the disclosure comprises a heavy chain CDR set/light chain CDR set selected from the group consisting of the heavy chain variable domain CDR set of SEQ ID NO: 67, SEQ ID NO: 69, and SEQ ID NO: 71/the light chain variable domain CDR set of SEQ ID NO: 56, SEQ ID NO: 58, and SEQ ID NO: 60.

One or more CDRs may be incorporated into a molecule either covalently or noncovalently to make it an antigen binding protein.

An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

In one embodiment, the present disclosure is directed to an antibody, or an antigen binding fragment thereof, having the antigen binding regions of any of the antibodies described in Table 2.

In one embodiment, the present disclosure is directed to an antibody, or an antigen binding fragment thereof, having antigen binding regions of antibody 10-1074. In one embodiment, the disclosure provides an antibody, or antigen-binding fragment thereof, comprising a heavy chain variable domain sequence as set forth in SEQ ID NO: 24 or 64, and a light chain variable domain sequence as set forth in SEQ ID NO: 23 or 53. In one embodiment, the disclosure is directed to an antibody having a heavy chain variable domain comprising the CDRs of SEQ ID NO: 24 or 64, and a light chain variable domain comprising the CDRs of SEQ ID NO: 23 or 53. In one embodiment, the disclosure features an isolated human antibody, or antigen-binding fragment thereof, that comprises a heavy chain variable region having an amino acid sequence that is at least about 70% identical, at least about 75% identical, at least about 80% identical, at least about 85% identical, at least about 90% identical, at least about 95% identical, at least about 96% identical, at least about 97% identical, at least about 98% identical, or at least about 99% identical to the sequence set forth in SEQ ID NO: 24 or 64, and comprises a light chain variable region having an amino acid sequence that is at least about 70% identical, at least about 75% identical, at least about 80% identical, at least about 85% identical, at least about 90% identical, at least about 95% identical, at least about 96% identical, at least about 97% identical, at least about 98% identical, or at least about 99% identical to the sequence set forth in SEQ ID NO: 23 or 53.

The bNAb antibody of the disclosure may be of an IgG class. The antibody of the disclosure may further be an IgG3 isotype.

In one embodiment, the light chain constant region IgG3 is encoded by a nucleic acid sequence shown in SEQ ID NO: 21, which encodes the amino acid sequence of SEQ ID NO: 78. In some embodiments, the light chain constant region is at least about 88%, 89%, 90%, 91%, 92%, 93%, 94%, 95%, 96%, 97%, 98%, 99% identical to SEQ ID NO: 21. In some embodiments, the light chain constant region is at least about 70%, 75%, 80%, 85%, 86%, 87% 88%, 89%, 90%, 91%, 92%, 93%, 94%, 95%, 96%, 97%, 98%, 99% identical to SEQ ID NO: 78.

In one embodiment, the heavy chain constant region is encoded by a nucleic acid sequence shown in SEQ ID NO: 22, which encodes the amino acid sequence of SEQ ID NO: 79. In some embodiments, the light chain constant region is at least about 88%, 89%, 90%, 91%, 92%, 93%, 94%, 95%, 96%, 97%, 98%, 99% identical to SEQ ID NO: 22. In some embodiments, the light chain constant region is at least about 70%, 75%, 80%, 85%, 86%, 87% 88%, 89%, 90%, 91%, 92%, 93%, 94%, 95%, 96%, 97%, 98%, 99% identical to SEQ ID NO: 79.

SEQ ID NO: 21
accgtggcggcgccgagcgtgtttatttttccgccgagcgatgaacagc
tgaaaagcggcaccgcgagcgtggtgtgcctgctgaacaacttttatcc
gcgcgaagcgaaagtgcagtggaaagtggataacgcgctgcagagcggc
aacagccaggaaagcgtgaccgaacaggatagcaaagatagcacctata
gcctgagcagcaccctgaccctgagcaaagcggattatgaaaaacataa
agtgtatgcgtgcgaagtgacccatcagggcctgagcagcccggtgacc
aaaagctttaaccgcggcgaatgc
SEQ ID NO: 78
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC
SEQ ID NO: 22
gcgagcaccaaaggcccgagcgtgtttccgctggcgccgtgcagccgca
gcaccagcggcggcaccgcggcgctgggctgcctggtgaaagattattt
tccggaaccggtgaccgtgagctggaacagcggcgcgctgaccagcggc
gtgcatacctaccggcggtgctgcagagcagcggcctgtatagcctgag
cagcgtggtgaccgtgccgagcagcagcctgggcacccagacctatacc
tgcaacgtgaaccataaaccgagcaacaccaaagtggataaacgcgtgg
aactgaaaaccccgctgggcgataccacccatacctgcccgcgctgccc
ggaaccgaaaagctgcgataccccgccgccgtgcccgcgctgcccggaa
ccgaaaagctgcgataccccgccgccgtgcccgcgctgcccggaaccga
aaagctgcgataccccgccgccgtgcccgcgctgcccggcgccggaact
gctgggcggcccgagcgtgtttctgtttccgccgaaaccgaaagatacc
ctgatgattagccgcaccccggaagtgacctgcgtggtggtggatgtga
gccatgaagatccggaagtgcagtttaaatggtatgtggatggcgtgga
agtgcataacgcgaaaaccaaaccgcgcgaagaacagtataacagcacc
tttcgcgtggtgagcgtgctgaccgtgctgcatcaggattggctgaacg
gcaaagaatataaatgcaaagtgagcaacaaagcgctgccggcgccgat
tgaaaaaaccattagcaaaaccaaaggccagccgcgcgaaccgcaggtg
tataccctgccgccgagccgcgaagaaatgaccaaaaaccaggtgagcc
tgacctgcctggtgaaaggcttttatccgagcgatattgcggtggaatg
ggaaagcagcggccagccggaaaacaactataacaccaccccgccgatg
ctggatagcgatggcagcttttttctgtatagcaaactgaccgtggata
aaagccgctggcagcagggcaacatttttagctgcagcgtgatgcatga
agcgctgcataaccgctttacccagaaaagcctgagcctgagcccgggc
aaa
SEQ ID NO: 79
ASTKGPSVFPLAPCSRSTSGGTAALGCLVKDYFPEPVTVSWNSGALTSG
VHTFPAVLQSSGLYSLSSVVTVPSSSLGTQTYTCNVNHKPSNTKVDKRV
ELKTPLGDTTHTCPRCPEPKSCDTPPPCPRCPEPKSCDTPPPCPRCPEP
KSCDTPPPCPRCPAPELLGGPSVFLFPPKPKDTLMISRTPEVTCVVVDV
SHEDPEVQFKWYVDGVEVHNAKTKPREEQYNSTFRVVSVLTVLHQDWLN
GKEYKCKVSNKALPAPIEKTISKTKGQPREPQVYTLPPSREEMTKNQVS
LTCLVKGFYPSDIAVEWESSGQPENNYNTTPPMLDSDGSFFLYSKLTVD
KSRWQQGNIFSCSVMHEALHNRFTQKSLSLSPGK

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Korff et al., 1997, Prot. Eng. 10:423; Kortt et al., 2001, Biomol. Eng. 18:95-108). By combining different VL and VH-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol. Biol. 178:379-87.

In some embodiments, the bNAb is an scFv.

In one embodiment, the scFv comprises a nucleic acid sequence that is at least about 88%, 89%, 90%, 91%, 92%, 93%, 94%, 95%, 96%, 97%, 98%, 99% identical to SEQ ID NO: 9. In one embodiment, the scFv comprises a nucleic acid sequence that is at least about 88%, 89%, 90%, 91%, 92%, 93%, 94%, 95%, 96%, 97%, 98%, 99% identical to SEQ ID NO: 10. In one embodiment, the scFv comprises a nucleic acid sequence that is at least about 88%, 89%, 90%, 91%, 92%, 93%, 94%, 95%, 96%, 97%, 98%, 99% identical to SEQ ID NO: 19. In one embodiment, the scFv comprises a nucleic acid sequence that is at least about 88%, 89%, 90%, 91%, 92%, 93%, 94%, 95%, 96%, 97%, 98%, 99% identical to SEQ ID NO: 20. In one embodiment, the scFv comprises an amino acid sequence this is at least about 70%, 75%, 80%, 85%, 86%, 87%, 88%, 89%, 90%, 91%, 92%, 93%, 94%, 95%, 96%, 97%, 98%, 99% identical to the amino acid sequence of SEQ ID NO: 75.

In one embodiment, the present disclosure provides a single chain human antibody, having a variable domain region from a heavy chain and a variable domain region from a light chain and a peptide linker connection the heavy chain and light chain variable domain regions, wherein the heavy chain variable domain sequence that is at least about 95% identical, at least about 96% identical, at least about 97% identical, at least about 98% identical, at least about 99% identical, or about 100% identical to SEQ ID NO: 24 or 64; and that has a light chain variable domain sequence that is at least about 95% identical, at least about 96% identical, at least about 97% identical, at least about 98% identical, at least about 99%, or about 100% identical to SEQ ID NO: 23 or 53. Preferably, the single chain antibody has both a heavy chain variable domain region and a light chain variable domain region, wherein the single chain human antibody has a heavy chain/light chain variable domain sequence of SEQ ID NO: 24/SEQ ID NO: 23 or SEQ ID NO: 64/SEQ ID NO: 53.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype (Lantto et al., 2002, Methods Mol. Biol. 178:303-16).

While the present disclosure provides antibodies structurally characterized by the amino acid sequences of their variable domain regions, it is understood that the amino acid sequences can undergo some changes while retaining their high degree of binding to their specific targets. More specifically, many amino acids in the variable domain region can be changed with conservative substitutions and it is predictable that the binding characteristics of the resulting antibody will not differ from the binding characteristics of the wild type antibody sequence. There are many amino acids in an antibody variable domain that do not directly interact with the antigen or impact antigen binding and are not critical for determining antibody structure. For example, a predicted nonessential amino acid residue in any of the disclosed antibodies is preferably replaced with another amino acid residue from the same class. Methods of identifying amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997), all of which are incorporated by reference in their entireties herein). Near et al. Mol. Immunol. 30:369-377, 1993 explains how to impact or not impact binding through site-directed mutagenesis. Near et al. only mutated residues that they thought had a high probability of changing antigen binding. Most had a modest or negative effect on binding affinity (Near et al. Table 3) and binding to different forms of digoxin (Near et al. Table 2).

A conservative modification or functional equivalent of a peptide, polypeptide, or protein disclosed in this disclosure (e.g., the hinge region or a heavy chain having the hinge region) refers to a polypeptide derivative of the peptide, polypeptide, or protein, e.g., a protein having one or more point mutations, insertions, deletions, truncations, a fusion protein, or a combination thereof. It retains substantially the activity to of the parent peptide, polypeptide, or protein (such as those disclosed in this disclosure). In general, a conservative modification or functional equivalent is at least about 60% (e.g., any number between 60% and 100%, inclusive, e.g., 60%, 70%, 75%, 80%, 85%, 90%, 95%, 96%, 97%, 98%, and 99%) identical to a parent (e.g., SEQ ID NO: 23 or SEQ ID NO: 24).

In one embodiment, the substitutions made within a heavy or light chain that is at least about 95% identical (or at least about 96% identical, or at least about 97% identical, or at least about 98% identical, or at least about 99% identical) are conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

A length and percent identity over that length for any nucleic acid or amino acid sequence is determined as follows. First, a nucleic acid or amino acid sequence is compared to the identified nucleic acid or amino acid sequence using the BLAST 2 Sequences (B12seq) program from the stand-alone version of BLASTZ containing BLASTN version 2.0.14 and BLASTP version 2.0.14. This stand-alone version of BLASTZ can be obtained from the State University of New York—Old Westbury campus library as well as at Fish & Richardson's web site (“www” dot “fr” dot “com”) or the U.S. govemment's National Center for Biotechnology Information web site (“www” dot “ncbi” dot “nlm” dot “nih” dot “gov”). Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -i; -r is set to 2; and all other options are left at their default setting. For example, the following command can be used to generate an output file containing a comparison between two sequences: C:\B112seq-i c:\seq1.txt-j c:\seq2.txt-p blastn-o c:\output.txt-q-1-r2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides or amino acid residues from the target sequence presented in alignment with sequence from the identified sequence starting with any matched position and ending with any other matched position. A matched position is any position where an identical nucleotide or amino acid residue is presented in both the target and identified sequence. Gaps presented in the target sequence are not counted since gaps are not nucleotides or amino acid residues. Likewise, gaps presented in the identified sequence are not counted since target sequence nucleotides or amino acid residues are counted, not nucleotides or amino acid residues from the identified sequence.

The percent identity over a determined length is determined by counting the number of matched positions over that length and dividing that number by the length followed by multiplying the resulting value by 100. For example, if (1) a 1000 nucleotide target sequence is compared to the sequence set forth in SEQ ID NO:4, (2) the B12seq program presents 200 nucleotides from the target sequence aligned with a region of the sequence set forth in SEQ ID NO: 1 where the first and last nucleotides of that 200 nucleotide region are matches, and (3) the number of matches over those 200 aligned nucleotides is 180, then the 1000 nucleotide target sequence contains a length of 200 and a percent identity over that length of 90 (i.e., 180±200*100=90).

It will be appreciated that a single nucleic acid or amino acid target sequence that aligns with an identified sequence can have many different lengths with each length having its own percent identity. For example, a target sequence containing a 20 nucleotide region that aligns with an identified sequence as follows has many different lengths

Additionally or alternatively, the nucleic acid or protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

Other modifications of the antibody are contemplated herein. For example, the antibody can be linked to one of a variety of nonproteinaceous polymers, for example, polyethylene glycol, polypropylene glycol, polyoxyalkylenes, or copolymers of polyethylene glycol and polypropylene glycol. The antibody also can be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in, for example, Remington's Pharmaceutical Sciences, 16th edition, Oslo, A., Ed., (1980).

Variant antibodies and salts thereof also are included within the scope of the disclosure. Variants of the sequences recited in the application also are included within the scope of the disclosure. Further variants of the antibody sequences having improved affinity can be obtained using methods known in the art and are included within the scope of the disclosure. For example, amino acid substitutions can be used to obtain antibodies with further improved affinity. Alternatively, codon optimization of the nucleotide sequence can be used to improve the efficiency of translation in expression systems for the production of the antibody. Variants may include non-natural amino acids up to a certain percentage. In some embodiments, the antibody comprises a variant amino acid sequence comprising about 1, 2, 3, 4, 5, 6, 7, 8, 9, 10, 11, 12, 13, 14, 15, 16, 17, 18, 19, 20 or more percent of non-natural amino acids.

### Antibody Modifications

Humanization and Primatization

In cases where the antibodies are non-human antibodies, the antibody can be “humanized” to reduce immunogenicity to a human recipient. Methods for humanizing non-human antibodies have been described in the art. See, e.g., Jones et al., Nature 321:522-525 (1986); Riechmann et al, Nature 332:323-327 (1988); Verhoeyen et al., Science 239: 1534-1536 (1988), and U.S. Pat. No. 4,816,567. Generally, residues from the variable domain of a non-human antibody are “imported” into a human immunoglobulin molecule, resulting in antibodies in which some hypervariable region residues and possibly some FR residues of a human antibody are substituted by residues from analogous sites of non-human antibodies. It is important to humanize a non-human antibody while retaining high affinity for the antigen. To this end, three dimensional immunoglobulin models are commonly available and suitable for use in analyzing proposed humanized sequences in comparison to the parental non-human antibodies. Such analysis permits identification of residues likely involved in recognition and binding of the antigen, and therefore rational design of humanized sequences that retain the specificity and affinity for the antigen.

In specific embodiments, antibodies are formed from anti-HIV human or humanized bNAbs.

Similarly, bNAbs can be “primatized” to reduce immunogenicity to another primate, non-human recipient, e.g., a rhesus recipient. Residues from the variable domain of a donor antibody (such as a non-primate antibody or an antibody of a primate species different from the recipient primate) are “imported” into a nonhuman primate recipient immunoglobulin molecule, resulting in antibodies in which some hypervariable region residues and possibly some FR residues of a nonhuman primate antibody are substituted by residues from analogous sites of donor antibodies. Alternatively, primatized antibodies can be made for use in a desirable primate species by using a recipient immunoglobulin having non-primate sequences or sequences from a different primate species by introducing the Fc fragment, and/or residues, including particularly framework region residues, from the desirable primate, into the recipient immunoglobulin. In some embodiments, the pharmaceutical composition comprises an antibody, antibody binding fragment or a salt thereof which is a humanized sequence.

Affinity Maturation

One or more hypervariable region residues of an antibody can be substituted to select for variants that have improved biological properties relative to the parent antibody by employing, e.g., affinity maturation using phage or yeast display. For example, the Fab region of an anti-HIV antibody can be mutated at several sites selected based on available structural information to generate all possible amino substitutions at each site. The antibody variants thus generated are displayed in a monovalent fashion from phage particles or on the surface of yeast cells. The displayed variants are then screened for their biological activity (e.g. binding affinity).

Modifications to the Fc Region

The antibody can be modified to improve certain biological properties of the antibody, e.g., to improve stability, to enhance or reduce effector functions such as antigen-dependent cell-mediated cytotoxicity (ADCC) and/or complement dependent cytotoxicity (CDC) of the antibody, improved or decreased internalization and/or recycling, among others.

For example, the Fc fragment of some antibodies (derived from human Ig4) can be replaced with human IgG1 that increases effector function mediated through FcRs (except FcRn). Such modification may improve the stability of the resulting antibody by about 5 fold. In another example, the IgG1 Fc fragment can be modified to improve the recycling of the antibody via the antibody salvage pathway.

Still another type of modification involves alteration of the glycosylation pattern of a parent antibody, including deletions of one or more carbohydrate moieties found in the parent antibody, or addition of one or more carbohydrates (via addition of one or more glycosylation sites) that are not present in the parent antibody.

### Methods of Transduction

### Cells

Non-limiting examples of cells that can be used in the methods described herein include T lymphocytes, dendritic cells (DC), placental stem cells (e.g., the placental stem cells disclosed in U.S. Pat. Nos. 7,468,276; 8,057,788 and 8,202,703, the disclosures of which are hereby incorporated by reference in their entireties), mesenchymal-like stem cells from umbilical cord blood, placental blood, peripheral blood, bone marrow, dental pulp, adipose tissue, osteochondral tissue, and the like; embryonic stem cells, embryonic germ cells, neural crest stem cells, neural stem cells, and differentiated cells (e.g., fibroblasts, etc.). The methods may also be used in tumor cell lines, e.g., for animal model experimental purposes. In a particular embodiment, the cells of the methods described herein may be primary cells. Primary cells are well known in the art and may include cells extracted from a subject (e.g., a human) that are cultured or expanded in vitro for an amount of time that does not lead to the onset of cellular senescence, and are not cultured or expanded in a manner that leads to immortalization of the cells. In a specific embodiment, the cells used in the methods described herein are human T lymphocytes. In another specific embodiment, the cells used in the methods described herein are not natural killer cells. In another specific embodiment, the cells used in the methods described herein are not T lymphocyte cell lines.

In one embodiment, the cells used in the methods provided herein are primary T lymphocytes (e.g., primary human T lymphocytes). The primary T lymphocytes used in the methods provided herein may be naive T lymphocytes or MHC-restricted T lymphocytes. In certain embodiments, the T lymphocytes are CD4+. In other embodiments, the T lymphocytes are CD8+. In certain embodiments, the primary T lymphocytes are tumor infiltrating lymphocytes (TILs). In certain embodiments, the primary T lymphocytes have been isolated from a tumor biopsy, or have been expanded from T lymphocytes isolated from a tumor biopsy. In certain embodiments, the primary T lymphocytes have been isolated from, or are expanded from T lymphocytes isolated from, peripheral blood, cord blood, or lymph. In certain embodiments, the T lymphocytes are allogeneic with respect to a particular individual, e.g., a recipient of said T lymphocytes. In certain other embodiments, the T lymphocytes are not allogeneic with respect to a certain individual, e.g., a recipient of said T lymphocytes. In certain embodiments, the T lymphocytes are autologous with respect to a particular individual, e.g., a recipient of said T lymphocytes.

In one embodiment, primary T lymphocytes are obtained from an individual, optionally expanded, and then transduced, using the methods described herein, with a nucleic acid encoding a bNAb (e.g. 10-1074), and optionally then expanded.

T lymphocytes can be expanded, for example, by contacting the T lymphocytes in culture with antibodies to CD3 and/or CD28, e.g., antibodies attached to beads, or to the surface of a cell culture plate; see, e.g., U.S. Pat. Nos. 5,948,893; 6,534,055; 6,352,694; 6,692,964; 6,887,466; and 6,905,681. In specific embodiments, the antibodies are anti-CD3 and/or anti-CD28, and the antibodies are not bound to a solid surface (e.g., the antibodies contact the T lymphocytes in solution). In other specific embodiments, either of the anti-CD3 antibody or anti-CD28 antibody is bound to a solid surface (e.g. bead, tissue culture dish plastic), and the other antibody is not bound to a solid surface (e.g., is present in solution).

Methods of isolating T lymphocytes are well known in the art. For example, T cells may be isolated from peripheral blood mononuclear cells (PBMC) by depleting B cells, NK cells, monocytes, platelets, dendritic cells, granulocytes and erythrocytes, according to https://www.thermofisher.com/us/en/home/references/protocols/proteins-expression-isolation-and-analysis/cell-separation-methods/human-cell-separation-protocols/isolation-of-untouched-human-t-cells-.html, which is incorporated by reference in its entirety. Exemplary isolation agents include, without limitation, Depletion Dynabeads®, Isolation buffer: Ca²⁺ and Mg²⁺ free phosphate buffered saline (PBS) (e.g. Gibco cat. no. 14190-094) supplemented with 0.1% BSA and 2 mM EDTA, heat inactivated Fetal Bovine Serum (FBS)/Fetal Calf Serum (FCS), Lymphoprep® for PBMC preparation (Axis Shield PoC, Norway), human serum albumin (HSA), 2% FBS/FCS, 0.6% sodium citrate, EDTA, and IgG antibodies against non-T cells.

In some embodiments, the disclosure relates to a method of manufacturing a T cell expressing a bNAb or fragment thereof specific for an HIV-1 epitope, the method comprising exposing an isolated T cell to one or a plurality of nucleic acid molecules comprising an expressible nucleic acid seqeunce encoding one or a plurality of bNAbs or fragments thereof. In some embodiments, the nucleic acid molecule is a plasmid, viral vector or cosmid. In some embodiments, the method further comprises exposing the one or plurality of T cells to at least one or a plurality of nucleic acid molecules encoding one or a plurality of bNAbs or fragments thereof for a time period sufficient to transduce or tranfect the T cells with one or a plurality of nucleic acid molecules.

### Transformation

As used herein, terms such as “transduction,” “transformation,” and “transfection” are used interchangeably, unless otherwise noted. Methods of transducing cells are well-known in the art. During transduction, small molecules and/or polymers may, for example, be added to cell cultures to facilitate the binding and/or uptake of the proteins and/or nucleic acids of interest. Particularly, small polar compounds can be added to culture conditions to facilitate the binding and transduction of viruses and nucleic acid(s) therein. Exemplary transformation reagents include, without limitation, Lipofectamine®, FuGENE®, calcium phosphate, diethylaminoethyl cellulose-dextran (DEAE-dextran or DD), and protamine sulfate. In certain embodiments of the methods described herein, transduction (e.g., retroviral transduction, for example lentiviral transduction) of T lymphocytes (e.g., primary human T lymphocytes) occurs in the presence of the DEAE-dextran or protamine sulfate. In particular embodiments of the methods described herein, transduction (e.g., retroviral transduction, for example lentiviral transduction) of T lymphocytes (e.g., primary human T lymphocytes) occurs in the presence of DEAE-dextran, e.g., 1(\hat{ }g/ml DEAE-dextran, or protamine sulfate, e.g., 1(\hat{ }g/ml protamine sulfate. In some embodiments, the viral Maloney viral vector with two LTR sequences comprise a multiple cloning site in which any onr or combination of nucleic acid sequence encoding the bnAb or antigen-binding fragment thereof or salt thereof.

### Culture Conditions and T Lymphocyte Activation

The cells described herein can be maintained under specific culturing conditions to facilitate or enhance transduction (e.g. viral transduction). In a particular embodiment, T lymphocytes (e.g., primary human T lymphocytes) are activated by an antigen or antigen-binding fragment that specifically binds to a T lymphocyte co-stimulatory molecule (e.g., CD28, CD3 and/or CD45) prior to or concurrently with transduction. In another embodiment, said antibody or antigen-binding fragment is coupled to a solid substrate (e.g., Dynabeads®). In a particular embodiment, T lymphocytes (e.g., primary human T lymphocytes) are stimulated by anti-CD3, anti-CD28, and/or anti-CD45 antibodies, or antigen binding fragment(s) thereof, coupled to Dynabeads® for 24 hours before transduction (e.g., viral transduction). In another particular embodiment, said antibody or antigen binding fragment(s) (e.g., of anti-CD3, anti-CD28 and/or anti-CD45 antibodies or antigen binding fragment(s) thereof) are not present on a solid substrate but are instead complexed with another compound or composition that allows presentation of the antibody or antigen binding fragment(s) to the cell, e.g., the antibody or antigen binding fragment(s) are complexed with a polymer, hydrogel, albumin, and/or a hydrophobic molecule. In particular embodiments, such molecule(s) complexed with the antibody or antigen binding fragment(s) thereof is not an adjuvant. In another embodiment, said contacting occurs at least about 48 hours, at least about 44 hours, at least about 40 hours, at least about 36 hours, at least about 32 hours, at least about 28 hours, at least about 24 hours, at least about 20 hours, at least about 16 hours, at least about 12 hours, at least about 8 hours or at least about 4 hours prior to transduction of said cells with a viral vector (e.g., retroviral transduction, for example lentiviral transduction). In yet another embodiment, said contacting occurs at least about 48 hours to about 40 hours, about 44 hours to about 36 hours, about 40 hours to about 32 hours, about 36 hours to about 28 hours, about 32 hours to about 24 hours, about 28 hours to about 20 hours, about 24 hours to about 16 hours, about 20 hours to about 12 hours, about 16 hours to about 8 hours, about 12 hours to about 4 hours or at least about 8 hours to about 1 hour prior to transduction of said cells with a viral vector (e.g., retroviral transduction, for example lentiviral transduction).

In another embodiment, cytokines and/or growth factors that stimulate T lymphocyte activation and/or proliferation can be added prior to or concurrently with transduction. In a particular embodiment, interleukin 2 (IL-2), e.g., 50 U/ml IL-2, is added to T lymphocyte cultures (e.g., primary human T lymphocyte cultures) prior to or concurrently with transduction (e.g., viral transduction). In another embodiment, interleukin 7 (IL-7), e.g., 10 ng/ml IL-7, is added to T lymphocyte cultures (e.g., primary human T lymphocyte cultures) prior to or concurrently with transduction (e.g., viral transduction). In another embodiment, interleukin 12 (IL-12), e.g., 10 ng/ml IL-12, is added to T lymphocyte cultures (e.g., primary human T lymphocyte cultures) prior to or concurrently with transduction (e.g., viral transduction). In another embodiment, interleukin 15 (IL-15), e.g., 10 ng/ml IL-15, is added to T lymphocyte cultures (e.g., primary human T lymphocyte cultures) prior to or concurrently with transduction (e.g., viral transduction). In yet another embodiment, interleukin 21 (IL-21), e.g., 25 ng/ml IL-21 is added to T lymphocyte cultures (e.g., primary human T lymphocyte cultures) prior to or concurrently with transduction (e.g., viral transduction).

### Viral Transduction

In one embodiment of the methods described herein, transduction (e.g., retroviral transduction, for example lentiviral transduction) of T lymphocytes (e.g., primary human T lymphocytes) can occur using viruses at various multiplicities of infection (MOI). In a specific embodiment of the methods described herein, transduction (e.g., retroviral transduction, for example lentiviral transduction) of T lymphocytes (e.g., primary human T lymphocytes) occurs at a viral multiplicity of infection (MOI) of 0.1, 0.2, 0.4, 0.6, 0.8, 1.0, 1.2, 1.4, 1.6, 1.8, 2.0, 2.2, 2.4, 2.6, 2.8, 3.0 or greater. In a specific embodiment of the methods described herein, transduction (e.g., retroviral transduction, for example lentiviral transduction) of T lymphocytes (e.g., primary human T lymphocytes) occurs at a viral multiplicity of infection (MOI) of 0.1 to 0.3, 0.2 to 0.4, 0.4 to 0.6, 0.6 to 0.8, 0.8 to 1.0, 1.0 to 1.2, 1.2 to 1.4, 1.4 to 1.6, 1.6 to 1.8, 1.8 to 2.0, 2.0 to 2.2, 2.2 to 2.4, 2.4 to 2.6, 2.6 to 2.8 or 2.8 to 3.0.

In a specific embodiment of the methods described herein, contacting T lymphocytes (e.g., primary human T lymphocytes) with a compound (e.g., BX795 or 2-AP) to increase transduction efficiency (e.g., lentiviral transduction efficiency) improves the transduction of isolated nucleic acid sequences (e.g., vectors encoding chimeric antigen receptors). For example, nucleic acid sequences of about 9 kilobases (kb) in length, about 10 kb in length, about 11 kb in length, about 12 kb in length, about 13 kb in length, about 14 kb in length, about 15 kb in length, about 16 kb in length, about 17 kb in length or about 18 kb in length or greater can be transduced into cells at greater efficiency as a result of the methods described herein (i.e., as compared to the efficiency of transduction in the absence of a compound described (e.g., BX795 or 2-AP)). In certain embodiments, the methods described herein result in improved transduction of nucleic acid molecules (e.g., vectors, for example, viral vectors such as retroviral, e.g., lentiviral, vectors, including vectors that encode one or more proteins, e.g., one or more chimeric antigen receptors), wherein said nucleic acid molecules are about 9 kilobases (kb) in length to about 10 kb in length, about 10 kb in length to about 11 kb in length, about 11 kb in length to about 12 kb in length, about 12 kb in length to about 13 kb in length, about 13 kb in length to about 14 kb in length, about 14 kb in length to about 15 kb in length, about 15 kb in length to about 16 kb in length, about 16 kb in length to about 17 kb in length, about 17 kb in length to about 18 kb in length, or about 9 to about 18 kb in length or about 10 to about 15 kb in length.

In a particular aspect of the methods described herein, T lymphocytes (e.g., primary human T lymphocytes) can be transduced (e.g., transduced a retrovirus, for example a lentivirus) with two or more different isolated nucleic acids, e.g., two, three, four or five nucleic acids of non-identical sequence. In a specific embodiment, contacting T lymphocytes (e.g., primary human T lymphocytes) with a compound to increase transduction efficiency (e.g., retroviral transduction efficiency, for example lentiviral transduction efficiency) improves the transduction of one, two, three, four or five different isolated nucleic acids (e.g., vectors, for example, viral vectors, such as retroviral, e.g., lentiviral, vectors, including vectors that encode one or more proteins, for example, encode one or more chimeric antigen receptors).

In a specific embodiment of the methods described herein, primary human T lymphocytes are stimulated for 24 hours with anti-CD3 and/or anti-CD28 antibodies, or antigen binding fragment(s) thereof, in the presence of 50 U/ml IL-2 and 10 μg/ml DEAE-Dextran, followed by treatment of said lymphocytes with BX795 for 3 hours, followed by lentiviral transduction of said lymphocytes, wherein the virus is at a multiplicity of infection (MOI) of 1.8 and wherein the human T lymphocytes are treated with 6 μM BX795 concurrently with the addition of the lentivirus for a further 6 hour period. [0042] In a specific embodiment of the methods described herein, primary human T lymphocytes are stimulated for 24 hours with anti-CD3 and/or anti-CD28 antibodies, or antigen binding fragment(s) thereof, in the presence of 50 U/ml IL-2 and 10 μg/ml DEAE-Dextran, followed by treatment of said lymphocytes with 2-AP for 5 hours, followed by lentiviral transduction of said lymphocytes, wherein the virus is at a multiplicity of infection (MOI) of 1.8 and wherein the human T lymphocytes are treated with 2.5-10 μM 2-AP concurrently with the addition of the lentivirus for a further 5 hour period.

In a specific embodiment of the methods described herein, primary human T lymphocytes are stimulated for 24 hours with anti-CD3 and/or anti-CD28 antibodies, or antigen binding fragment(s) thereof, in the presence of 50 U/ml IL-2 and 10 μg/ml protamine sulfate, followed by treatment of said lymphocytes with BX795 for 6 hours, followed by lentiviral transduction of said lymphocytes, wherein the virus is at a multiplicity of infection (MOI) of 1.8 and wherein the human T lymphocytes are treated with 6 μM BX795 concurrently with the addition of the lentivirus for a further 6 hour period.

In a specific embodiment of the methods described herein, primary human T lymphocytes are stimulated for 24 hours with anti-CD3 and/or anti-CD28 antibodies, or antigen binding fragment(s) thereof, in the presence of 50 U/ml IL-2 and 10 μg/ml protamine sulfate, followed by treatment of said lymphocytes with 2-AP for 5 hours, followed by lentiviral transduction of said lymphocytes, wherein the virus is at a multiplicity of infection (MOI) of 1.8 and wherein the human T lymphocytes are treated with 2.5-10 μM 2-AP concurrently with the addition of the lentivirus for a further 5 hour period.

### Isolated Nucleic Acids

One of skill in the art will appreciate that the methods described herein are not limited to transduction of any particular type of vector and that the transduced vectors are not limited with respect to the particular type of nucleic acid they comprise. Accordingly, it should be understood that compositions or vectors comprising nucleic acids used to transduce cells in accordance with the methods described herein may comprise, for example, any nucleic acid that encodes any protein or polypeptide of interest (e.g. bNAbs).

In certain embodiments, the nucleic acids may be contained within any polynucleotide vector suitable for the transduction of immune cells, e.g., T lymphocytes. For example, T lymphocytes may be transformed or transduced using synthetic vectors, retroviral vectors (e.g., lentiviral vectors), autonomously replicating plasmids, a virus (e.g., a retrovirus, lentivirus, adenovirus, or herpes virus), or the like, containing polynucleotides encoding polypeptides of interest (e.g., chimeric receptors).

In one embodiment, retroviral vectors, for example lentiviral vectors, are used in accordance with the methods described herein. Retroviral vectors, for example lentiviral vectors, suitable for transformation or transduction of T lymphocytes include, but are not limited to, e.g., the lentiviral vectors described in U.S. Pat. Nos. 5,994,136; 6,165,782; 6,428,953; 7,083,981; and 7,250,299, the disclosures of which are hereby incorporated by reference in their entireties.

Nucleic acids useful in the production of polypeptides, e.g., within a T lymphocyte, include DNA, RNA, or nucleic acid analogs. Nucleic acid analogs can be modified at the base moiety, sugar moiety, or phosphate backbone, and can include deoxyuridine substitution for deoxythymidine, 5-methyl-2′-deoxycytidine or 5-bromo-2′-deoxycytidine substitution for deoxycytidine. Modifications of the sugar moiety can include modification of the 2′ hydroxyl of the ribose sugar to form 2′-0-methyl or 2′-0-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7: 187-195; and Hyrup et al. (1996) Bioorgan. Med. Chain. 4:5-23. In addition, the deoxyphosphate backbone can be replaced with, for example, a phosphorothioate or phosphorodithioate backbone, a phosphoroamidite, or an alkyl phosphotriester backbone. In one embodiment, the nucleic acid is any nucleic acid set forth in Table 1.

In some embodiments, provided are compositions comprising expressible nucleic acid sequences encoding any antibody or antigen-binding fragment thereof disclosed herein. In some embodiments, provided are cells comprising expressible nucleic acid sequences encoding any antibody or antigen-binding fragment thereof disclosed herein. In some embodiments, the cells are T cells.

In some embodiments, provided are novel vectors and viral vectors capable of expressing exogenous gene or exogenous nucleic acid sequences in a target cell of interest, such as T cells. The present invention provides compositions and methods of use for novel viral vectors that have useful properties for gene delivery to cells, i.e., 1) efficient propagation in a packaging cell and 2) the safe and efficient expression of exogenous nucleic acid in a cell.

In some embodiments, the disclosure provides a vector comprising a viral vector, a viral vector nucleic acid, or a nucleic acid construct that comprises a viral vector nucleic acid sequence. The vector is capable of expressing an exogenous gene or exogenous nucleic acid sequences in a target cell of interest, preferably a T cell, the vector comprising a nucleic acid component or components.

In one embodiment, the vector comprises endogenous antibody signal sequence as a secretory signal. In another embodiment, cystatin-s as a secretory signal. In other embodiment, the vector comprises IL2 as a secretory signal. In another embodiment, the vector comprises TNFα as a secretory signal.

In one embodiment, the vector comprises both heavy and light chains from 10-1074 antibody. In further embodiments, the heavy and light chains are separated by 2A peptide cleavage sites.

In one embodiment, truncated CD19 can be added as a cell surface marker. In some embodiments, the antigen-binding fragment or antibody is free of any amino acid sequence that is a partial or complete cell surface marker. In some embodiments, truncated EGFR or QBEnd/10 can be used as a marker.

In one embodiment, the nucleic acid component or components comprise (i) one or more native promoter/enhancer regions in which at least one sequence segment has been modified, (ii) one or more non-native promoter/enhancers or a non-native promoter's gene or gene segment, and (iii) a native viral vector terminator or a processing signal or segment thereof, or both. Additionally, the aforementioned viral vector further comprises a non-native terminator or two or more modified sequence segments.

Such modifications may take various forms. For example, a native sequence segment can be substituted by a non-native sequence segment in the one or more promoter/enhancer regions of the vector. Further, the substitution can be of approximately the same size. In another aspect, the modification can comprise a mutation selected from any of the group members represented by a point mutation, a deletion, an insertion, and a substitution, or a combination of any of the foregoing.

In one embodiment, the viral vector is a retrovirus. In one embodiment, the retrovirus is Moloney murine leukemia virus (MMLV), or an reproductively deficient variant of the same comprising a regulatory sequence that is operably linked to a region designed to be expressed.

In another embodiment, the terminator, or processing signal, or both, as the case may be, can include a polyadenylation signal. In addition, such a viral vector can comprise a segment of the viral vector terminator or a segment of the processing signal, or both. Additionally, the function of the one or more promoter/enhancers will have been reduced, inhibited or eliminated in the present viral vector.

With respect to the one or more non-native promoters, these are capable of producing an RNA lacking a polyadenylation signal. A number of non-native promoters can be used in accordance with this invention. Simply by way of example, such non-native promoters can be selected from the group of genes represented by or designated as snRNA, tRNA, and rRNA, or a combination of any of the foregoing.

In some embodiments, in the viral vector described above, one or more non-native promoter's gene or gene segment sequence can or will have been modified. Such modifications can also take a number of forms, including the substitution or replacement of or addition to the one or more non-native promoter's gene sequence with the exogenous gene or an exogenous nucleic acid sequence.

Non-Native Vector Components useful for these purposes include non-native nucleic acid sequences in the vector. Such nucleic acid sequences can be derived from any biological system or can be chemically synthesized or can be prepared by recombinant DNA methods or by any combination of such methods. Such sequences can be approximately the same size as the vector virus sequences that are replaced. Thus, such sequences can range in size from approximately 2 to approximately 188 bases or base pairs in length, or longer. Such sequences can be used to replace one or more sequences in such regions of the virus vector as promoter and/or enhancer sequences, or any other native sequences in which its ability leads to cis effects. Such replacements can be carried out by the conventional methods of recombinant DNA (see Sambrook, J., Fritsch, E. F. and Maniatis, T. Molecular Cloning, 2nd ed. Cold Spring Laboratory, Cold Spring Harbor, N.Y., 1989, the contents of which textbook are incorporated herein by reference), and they can be conveniently performed on virus vector nucleic acid genomes or fragments thereof that are present as double stranded DNA in plasmids.

In some embodiments of the invention, to genetically engineer T cells to secrete a bNAb, a retroviral vector delivery system is used to create the transgene. In one embodiment, a transgene is made comprising the 10-1074 heavy and light chain sequence, as described herein. In one embodiment, the construct comprises antibody signal sequence followed by both heavy and light chains from 10-1074 antibody which were separated by 2A peptide cleavage sites. Truncated CD19 can be added as a cell surface marker allowing the measurement of transduction efficiency by flow cytometry. The sequences of the construct are shown below in Table 3.

TABLE 3

| SEQ | | |
|---|---|---|
| ID NO | Description | Sequence |
| 31 | CMV | CGTTACATAACTTACGGTAAATGGCCCGCCTGGCTGACCGCCCAACGACCCCC |
| | PROMOTER | GCCCATTGACGTCAATAATGACGTATGTTCCCATAGTAACGCCAATAGGGACT |
| | AND SPACER | TTCCATTGACGTCAATGGGTGGAGTATTTACGGTAAACTGCCCACTTGGCAGT |
| | REGIONS- | ACATCAAGTGTATCATATGCCAAGTACGCCCCCTATTGACGTCAATGACGGTA |
| | nucleic | AATGGCCCGCCTGGCATTATGCCCAGTACATGACCTTATGGGACTTTCCTACT |
| | acid | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCTCGTTTAGTGAACCGTCAGATCG |
| | | CCTGGAGACGCCATCCACGCTGTTTTGACCTCCATAGAAGACACCGGGACCGA |
| | | TCCAGCCTCCATCGGCTCGCATCTCTCCTTCACGCGCCCGCCGCCCTACCTGA |
| | | GGCCGCCATCCACGCCGGTTGAGTCGCGTTCTGCCGCCTCCCGCCTGTGGTGC |
| | | CTCCTGAACTGCGTCCGCCGTCTAGGTAAGTTTAAAGCTCAGGTCGAGACCGG |
| | | GCCTTTGTCCGGCGCTCCCTTGGAGCCTACCTAGACTCAGCCGGCTCTCCACG |
| | | CTTTGCCTGACCCTGCTTGCTCAACTCTAGTTAACGGTGGAGGGCAGTGTAGT |
| | | CTGAGCAGTACTCGTTGCTGCCGCGCGCGCCACCAGACATAATAGCTGACAGA |
| | | CTAACAGACTGTTCCTTTCCATGGGTCTTTTCTGCAGTCACCGTCGTCGACAC |
| | | GTGTGATCAGATATCGCGGCCGCTCTAGACCACC |
| 32 | ANTIBODY | atgggctggagctgtatcatcctgttcctggtggcaaccgcaacaggagtgca |
| | SIGNAL | cagc |
| | SEQUENCE | |
| 33 | FURIN | cgcaaacgccgc |
| | CLEAVAGE | |
| | SITE- | |
| | nucleic | |
| | acid | |
| 34 | FURIN | RKRR |
| | CLEAVAGE | |
| | SITE-amino | |
| | acid | |
| 35 | T2A | agagccgagggcaggggaagtcttctaacatgcggggacgtggaggaaaatcc |
| | | cgggccc |
| 36 | TRUNCATED | atgccacctcctcgcctcctcttcttcctcctcttcctcacccccatggaagt |
| | CD 19 | caggcccgaggaacctctagtggtgaaggtggaagagggagataacgctgtgc |
| | | tgcagtgcctcaaggggacctcagatggccccactcagcagctgacctggtct |
| | | cgggagtccccgcttaaacccttcttaaaactcagcctggggctgccaggcct |
| | | gggaatccacatgaggcccctggccatctggcttttcatcttcaacgtctctc |
| | | aacagatggggggcttctacctgtgccagccggggcccccctctgagaaggcc |
| | | tggcagcctggctggacagtcaatgtggagggcagcggggagctgttccggtg |
| | | gaatgtttcggacctaggtggcctgggctgtggcctgaagaacaggtcctcag |
| | | agggccccagctccccttccgggaagctcatgagccccaagctgtatgtgtgg |
| | | gccaaagaccgccctgagatctgggagggagagcctccgtgtctcccaccgag |
| | | ggacagcctgaaccagagcctcagccaggacctcaccatggcccctggctcca |
| | | cactctggctgtcctgtggggtaccccctgactctgtgtccaggggccccctc |
| | | tcctggacccatgtgcaccccaaggggcctaagtcattgctgagcctagagct |
| | | gaaggacgatcgcccggccagagatatgtgggtaatggagacgggtctgttgt |
| | | tgccccgggccacagctcaagacgctggaaagtattattgtcaccgtggcaac |
| | | ctgaccatgtcattccacctggagatcactgctcggccagtactatggcactg |
| | | gctgctgaggactggtggctggaaggtctcagctgtgactttggcttatctga |
| | | tcttctgcctgtgttcccttgtgggcattcttcatcttcaaagagccctggtc |
| | | ctgaggaggaaaagaaagcgaatgactgaccccaccaggagattc |

### Pharmaceutical Formulations

According to another aspect, the described invention provides a pharmaceutical composition comprising (i) one or plurality of T cells as described herein; and (ii) a pharmaceutically acceptable carrier. The pharmaceutical compositions of the described invention can further include one or more compatible active ingredients which are aimed at providing the composition with another pharmaceutical effect in addition to that provided by the cell product of the described invention. “Compatible” as used herein means that the active ingredients of such a composition are capable of being combined with each other in such a manner so that there is no interaction that would substantially reduce the efficacy of each active ingredient or the composition under ordinary use conditions.

Exemplary pharmaceutical compositions of the described invention may comprise a suspension or dispersion of cells in a nontoxic parenterally acceptable diluent or solvent. A solution generally is considered as a homogeneous mixture of two or more substances; it is frequently, though not necessarily, a liquid. In a solution, the molecules of the solute (or dissolved substance) are uniformly distributed among those of the solvent. A dispersion is a two-phase system, in which one phase (e.g., particles) is distributed in a second or continuous phase. A suspension is a dispersion in which a finely-divided species is combined with another species, with the former being so finely divided and mixed that it does not rapidly settle out. Among the acceptable vehicles and solvents that may be employed are water, Ringer's solution, and isotonic sodium chloride (saline) solution.

Additional compositions of the present invention can be readily prepared using technology which is known in the art such as described in Remington's Pharmaceutical Sciences, 18th or 19th editions, published by the Mack Publishing Company of Easton, Pa., which is incorporated herein by reference.

Formulations of the pharmaceutical composition may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the disclosure may be prepared/formulated, packaged, or sold in formulations suitable for oral, rectal, vaginal, parenteral, topical, pulmonary, intranasal, intra-lesional, buccal, ophthalmic, intravenous, intra-organ or another route of administration.

According to some embodiments, the pharmaceutical compositions of the described invention may be administered initially, and thereafter maintained by further administrations. For example, according to some embodiments, the pharmaceutical compositions of the described invention may be administered by one method of injection, and thereafter further administered by the same or by different method.

The pharmaceutical composition of the disclosure may be prepared, packaged, or sold in bulk, as a single unit dose, or as a plurality of single unit doses. As used herein, a “unit dose” is a discrete amount of the pharmaceutical composition comprising the cell product comprising a predetermined amount of the active ingredient, i.e., the one or plurality of T cells as described herein. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, according to some embodiments, a pharmaceutical composition of the disclosure may further comprise one or more additional pharmaceutically active agents, e.g., antiviral drugs, among many others. In one embodiment, the one or more additional pharmaceutically active agents include other antiviral medications used to inhibit HIV, for example nucleoside analog reverse transcriptase inhibitors, non-nucleoside reverse transcriptase inhibitors, and protease inhibitors. Among the available drugs that may be used as an additional pharmaceutically active agent are zidovudine or AZT (or Retrovir®), didanosine or DDI (or Videx®), stavudine or D4T (or Zerit®), lamivudine or 3TC (or Epivir®), zalcitabine or DDC (or Hivid®), abacavir succinate (or Ziagen”), tenofovir disoproxil fumarate salt (or Viread®), emtricitabine (or Emtriva®), Combivir® (contains 3TC and AZT), Trizivir® (contains abacavir, 3TC and AZT); three non-nucleoside reverse transcriptase inhibitors: nevirapine (or Viramune®), delavirdine (or Rescriptor®) and efavirenz (or Sustiva®), eight peptidomimetic protease inhibitors or approved formulations: saquinavir (or Invirase® or Fortovase”), indinavir (or Crixivan®), ritonavir (or Norvir®), nelfinavir (or Viracept”), amprenavir (or Agenerase®), atazanavir (Reyataz), fosamprenavir (or Lexiva), Kaletra® (contains lopinavir and ritonavir), and one fusion inhibitor enfuvirtide (or T-20 or Fuzeon®).

“Combination,” “coadministration,” “concurrent,” and similar terms referring to the administration of the pharmaceutical composition of the disclosure with an additional pharmaceutically active agents means that the components are part of a combination antiretroviral therapy or highly active antiretroviral therapy (HAART) as understood by practitioners in the field of AIDS and HIV infection.

According to some embodiments, a protein stabilizing agent can be added to the cell product comprising the one or plurality of T cells as described herein after manufacturing, for example albumin, which may act as a stabilizing agent. According to some embodiments, the albumin is human albumin. According to some embodiments, the albumin is recombinant human albumin. According to some embodiments, the minimum amounts of albumin employed in the formulation may be about 0.5% to about 25% w/w, i.e., about 0.5%, about 1.0%, about 2.0, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25% w/w, including intermediate values, such as about 12.5% w/w.

According to some embodiments, the pharmaceutical composition comprises a stabilizing amount of serum. The term “stabilizing amount” as used herein refers to the amount of serum that, when included in the formulation of the pharmaceutical composition of the described invention comprising one or plurality of T cells as described herein, enables these cells to retain their T cell effector activity. According to some embodiments, the serum is human serum autologous to a human patient. According to some embodiments, the serum is synthetic serum. According to some embodiments the stabilizing amount of serum is at least about 10% (v/v).

According to some embodiments, the methods of the present invention comprise the further step of preparing the pharmaceutical composition by adding a pharmaceutically acceptable excipient, in particular an excipient as described herein, for example a diluent, stabilizer and/or preservative.

The term “excipient” as employed herein is a generic term to cover all ingredients added to the one or plurality of T cells as described herein that do not have a biological or physiological function, which are nontoxic and do not interact with other components.

Once the final formulation of the pharmaceutical composition has been prepared it will be filled into a suitable container, for example an infusion bag or cryovial.

According to some embodiments, the methods according to the present disclosure comprises the further step of filling the pharmaceutical composition comprising the cell product containing the one or plurality of T cells as described herein or a pharmaceutical formulation thereof into a suitable container, such as an infusion bag and sealing the same to form the cell product.

According to some embodiments, the product comprising the container filled with the pharmaceutical composition comprising the cell product comprising the one or plurality of T cells as described herein of the present disclosure is frozen for storage and transport, for example at about −135° C., for example in the vapor phase of liquid nitrogen. According to some such embodiments, the formulation may also contain a cryopreservative, such as DMSO. The quantity of DMSO is generally about 20% or less, such as about 10%, 11%, 12%, 13%, 14%, 15%, 16%, 17%, 18%, 19%, or 20% v/v.

According to some embodiments, the process of the present disclosure comprises the further step of freezing the pharmaceutical composition, or the cell product comprising the one or plurality of T cells as described herein of the present disclosure. According to one embodiment, freezing occurs by a controlled rate freezing process, for example reducing the temperature by 1° C. per minute to ensure the crystals formed are small and do not disrupt cell structure. This process may be continued until the sample has reached about −100° C. Controlled- or sustained-release formulations of the pharmaceutical composition of the disclosure may be made by adapting otherwise conventional technology. The term “controlled release” as used herein is intended to refer to any drug-containing formulation in which the manner and profile of drug release from the formulation are controlled. This includes immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that preferably, although not necessarily, results in substantially constant levels of a drug over an extended time period. The term “delayed release” is used herein in its conventional sense to refer to a drug formulation in which there is a time delay between administration of the formulation and the release of the drug therefrom. “Delayed release” may or may not involve gradual release of drug over an extended period of time, and thus may or may not be “sustained release.” The term “long-term” release, as used herein, means that the drug formulation is constructed and arranged to deliver therapeutic levels of the active ingredient over a prolonged period of time, e.g., days.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations may include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. For parenteral application, suitable vehicles consist of solutions, e.g., oily or aqueous solutions, as well as suspensions, emulsions, or implants. Aqueous suspensions may contain substances, which increase the viscosity of the suspension and include, for example, sodium carboxymethyl cellulose, sorbitol and/or dextran.

According to some embodiments, the present disclosure provides a method of transporting a cell product comprising the one or plurality of T cells as described herein according to the present disclosure from the place of manufacture, or a convenient collection point, to a therapeutic facility. According to some embodiments, the temperature of the cell product is maintained during such transporting. According to some embodiments, for example, the pharmaceutical composition can be stored below 0° C., such as −135° C. during transit. According to some embodiments, temperature fluctuations of the pharmaceutical composition are monitored during storage and/or transport.

### Methods of Treatment and Prevention

In one aspect, the disclosure provides a method of treating and/or preventing an HIV infection, comprising administering to a subject in need thereof an effective amount of the cell(s) described herein (e.g. a cell comprising a nucleic acid sequence encoding any of the one or plurality of antibodies or antigen binding fragments described herein) or a pharmaceutical composition comprising the cell product comprising the one or plurality of T cells as described herein.

In one embodiment, the method further comprises administering to the subject one or a plurality of LRA molecules prior to, simultaneously with or after administering the cell or pharmaceutical composition. In one embodiment, the effective amount is sufficient to accomplish: one or any combination of (i) neutralization of one or a plurality of retroviruses in the subject; (ii) induction of NK cell recruitment to a cell infected with HIV in the subject; and (iii) antigen-specific cytotoxicity of a cell infected with HIV in the subject.

For purposes of the methods, wherein the cells or cell products as described herein are administered, the cells can be cells that are allogeneic or autologous to the mammal. Preferably, the cells are autologous to the mammal. As used herein, allogeneic means any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically. As used herein, “autologous” means any material derived from the same individual to whom it is later to be re-introduced into the individual.

According to another aspect, the present disclosure provides a method of reducing or preventing the establishment of a latent reservoir of HIV infected cells in a subject in need thereof (e.g., a subject infected with HIV or at risk of infection with HIV), thereby treating infection with a HIV infection, comprising administering to the subject a pharmaceutical composition comprising the cell, for example a T cell, comprising a nucleic acid sequence encoding any of the one or plurality of antibodies or antigen binding fragments described herein. The compositions of the disclosure can include other HIV neutralizing antibodies and/or active agent known in the art.

The disclosure also relates to a method of modifying one or a plurality of isolated T cells to secrete one or a plurality of bNAbs or fragments thereof specific for an epitope of HIV-1, the method comprising exposing the one or plurality of T cells to one or a nucleic acid molecule comprising at least a first expressible nucleic acid sequence, the nucleic acid seqeunce operably linked to at least one regulatory sequences, wherein the at least first expressible nucleic acid seqeunce encodes a bNAb specific or fragments thereof for an epitope of HIV-1. In some embodiments, the first expressible nucleic acid comprises a first nucleic acid sequence encoding a secretory signal and a second nucleic acid sequence encoding a bNAb specific or fragments thereof. In some embodiments the secretory signal is an IgG or IgE signal sequence.

### Subjects

The methods described herein are intended for use with any subject that may experience the benefits of these methods. Thus, “subjects,” “patients,” and “individuals” (used interchangeably) include humans as well as non-human subjects, particularly domesticated animals.

According to some embodiments, the subject and/or animal is a mammal, e g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal. According to some embodiments, the subject and/or animal is a human. According to some embodiments, the human is a pediatric human. According to other embodiments, the human is an adult human. According to other embodiments, the human is a geriatric human. According to other embodiments, the human may be referred to as a patient.

According to certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

According to some embodiments, the subject is a non-human animal, and therefore the disclosure pertains to veterinary use. According to some such embodiments, the non-human animal is a household pet. According to some such embodiments, the non-human animal is a livestock animal.

According to some embodiments, the subject is at risk for HIV-related diseases or disorders. Subjects at risk for HIV-related diseases or disorders include patients who have come into contact with an infected person or who have been exposed to HIV in some other way.

### Administering

The pharmaceutical compositions comprising the cells of the present invention (e.g. a cell, for example a T cell, comprising a nucleic acid sequence encoding any of the one or plurality of antibodies or antigen binding fragments described herein) may be administered in a manner appropriate to the disease to be treated. The quantity and frequency of administration will be determined by such factors as the condition of the patient, and the type and severity of the patient's disease, although appropriate dosages may be determined by clinical trials.

The administration of the pharmaceutical compositions containing the cell product may be carried out in any manner appropriate to the particular disease, including by aerosol inhalation, injection, ingestion, transfusion, implantation or transplantation. The pharmaceutical compositions of the present disclosure may be administered to a patient parenterally, e.g., subcutaneously, intradermally, intramuscularly, by intravenous (i.v.) injection, or intraperitoneally.

According to some embodiments, the pharmaceutical compositions of the described invention also can be administered to a subject by direct injection to a desired site, or systemically. For example, the pharmaceutical compositions may be injected directly into a tumor or lymph node.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. For example, parenteral administration is contemplated to include, but is not limited to, subcutaneous, intraperitoneal, intramuscular, intrasternal injection, and kidney dialytic infusion techniques. In some embodiments, the pharmaceutical compositions of the disclosure are administered intravenously.

The pharmaceutical composition comprising the cells of the present invention (e.g. a cell, for example a T cell, comprising a nucleic acid sequence encoding any of the one or plurality of antibodies or antigen binding fragments described herein) may be co-administered with various additional therapeutic agents, e.g., antiviral drugs, among others). Alternatively, the additional therapeutic agent(s) may be administered an hour, a day, a week, a month, or even more, in advance of the pharmaceutical compositions, or any permutation thereof. Further, the additional therapeutic agent(s) may be administered an hour, a day, a week, or even more, after administration of the pharmaceutical composition, or any permutation thereof. The frequency and administration regimen will be readily apparent to the skilled artisan and will depend upon any number of factors such as, but not limited to, the type and severity of the disease being treated, the age and health status of the animal, the identity of the additional therapeutic agent or agents being administered, the route of administration and the pharmaceutical composition comprising the cells of the present invention (e.g. a cell, for example a T cell, comprising a nucleic acid sequence encoding any of the one or plurality of antibodies or antigen binding fragments described herein), and the like.

According to some aspects, the present disclosure provides a method of destroying a cell in a subject infected by latent HIV infection comprising exposing the pharmaceutical composition described herein to the cell for a time period sufficient to cause cytotoxicity of the cell.

The cytotoxic activity may be assessed by any suitable technique known to those of skill in the art. For example, the cells of the present invention (e.g. a cell, for example a T cell, comprising a nucleic acid sequence encoding any of the one or plurality of antibodies or antigen binding fragments described herein) can be assayed for cytotoxic activity when in contact with a cell from a subject infected by latent HIV after an appropriate period of time, in a standard cytotoxic assay. Such assays may include, but are not limited to, the chromium release CTL assay and the ALAMAR BLUE fluorescence assay known in the art.

The term “therapeutically effective amount” mean a quantity sufficient to achieve a desired therapeutic and/or prophylactic effect, for example, an amount which results in the prevention or amelioration of or a decrease in the symptoms associated with a disease that is being treated. The amount of composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The regimen of administration can affect what constitutes an effective amount. The compound of the disclosure can be administered to the subject either prior to or after the onset of disease or disorder. Further, several divided dosages, as well as staggered dosages, can be administered daily or sequentially, or the dose can be continuously infused, or can be a bolus injection. Further, the dosages of the compound(s) of the disclosure can be proportionally increased or decreased as indicated by the exigencies of the therapeutic or prophylactic situation.

Embodiments of the disclosure relate to nucleic acid sequences encoding a first expressible amino acid sequence, wherein the first expressible amino acid seqeunce comprises an secretory signal and an antibody or antibody binding fragment disclosed herein. In some embodiments, the nucleic acid sequence comprises a coding region consisting of any one or a plurality of leader sequences. In some embodiments, the leader sequence is an IgE leader sequence: Met Asp Trp Thr Trp Ile Leu Phe Leu Val Ala Ala Ala Thr Arg Val or a leader sequence that is a functional fragment thereof comprising at least about 70%, 80%, 85%, 90%, 95%, 96%, 97%, 98% or 99% homologous to the IgE leader sequence identified in the aforementioned sentence. In some embodiments, the nucleic acid seqeunce or molecules of the disclosure relate to nucleic acid sequences comprising a leader with at least about 70%, 80%, 85%, 90%, 95%, 96%, 97%, 98% 99% or 100% sequence identity to an IgE or IgG leader sequence. In some embodiments, the leader sequence is an CD33 leader sequence: MPLLLLLPLLWAGALA. In some embodiments, the leader sequence is an IgG leader sequence: MAQVKLQESGTELAKPGAAVK or a leader sequence that is a functional fragment thereof comprising at least about 70%, 80%, 85%, 90%, 95%, 96%, 97%, 98% or 99% sequence identity to the leader sequences identified above.

According to some embodiments, the term “a therapeutically effective amount” or dose does not necessarily mean an amount that is immediately therapeutically effective, but includes a dose which is capable of expansion in vivo (after administration) to provide a therapeutic effect. According to some embodiments, “therapeutically effective” means the amount of agent required to provide a meaningful patient benefit as understood by practitioners in the field of AIDS and HIV infection. In general, the goals of treatment are suppression of viral load, restoration and preservation of immunologic function, improved quality of life, and reduction of HIV-related morbidity and mortality.

Thus, there is provided a method of administering to a patient a dose of the pharmaceutical composition comprising the cells described herein, that becomes a therapeutically effective amount after contact with a subject's cells (e.g. the cells from a subject infected with HIV or with latent HIV) in vivo to provide the desired therapeutic effect. According to some embodiments, such a dose is an amount that is less than the therapeutically effective amount.

Furthermore, the treatment or prevention provided by the method can include treatment or prevention of one or more conditions or symptoms of the disease, e.g., HIV infection, being treated or prevented.

Further, with respect to determining the effective level in a patient for treatment of HIV, in particular, suitable animal models are available and have been widely implemented for evaluating the in vivo efficacy against HIV of various therapy protocols. These models include mice, monkeys and cats. Even though these animals are not naturally susceptible to HIV disease, chimeric mice models (for example, SCID, bg/nu/xid, NOD/SCID, SCID-hu, immunocompetent SCID-hu, bone marrow-ablated BALB/c) reconstituted with human peripheral blood mononuclear cells (PBMCs), lymph nodes, fetal liver/thymus or other tissues can be infected with lentiviral vector or HIV, and employed as models for HIV pathogenesis. Similarly, the simian immune deficiency virus (SrV)/monkey model can be employed, as can the feline immune deficiency virus (FIV)/cat model. The pharmaceutical composition can contain other pharmaceuticals, in conjunction with a vector according to the disclosure, when used to therapeutically treat AIDS. These other pharmaceuticals can be used in their traditional fashion (i.e., as agents to treat HIV infection).

According to another embodiment, the present disclosure provides a pharmaceutical composition comprising the cells of the present invention (e.g. a cell, for example a T cell, comprising a nucleic acid sequence encoding any of the one or plurality of antibodies or antigen binding fragments described herein), which provides a prophylactic or therapeutic treatment choice to reduce the latent reservoir and infection of the HIV virus. The pharmaceutical compositions of the present disclosure may be formulated by any number of strategies known in the art (e.g., see McGoff and Scher, 2000, Solution Formulation of Proteins/Peptides: In McNally, E. J., ed. Protein Formulation and Delivery. New York, N.Y.: Marcel Dekker; pp. 139-158; Akers and Defilippis, 2000, Peptides and Proteins as Parenteral Solutions. In: Pharmaceutical Formulation Development of Peptides and Proteins. Philadelphia, Pa.: Talyor and Francis; pp. 145-177; Akers, et al., 2002, Pharm. Biotechnol. 14:47-127). A pharmaceutically acceptable composition suitable for patient administration will contain an effective amount of the cell, for example a T cell, comprising a nucleic acid sequence encoding any of the one or plurality of antibodies or antigen binding fragments described herein in a formulation which both retains biological activity while also promoting maximal stability during storage within an acceptable temperature range. The pharmaceutical compositions can also include, depending on the formulation desired, pharmaceutically acceptable diluents, pharmaceutically acceptable carriers and/or pharmaceutically acceptable excipients, or any such vehicle commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. The amount of an excipient that is useful in the pharmaceutical composition or formulation of this disclosure is an amount that serves to uniformly distribute the antibody throughout the composition so that it can be uniformly dispersed when it is to be delivered to a subject in need thereof. It may serve to dilute the cell, for example a T cell, comprising a nucleic acid sequence encoding any of the one or plurality of antibodies or antigen binding fragments described herein, or other active agent to a concentration which provides the desired beneficial palliative or curative results while at the same time minimizing any adverse side effects that might occur from too high a concentration. It may also have a preservative effect. Thus, for an active ingredient having a high physiological activity, more of the excipient will be employed. On the other hand, for any active ingredient(s) that exhibit a lower physiological activity, a lesser quantity of the excipient will be employed.

The above described cells, for example a T cell, comprising a nucleic acid sequence encoding any of the one or plurality of antibodies or antigen binding fragments described herein, can be administered for the prophylactic and therapeutic treatment of HIV viral infection.

Administration of a prophylactic agent can occur prior to the manifestation of symptoms characteristic of HIV-related disease or disorder, such that a disease or disorder is prevented or, alternatively, delayed in its progression.

For in vivo treatment of human and non-human patients, the patient is administered or provided a pharmaceutical formulation including cell, for example a T cell, comprising a nucleic acid sequence encoding any of the one or plurality of antibodies or antigen binding fragments described herein, of the disclosure. When used for in vivo therapy, the cells of the disclosure are administered to the patient in therapeutically effective amounts (i.e., amounts that eliminate or reduce the patient's latent viral reservoir). The cells are administered to a human patient, in accord with known methods, such as intravenous administration, for example, as a bolus or by continuous infusion over a period of time, by intramuscular, intraperitoneal, intracerobrospinal, subcutaneous, intra-articular, intrasynovial, intrathecal, oral, topical, or inhalation routes. The cells can be administered parenterally, when possible, at the target cell site, or intravenously. In some embodiments, the cells are administered by intravenous or subcutaneous administration. Therapeutic compositions of the disclosure may be administered to a patient or subject systemically, parenterally, or locally. The above parameters for assessing successful treatment and improvement in the disease are readily measurable by routine procedures familiar to a physician.

For parenteral administration, the cells may be formulated in a unit dosage injectable form (solution, suspension, emulsion) in association with a pharmaceutically acceptable, parenteral vehicle. Examples of such vehicles include, but are not limited, water, saline, Ringer's solution, dextrose solution, and 5% human serum albumin. Nonaqueous vehicles include, but are not limited to, fixed oils and ethyl oleate. Liposomes can be used as carriers. The vehicle may contain minor amounts of additives such as substances that enhance isotonicity and chemical stability, such as, for example, buffers and preservatives. The antibodies can be formulated in such vehicles at concentrations of about 1 mg/ml to about 10 mg/ml.

Other therapeutic regimens may be combined with the administration of the cells, for T cells, comprising a nucleic acid sequence encoding any of the one or plurality of antibodies or antigen binding fragments described herein, of the present disclosure. The combined administration includes coadministration, using separate formulations or a single pharmaceutical formulation, and consecutive administration in either order, wherein preferably there is a time period while both (or all) active agents simultaneously exert their biological activities. Such combined therapy can result in a synergistic therapeutic effect. The parameters for assessing successful treatment and improvement in the disease are also readily measurable by routine procedures familiar to a physician.

According to some embodiments, the pharmaceutical composition containing the cells of the present invention (e.g. a cell, for example a T cell, comprising a nucleic acid sequence encoding any of the one or plurality of antibodies or antigen binding fragments described herein) can be administered to a patient daily. According to some embodiments, the pharmaceutical composition containing the cells of the present invention (e.g. a cell, for example a T cell, comprising a nucleic acid sequence encoding any of the one or plurality of antibodies or antigen binding fragments described herein) can be administered to a patient by continuous infusion. According to some embodiments, the pharmaceutical composition containing the cells of the present invention (e.g. a cell, for example a T cell, comprising a nucleic acid sequence encoding any of the one or plurality of antibodies or antigen binding fragments described herein) can be administered to a patient twice daily. According to some embodiments, the pharmaceutical composition containing the cells of the present invention (e.g. a cell, for example a T cell, comprising a nucleic acid sequence encoding any of the one or plurality of antibodies or antigen binding fragments described herein) can be administered to a patient more than twice daily. According to some embodiments, the pharmaceutical composition containing the cells of the present invention (e.g. a cell, for example a T cell, comprising a nucleic acid sequence encoding any of the one or plurality of antibodies or antigen binding fragments described herein) can be administered to a patient every other day. According to some embodiments, the pharmaceutical composition containing the cells of the present invention (e.g. a cell, for example a T cell, comprising a nucleic acid sequence encoding any of the one or plurality of antibodies or antigen binding fragments described herein) can be administered to a patient twice a week. According to some embodiments, the pharmaceutical composition containing the cells of the present invention (e.g. a cell, for example a T cell, comprising a nucleic acid sequence encoding any of the one or plurality of antibodies or antigen binding fragments described herein) can be administered to a patient every other week. According to some embodiments, the pharmaceutical composition containing the cells of the present invention (e.g. a cell, for example a T cell, comprising a nucleic acid sequence encoding any of the one or plurality of antibodies or antigen binding fragments described herein) can be administered to a patient every 1, 2, 3, 4, 5, or 6 months.

According to some embodiments, the pharmaceutical composition comprising the cells of the present invention (e.g. a cell, for example a T cell, comprising a nucleic acid sequence encoding any of the one or plurality of antibodies or antigen binding fragments described herein) can be administered to a patient in a dosing regimen (dose and periodicity of administration) sufficient to maintain function of the administered cells (e.g. T cells) in the bloodstream of the patient over a period of about 2 weeks to about a year or more, e.g., about one month to about one year or longer, e.g., at least about 2 weeks, about 4 weeks, about 6 weeks, about 8 weeks, about 3 months, about 6 months, about a year, about 2 years.

In some embodiments, the disclosed composition is administered at a desired dosage, which in some aspects includes a desired dose or number of cells and/or a desired ratio of T-cell subpopulations. Thus, the dosage of cells in some embodiments is based on a total number of cells (or number per m² body surface area or per kg body weight) and a desired ratio of the individual populations or sub-types. In some embodiments, the dosage of cells is based on a desired total number (or number per m² body surface area or per kg of body weight) of cells in the individual populations or of individual cell types. In some embodiments, the dosage is based on a combination of such features, such as a desired number of total cells, desired ratio, and desired total number of cells in the individual populations.

In some embodiments, the disclosed composition is administered at or within a tolerated difference of a desired dose of total cells, such as a desired dose of T cells. In some aspects, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and a desired ratio, and/or based on a desired fixed dose of two or more, e.g., each, of the individual T-cell subpopulations. Thus, in some embodiments, the dosage is based on a desired fixed or minimum dose of T-cell subpopulations and a desired ratio thereof.

In certain embodiments, the disclosed composition is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual T-cell subpopulations of cells is within a range of between at or about 10⁴ and at or about 10⁹ cells/meter² (m²) body surface area, such as between 10⁵ and 10⁶ cells/m² body surface area, for example, at or about 1×10⁵ cells/m², 1.5×10⁵ cells/m², 2×10⁵ cells/m², or 1×10⁶ cells/m² body surface area. For example, in some embodiments, the cells are administered at, or within a certain range of error of, between at or about 10⁴ and at or about 10⁹ T cells/meter² (m²) body surface area, such as between 10⁵ and 10⁶ T cells/m² body surface area, for example, at or about 1×10⁵ T cells/m², 1.5×10⁵ T cells/m², 2×10⁵ T cells/m², or 1×10⁶ T cells/m² body surface area.

In some embodiments, the cells are administered at or within a certain range of error of between at or about 10⁴ and at or about 10⁹ cells/meter² (m²) body weight, such as between 10⁵ and 10⁶ cells/m² body weight, for example, at or about 1×10⁵ cells/m², 1.5×10⁵ cells/m², 2×10⁵ cells/kg, or 1×10⁶ cells/m² body surface area. In some embodiments, the cells are administered at or within a certain range of error of between at or about 10⁷ and at or about 5×10⁷ cells/m² body weight.

The frequency of the required dose will be readily apparent to the skilled artisan and will depend upon any number of factors, such as, but not limited to, the type and severity of the disease being treated, the type and age of the animal, etc.

Those in need of treatment include those already with the disorder as well as those prone to have the disorder or those in whom the disorder is to be prevented. A subject or mammal is successfully “treated” for an infection if, after receiving a therapeutic amount of an antibody according to the methods of the present disclosure, the patient shows observable and/or measurable reduction in or absence of one or more of the following: reduction in the number of infected cells or absence of the infected cells; reduction in the percent of total cells that are infected; and/or relief to some extent, one or more of the symptoms associated with the specific infection; reduced morbidity and mortality, and improvement in quality of life issues. The above parameters for assessing successful treatment and improvement in the disease are readily measurable by routine procedures familiar to a physician.

Eliminating the HIV-1 reservoir in chronic infection is key to curing the disease, but direct measurement of the latent reservoir to evaluate therapeutic eradication strategies remains difficult (Siliciano et al, Curr Opin HIV AIDS, 2013. 8(4): p. 318-25). Quantitative viral outgrowth assays and PCR-based assays of integrated DNA yield variable results (Eriksson et al., PLoS Pathog, 2013. 9(2): p. e1003174) in part because PCR cannot distinguish between inactive and permanently disabled proviruses, and outgrowth assays underestimate reservoir size (Ho et al., Cell, 2013. 155(3): p. 540-51). To that end, the most effective way to evaluate the reservoir in vivo is to measure viral rebound after terminating therapy as disclosed in the examples below.

Other aspects of the present disclosure relate to a method of producing any of the antibodies described herein. In some embodiments, the method includes a) culturing a host cell (e.g., any of the host cells described herein) comprising an isolated nucleic acid, vector, and/or vector system (e.g., any of the isolated nucleic acids, vectors, and/or vector systems described herein) under conditions such that the host cell expresses the antibody; and b) isolating the antibody from the host cell. Methods of culturing host cells under conditions to express a protein are well known to one of ordinary skill in the art. Methods of isolating proteins from cultured host cells are well known to one of ordinary skill in the art, including, for example, by affinity chromatography (e.g., two step affinity chromatography comprising protein A affinity chromatography followed by size exclusion chromatography).

All patent applications, issued patents and journal articles disclosed herein are incorporated by reference in their entireties.

### EXAMPLES

### Example 1: Generation of T_{bnAb} Cells

Several strategies have been tested for HIV elimination but they all suffer from similar challenges, including the heterogeneous and rapidly mutating nature of HIV, leading to viral escape, poor persistence in vivo, the potential of acquiring resistance. Thus far, these strategies have only shown transient efficacy in clinical trials.

| Therapy | Approach | Limitations |
|---|---|---|
| CD8 clones | Selection and expansion of | Short persistence |
| | high reactivity clones | No CD4 help |
| | | Limited specificity - |
| | | HIV escape |
| | | Minimal efficacy |
| Transdominant | Utilizes competitive | Short persistence |
| protein expression | inhibition of cognate viral | Limited reduction in |
| | protein counterparts to | HIV burden |
| | prevent viral replication | |
| CCR5/CXCR4 KO | Knockout HIV co-receptors | Off-target toxicity |
| | | Need for dual KO |
| High-affinity T cell | Find conserved epitopes and | HLA restricted |
| receptors (SLY) | express artificial receptors in | Generation of new |
| | T cells | receptor for every |
| | | HLA type |
| | | Off-target toxicity |
| CAR T cells | Utilizes CD4 scFv or | Limited reduction in |
| | Mab scFv to target HIV | HIV burden |
| | infected CD4 t-cells | Off-target toxicity |
| | | Potential for HIV |
| | | infection through |
| | | CD4 receptor |
| | | Inability to target |
| | | alternative HIV |
| | | infected cells |

HIV SPECIFIC T CELLS. An important role for HIV-specific cytotoxic T-cell responses in the control of viremia has been clearly defined (reviewed in²⁴). Strong cytotoxic T cell responses have been observed in patients with low viral loads and non-progressive HIV infection²⁵—so-called “HIV controllers.” These individuals have been shown capable of controlling viremia in the absence of ART, and were to have high frequencies of HIV-specific CD8+ T cells that were capable of suppressing viral infection.²⁶ Elite controllers are able to maintain undetectable levels of HIV, which has been associated with a significantly increased breadth of Gag-specific CD8+ T cell response, when compared to chronic progressors and individuals with ART suppressed HIV. Decrease in viremia occurs during peak CD8 T cell responses during initial control of primary HIV.²⁷ In patients who succumb to infection, dysregulated CD8+ T cell function has been observed—for example, lower levels of perforin have been noted in HIV-specific T cells (compared to CMV-specific cells) from HIV+ patients.²⁸ Studies have thus investigated the therapeutic consequences of stimulating or augmenting the CD8+ T cell response. Macaques given a vaccine that induced CD8+ T cell responses cleared SIV infection.²⁹ Infusion of autologous, ex vivo-expanded CTL targeting HIV epitopes within gp120, gag, and nef resulted in increased CD4 T cells in the first two weeks and decrease in plasma viremia, although longer term analysis of HIV infection showed no statistically significant improvements.³⁰ Even T cells from HAART treated patients were capable of conferring long-lasting immune responses both systemically and in the GI mucosa after they have been expanded ex vivo.³¹ However, HIV specific T cells alone are subject to limitations including immune escape and viral evasion mechanisms, such as the downregulation of MHC-I on infected cells. HIV-specific T cell also may be largely excluded from the B-cell follicles of lymphoid tissue that harbor HIV reservoirs in T-follicular helper cells, and have no capacity to target cell free virus, such as that which is trapped on the follicular dendritic cell network, also in lymphoid follicles³²⁻³⁴. Thus, it seems unlikely that HIV-specific T cells alone will be capable of achieving sterilizing cures.

BROADLY NEUTRALIZING ANTIBODIES. Early studies on HIV+ patient sera showed that neutralizing antibodies bind to the virus and prevent it from infecting cells. Antibodies from these patients recognized the gp120 glycoprotein in the HIV envelope and neutralized different HIV-1 virus isolates.³⁵ More recently, broadly neutralizing antibodies recognizing the CD4 binding site have been identified (VRC01),³⁶⁻³⁸ and they have already been used to engineer next-generation antibodies increased potency in vitro, and in vivo.³⁷ From a therapeutic standpoint, it is difficult to elicit production of broadly neutralizing antibodies in vivo,³⁹ and so passive immunization by direct administration of these broadly neutralizing antibodies have been attempted. One study showed that sustained administration of simianized neutralizing HIV-1 antibodies completely protected animals against viral challenge.⁴⁰ Unfortunately, ex vivo manufacture of these proteins are hampered by costs and issues in scale up.¹⁷ Antibodies also have short half-lives, and thus frequent reinfusions of broadly neutralizing antibodies are needed.⁴¹ For these reasons, investigators have looked at producing virus through a vector—for example, AAV encoding neutralizing antibodies and delivered intramuscularly resulted in endogenous synthesis of antibodies that resulted in long-lasting protection against intravenous infection with simian immunodeficiency virus in non human primates.⁴² Our related strategy should provide continued production of neutralizing antibodies at the anatomic sites of latency, addressing the need for manufacturing large quantities of antibody for passive immunization while also supplying cell-mediated immunity against reservoir cells. Against the viral reservoir, combination of broadly neutralizing antibody and LRA showed promising results in a humanized mouse model of HIV.⁴³ However, a closer look at the mechanisms of protection suggests that Fc-FcR-mediated events increase suppression of virus.⁴³ For this, antibody engineering to ensure antibody dependent cell cytotoxicity (ADCC) capabilities in our secreted antibody will help recruit this crucial arm of immunity.

ANTIBODY DEPENDENT CELL CYTOTOXICITY (ADCC). A role for ADCC in HIV control has been suggested by the presence of increased NK cell activity in patients that have been exposed to HIV but remain uninfected,⁴⁴ and by the high level of ADCC in elite controllers compared to viremic patients.⁴⁵ However, it was the partially successful RV144 vaccine trial that emphasized the key roles of antibody mechanisms beyond neutralization, specifically ADCC through natural killer (NK) cells correlate with protection.⁴⁶ Participants receiving ALVAC and AIDSVAX vaccine showed some measure of efficacy and modest benefits, with trends towards protection against HIV and vaccine efficacies ranging from 26.4 to 31.2%,⁴⁷ despite the absence of neutralizing antibody activity—pointing to ADCC as the process responsible for the clinical effects. In fact, the presence of antibodies mediating ADCC in vaccine recipients was associated with protection against HIV.⁴⁸ Certain antibody intrinsic properties confer ADCC activity; fortunately antibody engineering allow for modification of identified antibodies.⁴⁹ Our strategy will thus improve the chances of success by engineering broadly neutralizing antibodies to also mediate ADCC, and recruit NK cells to the site of the latent reservoir.

Caskey et al. (2017) have shown that broadly neutralizing antibodies, and 10-1074 specifically, are able to transiently decrease HIV RNA levels in a subset of the population.

Selecting the Optimal Antibody

We developed a method to perform paired testing of neutralization and infected-cell binding (predictive of ADCC⁶) and applied this to 36 viruses that we isolated from quantitative viral outgrowth assay (QVOA) supernatants, using a panel of 14 clinically relevant bnAbs.

We observed clear distinctions between viruses that were sensitive to infected cell binding by a given antibody, ex. 10-1074 binding to patient OM5162 virus isolate #1, and those that were resistant, ex. lack of 10-1074 binding to OM5162 virus isolate #3 (data not shown). The bnAb 10-1074 showed superior binding characteristics within this cohort based on a combination of breadth and potency (data not shown). 10-1074 also showed potent neutralization of 24/36 viruses (data not shown). While the CD4bs antibodies VRC07, 3BNC117, and N6 were superior with respect to neutralizing breadth, we felt it important to prioritize potency to maximize our chances of observing antiviral activity of T cell secreted bnAbs. Thus, we prioritized 10-1074 for initial T cell transductions. (Ren Y, Korom M, Truong R, Chan D, Huang S H, Kovacs C C, Benko E, Safrit J T, Lee J, Garbán H, Apps R, Goldstein H, Lynch R M, Jones R B. J Virol. 2018 Nov. 12; 92(23). pii: e00895-18. doi: 10.1128/JVI.00895-18. Print 2018 Dec. 1.).

Designing the Construct

We constructed transgenes according to the following principle: broadly neutralizing antibodies will function as expected if the full wildtype sequence was used. Antibody heavy and light chains were arranged with 2A sequences to allow expression of a functional antibody similar to its natural counterpart.⁷ We used the signal sequence of antibodies to allow secretion of the transgene. Following transfection of Phoenix Eco cells with retroviral plasmids encoding the transgene, supernatants were harvested and were used to infect PG13 producer cell lines. Producer cell lines expressing high levels of the CD19 receptor were isolated by flow sorting, and clones derived from single cells from this population were grown and tested for expression of CD19 and secretion of broadly neutralizing antibody (data not shown).

Expression of the Construct in HIV-Specific T Cells

Retrovirus-mediated transduction using a Moloney murine leukemia virus was used to modify our HIV-specific T cells, similar to the approach used to introduce chimeric antigen receptors onto T cells. HIV specific T cells were stimulated thrice and then subsequently infected with a retrovirus encoding the antibody constructs described above.⁸ Briefly to generate HXTC/dHXTC, matured DCs, generated from adherent monocytes following 7 days culture with GMCSF/IL4 and subsequent maturation, were pulsed with gag, nef, and pol pepmixes. Peptide compositions of pepmixes from JPT were selected to provide broad coverage across all HIV clades. These dendritic cells were then co-cultured with peripheral blood mononuclear cells. For the priming or the first stimulation, IL-7, IL-12, and IL-15 were added. For the second stimulation, T cells were re-stimulated with pepmix-pulsed autologous irradiated PHA blasts (T cells that were mitogenically stimulated with phytohemagutinnin to act as feeders that allow for antigen presentation). Irradiated co-stimulatory K562 cells were added to provide Costimulation. These artificial presenting cells were also added during subsequent stimulations. Throughout this manufacturing process, HXTC was grown in Raltegravir and Indinavir to prevent outgrowth of participant's autologous HIV reservoir. We then tested expression of the construct in T cells by looking at surface expression of CD19. Because CD19 is made as part of precursor protein and is cleaved afterwards at the 2A sequence, there is an approximate 1:1 stoichiometry between marker expression and antibody production.

Antibody Secretion in Gene-Modified T Cell

We then tested whether these gene-modified T cells were capable of secreting antibodies. As our GMP compliant protocol uses human AB serum, we are unable to measure total IgG to assess secretion of HIV-specific bnAbs from T cells (due to background IgG in human serum). We therefore utilized an HIV-specific ELISA where plates were coated with gp120 protein.⁹ This approach has the additional benefit of only quantifying antibody that is functionally competent with respect to binding HIV-gp120. For these experiments, cells are plated 5×10⁵ to 1×10⁶/mL (5×10⁵ to 1×10⁵/100 uL) and supernatants harvested between 1-3 days post transduction or stimulation. We initially tested constructs that expressed heavy chain and light chain separately in two retroviral vectors—requiring double transductions; although transduction efficiencies increase with two viral transductions, there was no detectable antibodies secreted. Recently, we developed a more robust transgene that featured 10-1074 heavy and light chains expressed in the same construct. T cells showed average 10-1074 antibody concentrations of 160 ng/mL in corresponding supernatants. We are also currently testing another retroviral constructs that has different configurations for the same antibody (10-1074): featuring an scFv for 10-1074 (variable heavy-linker-variable light) coupled to a linker that is subsequently coupled to an scFv for CD16 (essentially a modified Bispecific Killer Engager/BiKE).

Function of Secreted Antibody

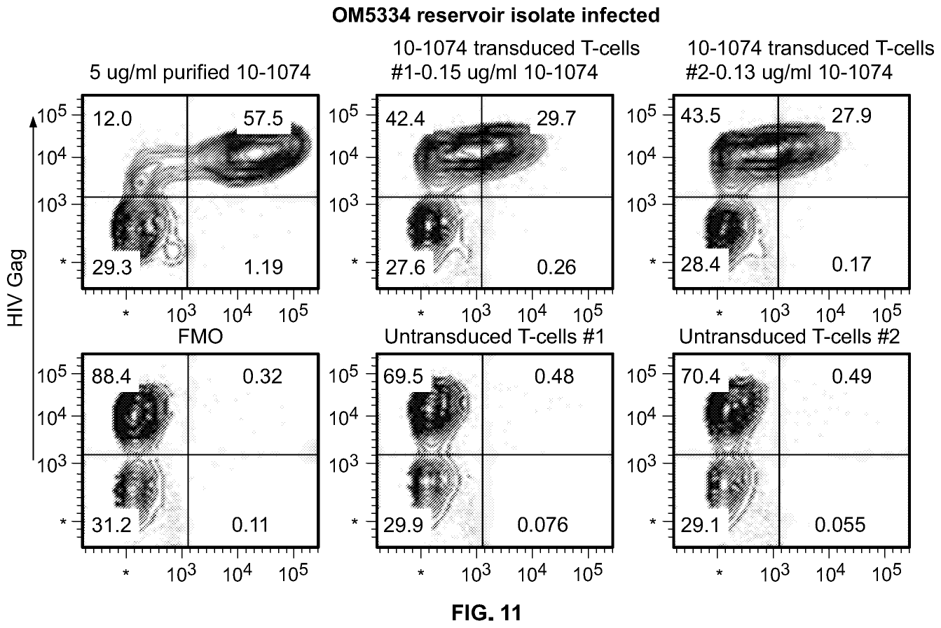

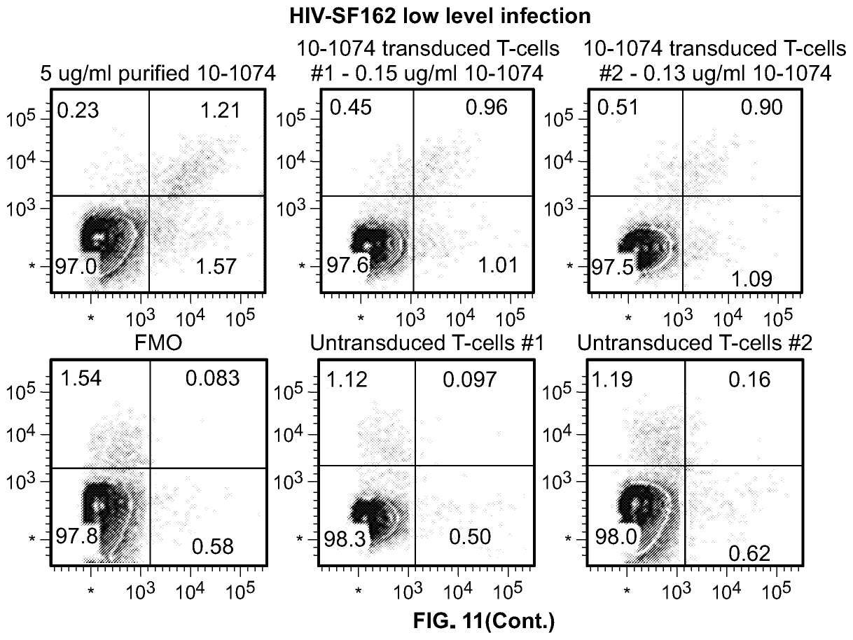

Binding of secreted 10-1074 bnAb to HIV-Infected CD4⁺ T-cells. We demonstrated efficient binding of purified 10-1074 to the surfaces of CD4⁺ T cells that had been infected with virus isolated from the latent reservoir of participant “OM5334” (data not shown). Here, we tested whether 10-1074 secreted from HIV-specific T_{bnAb} cell lines was also functional in this regard. Primary CD4+ T cells were infected with this virus, and then co-cultured with supernatants harvested directly from HIV-specific T_{bnAb} cell lines. These cells were then stained with a fluorochrome-conjugated anti-human-IgG1 antibody, and analyzed by flow cytometry. We observed specific binding of T_{bnAb} secreted 10-1074 to HIV-infected cells (FIG. 11). The level of binding observed with these supernatants was, however, less than with purified 10-1074; proportional to differences in bnAb concentration (5 ug/ml in purified versus 0.15-0.13 ug/ml in supernatants). We reasoned that this level of binding may have been negatively affected by the artificially high levels of HIV infection in these cultures (˜70% Gag⁺) which would be expected to adsorb significant amounts of bnAb. We therefore performed an additional experiment using a lower multiplicity of infection (MOI) of the HIV molecular clone SF162. At these more physiologically relevant infection levels, we observed similar magnitudes of binding between 5 ug/ml of purified 10-1074 and the 10-1074 in the supernatants of the T_{bnAb}. We have not yet been able to generate ADCC data due to ongoing troubleshooting on the NK cell effector side of the assay. However, it has been well-established that Ab-binding to infected cells is predictive of ADCC⁶. Thus, we feel that our data strongly support the potential for bnAbs secreted from our transduced T_{bnAb} cells to engage ADCC against HIV-infected cells.

Virus neutralization by secreted 10-1074. We also tested whether the supernatants secreted from T_{bnAb} are capable of neutralizing HIV by using a standard pseudovirus assay. Briefly, supernatants obtained from T_{bnAbs} were diluted five fold and incubated with pseudovirus. Target cells were then added, and a single-round of infection was allowed to proceed for 48 h¹. In certain lines, we observed 20-30% neutralization of virus at this single concentration (data not shown). The curves show 20-30% neutralization falling between 0.01-0.1 ug/ml of purified 10-1074 and the median IC₅₀ against reservoir viruses was 0.3 ug/ml. (data not shown). These 115 dilutions of T_{bnAb} supernatants contained 0.03 ug/ml of 10-1074. Thus, the data support that the 10-1074 in these supernatants is functionally equivalent to purified 10-1074 on a per concentration basis.

T Cell Function in Gene-Modified Cells

We then tested the ability of gene-modified cells to maintain their function following modification to express broadly neutralizing antibodies, by determining their ability to secrete IFNγ in response to HIV antigens gag, pol, and nef. T cell secretion of 10-1074 broadly neutralizing antibodies does not impair their ability to recognize HIV antigens presented in the context of MHC, suggesting maintenance of their T cell functions, and potential synergy against latently infected cells (where the T cell targets the cell directly through TCR-MHC interactions and their secreted antibodies target HIV). Antibody levels were measured from supernatants collected from cells that have been plated at a concentration of 1×106/mL (1×105/100 uL), and were harvested up to a maximum of five days post transduction or stimulation. HIV specific T cells secreting 10-1074 antibody constructs maintain their phenotype, as the majority of our cell product is CD3+ T cells, with an even split of CD4+ and CD8+ T cells (data not shown).

In order to have a lasting efficacy against HIV, a combination approach was used in which three anti-viral effector functions are elicited. In this approach, genetic modification of T cells to secrete broadly neutralizing antibodies (bnAbs) against HIV not only maintains their T cell effector functions through specific cytotoxicity against HIV infected target cells, but also engages the endogenous immune system through antibody-dependent cell-mediated cytotoxicity (ADCC) and directly neutralizing cell free virus. Thus the optimal construct can facilitate a tripartite (T cell killing, ADCC, neutralizing antibody) attack on the HIV reservoir.

We tested the transduction of multiple constitutive bnAb constructs into HIV-specific T cell lines to generate T_{bnAbs} and assessed the in vitro antiviral activities of these cells. The cellular platform we selected was our ex vivo expanded multi-HIV specific T cell, generated by repeated stimulations of isolated CD8+ T-cells with antigen presenting cells expressing a mix of peptides that span multiple HIV antigens (gag, pol, nef, and others). We optimized a GMP-compliant method for generating HIV-specific T cells from ARV-treated HIV-infected donors. These cells were generated from participants who donate leukapheresis samples 3 times per year, providing sufficient autologous target cells for the assays. Following ex vivo expansion against HIV antigens gag and nef, these cells demonstrated high specificity against all three antigens as measured by IFNγ ELISPOT.

We will also generate separate retroviral vectors encoding each of the HIV-specific bnAbs VRC01, VRC09, PGT121, and PG9, as well as an HCV-specific antibody to be used as a negative control. The sequences of these bnAbs are available on GenBank (e.g. DD257981.1 for HCV Ab). Other bnAbs that can potentially be used include 3BNC117, 3BNC60, 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al, Nature, 2012. 492(7427): p. 118-22, Horwitz et al, Proc Natl Acad Sci USA, 2013. 110(41): p. 16538-43, Scheid, et al. 2011. Science, 333: 1633-1637, Scheid, et al. 2009. Nature, 458:636-640, Eroshkin et al, Nucleic Acids Res. 2014 January; 42133-9, Mascola et al. Immunol Rev. 2013 July; 254(1):225-44. Antibody heavy and light chains are arranged with F2A sequences adjacent to modified furin cleavage sites, to allow expression of a functional antibody similar to its natural counterpart. Each antibody will be engineered to either enhance ADCC (substituting Fc domains with IgG1 GASDALIE variant) or, as a control for experiments, to minimize ADCC (substituting Fc domains with GRLR variant) by modifying the Fc region (see Table 4).

TABLE 4
List of initial constructs for potential testing.

| Leader Sequence | Antibody | Fc Domain |
|---|---|---|
| Immunoglobulin heavy chain | VRC01 | GASDALIE |
| Immunoglobulin heavy chain | VRC09 | GASDALIE |
| Immunoglobulin heavy chain | PGT121 | GASDALIE |
| Immunoglobulin heavy chain | PG9 | GASDALIE |
| Immunoglobulin heavy chain | HCV | GASDALIE |
| Immunoglobulin heavy chain | VRC01 | GRLR |
| Immunoglobulin heavy chain | VRC09 | GRLR |
| Immunoglobulin heavy chain | PGT121 | GRLR |
| Immunoglobulin heavy chain | PG9 | GRLR |
| Immunoglobulin heavy chain | HCV | GRLR |

Other Fc domains that can be used include, but of limited to, IgG1, IgG3, scFcv for CD16, and single domain antibody for CD16.

An immunoglobulin leader sequence previously used to allow T cell secretion of an antibody-like molecule (a bispecific T cell engager) is placed in the 5′ end of the entire sequence.¹⁶ Constitutive expression is driven by the LTR of a retroviral vector. Retrovirus-mediated transduction using a Moloney murine leukemia virus is used to modify our HIV-specific T cells, similar to the approach used to introduce chimeric antigen receptors onto T cells. HIV specific T cells are stimulated thrice and then subsequently infected with a retrovirus encoding the antibody constructs described above.⁵⁶ Following transductions, time-course experiments to assess the secretion of Abs from these T_{bnAb} as measured by IgG ELISAs are performed. T_{bnAbs} expressing each of the bnAbs in Table 4 are generated from cells from at least 5 ARV-treated HIV-infected donors. Whether the transduced T cells maintain their antiviral function, and simultaneously determine whether secreted antibodies neutralize HIV and mediate ADCC is tested. On the basis of performance in these experiments, a bnAb to proceed to future experiments is selected.

Assessing T Cell Response. Genetically modified T cells with their unmodified counterparts in terms of phenotype (staining for CD25, CD69, CD45RA, CD45RO, CD62L, CCR7, CD27, CD28, CD95, CD244, PD1, CTLA4, Tim3) are compared using flow cytometry, specificity (against HIV peptides) using IFNγ ELISPOT, function by analyzing cytokine secretion in response to the presence of HIV antigens, and cytotoxicity by performing chromium release assays. Cytotoxicity is assessed by exposing chromium-labeled autologous PBMC (expanded with PHA) to overlapping peptide libraries of different HIV antigens. The abilities of these cells to eliminate autologous productively HIV-infected cells are also tested.

Assessing Antibody Neutralization. We use a panel of HIV isolates representing different clades as test viruses. We first co-incubate different combinations of a strain of HIV, CD4 T cells, and equal concentrations of our different constructs (parent bnAb and T cell-secreted bnAb). After different time-points, we harvest the supernatant from these cultures and check for production of p24 protein (indicative of active HIV infection) using p24 ELISA. bnAbs that successfully neutralize the virus are unable to infect CD4 T cells and thus have the lowest levels of p24 protein.

Assessing NK Cell Activity. We use target cells expressing HIV env proteins to assess NK cell activity and determine whether secreted bnAbs mediate ADCC. Expi293 cells are transfected with the HIV env glycoprotein, which allows physiologic glycosylation and the creation of an artificial glycan shield surrounding these proteins. After successful transfection (checked by flow cytometry), cells are labeled with chromium. bnAbs secreted by T cells modified by different constructs, along with the parental bnAbs are allowed to bind to the env-expressing cells with the excess subsequently washed off. Two groups of NK cell populations (one expanded with IL2 and IL15 following CD56 selection of pheresis products, and another obtained directly from pheresis product and CD56 selection) are coincubated with env-expressing Expi293 and antibody. Non-env expressing Expi293 and Env-expressing 293 cells alone serve as negative controls. K562 cells (excellent targets for NK cells) serve as positive controls. Cytotoxicity is measured by calculating the amount of chromium release and normalizing with negative and positive controls. Constructs engineered to express the ADCC-associated GASDALIE modification are expected to perform the best in terms of cytotoxicity, while no ADCC is expected from Fc GRLR-modified T cell-secreted bnAbs. The HCV specific GRLR and GASDALIE antibodies can serve as additional negative controls.

Generation of T_{bnAb} Cells Secreting Antibody Under NF-kB Control

We will use HIV-specific T cells and CMV-specific T cells as our cellular platforms. CMV-specific T cells are generated in a similar fashion as HIV-specific T cells, albeit with different antigens (IE1 and pp65, instead of gag, pol, and nef). The best performing constructs identified in Example 1 (from Table 4) are used to modify these two cell populations. Similar to Example 1, antigen specific T cells will be stimulated thrice and then subsequently infected with a retrovirus encoding the antibody constructs described above. The retroviral construct follows the same schema as above—with the exception of the NF-kB promoter driving gene expression.

We will stimulate HIV-specific and CMV-specific T_{bnAbs} from 5 donors separately with anti-CD3/anti-CD28 beads at multiple cell:bead ratios, with peptide pulsed autologous BLCL using serially diluted concentrations of peptide, and with HIV-infected versus uninfected autologous CD4+ Tcells. Supernatants will be harvested daily for 5 days, and the production of bnAbs is measured by IgG ELISA assays.

### Example 2: Latency Reversing Agents (LRAs)

LRAs. An array of candidate LRAs are currently under various stages of development, with a number having entered into HIV clinical trials (reviewed in⁵⁰). A subset of LRAs, including IL-2, IL-15/IL-15SA, prostratin, bryostatin, T cell receptor agonists, and others function, at least in part, by activating the transcription factor NF-kB, a major element involved in the activation of LTR-dependent HIV transcription.⁵¹ By placing bnAb production under the control of an NF-kB promoter we design a system whereby the LRA, instead of merely acting as activator of the latent reservoir, now serves as a lynch pin of the therapeutic strategy: triggering the tripartite immune response coincident with reactivating quiescent HIV-infected cells. Additionally, by placing antibody secretion of genetically modified T cells under the control of NFkB, neutralizing antibodies are produced in the local disease environment immediately following reactivation of the reservoir—facilitating sequential and spatial integration of this therapy, and limiting any toxicity issues or T_{bnAbs} anergy that may result from constitute expression.

The proposed combination of latency reversing agents with tripartite immunotherapy offers a potentially very powerful approach to eradicating persistent HIV reservoirs. The use of LRAs to both reactivate HIV from resting CD4+ T cells and to stimulate bnAb production from genetically modified HIV-specific T cells (T_{bnAbs}), thus recruiting ADCC activity from endogenous NK cells, is a novel concept that allow for spatial and temporal co-ordination between latency reversal and immune attack. Coupling mAb production to T cells also serves to target mAb production to lymphoid tissues, which represent critical anatomical persistent viral reservoirs^{52,53}. We explore multiple LRAs, including IL-15SAs, which additionally enhances the survival and function of both T_{bnAbs} and NK cells^{19-21,54,55}. Furthermore, as NF-kB is also triggered by T cell receptor stimulation, any recognition of virus-infected cells by T_{bnAbs} amplifies bnAb production at these sites. This coupling between reactivation and immune stimulation may be the key towards improving “shock and kill” approaches for HIV eradication. Moreover, to our knowledge, the use of broadly neutralizing antibodies, multi antigen HIV-specific T cells, and antibody-dependent cell cytotoxicity through NK cells as the basis of a single therapeutic platform has never been previously explored.

Screening of LRAs. We test the following LRAs to determine their ability to stimulate T cell secretion of bnAb: IL-2, IL-15SA, bryostatin, and prostratin. Inducible T_{bnAbs} from at least 5 ARV-treated HIV-infected donors are cultured with these LRAs over a range of concentrations. Supernatants is harvested daily for 5 days, and the production of bnAbs is measured by IgG ELISA assays.

Assessment of TCR Activation Effects on Secretion. We stimulate HIV-specific and CMV-specific T_{bnAbs} from 5 donors separately with anti-CD3/anti-CD28 beads at multiple cell:bead ratios, with peptide pulsed autologous BLCL using serially diluted concentrations of peptide, and with HIV-infected versus uninfected autologous CD4+ T-cells. Supernatants is harvested daily for 5 days, and the production of bnAbs is measured by IgG ELISA assays.

We will confirm whether the optimized/identified antibody construct that has performed well in these parameters maintains those same functions. Therefore in this experiment, we will be comparing the parental bnAb, the constitutively secreted bnAb, and the NF-kB controlled bnAb.

Assessing Antiviral Activity of T_{bnAbs}. We will test the abilities of T_{bnAbs} from 5 HIV-infected ARV-treated subjects to eliminate productively HIV-infected cells in the presence of LRAs (to induce bnAb production), and will dissect the roles of T cell cytotoxicity, ADCC, and neutralization. We anticipate utilizing IL-15SA as our lead LRA. NF-kB stimulation occurs following treatment with LRAs like IL-15SA, and following recognition of cognate HIV antigen presented in the context of MHC. PBMC autologous to T_{bnAbs} will be stimulated with antibodies against CD3 and CD28, as well as with IL-2. These cells will then be superinfected with HIV LAI and co-cultured with the following T_{bnAbs} and controls: i) HIV-specific T_{bnAbs} with GASDALI HIV-specific Ab; ii) CMV-specific T_{bnAbs} with GRLR HCV-specific Ab (negative for all effector functions); iii) HIV-specific T_{bnAbs} with GRLR HIV-specific Ab (negative for ADCC); iv) HIV-specific T_{bnAbs} with GRLR HCV-specific Ab (negative for ADCC and neutralization); v) CMV-specific T_{bnAbs} GASDALI HIV-specific Ab (negative for T cell cytotoxicity); vi) CMV-specific T_{bnAbs} GRLR HIV-specific Ab (negative for T cell cytotoxicity and ADCC).

Co-cultures with effectors will be performed for 72 hours and at multiple effector:target ratios ranging from 1:1 to 1:100. Cells will then be stained and analyzed by flow cytometry, and levels of infection will be assessed by measuring the % of HIV-Gag+ cells within the viable CD3+CD8-population (to identify all CD4 cells, including HIV-infected cells which downregulate CD4) as previously demonstrated.⁶⁴ We anticipate observing the following hierarchy of viral suppression in these assays: no effector functions <neutralization <ADCC+neutralization <cytolytic function only <cytolytic function+neutralization <cytolytic function+neutralization+ADCC.

In the current example we will utilize a variation on an ex vivo HIV eradication assay to test the abilities of HIV-specific T_{bnAb} cell lines in combination with pharmacologically achievable concentrations of LRAs to eliminate natural HIV reservoirs from ex vivo patient samples. HIV-specific T_{bnAbs} given along with an LRA at pharmacologically achievable concentrations will detect and eliminate latently HIV-infected cells from the natural patient-derived reservoir as measured by decreases in both cell associated HIV DNA and inducible virus. We expect that this will involve contributions from T-cell cytotoxicity and from ADCC.

In vitro eradication assay. We have developed an in vitro HIV eradication assay that allows us to test the abilities of HIV-specific CTL clones to eliminate the latent reservoir from autologous CD4+ T-cells. Utilizing this assay, we have demonstrated that HIV-specific CTL clones, derived from ARV-treated HIV-infected subjects, given in combination with IL-15SA are capable of reducing the reservoir as measured by cell-associated HIV DNA and inducible virus (outgrowth assays).

Study Participants. We have a cohort of ARV-treated HIV-infected patients who contribute leukapheresis samples 3 times per year, and on whom we have banked at least 1×10⁹ cyropreserved PBMC/subject (see letter of support from Dr. Colin Kovacs). The HIV- and CMV-specific T_{bnAbs} cells utilized in all aspects of this study will have been derived from these individuals, thus giving us access to abundant target cells for in vitro eradication assays.

We will select HIV-specific T_{bnAb} cell lines with demonstrated antiviral activity (targeted against epitopes that are not escaped in the autologous reservoir) and autologous CMV-specific T_{bnAb} cell lines from 5 ARV-treated HIV-infected subjects. These will be tested in a modified version of the assay where HIV-specific T_{bnAb} cells will be added to whole autologous PBMC along with an LRA shown to induce bnAb secretion, and ARVs. By utilizing whole PBMC rather than purified CD4+ T-cells we will incorporate the NK cells and phagocytes needed to mediate ADCC and other mechanisms of clearing Ab-labeled target cells. Following 5 days of co-culture with T_{bnAb}, we will perform negative selection (Easysep, Stemcell Technologies) to isolate CD4+ T-cells. These will be subjected to an additional CD8-depletion step (Dynabeads, Life Technologies) to remove any residual T_{bnAb} cells. We will quantify the remaining viral reservoir (see data analysis, below) and compare between the following conditions: i) no treatment; ii) LRA only; iii) HIV-specific T_{bnAb} cell line; iv) HIV-specific T_{bnAb} (HIV-specific antibody) cell line+LRA; v) CMV-specific T_{bnAb} (irrelevant control antibody) cell line; vi) CMV-specific T_{bnAb} cell line+LRA. Given our previous success with IL-15SA used at a pharmacologically relevant concentration with conventional CTL, as well as the ability of IL-15SA to directly enhance survival and function of NK cells (reviewed in¹⁹) we anticipate focusing on this is our lead LRA. However, we can prioritize other LRAs based on superior abilities to induce Ab production from T_{bnAb} cell lines. PBMC from these assays will be obtained by leukapheresis, and we will have sufficient cell numbers to test two different LRAs in parallel in the above conditions. This experiment will determine whether or not T_{bnAb} cell lines are capable of reducing the natural reservoir in a manner that depends upon CTL recognition of targets, as well as will reveal heterogeneity between the efficacies of the different cell lines tested.

We will select 3 HIV-specific T_{bnAb} cell lines that exhibited significant reductions in the natural reservoir. We will generate paired cell lines for this specificity that: i) inducibly secrete HIV-specific ‘GASDALIE’ Fc variant (activates ADCC) bnAb, ii) inducibly secretes HIV-specific ‘GRLR’ Fc variant bnAb (negative control for ADCC), iii) inducibly secretes an irrelevant non-HIV-specific mAb with the GASDALIE Fc variant (negative control for ADCC and neutralization). We will perform ex vivo viral eradication assays, but for each cell line comparing the three variants described above. We will test each of these variants at effector:target ratios of 1:10, 1:25, 1:100 to allow us to gain insights into the relative potencies of these variants on a per cell basis. These experiments will provide insights into whether ADCC and/or neutralization enhance the reductions in viral reservoirs achieved by CTL alone. Regarding neutralization, these experiments will be performed in the presence of ARVs, however neutralization may prevent any cell-to-cell transmission of virus that could still occur in this context⁶⁶.

Impacts of the interventions on the reservoir will be measured. We will measure viral RNA in supernatants at the end of the 5 day co-culture period. We expect that the addition of IL-15SA (or other LRA) will be associated with the production of detectable virus.

The absence of cell-free virus following treatment with a combination of IL-15SA+T_{bnAb}, will be interpreted as supporting that T_{bnAb} eliminates or suppresses virus production from reactivated infected cells. Total cell-associated HIV DNA in purified CD4+ T-cells directly following co-culture will be quantified by digital droplet PCR.

Viral Outgrowth Assays. Remaining cells will be plated at 1×10⁶ cells/well, and activated with PHA and irradiated feeder in the presence of MOLT-4 CCR5 cells to amplify any virus produced. Viral production will be measured by p24 ELISA and by qPCR.

### Example 3: HIV-Specific T_{bnAbs} Improve Control and Eradicate the HIV Latent Reservoir in a Humanized Mouse Model (CATmice)

HIV-specific T_{bnAbs} in combination with an LRA will target and reduce the natural HIV reservoir in vivo in a humanized mouse model of persistence. We anticipate that both T-cell cytolytic activity, and ADCC will contribute to this effect, and that neutralization of virus may also play a role. We will utilize HIV-infected mice possessing a natural, patient-derived persistent HIV reservoir as a pre-clinical model to determine whether HIV-specific T_{bnAbs} exhibit potent anti-reservoir activity in vivo. This will require T_{bnAbs} to persist in vivo (with cytokine support) and to be effectively induced to produce bnAbs at sufficient concentrations to trigger ADCC Observation of substantially enhanced anti-reservoir activity of T_{bnAbs} as compared to unmodified T-cells would provide rationale to move towards clinical trials in future work.

We have developed a novel humanized mouse model of HIV persistence called the CD4 ARV treated mouse cAi-mouse. In this model, NSG mice (which lack murine T-cells, B-cells, and NK cells) are reconstituted with CD4+ T-cells from ARV-treated HIV-infected subjects. In the absence of ARV therapy, viremia rebounds from the natural HIV reservoir contained in these cells within weeks. Viral rebound can be suppressed by the administration of pediatric formulations of ARVs in drinking water and re-emerges upon cessation of ARV therapy. Virus can be reactivated from resting CD4+ T-cell splenocytes isolated from suppressed animals by LRAs in vitro.

The reconstitution of mice with CD4+ T-cells from HIV-infected adults allows for the testing of autologous natural HIV-specific CTL clones or lines in adoptive transfer experiments. We have observed that adoptive transfer of some HIV-specific CTL clones, given with IL-15SA as a supporting cytokine, can markedly delay viral rebound. This very likely represents reductions in the viral reservoir rather than ongoing suppression of viremia, as CTL have only been found to persist for up to 7 days with cytokine support. We will utilize a variation of this model incorporating NK cells to determine whether HIV-specific T_{bnAb} cells can target and reduce the HIV reservoir in vivo.

Mice will be reconstituted with CD4+ T-cells from ARV-treated HIV-infected subjects and maintained on ARV therapy for 2 weeks. Mice will then receive autologous human NK cells that have been activated in vitro with IL-12, IL-15, and IL-18. This treatment has previously been shown to upregulate the high-affinity IL-2 receptor IL-2RapY allowing for subsequent enhancement of cytotoxicity, cytokine production, and survival upon adoptive transfer into NSG mice by provision of low-dose IL-2 therapy⁶⁷. Initially, we will perform experiments using HIV-specific T_{bnAbs} cells from 5 subjects comparing the following groups with 5 mice each: i) no T-cells; ii) CMV-specific T_{bnAbs} expressing an irrelevant non-HIV-specific antibody; iii) HIV-specific T_{bnAbs} expressing a ‘GASDALIE’ variant HIV-specific bnAb (shown to be effective in vitro). All mice will receive daily injections of 0.2 mg/kg IL-15SA to serve the functions of: improving the survival of T_{bnAbs}, reversing HIV latency, inducing expression of bnAb from the NF-Kb promoter, and enhancing NK cell function, and of 75,000 IU IL-2/mouse to enhance NK cell survival.

We will select three HIV-specific T_{bnAbs} cell lines that exhibit delays in viral rebounds and further dissect the roles of CTL-killing, ADCC, and neutralization in this outcome. Mice will be divided into groups of 10 each to receive NK cells+: i) no T-cells ii) HIV-specific T_{bnAbs} expressing a ‘GASDALIE’ variant HIV-specific bnAb iii) HIV-specific T_{bnAbs} expressing a ‘GRLR’ variant HIV-specific bnAb (abrogates ADCC activity) iv) CMV-specific T_{bnAbs} expressing a ‘GASDALIE’ variant HIV-specific bnAb (negates CTL killing) v) HIV-specific T_{bnAbs} expressing an irrelevant mAb (negates both ADCC and neutralization). One week after administering cells both daily injections of cytokines and ARV treatments will be stopped, and animals will be bled weekly to assess HIV viral loads.

The primary end-point of these experiments will be time to first viral rebound to >10,000 copies/ml following cessation of ARV therapy. These data will be assessed by a Kaplan-Meier survival analysis and statistical significance will be evaluated by Log rank test and hazard ratios with 95% confidence intervals will be calculated. Throughout the experiment we will also monitor animals by weekly bleeding to assess the persistence of T_{bnAbs}, CD4+ T-cells, and NK cells as well as systemic levels of IL-15SA and of the Abs produced by T-cells.

### Example 4: In Vivo Clinical

We will generate sufficient preclinical data to justify scale-up for manufacturing this novel immunotherapy product for clinical translation. Following the successful demonstration of the efficacy of this approach, we will develop GMP-compliant methodologies to manufacture these antibody-secreting T cells, and apply for FDA approval for a phase I clinical study.

### Example 5: Engineered Antigen-Specific T Cells Secreting Broadly Neutralizing Antibodies

This example describes the generation of HIV-specific cytolytic T-cells (CTLs) that have been engineered to secrete the broadly neutralizing HIV-specific antibody (bnAb) 10-1074. The disclosed HIV-specific CTLs can be used in a therapeutic strategy involving a combination of cell therapy with HIV specific T cells and HIV-specific broadly neutralizing antibodies that elicit ADCC. With such an approach, both arms of immunity can be simultaneously recruited to mount an anti-HIV response with the ability to target the elimination of persistent HIV reservoirs from multiple fronts.

Materials and Methods

10-1074 Antibody Construct Design

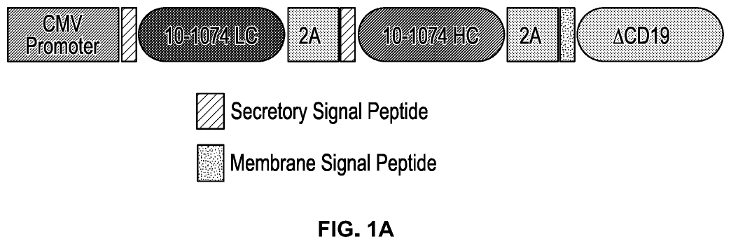

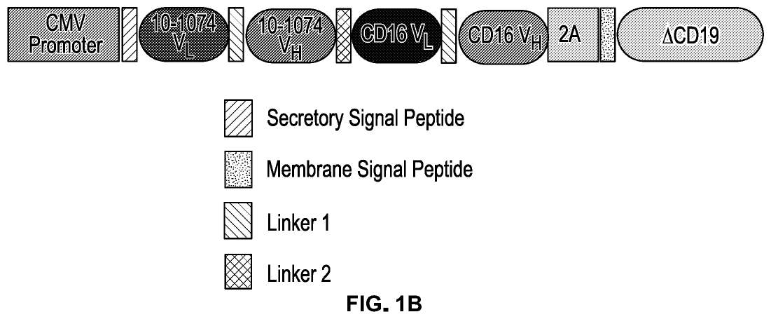

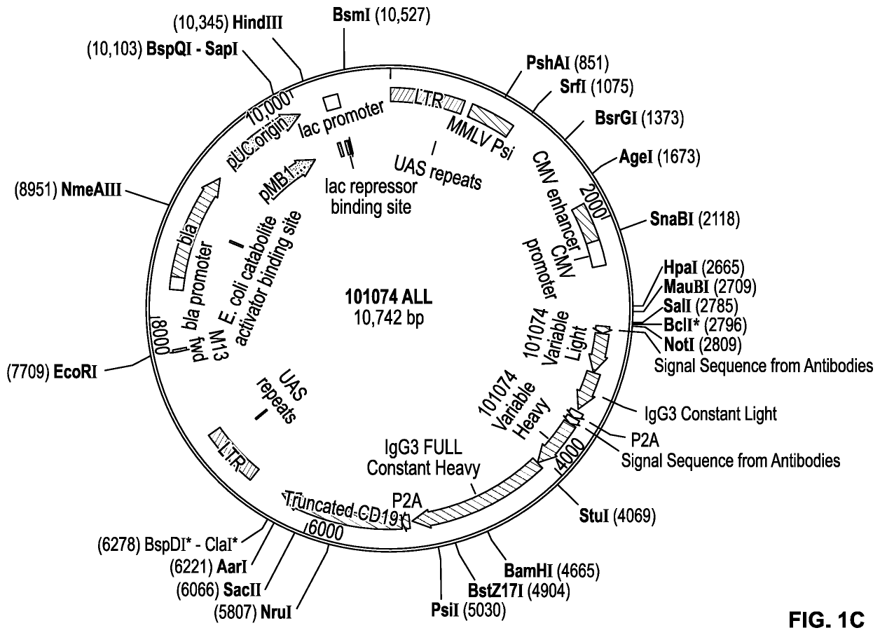

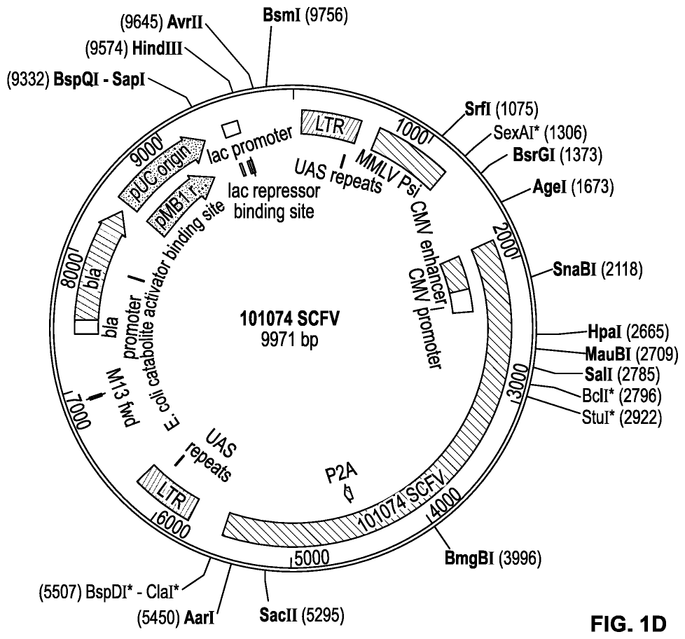

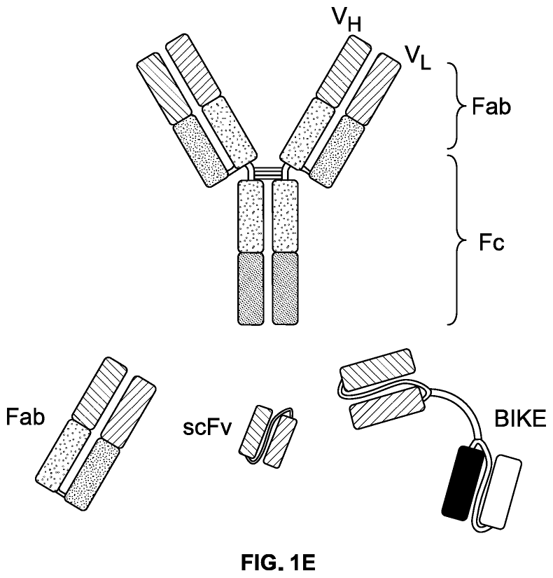

Two constructs were used for this work. The first one is a 10-1074 antibody construct (10-1074 Ab; FIG. 1A), which included the heavy and light chains of the 10-1074 antibody separated by 2A cleavage sequences (with the constant region of the heavy IgG1 chain substituted with the constant region of IgG3), 2A and furin cleavage site, and truncated CD19 (to quantify transduction efficiency). The second one is a 10-1074 bispecific killer engager construct (10-1074 BiKE; FIG. 1B), which included a single chain variable fragment (scFv) directed against HIV envelope derived by fusing the variable regions from the light and heavy chains of the 10-1074 antibody fused to an scFv directed against CD16, a 2A and furin cleavage site, and truncated CD19 (to quantify transduction efficiency). In each construct, IgG secretory signals preceded each sequence. The map of the entire plasmid comprising either construct, including the orientation of each component, is depicted in FIGS. 1C and 1D and the complete sequences of these two constructs are provided in SEQ ID NO: 80 and 84, respectively. The antibody structures processed from these constructs are depicted in FIG. 1E.

Production of Retroviral Vector

Plasmid constructs were synthesized by GenScript Biotech Corporation (Piscataway, N.J.), subcloned into a murine leukemia virus (MLV) retroviral backbone, and expanded using the Qiagen® Endofree® Plasmid Maxi Kits (Qiagen, #12362). Construct DNA (2.5 μg) was transfected into Phoenix Eco cells (at 70% confluency) using Lipofectamine® 3000 kits (ThermoFisher, #L3000001), as per manufacturer's protocol. Five hours after transfection, media containing DNA solution was replaced with fresh media. Supernatant was collected at hour 24, 48, and 52, and used to transduce PG13 producer cell lines (ATCC, #CRL-10686). PG13 transduced with constructs were single cell sorted by flow cytometry (using CD19 as marker of transduced cells) and clones were expanded and cryopreserved.

Peripheral Blood Samples

Peripheral blood samples were obtained from deidentified buffy coats from the National Institutes of Health through Dr. John Barrett of NIH or commercially from AllCells (Alameda, Calif.). Peripheral blood samples were processed within 24 hours of receipt or 3 days of collection, using Ficoll® Paque Plus Density Gradient Media (GE Life Science, #17-1440-02) to obtain peripheral blood mononuclear cells (PBMC). PBMC layer was obtained and washed in equal parts 1×dPBS at 500 g for 12 minutes. PBMCs were either frozen for future use or immediately used for monocyte isolation.

Manufacture of HIV-Specific T Cells

Monocytes were separated from PBMCs by adherence as previously described.⁶⁹ Briefly, after two-hour adherence on plates, non-adherent cells were collected and cryopreserved to be used as the T cell fraction at the first stimulation. Adherent cells were fed with GM-CSF (R&D, #215-GM-500) and IL4 (R&D, #204-IL-500) and incubated for 72 hours at 37° C. One day prior to stimulation DCs were matured with 2.5 μg/mL Gag, Nef, Pol overlapping peptide and GM-CSF, TNFα, IL1β, IL4, IL6, PGE-2, and LPS or GM-CSF, INF-γ, IL-4, LPS. Sixteen hours following maturation, DCs were irradiated at 30 Gy and cocultured with the non-adherent fraction at a ratio of 1:10. Subsequent stimulations used autologous PHA blasts, made from phytohemagglutinin and IL-2 stimulated autologous PBMCs, and K562 feeder cells (ATCC, #CCL-243). PHA blasts and K562s were irradiated at 30 Gy and 200 Gy, respectively. T cells were stimulated at a ratio of 1:4 PHA blast to T cell for the second stimulation and 1:1:4 of T cells:PHA blasts:K562 for the third stimulation.⁷⁰

Alternatively, HIV-specific T cells can be substituted with other antigen-specific T cells relevant for treating HIV patients, and HIV patients with malignancy. These include T cells made specific for endogenous retroviruses, repetitive elements, HPV, EBV, and HHV8.

Transduction of HIV-Specific T Cells

Viral transduction of antigen-specific T cells was performed as previously described⁷¹ with some modifications. Three days post stimulation 2, T cells were transduced with retroviral supernatant, collected fresh 24-48 hours after subculturing transduced PG13 or frozen retroviral supernatant concentrated 1:3 with RetroX™ concentrator (Takara, #631455). Non-tissue culture plated were treated with 50 μg/mL RetroNectin® (Takara, #T100A/B) overnight at 4° C. 2 mL of retroviral supernatant was added to each well and centrifuged at 2000 g for 2 hours. Following viral centrifugation, cells were plated at 5e5 cells/well with the addition of 50 U/ml IL2 (R&D, #202-IL-500). Supernatants were collected two to three days following transduction and frozen for functional assays.

Five to seven days following the third stimulation, cells were collected for functional assays and cryopreserved in freeze media containing 50% FBS, 40% RPMI, and 10% Dimethyl Sulfoxide (Sigma-Aldrich, #472301).

Flow Cytometry

Cell phenotype and transduction efficiency were determined by flow cytometry, using the following cell surface markers: CD3 PE Cy7 (BioLegend, #344816), CD19 APC (Miltenyi, #130-110-250), CD4, CD8. Stained cells were run on a Beckman Coulter Cytoflex. Data was analyzed using the FlowJo software.

INF-γ ELISpot

Specificity to HIV peptides Gag, Nef, and Pol were determined by INF-γ ELISpot assay. Media (no peptide) and an irrelevant peptide (actin) were used as negative controls and Staphylococcus enterotoxin B (SEB) was used as positive control. Specificity to Gag, Nef, and Pol, as a combination of the three peptides (GNP) was determined. Positive results were defined as double the number of INF-γ spot forming units than that obtained in the negative control and at least 25 SFU/1×10⁵ cells plated. Elispot plates were scanned and analyzed by Zellnet.

Cytokine Secretion

Cytokine secretion of virus naïve donor derived HIV-specific T cells secreting 10-1074 broadly neutralizing antibodies was determined using Bio-plex Pro™ Human Cytokine 17-plex Assay (BioRad, #M5000031YV). Cellular supernatant was collected 3 days following retroviral transduction and cryopreserved until assay was performed. T cell secretion of GM-CSF, TNF-α, MCP-1, IL-4, IL-5, IL-13, and IL-17 were measured.

10-1074 Antibody ELISA

Secretion of antibody by dHXTCs were tested by 10-1074 ELISA. HIV-1 env gp120 recombinant human protein (mybiosource.com, #MBS636028) was used to coat high binding microplates (Sigma, #M4561-40E). Supernatant collected from both dHXTC and PHA blasts was used as primary antibody as the 10-1074 variable region would bind the gp120 protein coated plate. Goat anti-Human IgG (H+L) cross-absorbed secondary antibody, HRP labeled (ThermoFisher, #62-8420) was used to detect primary antibody bound to the plate by binding to the Fc portion of the construct.

HIV Binding

HIV envelope expressing HeLa cells were obtained from NIH AIDS Reagent Program (69T1 RevEnv Cells, #3336). HeLa Envelope cells were fixed with 4.2% paraformaldehyde (BD, #554655) for 20 minutes at 4° C. Cells were washed in chilled Facs Buffer (PBS+2% FBS) and incubated in supernatant containing secreted antibody from transduced Jurkat T cells or non-specific antibodies from nontransduced Jurkat T cells for 1 hour at room temperature. Cells were washed an additional two times in Facs Buffer and incubated with goat anti-human IgG (H+L) FITC (Life Technologies, #H10301) for 30 minutes at room temperature.

ADCC

HIV envelope expressing HeLa cells (from the AIDS Reagent program) were used as target cells. Target cells were labeled overnight with T cell derived antibody and europium cytotoxicity assays (Perkin Elmer) were performed, using primary NK cells as effectors. NK cells were expanded from PBMC as previously described.^{72,73}

Viral Inhibition Assay (p24)

CD4+ selected PBMCs (target cells) were activated with IL-2 and PHA for 72 hours before infection with a laboratory strain of HIV SF162. Infected target cells were cocultured with genetically modified HIV-specific T cells at a ratio of 10:1 effector to target cells. Supernatant was collected and measured for HIV p24 levels on days 3, 5, and 8 post infection and coculture. P24 levels were quantified by p24 Elisa (ABL, Inc, #5447). P24 levels in experimental conditions were normalized to infected CD4+ target cells alone.

Statistics

Data presented is summarized as mean±standard deviation. We used paired t tests to detect differences between transduced and non-transduced T cells, and p-values less than 0.05 were used to determine significance. Analysis was performed using GraphPad PRISM.

Results

T cells were modified to express the broadly neutralizing antibody 10-1074 (engineered to increase ADCC by replacing the IgG1 Fc with IgG3). The anti-HIV functions of the T cells and their secreted product were tested, and assessed for synergistic activity of individual components of the platform.

Antibody Construct and Gene Modification of T Cells

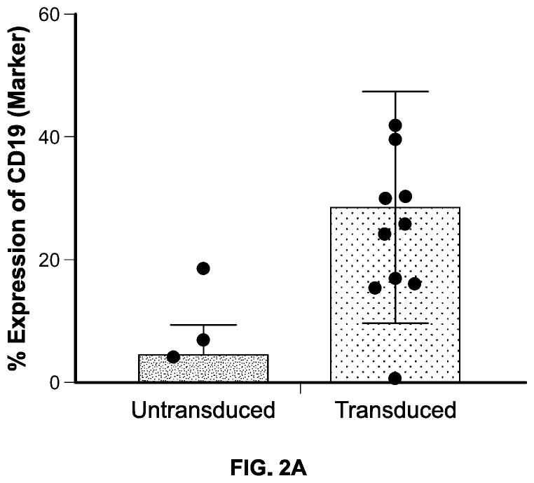

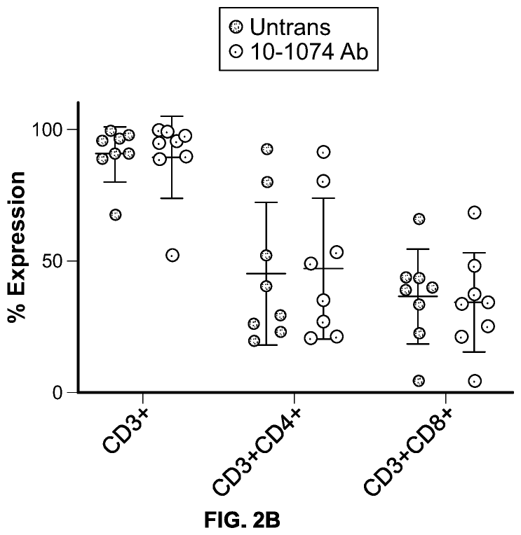

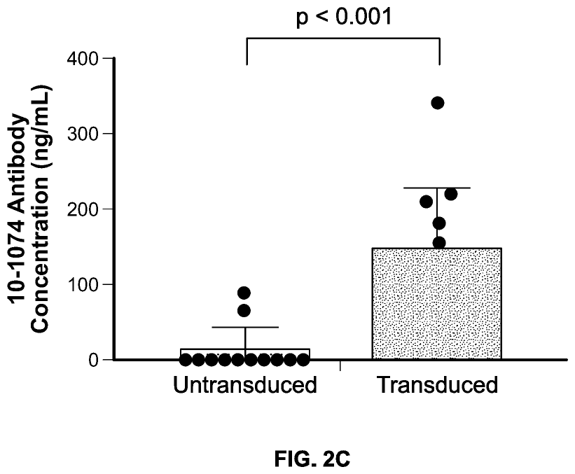

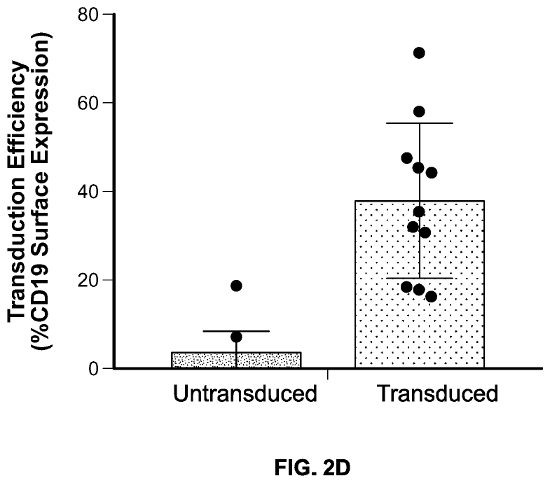

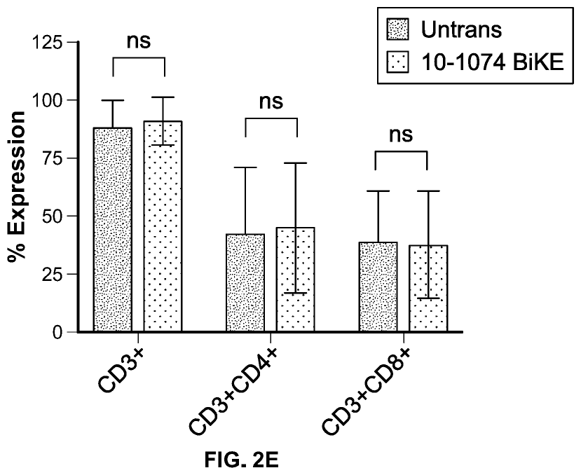

We designed a retroviral vector named “10-1074 Ab” that contains the light chain and heavy chain variable regions of the 10-1074 antibody separated by a 2A cleavage site. Both chains followed an endogenous immunoglobulin secretory signal. To determine transduction efficiency, we coupled antibody expression to expression of a truncated CD19 receptor without a cytoplasmic signaling domain (the receptor is absent in T cells). This marker is part of the transgene, separated from the antibody by furin and 2A cleavage sites (FIG. 1A). We then tested whether T cells could be modified to express these antibodies, by transducing non-specifically activated cells from healthy donors. Following gene modification with our retroviral vectors, we observed median transduction efficiencies of 25.900 (mean 28.6±18.8, range 0.9 to 73.1, n=11, FIG. 2A). Products in transduced and nontransduced cells contained mixed populations of CD4+ T cells and CD8+ T cells (FIG. 2B). For transduced cells, we detected a median of 121.2 ng/mL of antibody in the supernatant collected after 24 hours from T cells plated at 1×10⁶/mL (mean 147.2±80.1 ng/mL, range 66.7 to 341, n=12, FIG. 2C).

We designed a second retroviral vector that is a bispecific killer cell engager or BiKE molecule (“10-1074 BiKE”; FIG. 1B). The 10-1074 BiKE features a significantly shorter sequence and does not rely on extracellular assembly to produce a functional product. This construct is composed of the 10-1074 single chain variable fragment and CD16 single chain variable fragment, coupled together by a short glycine-serine linker. We observed a similar transduction efficiencies (FIG. 2D) as well as T cell phenotype (FIG. 2E).

T Cell Secreted Antibodies Bind to HIV Envelope Expressed on Cells

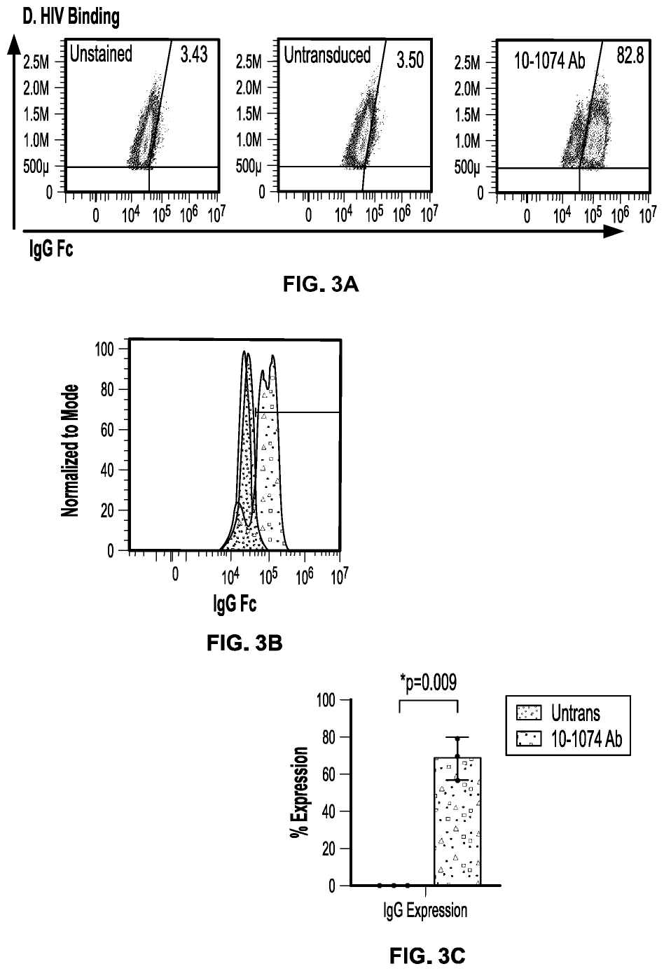

To test whether the T cell secreted antibodies maintain their ability to recognize HIV envelope, we used HeLa cells expressing Env obtained from the AIDS reagent program. Using flow cytometry, we determined that our T cell-secreted antibodies (obtained from the supernatant of cells transduced by the 10-1074 Ab construct) bind to envelope-expressing cells but not non-expressing cells (FIG. 3). As expected, supernatants from non-transduced cells did not exhibit binding to these HIV Env expressing cells (FIG. 3).

HIV-Specific T Cells can be Modified to Secrete 10-1074 Antibodies

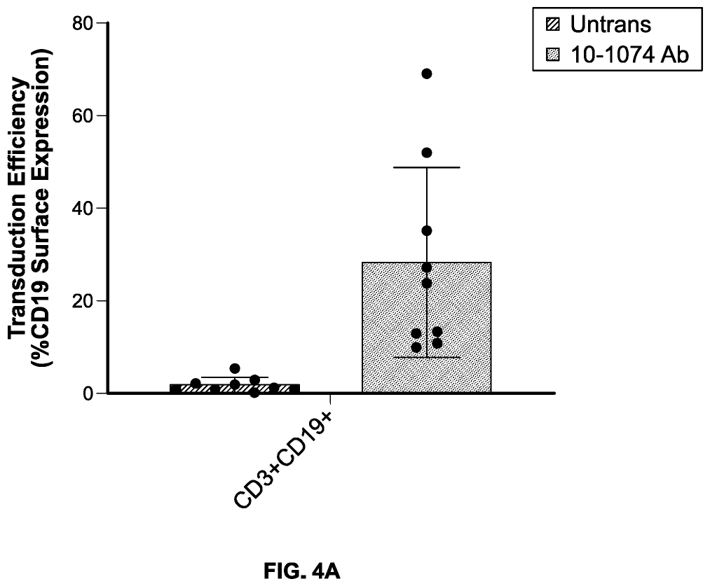

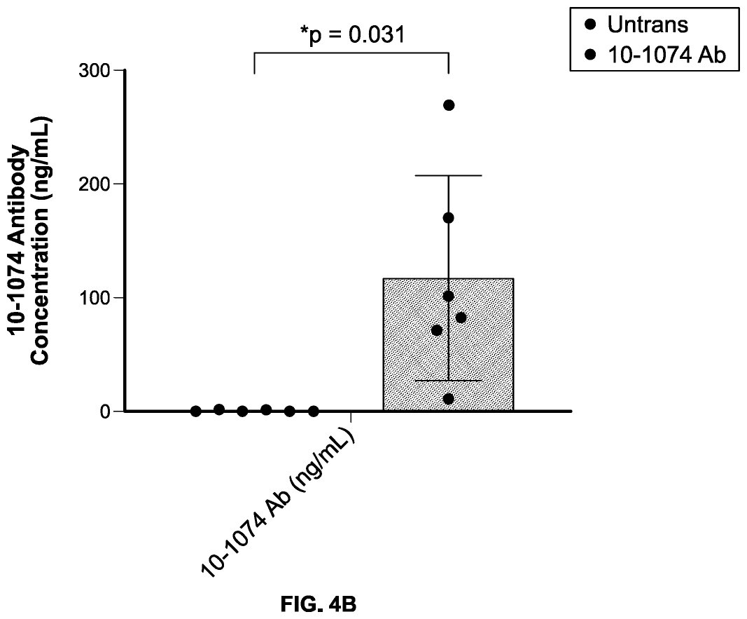

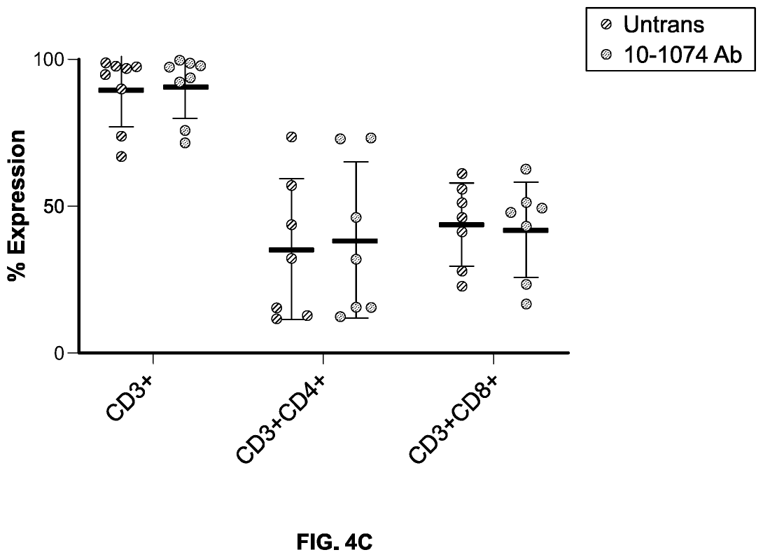

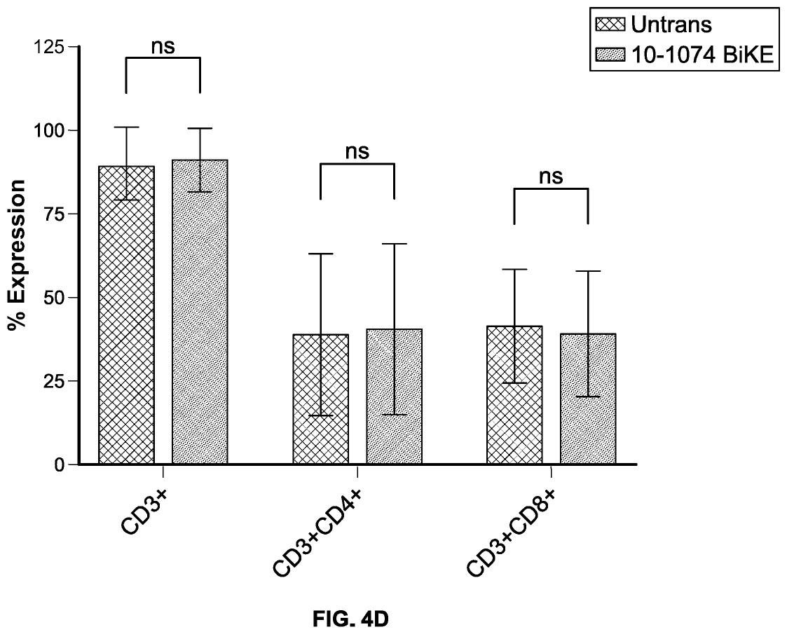

We then tested whether we could combine anti-HIV activity from T cells and ADCC-inducing broadly neutralizing antibodies into one platform by genetically modifying HIV-specific T cell lines. Cells that were expanded to recognize the HIV antigens g=Gag, Pol, and Nef were modified by our retroviral vector 10-1074 Ab (FIG. 4A) to express 10-1074 antibodies (FIG. 4B). Genetic modification did not significantly alter the makeup of CD4⁺ vs CD8⁺ populations within the T cell populations (FIG. 4C). Similar results were observed with our retroviral vector 10-1074 BiKE (FIG. 4D).

Transduced HIV-Specific T Cells Maintain Antigen-Specific T Cell Functions

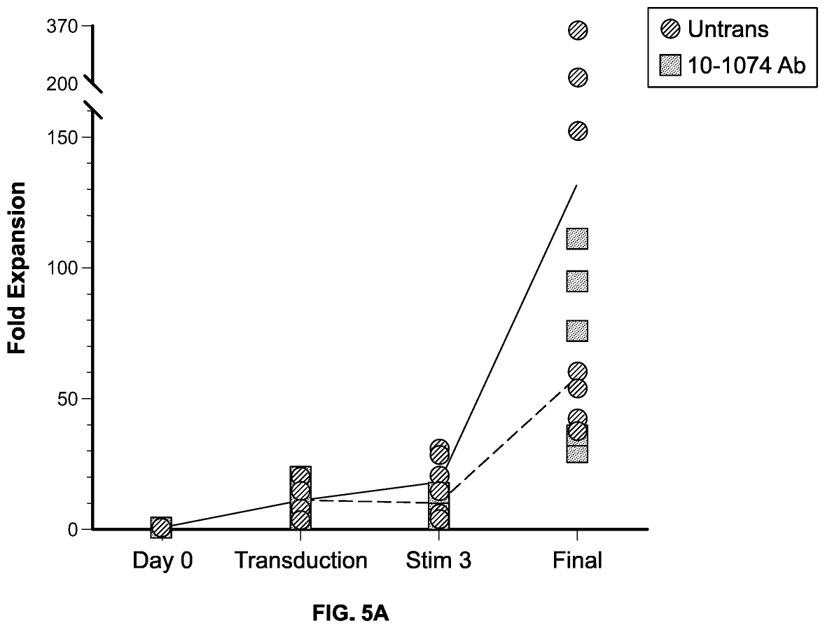

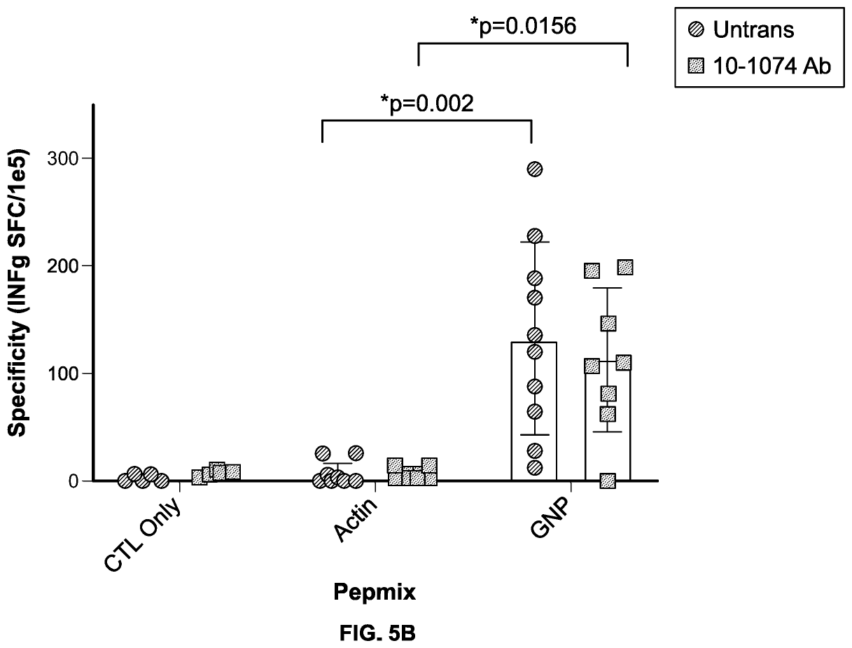

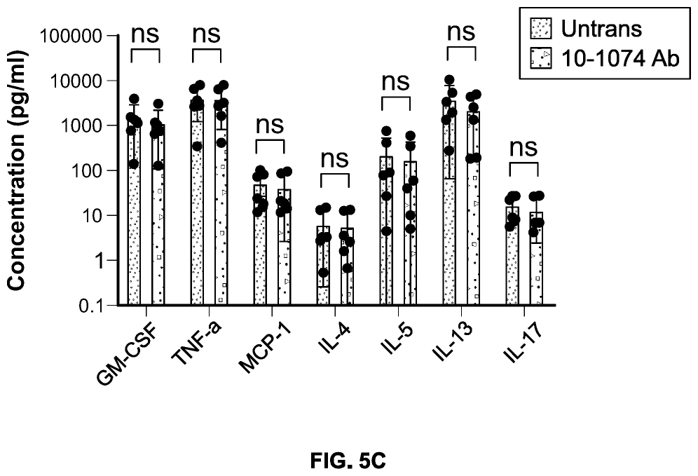

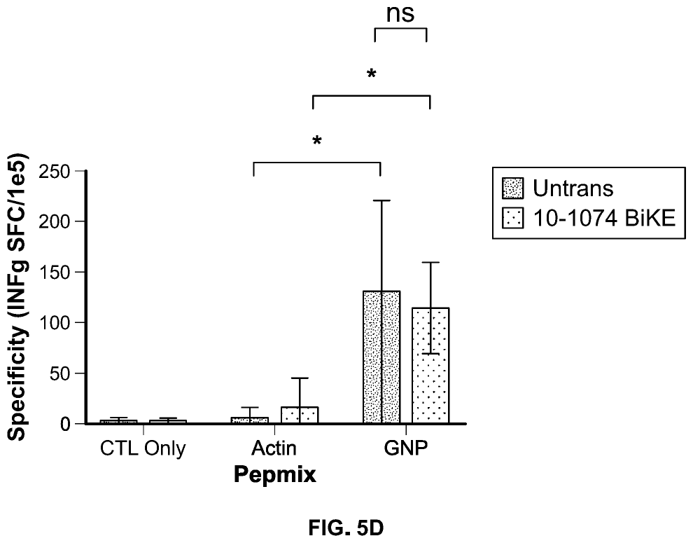

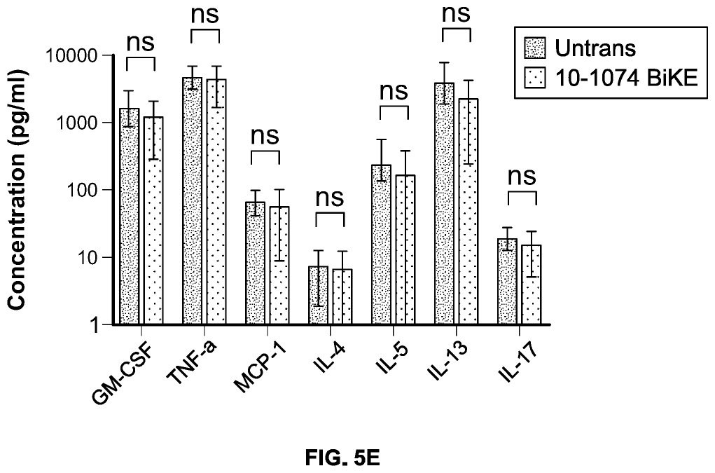

To determine whether genetic modification of HIV specific T cells to secrete the bNAb 10-1074 negatively affected their T cell effector function, we tested the secretion of multiple cytokines and chemokines of these cells in response to antigen-specific stimulation. Genetic modification of these cells with our retroviral vector 10-1074 Ab did not significantly affect their abilities to expansion in response to antigenic stimulation with gag, pol, and nef peptides (mean expansion of 18.7±9.8 in nontransduced post third stimulation vs 9.9±4.9 in transduced cells, p=ns, n=9, FIG. 5A). These genetically modified T cell lines also retained specificity to HIV peptides Gag, Nef, and Pol, as measured by IFNγ ELISPOT (mean of 131.0±88.7 IFNγ SFC/1×10⁵ cells, n=10, in response to Gag/Nef/Pol peptide pools in nontransduced vs 111.9±67.1 IFNγ SFC/1×10⁵ cells, n=8, in response to Gag/Nef/Pol antigens in transduced cells, p=0.02 and p=0.01 for each, respectively, when comparing against negative (actin) controls, but p=0.6213 when compared with each other, FIG. 5B). Finally, no significant differences in the secretion of T cell cytokines including GM-CSF (1525.4±1374.5 pg/mL nontransduced vs 1142.4±1030 pg/mL transduced, p=ns, n=6), TNFα (4003.7±2777.3 pg/mL nontransduced vs 3774±2958.8 pg/mL transduced, p=ns, n=6), IL-17 (16.7±10.4 pg/mL nontransduced vs 12.982±10.620 pg/mL transduced, p=ns, n=6), and the monocyte chemoattractant protein 1 (51.699±37.784 nontransduced pg/mL vs 41.2±38.6 pg/mL transduced, p=ns, n=6) were observed between nontransduced and transduced T cells (FIG. 5C). Similar results were observed with our retroviral vector 10-1074 BiKE (FIG. 5D and FIG. 5E). These results support that genetic modification of HIV-specific T cells does not alter the effector function of the T cells while conferring the new functionality of antibody secretion.

T Cell-Secreted Antibodies from HIV-Specific T Cells Elicit ADCC

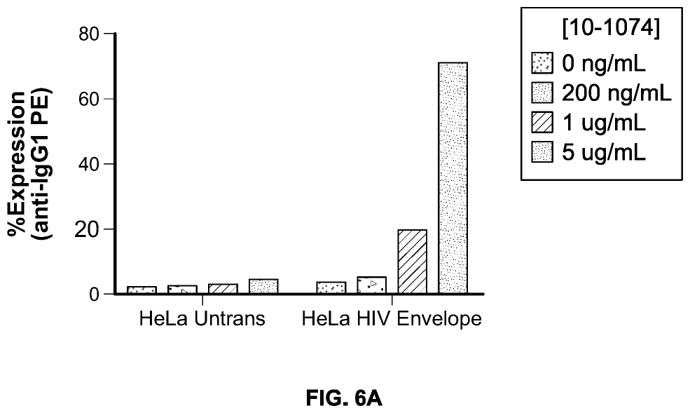

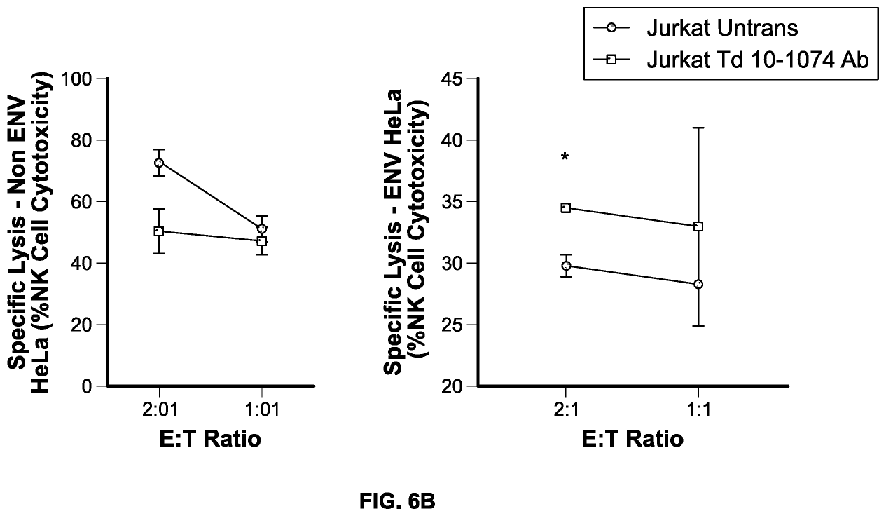

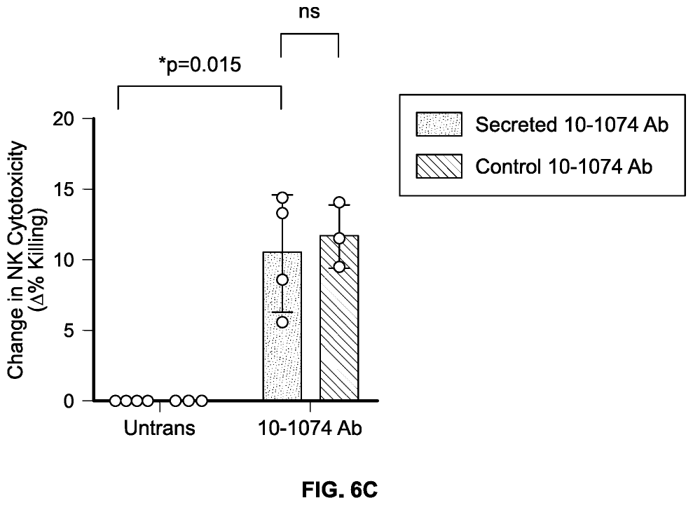

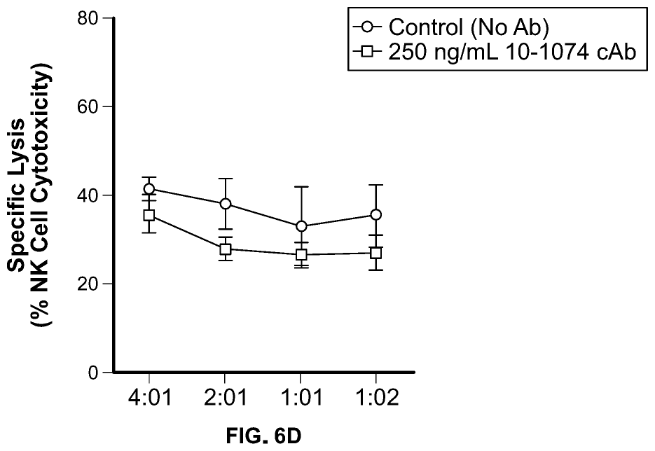

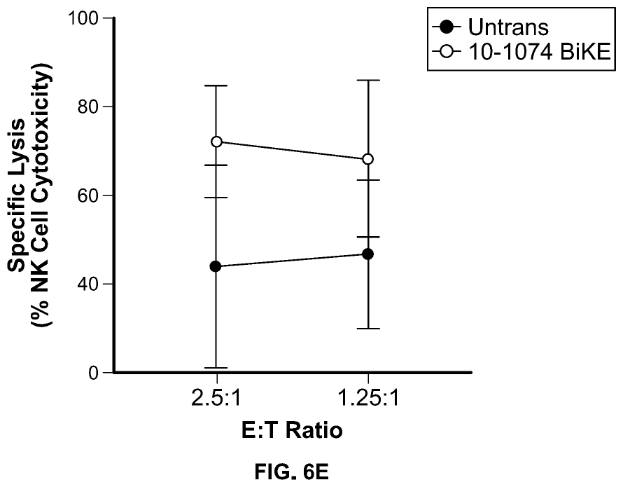

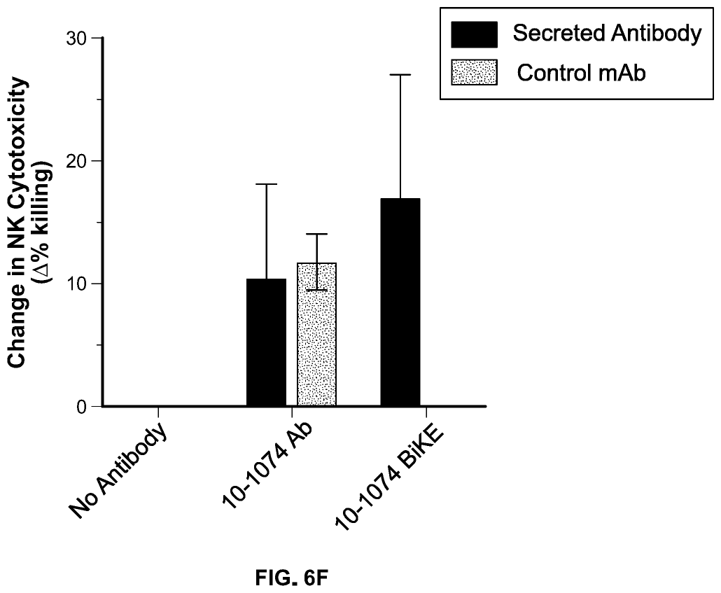

To determine whether 10-1074 antibody derived from HIV-specific T cells retained its ability to elicit ADCC, we first tested their ability to increase NK-mediated killing of HeLa cells. We used Env-transduced and non-transduced HeLa cells as targets and observed that the transduced cells bound antibody while the nontransduced cells did not (FIG. 6A). As expected, no increase in NK cell killing is seen when targeting non-HIV-envelope expressing HeLa cells, comparing the supernatants from nontransduced and transduced cells. In contrast, a significant increase in NK cell killing is seen when these supernatants were used to target HIV-envelope expressing HeLa cells (34.5±0.3 in the presence of 10-1074 Ab vs 29.8±0.8 in the presence of supernatant from nontransduced cells, p=0.017, n=2, FIG. 6B). The increase in killing from ADCC was observed using supernatants from multiple transduced lines (10.5±4.1%, p=0.015, n=4), comparable to the control, a purified 10-1074 antibody which had been produced from 1×10⁶ cells/mL (FIG. 6C). We further confirmed the specificity of this increase in cytotoxicity using control 10-1074 targeting non-Env expressing HeLa cells (FIG. 6D). Thus, the 10-1074 antibody produced from engineered T cells exhibits similar ability to elicit ADCC as a corresponding control 10-1074 antibody produced by 1×10⁶ cells/mL transduced cells. While the above results were observed from T cells transduced with the construct 10-1074 Ab, we observed similar results from T cells transduced with the construct 10-1074 BiKE (FIG. 6E and FIG. 6F).

Genetic Modification of HIV-Specific T Cells to Secrete 10-1074 Antibody Increases Anti-Viral Efficacy Against HIV-Infected Targets

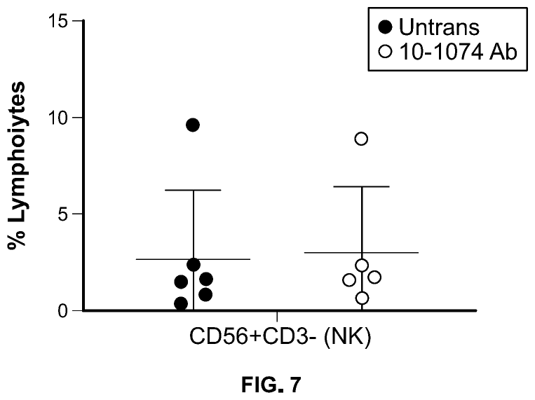

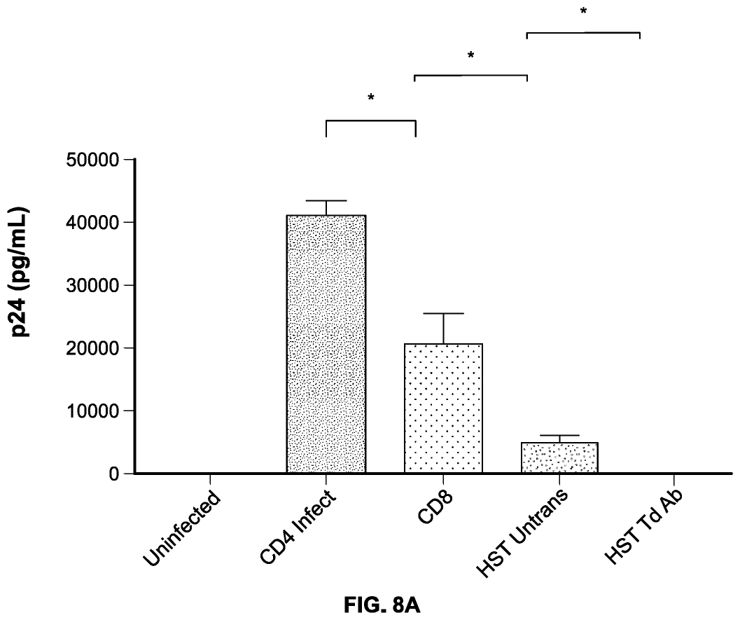

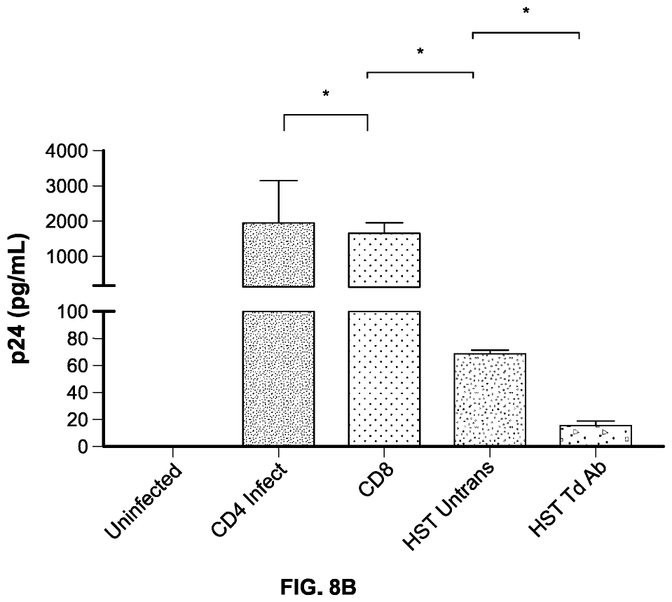

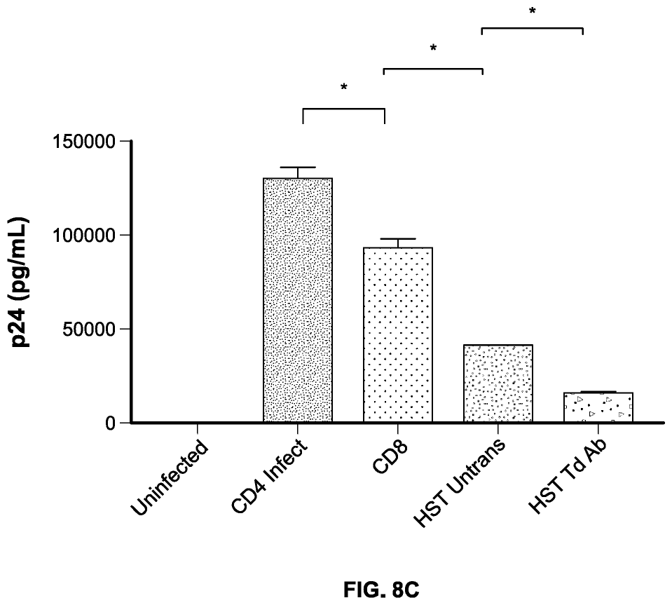

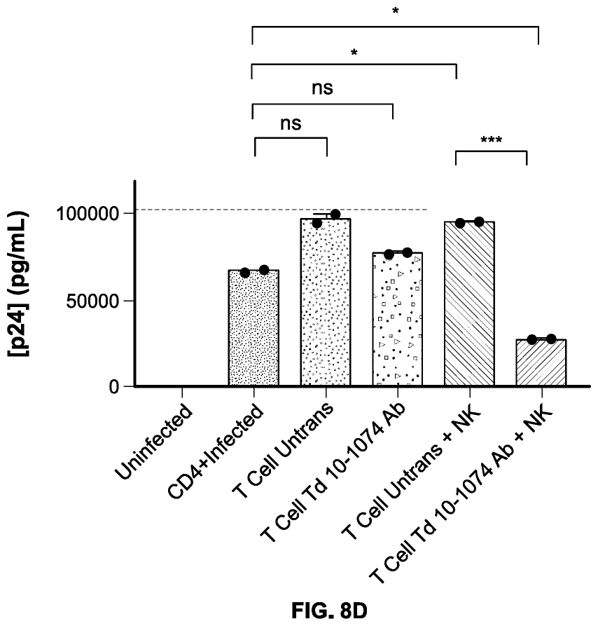

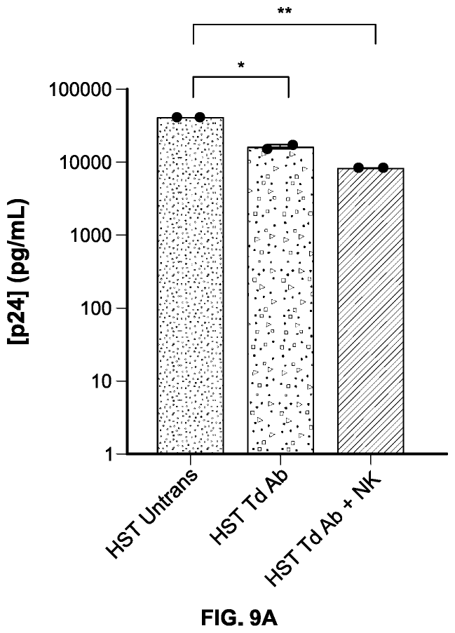

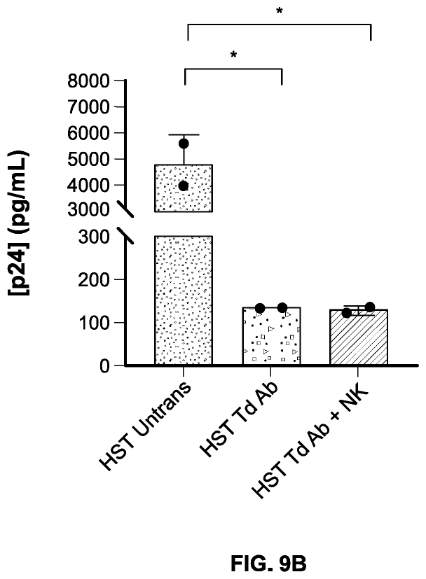

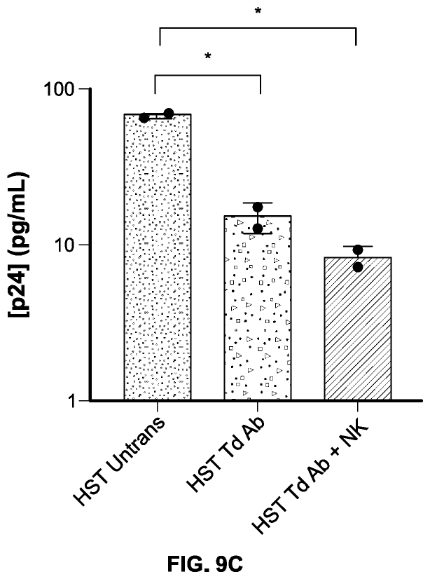

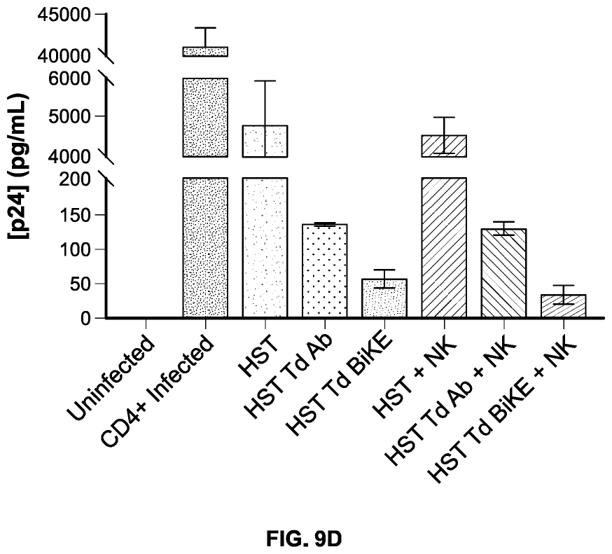

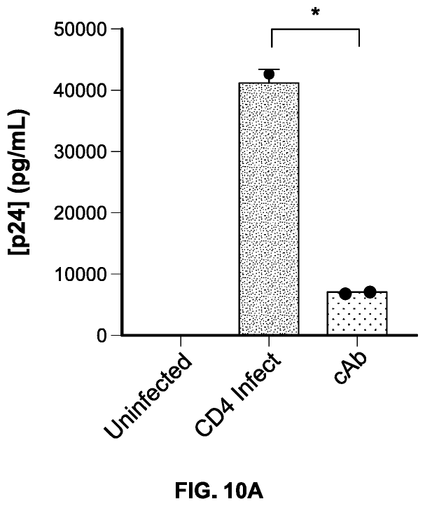

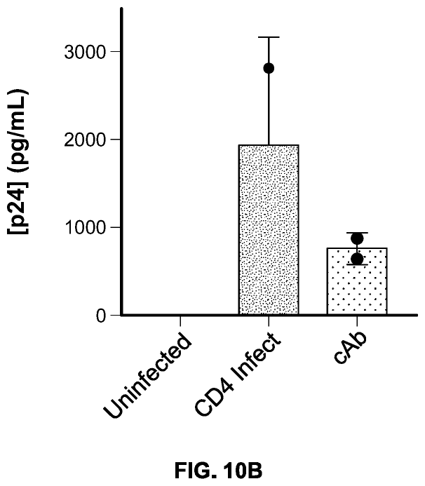

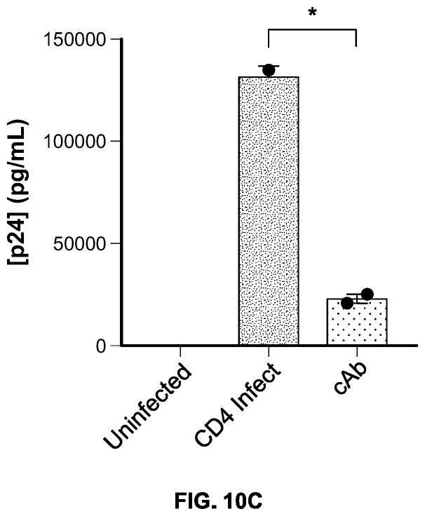

Finally, to test whether we successfully combined innate (ADCC) and adaptive (T cell-mediated killing) immunity to HIV in a single platform, we measured the anti-viral efficacy of engineered cells (transduced with 10-1074 Ab construct) against autologous HIV-infected CD4+ T cells over five days viral inhibition assays. We compared viral inhibition of 10-1074 antibody-secreting T cell lines (which contain between 1-10% of CD3-CD56+NK cells, FIG. 7) targeting autologous, infected CD4+ T cells to (a) CD8+, nonspecific T cells, (b) non-transduced HIV-specific T cells, and (c) 10-1074 control antibody. We show in each donor (FIG. 8A, 8B, 8C) significantly increased inhibition of viral replication by HIV-specific T cells over CD8+ nonspecific T cells (4785.2±1157.3 vs 20680.2±4785.3, n=2 replicates, p=0.0448, FIG. 8A; 67.4±3.1 vs 1653.5±248.4, n=2 replicates, p=0.012, FIG. 8B; 41457.8±59.6 vs 94336.9±3996.9, n=2 replicates p=0.003, FIG. 8C), as we have previously reported.⁶⁹ Of central importance to the current study, we observed greater inhibition of viral replication by cells that had been transduced to secrete 10-1074, as compared to their nontransduced counterparts (133.634±2.343 vs 4785.174±1157.271, n=2 replicates, p=0.029, FIG. 8A; 15.190±3.401 vs 67.373±3.088, n=2 replicates, p=0.004, FIG. 8B; 16226.950±1333.975 vs 41457.831±59.636, n=2 replicates p=0.001, FIG. 8C)), suggesting that the addition of secreted 10-1074 antibody effector function improves anti-HIV function of HIV-specific T cells by engaging passenger NK cells. Interestingly, the addition of autologous NK cells to the product did not seem to significantly alter viral inhibition in two of the three evaluable lines (although there is a trend towards decreased amounts of p24 in all three—127.849±9.867 vs 133.634±2.343, n=2 replicates, p=ns, FIG. 9A; 8.259±1.566 vs 15.190±3.401, n=2 replicates, p=ns, FIG. 9B; 8378.014±117.350 vs 16226.950±1333.975, n=2 replicates, p=0.0142, FIG. 9C), suggesting that a small amount of NK cells (<10% of the population) is sufficient to mediate killing via ADCC. Similar results were also observed from T cells transduced with the construct 10-1074 BiKE (FIG. 9D). Addition of control 10-1074 antibody alone (in the absence of NK cells) (6892.442±168.555 vs 41130.814±2240.542, n=2 replicates, p=0.002, FIG. 10A; 757.911±163.351 vs 1939.873±1230.724, n=2 replicates, p=ns, FIG. 10B; 22914.671±2305.507 vs 131692.771±5426.832, n=2 replicates, p=0.001, FIG. 10C) did decrease viral inhibition (in two of three evaluable lines) above that observed with uninfected cells, likely as a result of neutralization of virus and prevention of re-infection. Also of note, where non-HIV-specific T cells were used as the platform, no viral inhibition was seen in the nontransduced cells (67645.833±1060.660 CD4 T cell targets alone vs 98002±3532.705 with CD4 T cell targets with nonspecific T cells, n=2 replicates, p=0.007, FIG. 8D), emphasizing the importance of HIV-specific T cells in control of viral-infected cells. Indeed, in these conditions we observed there is a statistically significant increase in p24, likely the result of reinfection of the nonspecific T cells (which contain CD4+ cells). Thus, each component of this approach: HIV-specific T cells, antibody, and NK cell effectors, contribute to the overall viral inhibition.

### Example 6: Alternate Constructs

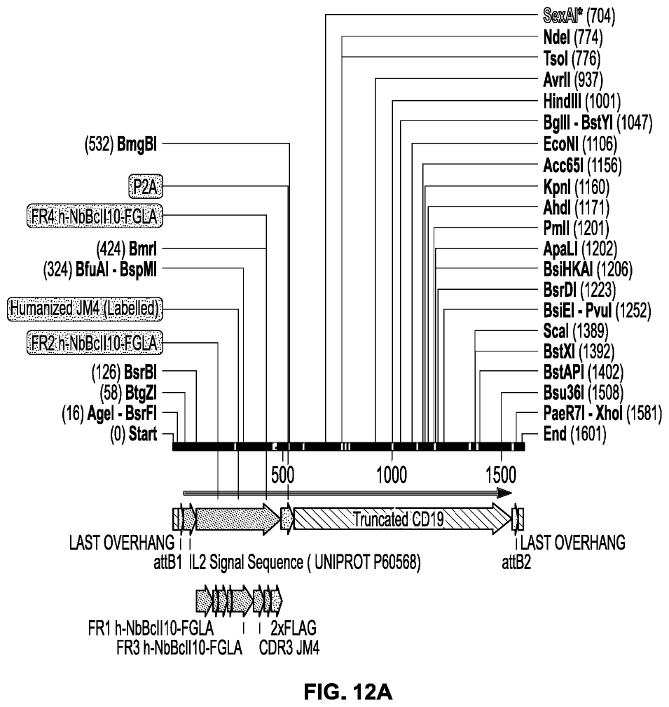

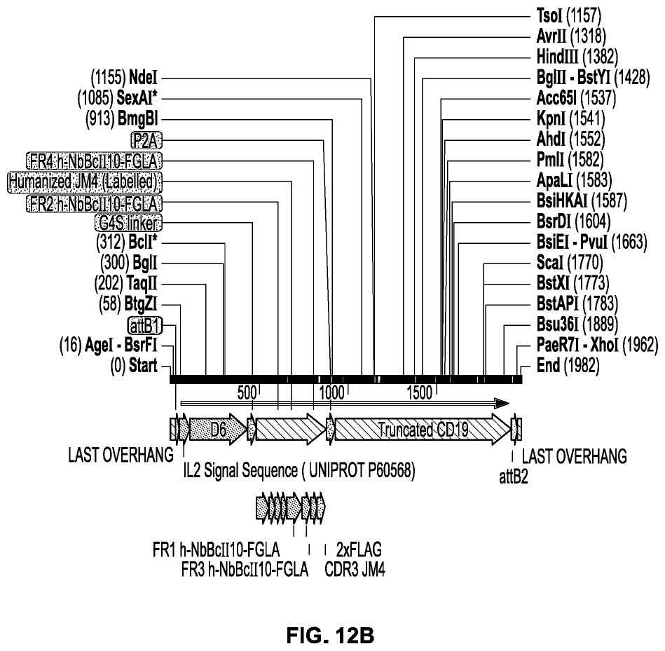

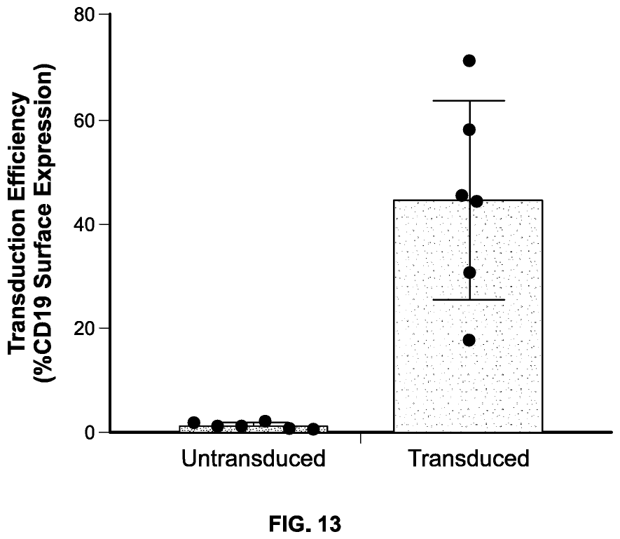

We have generated bispecific killer engager (BiKE)-based constructs (see e.g., FIG. 1D). The results obtained with one of such BiKE constructs are provided in, for example, FIG. 2D, FIG. 2E, FIG. 4D, FIG. 5D, FIG. 5E, FIG. 6E, FIG. 6F, FIG. 9D, and FIG. 13. Other additional constructs were also generated for practicing the present disclosure, which include, but not limited to, Genesis 605a and Genesis 605b. Both construct contain a human-codon-optimized nucleic acid sequence encoding the HIV-1 neutralizing single domain antibody JM1 (Matz J, Kessler P, Bouchet J, Combes O, Ramos O H, Barin F, Baty D, Martin L, Benichou S, Chames P. Straightforward selection of broadly neutralizing single-domain antibodies targeting the conserved CD4 and coreceptor binding sites of HIV-1 gp120. J Virol. 2013 January; 87(2):1137-49. doi: 10.1128/JVI.00461-12. Epub 2012 Nov. 14.). The schematics for these two constructs are provided in FIG. 12A (Genesis 605a) and FIG. 12B (Genesis 605b) and their corresponding sequence is provided as SEQ ID NO: 76 (Genesis 605a) and SEQ ID NO: 77 (Genesis 605b).

### REFERENCES

1. Kober, L., Zehe, C. & Bode, J. Optimized signal peptides for the development of high expressing CHO cell lines. Biotechnology and bioengineering 110, 1164-1173 (2013).
2. Smith, P., DiLillo, D. J., Bournazos, S., Li, F. & Ravetch, J. V. Mouse model recapitulating human Fcgamma receptor structural and functional diversity. Proceedings of the National Academy of Sciences of the United States of America 109, 6181-6186 (2012).
3. Chun, T. W., Davey, R. T., Jr., Engel, D., Lane, H. C. & Fauci, A S. Re-emergence of HIV after stopping therapy. Nature 401, 874-875 (1999).
4. Finzi, D., et al. Identification of a reservoir for HIV-1 in patients on highly active antiretroviral therapy. Science 278, 1295-1300 (1997).
5. Sgarbanti, M. & Battistini, A. Therapeutics for HIV-1 reactivation from latency. Curr Opin Virol 3, 394401 (2013).
6. Archin, N. M., et al. Eradicating HIV-1 infection: seeking to clear a persistent pathogen. Nat Rev Microbiol 12, 750-764 (2014).
7. Archin, N. M., et al. Expression of latent HIV induced by the potent HDAC inhibitor suberoylanilide hydroxamic acid. AIDS research and human retroviruses 25, 207-212 (2009).
8. Xing, S., et al. Disulfiram reactivates latent HIV-1 in a Bcl-2-transduced primary CD4+ T cell model without inducing global T cell activation. Journal of virology 85, 6060-6064 (2011).
9. Spivak, A. M., et al. A pilot study assessing the safety and latency-reversing activity of disulfiram in HIV-infected adults on antiretroviral therapy. Clin Infect Dis 58, 883-890 (2014).
10. Shan, L., et al. Stimulation of HIV-1-specific cytolytic T lymphocytes facilitates elimination of latent viral reservoir after virus reactivation. Immunity 36, 491-501 (2012).
11. Khaitan, A. & Unutmaz, D. Revisiting immune exhaustion during HIV infection. Curr HIV/AIDS Rep 8, 411 (2011).
12. Deeks, S. G. HIV: Shock and kill. Nature 487, 439-440 (2012).
13. Carmichael, A., Jin, X., Sissons, P. & Borysiewicz, L. Quantitative analysis of the human immunodeficiency virus type 1 (HIV-1)-specific cytotoxic T lymphocyte (CTL) response at different stages of HIV-1 infection: differential CTL responses to HIV-1 and Epstein-Barr virus in late disease. The Journal of experimental medicine 177, 249-256 (1993).
14. Barouch, D. H. & Deeks, S. G. Immunologic strategies for HIV-1 remission and eradication. Science 345, 169-174 (2014).
15. Sung, J. A., et al. Expanded Cytotoxic T-cell Lymphocytes Target the Latent HIV Reservoir. J Infect Dis (2015).
16. Iwahori, K., et al. Engager T cells: a new class of antigen-specific T cells that redirect bystander T cells. Mol Ther 23, 171-178 (2015).
17. Boesch, A. W., Alter, G. & Ackerman, M. E. Prospects for engineering HIV-specific antibodies for enhanced effector function and half-life. Current opinion in HIV and AIDS (2015).
18. Seay, K., et al. In Vivo Activation of Human N K cells by Treatment with an IL-15 Superagonist Potently Inhibits Acute In Vivo HIV-1 Infection in Humanized Mice. Journal of virology (2015).
19. Romee, R., Leong, J. W. & Fehniger, T. A. Utilizing cytokines to function-enable human N K cells for the immunotherapy of cancer. Scientifica 2014, 205796 (2014).
20. Zeng, R., et al. Synergy of IL-21 and IL-15 in regulating CD8+ T cell expansion and function. The Journal of experimental medicine 201, 139-148 (2005).
21. Croce, M., Orengo, A. M., Azzarone, B. & Ferrini, S. Immunotherapeutic applications of IL-15. Immunotherapy 4, 957-969 (2012).
22. Diab, A., Cohen, A D., Alpdogan, O. & Perales, M. A. IL-15: targeting CD8+ T cells for immunotherapy. Cytotherapy 7, 23-35 (2005).
23. Mastroianni, C. M., et al. Teaching tired T cells to fight HIV: time to test IL-15 for immunotherapy? Trends in immunology 25, 121-125 (2004).
24. Walker, B. & McMichael, A. The T-cell response to HIV. Cold Spring Harbor perspectives in medicine 2(2012).
25. Harrer, T., et al. Strong cytotoxic T cell and weak neutralizing antibody responses in a subset of persons with stable nonprogressing HIV type 1 infection. AIDS research and human retroviruses 12, 585-592 (1996).
26. Saez-Cirion, A., et al. HIV controllers exhibit potent CD8 T cell capacity to suppress HIV infection ex vivo and peculiar cytotoxic T lymphocyte activation phenotype. Proceedings of the National Academy of Sciences of the United States of America 104, 6776-6781 (2007).
27. Koup, R. A., et al. Temporal association of cellular immune responses with the initial control of viremia in primary human immunodeficiency virus type 1 syndrome. Journal of virology 68, 4650-4655 (1994).
28. Appay, V., et al. HIV-specific CD8(+) T cells produce antiviral cytokines but are impaired in cytolytic function. The Journal of experimental medicine 192, 63-75 (2000).
29. Hansen, S. G., et al. Immune clearance of highly pathogenic SIV infection. Nature 502, 100-104 (2013).
30. Lieberman, J., et al. Safety of autologous, ex vivo-expanded human immunodeficiency virus (HIV)-specific cytotoxic T-lymphocyte infusion in HIV-infected patients. Blood 90, 2196-2206 (1997).
31. Chapuis, A. G., et al. HIV-specific CD8+ T cells from HIV+ individuals receiving HAART can be expanded ex vivo to augment systemic and mucosal immunity in vivo. Blood 117, 5391-5402 (2011).
32. Zhang, J. & Perelson, A. S. Contribution of follicular dendritic cells to persistent HIV viremia. Journal of virology 87, 7893-7901 (2013).
33. Hlavacek, W. S., Stilianakis, N. I. & Perelson, A. S. Influence of follicular dendritic cells on HIV dynamics. Philosophical transactions of the Royal Society of London. Series B, Biological sciences 355, 10511058 (2000).
34. Cavert, W., et al. Kinetics of response in lymphoid tissues to antiretroviral therapy of HIV-1 infection. Science 276, 960-964 (1997).
35. Steimer, K S., et al. Neutralization of divergent HIV-1 isolates by conformation-dependent human antibodies to Gp120. Science 254, 105-108 (1991).
36. Georgiev, I. S., et al. Antibodies VRC01 and 10E8 neutralize HIV-1 with high breadth and potency even with Ig-framework regions substantially reverted to germline. J Immunol 192, 1100-1106 (2014).
37. Ko, S. Y., et al. Enhanced neonatal Fc receptor function improves protection against primate SHIV infection. Nature 514, 642-645 (2014).
38. Rudicell, R. S., et al. Enhanced potency of a broadly neutralizing HIV-1 antibody in vitro improves protection against lentiviral infection in vivo. Journal of virology 88, 12669-12682 (2014).
39. Mascola, J. R. & Haynes, B. F. HIV-1 neutralizing antibodies: understanding nature's pathways. Immunological reviews 254, 225-244 (2013).
40. Saunders, K O., et al. Sustained delivery of a broadly neutralizing antibody in non-human primates confers long-term protection against SHIV infection. Journal of virology (2015).
41. Keizer, R. J., Huitema, A. D., Schellens, J. H. & Beijnen, J. H. Clinical pharmacokinetics of therapeutic monoclonal antibodies. Clinical pharmacokinetics 49, 493-507 (2010).
42. Johnson, P. R., et al. Vector-mediated gene transfer engenders long-lived neutralizing activity and protection against SIV infection in monkeys. Nature medicine 15, 901-906 (2009).
43. Halper-Stromberg, A., et al. Broadly neutralizing antibodies and viral inducers decrease rebound from HIV-1 latent reservoirs in humanized mice. Cell 158, 989-999 (2014).
44. Scott-Algara, D., et al. Cutting edge: increased NK cell activity in HIV-1-exposed but uninfected Vietnamese intravascular drug users. Journal of immunology 171, 5663-5667 (2003).
45. Lambotte, O., et al. Heterogeneous neutralizing antibody and antibody-dependent cell cytotoxicity responses in HIV-1 elite controllers. Aids 23, 897-906 (2009).
46. Karnasuta, C., et al. Antibody-dependent cell-mediated cytotoxic responses in participants enrolled in a phase I/II ALVAC-HIV/AIDSVAX B/E prime-boost HIV-1 vaccine trial in Thailand. Vaccine 23, 25222529 (2005).
47. Rerks-Ngarm, S., et al. Vaccination with ALVAC and AIDSVAX to prevent HIV-1 infection in Thailand. The New England journal of medicine 361, 2209-2220 (2009).
48. Haynes, B. F., et al. Immune-correlates analysis of an HIV-1 vaccine efficacy trial. The New England journal of medicine 366, 1275-1286 (2012).
49. Wren, L. H., Stratov, I., Kent, S. J. & Parsons, M. S. Obstacles to ideal anti-HIV antibody-dependent cellular cytotoxicity responses. Vaccine 31, 5506-5517 (2013).
50. Xing, S. & Siliciano, R. F. Targeting HIV latency: pharmacologic strategies toward eradication. Drug discovery today 18, 541-551 (2013).
51. Nabel, G. & Baltimore, D. An inducible transcription factor activates expression of human immunodeficiency virus in T cells. Nature 326, 711-713 (1987).
52. Horiike, M., et al. Lymph nodes harbor viral reservoirs that cause rebound of plasma viremia in SIV-infected macaques upon cessation of combined antiretroviral therapy. Virology 423, 107-118 (2012).
53. North, T. W., et al. Viral sanctuaries during highly active antiretroviral therapy in a nonhuman primate model for AIDS. Journal of virology 84, 2913-2922 (2010).
54. Lu, J., et al. Interleukin 15 promotes antigen-independent in vitro expansion and long-term survival of antitumor cytotoxic T lymphocytes. Clinical cancer research: an official journal of the American Association for Cancer Research 8, 3877-3884 (2002).
55. Rubinstein, M. P., et al. Converting IL-15 to a superagonist by binding to soluble IL-15R{alpha}. Proceedings of the National Academy of Sciences of the United States of America 103, 9166-9171 (2006).
56. Cruz, C. R., et al. Infusion of donor-derived CD19-redirected-virus-specific T cells for B-cell malignancies relapsed after allogeneic stem cell transplant: a phase I study. Blood (2013).
57. Cruz, C. R., et al. Improving T-cell therapy for relapsed EBV-negative Hodgkin lymphoma by targeting unregulated MAGE-A4. Clin Cancer Res 17, 7058-7066 (2011).
58. Lam, S., et al. Broadly-specific cytotoxic T cells targeting multiple HIV antigens are expanded from HIV+ patients: implications for immunotherapy. Mol Ther 23, 387-395 (2015).
59. Lam, S., et al. Ex vivo expanded multi-specific cytotoxic T lymphocytes derived from HIV+ patients and HIV negative donors using GMP compliant methodologies recognize multiple HIV antigens and suppress HIV replication. in American Society for Blood and Marrow Transplantation Annual Meeting 2015, Vol. 21 Suppl S64-65 (Biology of blood and marrow transplantation: journal of the American Society for Blood and Marrow Transplantation, San Diego, Calif., 2015).
60. Rosenberg, Y., et al. Pharmacokinetics and Immunogenicity of Broadly Neutralizing HIV Monoclonal Antibodies in Macaques. PloS one 10, e0120451 (2015).
61. Walker, L. M., et al. Broad neutralization coverage of HIV by multiple highly potent antibodies. Nature 477, 466-470 (2011).
62. Fang, J., et al. Stable antibody expression at therapeutic levels using the 2A peptide. Nature biotechnology 23, 584-590 (2005).
63. Bournazos, S., et al. Broadly neutralizing anti-HIV-1 antibodies require Fc effector functions for in vivo activity. Cell 158, 1243-1253 (2014).
64. Jones, R. B., et al. Histone deacetylase inhibitors impair the elimination of HIV-infected cells by cytotoxic T-lymphocytes. PLoS pathogens 10, e1004287 (2014).
65. Smalls-Mantey, A., et al. Antibody-dependent cellular cytotoxicity against primary HIV-infected CD4+ T cells is directly associated with the magnitude of surface IgG binding. Journal of virology 86, 8672-8680 (2012).
66. Sigal, A., et al. Cell-to-cell spread of HIV permits ongoing replication despite antiretroviral therapy. Nature 477, 95-98 (2011).
67. Leong, J. W., et al. Preactivation with IL-12, IL-15, and IL-18 induces CD25 and a functional high-affinity IL-2 receptor on human cytokine-induced memory-like natural killer cells. Biology of blood and marrow transplantation: journal of the American Society for Blood and Marrow Transplantation 20, 463-473 (2014).
68. Thane, J., Dabak, V., Williams, M. B., Braciale, T. J. & Ley, K. LFA-1 is required for retention of effector CD8 T cells in mouse lungs. Blood 101, 4916-4922 (2003).
69. Patel, S, Lam, S, Cruz, C R, Wright, K, Cochran, C, Ambinder, R F, et al. (2016). Functionally Active HIV-Specific T Cells that Target Gag and Nef Can Be Expanded from Virus-Naive Donors and Target a Range of Viral Epitopes: Implications for a Cure Strategy after Allogeneic Hematopoietic Stem Cell Transplantation. Biol Blood Marrow Transplant 22: 536-541.
70. Ngo, M C, Ando, J, Leen, A M, Ennamuri, S, Lapteva, N, Vera, J F, et al. (2014). Complementation of antigen-presenting cells to generate T lymphocytes with broad target specificity. J Immunother 37: 193-203.
71. Cruz, C R, Micklethwaite, K P, Savoldo, B, Ramos, C A, Lam, S, Ku, S, et al. (2013). Infusion of donor-derived CD19-redirected virus-specific T cells for B-cell malignancies relapsed after allogeneic stem cell transplant: a phase 1 study. Blood 122: 2965-2973.
72. Kellner, J N, Cruz, C R, Bollard, C M, and Yvon, E S (2016). Gene Modification of Human Natural Killer Cells Using a Retroviral Vector. Methods Mol Biol 1441: 203-213.
73. Yvon, E S, Burga, R, Powell, A, Cruz, C R, Fernandes, R, Barese, C, et al. (2017). Cord blood natural killer cells expressing a dominant negative TGF-beta receptor: Implications for adoptive immunotherapy for glioblastoma. Cytotherapy 19: 408-418.
74. Luo, X M, Maarschalk, E, O'Connell, R M, Wang, P, Yang, L, and Baltimore, D (2009). Engineering human hematopoietic stem/progenitor cells to produce a broadly neutralizing anti-HIV antibody after in vitro maturation to human B lymphocytes. Blood 113: 1422-1431.
75. Kuhlmann, A S, Haworth, K G, Barber-Axthelm, I M, Ironside, C, Giese, M A, Peterson, C W, et al. (2019). Long-Term Persistence of Anti-HIV Broadly Neutralizing Antibody-Secreting Hematopoietic Cells in Humanized Mice. Mol Ther 27: 164-177.
76. Tsai, A K, and Davila, E (2016). Producer T cells: Using genetically engineered T cells as vehicles to generate and deliver therapeutics to tumors. Oncoimmunology 5: e1122158.
77. Suarez, E R, Chang de, K, Sun, J, Sui, J, Freeman, G J, Signoretti, S, et al. (2016). Chimeric antigen receptor T cells secreting anti-PD-L1 antibodies more effectively regress renal cell carcinoma in a humanized mouse model. Oncotarget 7: 34341-34355.
78. Bruel, T, Guivel-Benhassine, F, Amraoui, S, Malbec, M, Richard, L, Bourdic, K, et al. (2016). Elimination of HIV-1-infected cells by broadly neutralizing antibodies. Nat Commun 7: 10844.
79. Lee, W S, and Kent, S J (2018). Anti-HIV-1 antibody-dependent cellular cytotoxicity: is there more to antibodies than neutralization? Curr Opin HIV AIDS 13: 160-166.
80. Mayr, L M, Decoville, T, Schmidt, S, Laumond, G, Klingler, J, Ducloy, C, et al. (2017). Non-neutralizing Antibodies Targeting the V1V2 Domain of HIV Exhibit Strong Antibody-Dependent Cell-mediated Cytotoxic Activity. Sci Rep 7: 12655.
81. Saunders, K O, Pegu, A, Georgiev, I S, Zeng, M, Joyce, M G, Yang, Z Y, et al. (2015). Sustained Delivery of a Broadly Neutralizing Antibody in Nonhuman Primates Confers Long-Term Protection against Simian/Human Immunodeficiency Virus Infection. J Virol 89: 5895-5903.
82. Tedla, N, Palladinetti, P, Kelly, M, Kumar, R K, DiGirolamo, N, Chattophadhay, U, et al. (1996). Chemokines and T lymphocyte recruitment to lymph nodes in HIV infection. Am J Pathol 148: 1367-1373.
83. Horiike, M, Iwami, S, Kodama, M, Sato, A, Watanabe, Y, Yasui, M, et al. (2012). Lymph nodes harbor viral reservoirs that cause rebound of plasma viremia in SIV-infected macaques upon cessation of combined antiretroviral therapy. Virology 423: 107-118.
84. North, T W, Higgins, J, Deere, J D, Hayes, T L, Villalobos, A, Adamson, L, et al. (2010). Viral sanctuaries during highly active antiretroviral therapy in a nonhuman primate model for AIDS. J Virol 84: 2913-2922.
85. Archin, N M, Sung, J M, Garrido, C, Soriano-Sarabia, N, and Margolis, D M (2014). Eradicating HIV-1 infection: seeking to clear a persistent pathogen. Nat Rev Microbiol 12: 750-764.

What is claimed is:

1. An antibody, or an antigen-binding fragment thereof, comprising:
a) a first light chain comprising a first light chain variable region (VL) and a first heavy chain comprising a first heavy chain variable region (VH), wherein the first light chain and the first heavy chain are derived from a first antibody or an antigen-binding fragment thereof; and
b) a second light chain comprising a second light chain variable region (VL) and a second heavy chain comprising a second heavy chain variable region (VH), wherein the second light chain and the second heavy chain are derived from a second antibody or an antigen-binding fragment thereof,
wherein the first light chain binds epitopes of the envelope protein of human immunodeficiency virus-1 (HIV-1).

2. The antibody or antigen binding fragment of claim 1, wherein either VH and/or VL region at least partially binds to V3 glycan supersite of the HIV envelope protein.

3. The antibody or antigen binding fragment of claim 1 or 2, wherein the VH and the VL are positioned non-contiguously and connected by at least one hinge sequence.

4. The antibody or antigen binding fragment of any of claims 1 through 3 further comprising one or a plurality of amino acid sequences encoded by a nucleic acid sequence having at least about 70% sequence identity to SEQ ID NO: 21 and/or SEQ ID NO: 22.

5. The antibody or antigen binding fragment of any of claims 1 through 4 further comprising at least one furin linker.

6. The antibody or antigen binding fragment of claims 1 through 5 further comprising at least one or more self-cleaving amino acid sequences chosen from: FMDV 2A (abbreviated herein as F2A), equine rhinitis A virus (ERAV) 2A (E2A), porcine teschovirus-1 2A (P2A) and Thoseaasigna virus 2A (T2A), or at least one internal ribosome entry sequence (IRES) separating construct domains.

7. The antibody or antigen binding fragment of any of claims 1 through 6, wherein the VL comprises an amino acid sequence encoded by a nucleic acid having at least about 70% sequence identity to SEQ ID NO: 14.

8. The antibody or antigen binding fragment of any of claims 1 through 7, wherein the VH comprises an amino acid sequence encoded by a nucleic acid having at least about 70% sequence identity to SEQ ID NO: 16.

9. The antibody or antigen binding fragment of any of claims 1 through 8 further comprising at least one linker that is a single glycine (Gly) residue; a diglycine peptide (Gly-Gly); a tripeptide (Gly-Gly-Gly); a peptide with four glycine residues (Gly-Gly-Gly-Gly; SEQ ID NO: 37); a peptide with five glycine residues (Gly-Gly-Gly-Gly-Gly; SEQ ID NO; 38); a peptide with six glycine residues (Gly-Gly-Gly-Gly-Gly-Gly; SEQ ID NO: 39); a peptide with seven glycine residues (Gly-Gly-Gly-Gly-Gly-Gly-Gly; SEQ ID NO: 40); a peptide with eight glycine residues (Gly-Gly-Gly-Gly-Gly-Gly-Gly-Gly; SEQ ID NO: 41), the peptide Gly-Gly-Gly-Gly-Ser (SEQ ID NO: 42), the peptide Gly-Gly-Gly-Gly-Ser-Gly-Gly-Gly-Gly-Ser (SEQ ID NO: 43), the peptide Gly-Gly-Gly-Gly-Ser-Gly-Gly-Gly-Gly-Ser-Gly-Gly-Gly-Gly-Ser (SEQ ID NO: 44), a single Ser, a single Val, the dipeptide Arg-Thr, Gln-Pro, Ser-Ser, Thr-Lys, and Ser-Leu; Thr-Lys-Gly-Pro-Ser (SEQ ID NO: 45), Thr-Val-Ala-Ala-Pro (SEQ ID NO: 46), Gln-Pro-Lys-Ala-Ala (SEQ ID NO: 47), Gln-Arg-Ile-Glu-Gly (SEQ ID NO: 48), Ala-Ser-Thr-Lys-Gly-Pro-Ser (SEQ ID NO: 49), Arg-Thr-Val-Ala-Ala-Pro-Ser (SEQ ID NO: 50), Gly-Gln-Pro-Lys-Ala-Ala-Pro (SEQ ID NO: 51), and His-Ile-Asp-Ser-Pro-Asn-Lys (SEQ ID NO: 52).

10. The antibody or antigen binding fragment of any of claims 1 through 9, wherein the VL binds one of the following epitopes: the CD4-binding site, the V1N2-glycan region, the V3-glycan region, the gp41 membrane proximal external region (MPER), or the gp120/gp41 interface of the envelope protein.

11. The antibody or antigen binding fragment of any of claims 1 through 10, wherein the VL comprises one of more of complementarity-determining regions (CDRs) that are at least about 70% identical to the amino acid sequences of SEQ ID NO: 25, SEQ ID NO: 26, SEQ ID NO: 27, SEQ ID NO: 56, SEQ ID NO: 58, and SEQ ID NO: 60.

12. The antibody or antigen binding fragment of any of claims 1 through 11, wherein the VH comprises one of more of complementarity-determining regions (CDRs) that are at least about 70% identical to the amino acid sequences of SEQ ID NO: 28, SEQ ID NO: 29, SEQ ID NO: 30, SEQ ID NO: 67, SEQ ID NO:69, and SEQ ID NO: 71.

13. The antibody or antigen binding fragment of any of claims 1 through 12, wherein the antibody or antibody fragment is encoded by a nucleic acid sequence having at least about 70% sequence identity to SEQ ID NO: 11 and/or SEQ ID NO: 12.

14. The antibody or antigen binding fragment of any of claims 1 through 13, wherein the antigen binding fragment is a scFv of 10-1074.

15. The antibody or antigen binding fragment of any of claims 1 through 14, wherein the antibody or antigen binding fragment is free of a CD19 signal sequence.

16. A cell comprising a nucleic acid sequence encoding one or plurality of antibodies or antigen binding fragments of any of claims 1 through 15.

17. The cell of claim 16, wherein the cell is a T cell.

18. The cell of claim 16 or 17, wherein the cell further comprises a costimulatory molecule capable of binding an HIV antigen.

19. The cell of any of claims 16 through 18, wherein the cell is isolated form a subject diagnosed with or suspected of being infected with HIV.

20. A pharmaceutical composition comprising: (i) one or plurality of the cells of any of claims 16 through 19; and (ii) a pharmaceutically acceptable carrier.

21. A method of treating and/or preventing an HIV infection, comprising administering to a subject in need thereof an effective amount of the cell of any of claims 16 through 19 or the pharmaceutical composition of claim 20.

22. The method of claim 21 further comprising administering to the subject one or a plurality of latency reversing agent (LRA) molecules prior to, simultaneously with or after administering the cell or pharmaceutical composition.

23. The method of claim 21 or 22, wherein the effective amount is sufficient to accomplish one or any combination of: (i) neutralization of one or a plurality of retroviruses in the subject; (ii) induction of NK cell recruitment to a cell in the subject infected with HIV; and (iii) antigen-specific cytotoxicity of a cell infected with HIV in the subject.

24. A nucleic acid encoding the antibody or antigen binding fragment of any of claims 1 through 15.

25. A vector comprising the nucleic acid of claim 24.

26. A method for the preparation of a cell expressing the antigen or antigen-binding fragment of any of claims 1 through 15, comprising the step of culturing the cell under conditions that allow transduction of the cell with the vector of claim 25.

27. The method of claim 26 further comprising the step of isolating the cell by cell sorting.

28. An immunoconjugate comprising the antibody or antibody binding fragment of any of claims 1 through 15 coupled to a cytotoxic agent.

29. A method of destroying a cell in a subject infected by latent HIV infection comprising exposing an effective amount of the pharmaceutical composition of claim 20 to the cell for a time period sufficient to cause cytotoxicity of the cell.

30. The method of claim 29, wherein the cell is contemporaneously exposed to one or a plurality of LRAs.

31. The method of claim 30, wherein the one or plurality of LRAs are chosen from: sIL-2, IL-15SA, bryostatin, and prostratin, or a salt or functional fragment thereof.

32. A composition comprising an expressible nucleic acid sequence encoding an antibody or an antigen-binding fragment thereof, wherein the antibody or the antigen-binding fragment thereof comprises:
(i) a light chain comprising a first secretory signal followed by a light chain variable region (VL) of an anti-human immunodeficiency virus-1 (HIV-1) broadly neutralizing antibody;
(ii) a heavy chain comprising a second secretory signal followed by a heavy chain variable region (VH) of said anti-HIV-1 broadly neutralizing antibody, wherein the VL and the VH are positioned non-contiguously and connected by at least one self-cleaving amino acid sequence, and wherein the VL binds epitopes of the envelope protein of human immunodeficiency virus-1 (HIV-1).

33. The composition of claim 32, wherein the light chain further comprises a light chain constant region of an immunoglobulin G (IgG).

34. The composition of claim 32 or 33, wherein the heavy chain further comprises a heavy chain constant region of an IgG.

35. The composition of any of claims 32 through 34, wherein the light chain constant region of an IgG comprises an amino acid sequence having at least about 70% sequence identity to the amino acid sequence of SEQ ID NO: 78.

36. The composition of any of claims 32 through 35, wherein the heavy chain constant region of an IgG comprises an amino acid sequence having at least about 70% sequence identity to the amino acid sequence of SEQ ID NO: 79.

37. The composition of claim 32, wherein the expressible nucleic acid sequence further comprises a nucleic acid sequence encoding a VL of CD16.

38. The composition of claim 32 or 37, wherein the expressible nucleic acid sequence further comprises a nucleic acid sequence encoding a VH of CD16.

39. The composition of any of claims 32 through 38, wherein the VL comprises at least one complementarity-determining region (CDR) selected from the group consisting of SEQ ID NO: 25, SEQ ID NO: 26, SEQ ID NO: 27, SEQ ID NO: 56, SEQ ID NO: 58, and SEQ ID NO: 60.

40. The composition of any of claims 32 through 39, wherein the VH comprises at least one complementarity-determining region (CDR) selected from the group consisting of SEQ ID NO: 28, SEQ ID NO: 29, SEQ ID NO: 30, SEQ ID NO: 67, SEQ ID NO: 69, and SEQ ID NO: 71.

41. The composition of any of claims 32 through 40, wherein the VL comprises:
a) a first CDR comprising the amino acid sequence of SEQ ID NO: 25 or 56, and at least a second CDR comprising the amino acid sequence of SEQ ID NO: 26, SEQ ID NO: 27, SEQ ID NO: 58, or SEQ ID NO: 60;
b) a first CDR comprising the amino acid sequence of SEQ ID NO: 26 or 58, and at least a second CDR comprising the amino acid sequence of SEQ ID NO: 25, SEQ ID NO: 27, SEQ ID NO: 56, or SEQ ID NO: 60;
c) a first CDR comprising the amino acid sequence of SEQ ID NO: 27 or 60, and at least a second CDR comprising the amino acid sequence of SEQ ID NO: 25, SEQ ID NO: 26, SEQ ID NO: 56, or SEQ ID NO: 58, or
d) a first CDR comprising the amino acid sequence of SEQ ID NO: 25 or 56, a second CDR comprising the amino acid sequence of SEQ ID NO: 26 or 58, and a third CDR comprising the amino acid sequence of SEQ ID NO: 27 or 60.

42. The composition of any of claims 32 through 41, wherein the VH comprises:
a) a first CDR comprising the amino acid sequence of SEQ ID NO: 28 or 67, and at least a second CDR comprising the amino acid sequence of SEQ ID NO: 29, SEQ ID NO: 30, SEQ ID NO: 69, or SEQ ID NO: 71;
b) a first CDR comprising the amino acid sequence of SEQ ID NO: 29 or 69, and at least a second CDR comprising the amino acid sequence of SEQ ID NO: 28, SEQ ID NO: 30, SEQ ID NO: 67, or SEQ ID NO: 71;
c) a first CDR comprising the amino acid sequence of SEQ ID NO: 30 or 71, and at least a second CDR comprising the amino acid sequence of SEQ ID NO: 28, SEQ ID NO: 29, SEQ ID NO: 67, or SEQ ID NO: 69; or
d) a first CDR comprising the amino acid sequence of SEQ ID NO: 28 or 67, a second CDR comprising the amino acid sequence of SEQ ID NO: 29 or 69, and a third CDR comprising the amino acid sequence of SEQ ID NO: 30 or 71.

43. The composition of any of claims 32 through 42, wherein the VL further comprises at least one framework region (FR) selected from the group consisting of SEQ ID NO: 55, SEQ ID NO: 57, SEQ ID NO: 59, and SEQ ID NO: 61.

44. The composition of any of claims 32 through 43, wherein the heavy chain further comprises at least one FR selected from the group consisting of SEQ ID NO: 66, SEQ ID NO: 68, SEQ ID NO: 70, and SEQ ID NO: 72.

45. The composition of any of claims 32 through 44, wherein the VL comprises an amino acid sequence having at least about 70% sequence identity to the amin acid sequence of SEQ ID NO: 23 or 53.

46. The composition of any of claims 32 through 45, wherein the VH comprises an amino acid sequence having at least about 70% sequence identity to the amin acid sequence of SEQ ID NO: 24 or 64.

47. The composition of any of claims 32 through 46, wherein the light chain further comprises at least one amino acid sequence having at least about 70% sequence identity to SEQ ID NO: 78.

48. The composition of any of claims 32 through 47, wherein the heavy chain further comprises at least one amino acid sequence having at least about 70% sequence identity to SEQ ID NO: 79.

49. The composition of any of claims 32 through 48, wherein the antibody or the antigen-binding fragment thereof further comprises at least one furin linker.

50. The composition of any of claims 32 through 49, wherein the at least one self-cleaving amino acid sequence is selected from the group consisting of FMDV 2A (F2A), equine rhinitis A virus (ERAV) 2A (E2A), porcine teschovirus-1 2A (P2A), and Thoseaasigna virus 2A (T2A), or at least one internal ribosome entry sequence (IRES) separates construct domains.

51. The composition of any of claims 32 through 50, wherein the light chain comprises an amino acid sequence having at least about 70% sequence identity to the amino acid sequence encoded by the nucleic acid sequence of SEQ ID NO: 14.

52. The composition of any of claims 32 through 51, wherein the heavy chain comprises an amino acid sequence having at least about 70% sequence identity to the amino acid sequence encoded by the nucleic acid sequence of SEQ ID NO: 16.

53. The composition of any of claims 32 through 52, wherein either the VL and/or VH at least partially binds to V3 glycan supersite of the HIV envelope protein.

54. The composition of any of claims 32 through 53, wherein the expressible nucleic acid sequence further comprises at least one nucleic acid sequence encoding a linker selected from the group consisting of a single glycine (Gly) residue, a diglycine peptide (Gly-Gly), a tripeptide (Gly-Gly-Gly), a single Ser, a single Val, the dipeptide Arg-Thr, Gln-Pro, Ser-Ser, Thr-Lys, and Ser-Leu, and the amino acid sequences of SEQ ID NO: 37, 38, 39, 40, 41, 42, 43, 44, 45, 46, 47, 48, 49, 50, 51, and 52.

55. The composition of any of claims 32 through 54, wherein the VL binds one of the following epitopes: the CD4-binding site, the V1/V2-glycan region, the V3-glycan region, the gp41 membrane proximal external region (MPER), or the gp120/gp41 interface of the envelope protein.

56. The composition of any of claims 32 through 55, wherein the antigen binding fragment is a single-chain variable fragment (scFv) of antibody 10-1074 and comprises an amino acid sequence having at least about 70% sequence identity with the amino acid sequence of SEQ ID NO: 75.

57. The composition of any of claims 32 through 56, wherein the the expressible nucleic acid sequence further comprises a CD19 signal sequence.

58. The composition of any of claims 32 through 57, wherein the antibody or the antigen binding fragment is not the full length of antibody 10-1074 encoded by the nucleic acid sequence of SEQ ID NO: 12.

59. A cell comprising the composition of any of claims 32 through 58.

60. The cell of claim 59, wherein the cell is a T cell.

61. The cell of claim 59 or 60, wherein the cell further comprises a costimulatory molecule capable of binding an HIV antigen.

62. The cell of any of claims 59 through 61, wherein the cell is isolated form a subject diagnosed with or suspected of being infected with HIV.

63. A pharmaceutical composition comprising: (i) one or plurality of the cells of any of claims 59 through 63; and (ii) a pharmaceutically acceptable carrier.

64. A method of treating and/or preventing an HIV infection, comprising administering to a subject in need thereof an effective amount of the cell of any of claims 59 through 63 or the pharmaceutical composition of claim 63.

65. The method of claim 64 further comprising administering to the subject one or a plurality of latency reversing agent (LRA) molecules prior to, simultaneously with or after administering the cell or pharmaceutical composition.

66. The method of claim 64 or 65, wherein the effective amount is sufficient to accomplish one or any combination of: (i) neutralization of one or a plurality of retroviruses in the subject; (ii) induction of NK cell recruitment to a cell in the subject infected with HIV; and (iii) antigen-specific cytotoxicity of a cell infected with HIV in the subject.

67. A method for the preparation of the cell of any of claims 59 through 63 comprising the step of culturing the cell under conditions that allow transduction of the cell with the composition comprising the expressible nucleic acid sequence.

68. The method of claim 67 further comprising the step of isolating the cell by cell sorting.

69. An immunoconjugate comprising the antibody or antibody binding fragment encoded by the composition of any of claims 32 through 58.

70. A method of destroying a cell in a subject infected by latent HIV infection comprising exposing an effective amount of the pharmaceutical composition of claim 63 to the cell for a time period sufficient to cause cytotoxicity of the cell.

71. The method of claim 70, wherein the cell is contemporaneously exposed to one or a plurality of LRAs.

72. The method of claim 71, wherein the one or plurality of LRAs are chosen from: sIL-2, IL-15SA, bryostatin, and prostratin, or a salt or functional fragment thereof.

73. The cell of claims 16, 17, 59, and 60, where the cell is a T cell recognizing HIV antigens in the following combinations: (1) gag, (2) nef, (3) pol, (4) gag and nef, (5) gag and pol, (6) nef and pol, (7) gag, nef, and pol.

74. The cell of claim 73, where the T cell recognizes only a subset of antigens from HIV gag, nef, and pol.

75. The cell of claims 16, 17, 59, and 60, where the cell is a T cell recognizing EBV antigens in the following combinations: (1) BARF1, (2) BMLF1, (3) BMRF1, (4) BRLF1, (5) BZLF1, (6) EBNA-LP, (7) EBNA1, (8) EBNA2, (9) EBNA3a, (10) EBNA3b, (11) EBNA3c, (12) GP350, (13) GP340, (14) LMP1, (15) LMP2, (16) EBNA-LP, EBNA1, EBNA2, EBNA3a, EBNA3b, EBNA3c, (17) LMP1, LMP2, (18) BARF1, BMLF1, BMRF1, BRLF1, BZLF1, (19) EBNA-LP, (20) EBNA1, LMP2, and BZLF1, (21) EBNA1, EBNA2, BZLF1 LMP1, and LMP2, (22) EBNA-LP, EBNA1, EBNA2, EBNA3a, EBNA3b, EBNA3c, LMP1, LMP2, BARF1, BMLF1, BMRF1, BRLF1, BZLF1.

76. The cell of claim 76, where the T cell recognizes only a subset of antigens from EBV EBNA-LP, EBNA1, EBNA2, EBNA3a, EBNA3b, EBNA3c, LMP1, LMP2, BARF1, BMLF1, BMRF1, BRLF1, BZLF1.

77. The cell of claims 16, 17, 59, and 60, where the cell is a T cell recognizing HPV serotype 16, 18, or 31 antigens in the following combinations: (1) E6, (2) E7, (3) L1, (4) L2, (5) E1, (6) E4, (7) E5, (8) E6 and E6, (9) E1, E4, E5, E6, E7 L1, L2.

78. The cell of claim 77, where the T cell recognizes only a subset of antigens from HPV 16, 18, or 31 E1, E4, E5, E6, E7 L1, L2.

79. The cell of claims 16, 17, 59, and 60, where the cell is a T cell recognizing HHV8/KSHV antigens in the following combinations: (1) ORF8, (2) ORF11, (3) ORF25, (4) ORF33, (5) ORF37, (6) ORF41, (7) ORF46, (8) ORF47, (9) ORF57, (10) LANAI, (11) v-cyclin, (12) v-IL6, (13) v-GPCR, (14) v-FLIP, (15) v-IRF3, (16) ORF8, ORF11, ORF25, ORF33, ORF37, ORF41, ORF46, ORF47, ORF57, (17) ORF8, ORF11, ORF57, (18) ORF8 and ORF11, (19) LANAI, v-cyclin, v-IL6, v-GPCR, v-FLIP, v-IRF3, (20) VFLIP, VIRF3, V cyclin, VIL6, V GPCR, (21) ORF8, ORF11, ORF25, ORF33, ORF37, ORF41, ORF46, ORF47, ORF57, LANAI, v-cyclin, v-IL6, v-GPCR, v-FLIP, v-IRF3.

80. The cell of claim 77, where the T cell recognizes only a subset of antigens from HHV8/KSHV 16, 18, or 31 ORF8, ORF11, ORF25, ORF33, ORF37, ORF41, ORF46, ORF47, ORF57, LANAI, v-cyclin, v-IL6, v-GPCR, v-FLIP, v-IRF3.

81. The cell of claims 16, 17, 59, and 60, where the cell is a T cell recognizing endogenous retrovirus sequences from HERV-HF, HERV-H, HERV-F, HERV-RW, HERV-W, ERV9, HuERS-P, HuRRS-P, HERV-ER1, 4-1, 5-1, ERV3, RRHERV-I, HERV-T, S71, CRTK1, CRTK6, HERV-IP, RTVL-I, ERV-FTD, ERV-FRD, class II HERVs, HERV-K.

82. The cell of claim 81, where the T cell recognizes only a subset of antigens from HERV-HF, HERV-H, HERV-F, HERV-RW, HERV-W, ERV9, HuERS-P, HuRRS-P, HERV-ER1, 4-1, 5-1, ERV3, RRHERV-I, HERV-T, S71, CRTK1, CRTK6, HERV-IP, RTVL-I, ERV-FTD, ERV-FRD, class II HERVs, HERV-K.


SEQUENCE LISTING (86 sequences)

SEQ ID NO: 1 - DNA, 4444 bp, Artificial Sequence
  PG9 Full length Antibody- nucleic acid
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcacagtctgccctgactcagcctgcctccgtgtctgggtctcctggacagtcgatcaccatctcctgcaatggaaccagcaatgatgttggtggctatgaatctgtctcctggtaccaacaacatcccggcaaagcccccaaagtcgtgatttatgatgtcagtaaacggccctcaggggtttctaatcgcttctctggctccaagtccggcaacacggcctccctgaccatctctgggctccaggctgaggacgagggtgactattactgcaagtctctgacaagcacgagacgtcgggttttcggcactgggaccaagctgaccgttctaaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccgcaaacgccgcggcagcggcgcgaccaactttagcctgctgaaacaggcgggcgatgtggaagaaaacccgggcccgatgggatggtcatgtatcatcctttttctagtagcaactgcaaccggtgtacattcacagcgattagtggagtctgggggaggcgtggtccagcctgggtcgtccctgagactctcctgtgcagcgtccggattcgacttcagtagacaaggcatgcactgggtccgccaggctccaggccaggggctggagtgggtggcatttattaaatatgatggaagtgagaaatatcatgctgactccgtatggggccgactcagcatctccagagacaattccaaggatacgctttatctccaaatgaatagcctgagagtcgaggacacggctacatatttttgtgtgagagaggctggtgggcccgactaccgtaatgggtacaactattacgatttctatgatggttattataactaccactatatggacgtctggggcaaagggaccacggtcaccgtctcgagcgcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 2 - DNA, 3456 bp, Artificial Sequence
  G9 Full length Antibody- nucleic acid encoded region
atgggatggtcatgtatcatcctttttctagtagcaactgcaaccggtgtacattcacagtctgccctgactcagcctgcctccgtgtctgggtctcctggacagtcgatcaccatctcctgcaatggaaccagcaatgatgttggtggctatgaatctgtctcctggtaccaacaacatcccggcaaagcccccaaagtcgtgatttatgatgtcagtaaacggccctcaggggtttctaatcgcttctctggctccaagtccggcaacacggcctccctgaccatctctgggctccaggctgaggacgagggtgactattactgcaagtctctgacaagcacgagacgtcgggttttcggcactgggaccaagctgaccgttctaaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccgcaaacgccgcggcagcggcgcgaccaactttagcctgctgaaacaggcgggcgatgtggaagaaaacccgggcccgatgggatggtcatgtatcatcctttttctagtagcaactgcaaccggtgtacattcacagcgattagtggagtctgggggaggcgtggtccagcctgggtcgtccctgagactctcctgtgcagcgtccggattcgacttcagtagacaaggcatgcactgggtccgccaggctccaggccaggggctggagtgggtggcatttattaaatatgatggaagtgagaaatatcatgctgactccgtatggggccgactcagcatctccagagacaattccaaggatacgctttatctccaaatgaatagcctgagagtcgaggacacggctacatatttttgtgtgagagaggctggtgggcccgactaccgtaatgggtacaactattacgatttctatgatggttattataactaccactatatggacgtctggggcaaagggaccacggtcaccgtctcgagcgcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 3 - DNA, 2770 bp, Artificial Sequence
  PG9 light chain nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcacagtctgccctgactcagcctgcctccgtgtctgggtctcctggacagtcgatcaccatctcctgcaatggaaccagcaatgatgttggtggctatgaatctgtctcctggtaccaacaacatcccggcaaagcccccaaagtcgtgatttatgatgtcagtaaacggccctcaggggtttctaatcgcttctctggctccaagtccggcaacacggcctccctgaccatctctgggctccaggctgaggacgagggtgactattactgcaagtctctgacaagcacgagacgtcgggttttcggcactgggaccaagctgaccgttctaaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 4 - DNA, 1782 bp, Artificial Sequence
  PG9 light chain nucleic acid sequence- encoded region
atgggatggtcatgtatcatcctttttctagtagcaactgcaaccggtgtacattcacagtctgccctgactcagcctgcctccgtgtctgggtctcctggacagtcgatcaccatctcctgcaatggaaccagcaatgatgttggtggctatgaatctgtctcctggtaccaacaacatcccggcaaagcccccaaagtcgtgatttatgatgtcagtaaacggccctcaggggtttctaatcgcttctctggctccaagtccggcaacacggcctccctgaccatctctgggctccaggctgaggacgagggtgactattactgcaagtctctgacaagcacgagacgtcgggttttcggcactgggaccaagctgaccgttctaaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 5 - DNA, 3661 bp, Artificial Sequence
  PG9 heavy chain nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcacagcgattagtggagtctgggggaggcgtggtccagcctgggtcgtccctgagactctcctgtgcagcgtccggattcgacttcagtagacaaggcatgcactgggtccgccaggctccaggccaggggctggagtgggtggcatttattaaatatgatggaagtgagaaatatcatgctgactccgtatggggccgactcagcatctccagagacaattccaaggatacgctttatctccaaatgaatagcctgagagtcgaggacacggctacatatttttgtgtgagagaggctggtgggcccgactaccgtaatgggtacaactattacgatttctatgatggttattataactaccactatatggacgtctggggcaaagggaccacggtcaccgtctcgagcgcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 6 - DNA, 2673 bp, Artificial Sequence
  PG9 heavy chain nucleic acid sequence-encoded region
atgggatggtcatgtatcatcctttttctagtagcaactgcaaccggtgtacattcacagcgattagtggagtctgggggaggcgtggtccagcctgggtcgtccctgagactctcctgtgcagcgtccggattcgacttcagtagacaaggcatgcactgggtccgccaggctccaggccaggggctggagtgggtggcatttattaaatatgatggaagtgagaaatatcatgctgactccgtatggggccgactcagcatctccagagacaattccaaggatacgctttatctccaaatgaatagcctgagagtcgaggacacggctacatatttttgtgtgagagaggctggtgggcccgactaccgtaatgggtacaactattacgatttctatgatggttattataactaccactatatggacgtctggggcaaagggaccacggtcaccgtctcgagcgcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 7 - DNA, 330 bp, Artificial Sequence
  Homo sapiens isolate PG9 anti-HIV immunoglobulin light chain variable region mRNA
cagtctgccctgactcagcctgcctccgtgtctgggtctcctggacagtcgatcaccatctcctgcaatggaaccagcaatgatgttggtggctatgaatctgtctcctggtaccaacaacatcccggcaaagcccccaaagtcgtgatttatgatgtcagtaaacggccctcaggggtttctaatcgcttctctggctccaagtccggcaacacggcctccctgaccatctctgggctccaggctgaggacgagggtgactattactgcaagtctctgacaagcacgagacgtcgggttttcggcactgggaccaagctgaccgttcta

SEQ ID NO: 8 - DNA, 408 bp, Artificial Sequence
  Homo sapiens isolate PG9 anti-HIV immunoglobulin heavy chain variable region mRNA
cagcgattagtggagtctgggggaggcgtggtccagcctgggtcgtccctgagactctcctgtgcagcgtccggattcgacttcagtagacaaggcatgcactgggtccgccaggctccaggccaggggctggagtgggtggcatttattaaatatgatggaagtgagaaatatcatgctgactccgtatggggccgactcagcatctccagagacaattccaaggatacgctttatctccaaatgaatagcctgagagtcgaggacacggctacatatttttgtgtgagagaggctggtgggcccgactaccgtaatgggtacaactattacgatttctatgatggttattataactaccactatatggacgtctggggcaaagggaccacggtcaccgtctcgagc

SEQ ID NO: 9 - DNA, 3673 bp, Artificial Sequence
  PG9 scFv nucleic acid
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatggattggatttggcgcattctgtttctggtgggcgcggcgaccggcgcgcatagcgaagtgcagctggtggaaagcggcggcggcgtggtgcgcccgggcggcagcctgcgcctgagctgcgcggcgagcggctttacctttgatgattatggcatgagctgggtgcgccaggcgccgggcaaaggcctggaatgggtgagcggcattaactggaacggcggcagcaccggctatgcggatagcgtgaaaggccgctttaccattagccgcgataacgcgaaaaacagcctgtatctgcagatgaacagcctgcgcgcggaagataccgcggtgtattattgcgcgcgcggccgcagcctgctgtttgattattggggccagggcaccctggtgaccgtgagccgcggcggcggcggcagcggcggcggcggcagcggcggcggcggcagcggcggcggcggcagcagcagcgaactgacccaggatccggcggtgagcgtggcgctgggccagaccgtgcgcattacctgccagggcgatagcctgcgcagctattatgcgagctggtatcagcagaaaccgggccaggcgccggtgctggtgatttatggcaaaaacaaccgcccgagcggcattccggatcgctttagcggcagcagcagcggcaacaccgcgagcctgaccattaccggcgcgcaggcggaagatgaagcggattattattgcaacagccgcgatagcagcggcaaccatgtggtgtttggcggcggcaccaaactgaccgtgggcagcggcggcggcggcagccagcgattagtggagtctgggggaggcgtggtccagcctgggtcgtccctgagactctcctgtgcagcgtccggattcgacttcagtagacaaggcatgcactgggtccgccaggctccaggccaggggctggagtgggtggcatttattaaatatgatggaagtgagaaatatcatgctgactccgtatggggccgactcagcatctccagagacaattccaaggatacgctttatctccaaatgaatagcctgagagtcgaggacacggctacatatttttgtgtgagagaggctggtgggcccgactaccgtaatgggtacaactattacgatttctatgatggttattataactaccactatatggacgtctggggcaaagggaccacggtcaccgtctcgagcggcggcggcggcagcggcggcggcggcagcggcggcggcggcagcggcggcggcggcagccagtctgccctgactcagcctgcctccgtgtctgggtctcctggacagtcgatcaccatctcctgcaatggaaccagcaatgatgttggtggctatgaatctgtctcctggtaccaacaacatcccggcaaagcccccaaagtcgtgatttatgatgtcagtaaacggccctcaggggtttctaatcgcttctctggctccaagtccggcaacacggcctccctgaccatctctgggctccaggctgaggacgagggtgactattactgcaagtctctgacaagcacgagacgtcgggttttcggcactgggaccaagctgaccgttctacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 10 - DNA, 2685 bp, Artificial Sequence
  PG9 scFv nucleic acid- encoded region
atggattggatttggcgcattctgtttctggtgggcgcggcgaccggcgcgcatagcgaagtgcagctggtggaaagcggcggcggcgtggtgcgcccgggcggcagcctgcgcctgagctgcgcggcgagcggctttacctttgatgattatggcatgagctgggtgcgccaggcgccgggcaaaggcctggaatgggtgagcggcattaactggaacggcggcagcaccggctatgcggatagcgtgaaaggccgctttaccattagccgcgataacgcgaaaaacagcctgtatctgcagatgaacagcctgcgcgcggaagataccgcggtgtattattgcgcgcgcggccgcagcctgctgtttgattattggggccagggcaccctggtgaccgtgagccgcggcggcggcggcagcggcggcggcggcagcggcggcggcggcagcggcggcggcggcagcagcagcgaactgacccaggatccggcggtgagcgtggcgctgggccagaccgtgcgcattacctgccagggcgatagcctgcgcagctattatgcgagctggtatcagcagaaaccgggccaggcgccggtgctggtgatttatggcaaaaacaaccgcccgagcggcattccggatcgctttagcggcagcagcagcggcaacaccgcgagcctgaccattaccggcgcgcaggcggaagatgaagcggattattattgcaacagccgcgatagcagcggcaaccatgtggtgtttggcggcggcaccaaactgaccgtgggcagcggcggcggcggcagccagcgattagtggagtctgggggaggcgtggtccagcctgggtcgtccctgagactctcctgtgcagcgtccggattcgacttcagtagacaaggcatgcactgggtccgccaggctccaggccaggggctggagtgggtggcatttattaaatatgatggaagtgagaaatatcatgctgactccgtatggggccgactcagcatctccagagacaattccaaggatacgctttatctccaaatgaatagcctgagagtcgaggacacggctacatatttttgtgtgagagaggctggtgggcccgactaccgtaatgggtacaactattacgatttctatgatggttattataactaccactatatggacgtctggggcaaagggaccacggtcaccgtctcgagcggcggcggcggcagcggcggcggcggcagcggcggcggcggcagcggcggcggcggcagccagtctgccctgactcagcctgcctccgtgtctgggtctcctggacagtcgatcaccatctcctgcaatggaaccagcaatgatgttggtggctatgaatctgtctcctggtaccaacaacatcccggcaaagcccccaaagtcgtgatttatgatgtcagtaaacggccctcaggggtttctaatcgcttctctggctccaagtccggcaacacggcctccctgaccatctctgggctccaggctgaggacgagggtgactattactgcaagtctctgacaagcacgagacgtcgggttttcggcactgggaccaagctgaccgttctacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 11 - DNA, 4438 bp, Artificial Sequence
  10-1074 full length nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcatcctatgtcaggccactgtccgtcgcactgggggagaccgcaagaattagctgtgggaggcaggcactggggagcagggctgtccagtggtaccagcaccgaccaggacaggcaccaatcctgctgatctacaacaatcaggaccggccttcaggcatccccgagagattcagcggaacacccgatattaactttggcactagagctaccctgacaatcagcggagtggaggcaggcgacgaagccgattactattgccatatgtgggactccaggtctgggttcagttggtcatttggcggagcaactcgactgaccgtgctgaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccgcaaacgccgcggcagcggcgcgaccaactttagcctgctgaaacaggcgggcgatgtggaagaaaacccgggcccgatgggatggtcatgtatcatcctttttctagtagcaactgcaaccggtgtacattcacaggtgcagctgcaggaatctgggcctggactggtcaaaccctccgagactctgagcgtcacttgttctgtgagcggcgactctatgaacaattactattggacatggatccgacagagcccaggcaaggggctggagtggatcggctacatttctgacagagaaagtgctacttataaccctagcctgaattccagggtggtcatttcacgcgacaccagcaagaaccagctgtccctgaaactgaattctgtgacccccgcagatacagccgtctactattgcgccaccgctcggagaggacagcggatctacggcgtggtcagcttcggggagttcttttactactactcaatggatgtctgggggaaggggactacagtgaccgtctcaagcgcctcgaccaaggcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 12 - DNA, 3450 bp, Artificial Sequence
  10-1074 full length nucleic acid sequence- encoded region
atgggatggtcatgtatcatcctttttctagtagcaactgcaaccggtgtacattcatcctatgtcaggccactgtccgtcgcactgggggagaccgcaagaattagctgtgggaggcaggcactggggagcagggctgtccagtggtaccagcaccgaccaggacaggcaccaatcctgctgatctacaacaatcaggaccggccttcaggcatccccgagagattcagcggaacacccgatattaactttggcactagagctaccctgacaatcagcggagtggaggcaggcgacgaagccgattactattgccatatgtgggactccaggtctgggttcagttggtcatttggcggagcaactcgactgaccgtgctgaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccgcaaacgccgcggcagcggcgcgaccaactttagcctgctgaaacaggcgggcgatgtggaagaaaacccgggcccgatgggatggtcatgtatcatcctttttctagtagcaactgcaaccggtgtacattcacaggtgcagctgcaggaatctgggcctggactggtcaaaccctccgagactctgagcgtcacttgttctgtgagcggcgactctatgaacaattactattggacatggatccgacagagcccaggcaaggggctggagtggatcggctacatttctgacagagaaagtgctacttataaccctagcctgaattccagggtggtcatttcacgcgacaccagcaagaaccagctgtccctgaaactgaattctgtgacccccgcagatacagccgtctactattgcgccaccgctcggagaggacagcggatctacggcgtggtcagcttcggggagttcttttactactactcaatggatgtctgggggaaggggactacagtgaccgtctcaagcgcctcgaccaaggcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 13 - DNA, 2764 bp, Artificial Sequence
  10-1074 light chain nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcatcctatgtcaggccactgtccgtcgcactgggggagaccgcaagaattagctgtgggaggcaggcactggggagcagggctgtccagtggtaccagcaccgaccaggacaggcaccaatcctgctgatctacaacaatcaggaccggccttcaggcatccccgagagattcagcggaacacccgatattaactttggcactagagctaccctgacaatcagcggagtggaggcaggcgacgaagccgattactattgccatatgtgggactccaggtctgggttcagttggtcatttggcggagcaactcgactgaccgtgctgaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 14 - DNA, 1776 bp, Artificial Sequence
  10-1074 light chain nucleic acid sequence- encoded region
atgggatggtcatgtatcatcctttttctagtagcaactgcaaccggtgtacattcatcctatgtcaggccactgtccgtcgcactgggggagaccgcaagaattagctgtgggaggcaggcactggggagcagggctgtccagtggtaccagcaccgaccaggacaggcaccaatcctgctgatctacaacaatcaggaccggccttcaggcatccccgagagattcagcggaacacccgatattaactttggcactagagctaccctgacaatcagcggagtggaggcaggcgacgaagccgattactattgccatatgtgggactccaggtctgggttcagttggtcatttggcggagcaactcgactgaccgtgctgaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 15 - DNA, 3661 bp, Artificial Sequence
  10-1074 heavy chain nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggatggtcatgtatcatcctttttctagtagcaactgcaaccggtgtacattcacaggtgcagctgcaggaatctgggcctggactggtcaaaccctccgagactctgagcgtcacttgttctgtgagcggcgactctatgaacaattactattggacatggatccgacagagcccaggcaaggggctggagtggatcggctacatttctgacagagaaagtgctacttataaccctagcctgaattccagggtggtcatttcacgcgacaccagcaagaaccagctgtccctgaaactgaattctgtgacccccgcagatacagccgtctactattgcgccaccgctcggagaggacagcggatctacggcgtggtcagcttcggggagttcttttactactactcaatggatgtctgggggaaggggactacagtgaccgtctcaagcgcctcgaccaaggcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 16 - DNA, 2673 bp, Artificial Sequence
  10-1074 heavy chain nucleic acid sequence- encoded region
atgggatggtcatgtatcatcctttttctagtagcaactgcaaccggtgtacattcacaggtgcagctgcaggaatctgggcctggactggtcaaaccctccgagactctgagcgtcacttgttctgtgagcggcgactctatgaacaattactattggacatggatccgacagagcccaggcaaggggctggagtggatcggctacatttctgacagagaaagtgctacttataaccctagcctgaattccagggtggtcatttcacgcgacaccagcaagaaccagctgtccctgaaactgaattctgtgacccccgcagatacagccgtctactattgcgccaccgctcggagaggacagcggatctacggcgtggtcagcttcggggagttcttttactactactcaatggatgtctgggggaaggggactacagtgaccgtctcaagcgcctcgaccaaggcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaacgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 17 - DNA, 324 bp, Artificial Sequence
  10-1074-LC_1012F- light chain variable region
tcctatgtcaggccactgtccgtcgcactgggggagaccgcaagaattagctgtgggaggcaggcactggggagcagggctgtccagtggtaccagcaccgaccaggacaggcaccaatcctgctgatctacaacaatcaggaccggccttcaggcatccccgagagattcagcggaacacccgatattaactttggcactagagctaccctgacaatcagcggagtggaggcaggcgacgaagccgattactattgccatatgtgggactccaggtctgggttcagttggtcatttggcggagcaactcgactgaccgtgctg

SEQ ID NO: 18 - DNA, 408 bp, Artificial Sequence
  10-1074-LC_1012F- heavy chain variable region
caggtgcagctgcaggaatctgggcctggactggtcaaaccctccgagactctgagcgtcacttgttctgtgagcggcgactctatgaacaattactattggacatggatccgacagagcccaggcaaggggctggagtggatcggctacatttctgacagagaaagtgctacttataaccctagcctgaattccagggtggtcatttcacgcgacaccagcaagaaccagctgtccctgaaactgaattctgtgacccccgcagatacagccgtctactattgcgccaccgctcggagaggacagcggatctacggcgtggtcagcttcggggagttcttttactactactcaatggatgtctgggggaaggggactacagtgaccgtctcaagcgcctcgaccaag

SEQ ID NO: 19 - DNA, 3667 bp, Artificial Sequence
  10-1074 scFv nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatggattggatttggcgcattctgtttctggtgggcgcggcgaccggcgcgcatagcgaagtgcagctggtggaaagcggcggcggcgtggtgcgcccgggcggcagcctgcgcctgagctgcgcggcgagcggctttacctttgatgattatggcatgagctgggtgcgccaggcgccgggcaaaggcctggaatgggtgagcggcattaactggaacggcggcagcaccggctatgcggatagcgtgaaaggccgctttaccattagccgcgataacgcgaaaaacagcctgtatctgcagatgaacagcctgcgcgcggaagataccgcggtgtattattgcgcgcgcggccgcagcctgctgtttgattattggggccagggcaccctggtgaccgtgagccgcggcggcggcggcagcggcggcggcggcagcggcggcggcggcagcggcggcggcggcagcagcagcgaactgacccaggatccggcggtgagcgtggcgctgggccagaccgtgcgcattacctgccagggcgatagcctgcgcagctattatgcgagctggtatcagcagaaaccgggccaggcgccggtgctggtgatttatggcaaaaacaaccgcccgagcggcattccggatcgctttagcggcagcagcagcggcaacaccgcgagcctgaccattaccggcgcgcaggcggaagatgaagcggattattattgcaacagccgcgatagcagcggcaaccatgtggtgtttggcggcggcaccaaactgaccgtgggcagcggcggcggcggcagccaggtgcagctgcaggaatctgggcctggactggtcaaaccctccgagactctgagcgtcacttgttctgtgagcggcgactctatgaacaattactattggacatggatccgacagagcccaggcaaggggctggagtggatcggctacatttctgacagagaaagtgctacttataaccctagcctgaattccagggtggtcatttcacgcgacaccagcaagaaccagctgtccctgaaactgaattctgtgacccccgcagatacagccgtctactattgcgccaccgctcggagaggacagcggatctacggcgtggtcagcttcggggagttcttttactactactcaatggatgtctgggggaaggggactacagtgaccgtctcaagcgcctcgaccaagggcggcggcggcagcggcggcggcggcagcggcggcggcggcagcggcggcggcggcagctcctatgtcaggccactgtccgtcgcactgggggagaccgcaagaattagctgtgggaggcaggcactggggagcagggctgtccagtggtaccagcaccgaccaggacaggcaccaatcctgctgatctacaacaatcaggaccggccttcaggcatccccgagagattcagcggaacacccgatattaactttggcactagagctaccctgacaatcagcggagtggaggcaggcgacgaagccgattactattgccatatgtgggactccaggtctgggttcagttggtcatttggcggagcaactcgactgaccgtgctgcgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 20 - DNA, 2679 bp, Artificial Sequence
  10-1074 scFv nucleic acid sequence- encoded region
atggattggatttggcgcattctgtttctggtgggcgcggcgaccggcgcgcatagcgaagtgcagctggtggaaagcggcggcggcgtggtgcgcccgggcggcagcctgcgcctgagctgcgcggcgagcggctttacctttgatgattatggcatgagctgggtgcgccaggcgccgggcaaaggcctggaatgggtgagcggcattaactggaacggcggcagcaccggctatgcggatagcgtgaaaggccgctttaccattagccgcgataacgcgaaaaacagcctgtatctgcagatgaacagcctgcgcgcggaagataccgcggtgtattattgcgcgcgcggccgcagcctgctgtttgattattggggccagggcaccctggtgaccgtgagccgcggcggcggcggcagcggcggcggcggcagcggcggcggcggcagcggcggcggcggcagcagcagcgaactgacccaggatccggcggtgagcgtggcgctgggccagaccgtgcgcattacctgccagggcgatagcctgcgcagctattatgcgagctggtatcagcagaaaccgggccaggcgccggtgctggtgatttatggcaaaaacaaccgcccgagcggcattccggatcgctttagcggcagcagcagcggcaacaccgcgagcctgaccattaccggcgcgcaggcggaagatgaagcggattattattgcaacagccgcgatagcagcggcaaccatgtggtgtttggcggcggcaccaaactgaccgtgggcagcggcggcggcggcagccaggtgcagctgcaggaatctgggcctggactggtcaaaccctccgagactctgagcgtcacttgttctgtgagcggcgactctatgaacaattactattggacatggatccgacagagcccaggcaaggggctggagtggatcggctacatttctgacagagaaagtgctacttataaccctagcctgaattccagggtggtcatttcacgcgacaccagcaagaaccagctgtccctgaaactgaattctgtgacccccgcagatacagccgtctactattgcgccaccgctcggagaggacagcggatctacggcgtggtcagcttcggggagttcttttactactactcaatggatgtctgggggaaggggactacagtgaccgtctcaagcgcctcgaccaagggcggcggcggcagcggcggcggcggcagcggcggcggcggcagcggcggcggcggcagctcctatgtcaggccactgtccgtcgcactgggggagaccgcaagaattagctgtgggaggcaggcactggggagcagggctgtccagtggtaccagcaccgaccaggacaggcaccaatcctgctgatctacaacaatcaggaccggccttcaggcatccccgagagattcagcggaacacccgatattaactttggcactagagctaccctgacaatcagcggagtggaggcaggcgacgaagccgattactattgccatatgtgggactccaggtctgggttcagttggtcatttggcggagcaactcgactgaccgtgctgcgcaaacgccgcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 21 - DNA, 318 bp, Artificial Sequence
  light chain constant region IgG3
accgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 22 - DNA, 1131 bp, Artificial Sequence
  heavy chain constant region
gcgagcaccaaaggcccgagcgtgtttccgctggcgccgtgcagccgcagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatacctgcaacgtgaaccataaaccgagcaacaccaaagtggataaacgcgtggaactgaaaaccccgctgggcgataccacccatacctgcccgcgctgcccggaaccgaaaagctgcgataccccgccgccgtgcccgcgctgcccggaaccgaaaagctgcgataccccgccgccgtgcccgcgctgcccggaaccgaaaagctgcgataccccgccgccgtgcccgcgctgcccggcgccggaactgctgggcggcccgagcgtgtttctgtttccgccgaaaccgaaagataccctgatgattagccgcaccccggaagtgacctgcgtggtggtggatgtgagccatgaagatccggaagtgcagtttaaatggtatgtggatggcgtggaagtgcataacgcgaaaaccaaaccgcgcgaagaacagtataacagcacctttcgcgtggtgagcgtgctgaccgtgctgcatcaggattggctgaacggcaaagaatataaatgcaaagtgagcaacaaagcgctgccggcgccgattgaaaaaaccattagcaaaaccaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcagcggccagccggaaaacaactataacaccaccccgccgatgctggatagcgatggcagcttttttctgtatagcaaactgaccgtggataaaagccgctggcagcagggcaacatttttagctgcagcgtgatgcatgaagcgctgcataaccgctttacccagaaaagcctgagcctgagcccgggcaaa

SEQ ID NO: 23 - protein, 108 aa, Artificial Sequence
  10-1074 variable light (VL) chain amino acid sequence
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 24 - protein, 132 aa, Artificial Sequence
  10-1074 variable heavy (VH) chain amino acid sequence
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 25 - protein, 11 aa, Artificial Sequence
  VL-CDR1.1
GRQALGSRAVQ

SEQ ID NO: 26 - protein, 7 aa, Artificial Sequence
  VL-CDR2.1
NNQDRPS

SEQ ID NO: 27 - protein, 12 aa, Artificial Sequence
  VL-CDR3.1
HMWDSRSGFSWS

SEQ ID NO: 28 - protein, 5 aa, Artificial Sequence
  VH-CDR1.1
NYYWT

SEQ ID NO: 29 - protein, 16 aa, Artificial Sequence
  VH-CDR2.1
YISDRESATYNPSLNS

SEQ ID NO: 30 - protein, 24 aa, Artificial Sequence
  VH-CDR3.1
ARRGQRIYGVVSFGEFFYYYSMDV

SEQ ID NO: 31 - DNA, 988 bp, Artificial Sequence
  CMV promoter and spacer regions- Nucleic Acid
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccacc

SEQ ID NO: 32 - DNA, 57 bp, Artificial Sequence
  ANTIBODY SIGNAL SEQUENCE
atgggctggagctgtatcatcctgttcctggtggcaaccgcaacaggagtgcacagc

SEQ ID NO: 33 - DNA, 12 bp, Artificial Sequence
  FURIN CLEAVAGE SITE- nucleic acid
cgcaaacgccgc

SEQ ID NO: 34 - protein, 4 aa, Artificial Sequence
  FURIN CLEAVAGE SITE- amino acid
RKRR

SEQ ID NO: 35 - DNA, 60 bp, Artificial Sequence
  T2A
agagccgagggcaggggaagtcttctaacatgcggggacgtggaggaaaatcccgggccc

SEQ ID NO: 36 - DNA, 999 bp, Artificial Sequence
  TRUNCATED CD 19
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattc

SEQ ID NO: 37 - protein, 4 aa, Artificial Sequence
  Linker
GGGG

SEQ ID NO: 38 - protein, 5 aa, Artificial Sequence
  Linker
GGGGG

SEQ ID NO: 39 - protein, 6 aa, Artificial Sequence
  Linker
GGGGGG

SEQ ID NO: 40 - protein, 7 aa, Artificial Sequence
  Linker
GGGGGGG

SEQ ID NO: 41 - protein, 8 aa, Artificial Sequence
  Linker
GGGGGGGG

SEQ ID NO: 42 - protein, 5 aa, Artificial Sequence
  Linker
GGGGS

SEQ ID NO: 43 - protein, 10 aa, Artificial Sequence
  Linker
GGGGSGGGGS

SEQ ID NO: 44 - protein, 15 aa, Artificial Sequence
  Linker
GGGGSGGGGSGGGGS

SEQ ID NO: 45 - protein, 5 aa, Artificial Sequence
  Linker
TKGPS

SEQ ID NO: 46 - protein, 5 aa, Artificial Sequence
  Linker
TVAAP

SEQ ID NO: 47 - protein, 5 aa, Artificial Sequence
  Linker
QPKAA

SEQ ID NO: 48 - protein, 5 aa, Artificial Sequence
  Linker
QRIEG

SEQ ID NO: 49 - protein, 7 aa, Artificial Sequence
  Linker
ASTKGPS

SEQ ID NO: 50 - protein, 7 aa, Artificial Sequence
  Linker
RTVAAPS

SEQ ID NO: 51 - protein, 7 aa, Artificial Sequence
  Linker
GQPKAAP

SEQ ID NO: 52 - protein, 7 aa, Artificial Sequence
  Linker
HIDSPNK

SEQ ID NO: 53 - protein, 85 aa, Artificial Sequence
  10-1074 variable light (VL) chain amino acid sequence - short version
SYVRPLSVALGETARISCGRQALGSRAVQWYQHRPGQAPILLIYNNQDRPSGIPERFSGTPDINFGTRATLTISGVEAGDEADYY

SEQ ID NO: 54 - DNA, 324 bp, Artificial Sequence
  Nucleic acid encoding short version of 10-1074 variable light (VL) chain
tcctacgtgcggccactgtccgtggccctgggagagaccgcaaggatctcctgcggcagacaggccctgggatctagggccgtgcagtggtatcagcacaggccaggacaggcaccaatcctgctgatctacaacaatcaggaccggccttctggcatcccagagagattcagcggcacccccgatatcaactttggcacaagagccaccctgacaatcagcggagtggaggcaggcgacgaggcagattactattgtcacatgtgggacagcaggtccggcttctcttggagctttggcggagcaacaaggctgaccgtgctg

SEQ ID NO: 55 - protein, 21 aa, Artificial Sequence
  VL-FR1
SYVRPLSVALGETARISCGRQ

SEQ ID NO: 56 - protein, 6 aa, Artificial Sequence
  VL-CDR1.2
ALGSRA

SEQ ID NO: 57 - protein, 17 aa, Artificial Sequence
  VL-FR2
VQWYQHRPGQAPILLIY

SEQ ID NO: 58 - protein, 3 aa, Artificial Sequence
  VL-CDR2.2
NNQ

SEQ ID NO: 59 - protein, 36 aa, Artificial Sequence
  VL-FR3
DRPSGIPERFSGTPDINFGTRATLTISGVEAGDEAD

SEQ ID NO: 60 - protein, 15 aa, Artificial Sequence
  VL-CDR3.2
YYCHMWDSRSGFSWS

SEQ ID NO: 61 - protein, 10 aa, Artificial Sequence
  VL-FR4
FGGATRLTVL

SEQ ID NO: 62 - DNA, 315 bp, Artificial Sequence
  Nucleic acid encoding 10-1074 constant light (CL) chain
gtggcagcaccatccgtgttcatctttcccccttctgatgagcagctgaagtccggcaccgcctctgtggtgtgcctgctgaacaatttctatcctagggaggccaaggtgcagtggaaggtggacaacgccctgcagagcggcaattcccaggagtctgtgaccgagcaggacagcaaggattccacatactctctgtctagcaccctgacactgagcaaggccgattatgagaagcacaaggtgtacgcctgtgaggtgacccaccagggcctgtcctctcctgtgacaaagtccttcaacaggggagagtgc

SEQ ID NO: 63 - protein, 105 aa, Artificial Sequence [sequence omitted]

SEQ ID NO: 64 - protein, 136 aa, Artificial Sequence
  10-1074 variable heavy (VH) chain amino acid sequence - long version
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTK

SEQ ID NO: 65 - DNA, 408 bp, Artificial Sequence
  Nucleic acid encoding long version of 10-1074 variable heavy (VH) chain
caggtgcagctgcaggagtccggaccaggactggtgaagcctagcgagaccctgtccgtgacatgctccgtgtctggcgatagcatgaacaattactattggacctggatcaggcagtcccctggcaagggactggagtggatcggctatatctctgacagagagagcgccacctacaacccaagcctgaatagccgggtggtcatctcccgcgatacatctaagaaccagctgtctctgaagctgaatagcgtgacccccgccgacacagccgtgtactattgcgcaacagcaaggaggggacagaggatctatggcgtggtgagcttcggcgagttcttttactattactccatggacgtgtggggcaagggcaccacagtgaccgtgagctccgccagcaccaag

SEQ ID NO: 66 - protein, 25 aa, Artificial Sequence
  VH-FR1
QVQLQESGPGLVKPSETLSVTCSVS

SEQ ID NO: 67 - protein, 8 aa, Artificial Sequence
  VH-CDR1.2
GDSMNNYY

SEQ ID NO: 68 - protein, 17 aa, Artificial Sequence
  VH-FR2
WTWIRQSPGKGLEWIGY

SEQ ID NO: 69 - protein, 7 aa, Artificial Sequence
  VH-CDR2.2
ISDRESA

SEQ ID NO: 70 - protein, 38 aa, Artificial Sequence
  VH-FR3
TYNPSLNSRVVISRDTSKNQLSLKLNSVTPADTAVYYC

SEQ ID NO: 71 - protein, 26 aa, Artificial Sequence
  VH-CDR3.2
ATARRGQRIYGVVSFGEFFYYYSMDV

SEQ ID NO: 72 - protein, 11 aa, Artificial Sequence
  VH-FR4
WGKGTTVTVSS

SEQ ID NO: 73 - DNA, 1131 bp, Artificial Sequence
  Nucleic acid encoding 10-1074 constant heavy (CH) chain
gcctccacaaagggccctagcgtgtttccactggcaccatgcagccgctccacctctggaggcacagccgccctgggctgtctggtgaaggactacttccccgagcctgtgaccgtgtcttggaacagcggcgccctgaccagcggagtgcacacatttccagccgtgctgcagtctagcggcctgtattccctgtcctctgtggtgacagtgcccagctcctctctgggcacccagacatacacctgtaacgtgaatcacaagcctagcaataccaaggtggacaagagggtggagctgaagacccctctgggcgataccacacacacatgcccacggtgtccagagcccaagtcttgcgacaccccacccccttgccccagatgtcctgagccaaagagctgtgatacaccacccccttgccctaggtgtcccgagcctaagtcctgcgacaccccaccaccttgcccaaggtgtccagcaccagagctgctgggaggaccatccgtgttcctgtttccacccaagcctaaggatacactgatgatctctcgcaccccagaggtgacatgcgtggtggtggacgtgagccacgaggatcccgaggtgcagttcaagtggtacgtggacggcgtggaggtgcacaacgccaagaccaagccccgggaggagcagtacaattccacctttagagtggtgtctgtgctgacagtgctgcaccaggattggctgaacggcaaggagtacaagtgtaaggtgtccaataaggccctgcctgccccaatcgagaagaccatctctaagacaaagggccagcctcgggagccacaggtgtataccctgcctccatccagagaggagatgaccaagaaccaggtgtctctgacatgcctggtgaagggcttctaccccagcgatatcgcagtggagtgggagagctccggacagcctgagaacaattataataccacaccccctatgctggactccgatggctctttctttctgtactctaagctgaccgtggacaagagccggtggcagcagggcaacatcttcagctgttccgtgatgcacgaggccctgcacaatcggtttacacagaagtctctgagcctgtcccccggcaag

SEQ ID NO: 74 - protein, 377 aa, Artificial Sequence
  10-1074 constant heavy (HL) chain amino acid sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 75 - protein, 241 aa, Artificial Sequence
  Variant BiKE amino acid sequence (containing 10-1074 scFv)
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGGGGSGGGGSGGGGSGGGGSSYVRPLSVALGETARISCGRQALGSRAVQWYQHRPGQAPILLIYNNQDRPSGIPERFSGTPDINFGTRATLTISGVEAGDEADYY

SEQ ID NO: 76 - DNA, 1601 bp, Artificial sequence
  Nucleic acid sequence of construct Genesis 605a
ggattagtccaatttaccggtacaagtttgtacaaaaaagcaggctatgtacagaatgcagctgctgagctgcatcgccctgagcctggccctggtgaccaacagccaggtgcagctggtggagagcggcggcggcctggtgcagcccggcggcagcctgagactgagctgcgccgccagcggcttcaccctggactactacagctggttcagacaggcccccggccagggcctggaggccgtggcctgcatcagcgacagcgacggcagaaccagattcacaatcagcagagacaacagcaagaacaccctgtacctgcagatgaacagcctgagagccgaggacaccgccgtgtactactgcgccgccaccgactgcaccgtggaccccagcctgctgtacgtgatggactactggggccagggcaccctggtgaccgtgagcagcgattacaaggacgacgacgataaggattacaaggatgatgatgataaggcgaccaactttagcctgctgaaacaggcgggcgacgtggaagaaaacccgggcccgatgccacctcctcgcctcctcttcttcctcctcttcctcacccctatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccatatgaggcccctggctatctggctttttatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagcttatgagccccaagctgtatgtgtgggccaaagaccgccctgagatctgggagggagagcctccgtgtctcccaccgagggacagcctgaaccagagcctcagccaggacctcacaatggcccctggctccacactctggctgtcctgtggggtaccccctgactctgtgtccaggggccccctctcctggacccacgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtccttccacctggagatcactgctcggccagtactctggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattctaaaccactttgtacaagaaagctgggtctcgagggattagtccaattt

SEQ ID NO: 77 - DNA, 1982 bp, Artificial sequence
  Nucleic acid sequence of construct Genesis 605b
ggattagtccaatttaccggtacaagtttgtacaaaaaagcaggctatgtacagaatgcagctgctgagctgcatcgccctgagcctggccctggtgaccaacagcgaggtgcagctggtggagagcggcggcggcctggtgcagcccggcggcagcctgagactgagctgcgccgccagcggcttcaccttcagcaactacggcatgagctgggtgagacaggcccccggcaagggcctggagtggatcggcagcctgtactacagcggcggcagcaccaactacaaccccagcctgaagggcagcctggtgatcagcagagacaacagcaagaacaccctgtacctgcagatgaacagcctggccgaggacaccgccacctactactgcgccagagagagcatcgactactggggccagggcaccctggtgaccgtgagcagcggtggcggatcaggaggcggaggttctggaggaggtgggagtcaggtgcagctggtggagagcggcggcggcctggtgcagcccggcggcagcctgagactgagctgcgccgccagcggcttcaccctggactactacagctggttcagacaggcccccggccagggcctggaggccgtggcctgcatcagcgacagcgacggcagaaccagattcacaatcagcagagacaacagcaagaacaccctgtacctgcagatgaacagcctgagagccgaggacaccgccgtgtactactgcgccgccaccgactgcaccgtggaccccagcctgctgtacgtgatggactactggggccagggcaccctggtgaccgtgagcagcgattacaaggacgacgacgataaggattacaaggatgatgatgataaggcgaccaactttagcctgctgaaacaggcgggcgacgtggaagaaaacccgggcccgatgccacctcctcgcctcctcttcttcctcctcttcctcacccctatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccatatgaggcccctggctatctggctttttatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagcttatgagccccaagctgtatgtgtgggccaaagaccgccctgagatctgggagggagagcctccgtgtctcccaccgagggacagcctgaaccagagcctcagccaggacctcacaatggcccctggctccacactctggctgtcctgtggggtaccccctgactctgtgtccaggggccccctctcctggacccacgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtccttccacctggagatcactgctcggccagtactctggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattctaaaccactttgtacaagaaagctgggtctcgagggattagtccaattt

SEQ ID NO: 78 - protein, 106 aa, Artificial Sequence
  light chain constant region IgG3 (amino acid)
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 377 aa, Artificial Sequence; identical to SEQ ID NO: 74
  heavy chain constant region (amino acid)

SEQ ID NO: 80 - DNA, 10742 bp, Artificial sequence
  Synthetic DNA construct 101074 ALL
  LTR (1)..(592): long terminal repeat from Moloney murine leukemia virus
  misc_feature (455)..(462): UAS repeats
  misc_feature (655)..(1012): MMLV Psi, packaging signal of Moloney murine leukemia virus (MMLV)
  misc_feature (1833)..(6282): 101074 ALL
  enhancer (1839)..(2142)
  promoter (2143)..(2346)
  CDS (3547)..(3603): 2A peptide from porcine teschovirus-1 polyprotein (P2A)
  CDS (5221)..(5277): 2A peptide from porcine teschovirus-1 polyprotein (P2A)
  LTR (6547)..(7016): long terminal repeat from Moloney murine leukemia virus
  misc_feature (6881)..(6888): UAS repeats
  primer_bind (7715)..(7731): M13 fwd, complement
  promoter (8206)..(8310): bla promoter
  CDS (8311)..(9171): beta-lactamase
  misc_feature (8729)..(8736): UAS repeats
  rep_origin (9316)..(9989): pUC origin
  rep_origin (9342)..(9936): pMB1 replication origin
  promoter (10206)..(10327): lac promoter
  protein_bind (10218)..(10239): E. coli catabolite activator protein binding site
  protein_bind (10290)..(10310): lac repressor binding site
  primer_bind (10316)..(10332): M13 rev
  LTR (10741)..(10742): long terminal repeat from Moloney murine leukemia virus
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatgggctggagctgtatcatcctgttcctggtggcaaccgcaacaggagtgcacagctcctacgtgcggccactgtccgtggccctgggagagaccgcaaggatctcctgcggcagacaggccctgggatctagggccgtgcagtggtatcagcacaggccaggacaggcaccaatcctgctgatctacaacaatcaggaccggccttctggcatcccagagagattcagcggcacccccgatatcaactttggcacaagagccaccctgacaatcagcggagtggaggcaggcgacgaggcagattactattgtcacatgtgggacagcaggtccggcttctcttggagctttggcggagcaacaaggctgaccgtgctgacagtggcagcaccatccgtgttcatctttcccccttctgatgagcagctgaagtccggcaccgcctctgtggtgtgcctgctgaacaatttctatcctagggaggccaaggtgcagtggaaggtggacaacgccctgcagagcggcaattcccaggagtctgtgaccgagcaggacagcaaggattccacatactctctgtctagcaccctgacactgagcaaggccgattatgagaagcacaaggtgtacgcctgtgaggtgacccaccagggcctgtcctctcctgtgacaaagtccttcaacaggggagagtgcaggaagaggagaggatctggagcaaccaactttagcctgctgaagcaggcaggcgacgtggaggagaatcctggaccaatgggatggtcctgtatcatcctgtttctggtcgccactgccacaggagtgcacagccaggtgcagctgcaggagtccggaccaggactggtgaagcctagcgagaccctgtccgtgacatgctccgtgtctggcgatagcatgaacaattactattggacctggatcaggcagtcccctggcaagggactggagtggatcggctatatctctgacagagagagcgccacctacaacccaagcctgaatagccgggtggtcatctcccgcgatacatctaagaaccagctgtctctgaagctgaatagcgtgacccccgccgacacagccgtgtactattgcgcaacagcaaggaggggacagaggatctatggcgtggtgagcttcggcgagttcttttactattactccatggacgtgtggggcaagggcaccacagtgaccgtgagctccgccagcaccaaggcctccacaaagggccctagcgtgtttccactggcaccatgcagccgctccacctctggaggcacagccgccctgggctgtctggtgaaggactacttccccgagcctgtgaccgtgtcttggaacagcggcgccctgaccagcggagtgcacacatttccagccgtgctgcagtctagcggcctgtattccctgtcctctgtggtgacagtgcccagctcctctctgggcacccagacatacacctgtaacgtgaatcacaagcctagcaataccaaggtggacaagagggtggagctgaagacccctctgggcgataccacacacacatgcccacggtgtccagagcccaagtcttgcgacaccccacccccttgccccagatgtcctgagccaaagagctgtgatacaccacccccttgccctaggtgtcccgagcctaagtcctgcgacaccccaccaccttgcccaaggtgtccagcaccagagctgctgggaggaccatccgtgttcctgtttccacccaagcctaaggatacactgatgatctctcgcaccccagaggtgacatgcgtggtggtggacgtgagccacgaggatcccgaggtgcagttcaagtggtacgtggacggcgtggaggtgcacaacgccaagaccaagccccgggaggagcagtacaattccacctttagagtggtgtctgtgctgacagtgctgcaccaggattggctgaacggcaaggagtacaagtgtaaggtgtccaataaggccctgcctgccccaatcgagaagaccatctctaagacaaagggccagcctcgggagccacaggtgtataccctgcctccatccagagaggagatgaccaagaaccaggtgtctctgacatgcctggtgaagggcttctaccccagcgatatcgcagtggagtgggagagctccggacagcctgagaacaattataataccacaccccctatgctggactccgatggctctttctttctgtactctaagctgaccgtggacaagagccggtggcagcagggcaacatcttcagctgttccgtgatgcacgaggccctgcacaatcggtttacacagaagtctctgagcctgtcccccggcaagagaaagcggagaggcagcggcgccaccaacttctccctgctgaagcaggccggcgacgtggaagaaaatccaggacctatgccaccacctaggctgctgttctttctgctgtttctgacaccaatggaggtgagacccgaggagcctctggtggtgaaggtggaggagggcgacaacgccgtgctgcagtgcctgaagggcaccagcgatggaccaacccagcagctgacatggagccgggagtccccactgaagccctttctgaagctgtccctgggactgccaggactgggcatccacatgagacccctggccatctggctgttcatcttcaacgtgagccagcagatgggaggcttctacctgtgccagccaggaccaccatccgagaaggcatggcagccaggatggaccgtgaacgtggagggatctggcgagctgtttaggtggaatgtgagcgatctgggaggactgggatgcggcctgaagaaccgctctagcgagggcccttcctctccatccggcaagctgatgtctcctaagctgtacgtgtgggccaaggacaggccagagatctgggagggagagcctccatgtctgccacctcgcgacagcctgaatcagtctctgagccaggatctgaccatggccccaggcagcacactgtggctgtcctgcggagtgccaccagattccgtgtctcggggcccactgtcctggacccatgtgcaccccaagggccctaagtctctgctgagcctggagctgaaggacgatcggcctgccagagatatgtgggtcatggagaccggactgctgctgccaagggccacagcacaggacgccggcaagtattactgccaccgcggcaacctgaccatgagcttccacctggagatcacagcaaggcccgtgctgtggcactggctgctgaggaccggaggatggaaggtgagcgccgtgacactggcctacctgatcttctgcctgtgctccctggtgggcatcctgcacctgcagagagccctggtgctgaggcgcaagaggaagcgcatgaccgaccctacacggagatttatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaa

SEQ ID NO: 81 - protein, 19 aa, Artificial sequence
  2A peptide from porcine teschovirus-1 polyprotein (P2A)
ATNFSLLKQAGDVEENPGP

SEQ ID NO: 82 - protein, 19 aa, Artificial sequence
  2A peptide from porcine teschovirus-1 polyprotein (P2A)
ATNFSLLKQAGDVEENPGP

SEQ ID NO: 83 - protein, 286 aa, Artificial sequence
  beta-lactamase
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 84 - DNA, 9971 bp, Artificial sequence
  Synthetic DNA construct 101074 BIKE
  LTR (1)..(592): long terminal repeat from Moloney murine leukemia virus
  misc_feature (455)..(462): UAS repeats
  misc_feature (655)..(1012): MMLV Psi, packaging signal of Moloney murine leukemia virus (MMLV)
  misc_feature (1833)..(5511): 101074 SCFV
  enhancer (1839)..(2142): CMV enhancer
  promoter (2143)..(2346): CMV promoter
  CDS (4450)..(4506): 2A peptide from porcine teschovirus-1 polyprotein (P2A)
  LTR (5776)..(6245): long terminal repeat from Moloney murine leukemia virus
  misc_feature (6110)..(6117): UAS repeats
  primer_bind (6944)..(6960): M13 fwd, complement
  promoter (7435)..(7539): bla promoter
  CDS (7540)..(8400): beta-lactamase
  misc_feature (7958)..(7965): UAS repeats
  rep_origin (8545)..(9218): pUC origin
  rep_origin (8571)..(9165): pMB1 replication origin
  promoter (9435)..(9556): lac promoter
  protein_bind (9447)..(9468): E. coli catabolite activator protein binding site
  protein_bind (9519)..(9539): lac repressor binding site
  primer_bind (9545)..(9561): M13 rev
  LTR (9970)..(9971): long terminal repeat from Moloney murine leukemia virus
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagaccaccatggactggatctggaggatcctgttcctggtgggagcagcaaccggagcacactccgaggtgcagctggtggagtctggaggaggagtggtgaggcctggaggatccctgaggctgtcttgcgcagcaagcggcttcacatttgacgattacggaatgtcttgggtgcgccaggcaccaggcaagggactggagtgggtgagcggcatcaactggaatggaggaagcaccggatatgcagactccgtgaagggcaggttcacaatcagccgcgataacgccaagaattccctgtacctgcagatgaactctctgcgggccgaggacaccgccgtgtactattgcgcccggggcagatccctgctgtttgattactggggccagggcaccctggtgacagtgtctagaggcggcggcggcagcggcggcggcggctctggaggaggaggaagcggaggaggaggaagctcctctgagctgacacaggacccagccgtgagcgtggccctgggacagaccgtgaggatcacatgtcagggcgattctctgcgcagctactatgcctcctggtatcagcagaagccaggacaggcacccgtgctggtcatctacggcaagaacaatcggccaagcggcatccccgacagattctccggcagctcctctggcaataccgcctctctgaccatcacaggagcacaggcagaggacgaggcagattactattgcaactccagggatagctccggcaatcatgtggtgttcggcggcggcaccaagctgacagtgggatccggaggaggaggatctcaggtgcagctgcaggagagcggaccaggactggtgaagccatccgagaccctgtctgtgacatgttccgtgtctggcgactccatgaacaattactattggacctggatccggcagtctccaggcaagggcctggagtggatcggctatatcagcgatagagagtccgccacctacaacccctccctgaatagccgggtggtcatctctagagacacaagcaagaaccagctgagcctgaagctgaattccgtgacccccgccgatacagccgtgtactattgtgccacagcccggagaggccagcggatctatggcgtggtgagcttcggcgagttcttttactattactccatggacgtgtggggcaagggcaccacagtgaccgtgtctagcgccagcacaaagggaggaggaggatcaggcggcggcggcagtggcggcggcggctccggaggaggaggatcctcttacgtgagacctctgtccgtggccctgggagagaccgcaaggatctcctgcggccgccaggccctgggatctcgggccgtgcagtggtatcagcacagacctggccaggccccaatcctgctgatctacaacaatcaggacaggccctctggaatccctgagcggttcagcggaaccccagatatcaactttggcacaagggccaccctgacaatcagcggagtggaggcaggcgacgaggcagattattactgtcacatgtgggacagccgctccggcttctcttggagctttggcggagcaaccaggctgacagtgctgagaaagaggaggggatctggagcaaccaacttcagcctgctgaagcaggccggcgatgtggaggagaatccaggaccaatgccacctccaaggctgctgttctttctgctgtttctgacccccatggaggtgagacctgaggagccactggtggtgaaggtggaggagggcgacaacgccgtgctgcagtgcctgaagggcacaagcgatggccctacccagcagctgacatggagccgggagtcccccctgaagcctttcctgaagctgtccctgggactgcctggactgggcatccacatgagaccactggccatctggctgttcatctttaatgtgagccagcagatgggaggcttctatctgtgccagccaggaccaccttccgagaaggcatggcagcctggatggaccgtgaacgtggagggctctggcgagctgtttaggtggaatgtgagcgacctgggaggactgggatgtggcctgaagaaccgcagctccgagggaccatctagcccatctggcaagctgatgagcccaaagctgtacgtgtgggccaaggataggccagagatctgggagggagagccaccatgcctgcctccacgcgacagcctgaatcagtccctgtctcaggatctgaccatggcccccggctccacactgtggctgtcttgtggagtgccacctgacagcgtgtccaggggccctctgtcctggacccatgtgcaccctaagggcccaaagtctctgctgagcctggagctgaaggacgatcggcctgccagagacatgtgggtcatggagaccggactgctgctgccaagggccacagcacaggatgccggcaagtattactgtcaccgcggcaacctgaccatgagctttcacctggagatcacagcaaggccagtgctgtggcactggctgctgagaaccggaggatggaaggtgagcgccgtgacactggcctacctgatcttctgcctgtgctccctggtgggaatcctgcacctgcagcgcgccctggtgctgcggagaaagaggaagcgcatgaccgatcccacaaggcgctttatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaa

SEQ ID NO: 85 - protein, 19 aa, Artificial sequence
  2A peptide from porcine teschovirus-1 polyprotein (P2A)
ATNFSLLKQAGDVEENPGP

SEQ ID NO: 86 - protein, 286 aa, Artificial sequence; identical to SEQ ID NO: 83
  beta-lactamase